(19) United States
(12) Patent Application Publication
(10) Pub. No.: US 2025/0275353 A1
(43) Pub. Date: Aug. 28, 2025

(54) COMPOUND, LIGHT EMITTING DEVICE, AND DISPLAYING DEVICE

(71) Applicant: BOE Technology Group Co., Ltd., Beijing (CN)

(72) Inventors: Rongrong Gao, Beijing (CN); Xianwen Meng, Beijing (CN); Dongxu Zhang, Beijing (CN); Wenzhu Jing, Beijing (CN); Lei Chen, Beijing (CN); Xueqin Chen, Beijing (CN)

(73) Assignee: BOE Technology Group Co., Ltd., Beijing (CN)

(21) Appl. No.: 18/858,784

(22) PCT Filed: Jan. 17, 2024
(86) PCT No.: PCT/CN2024/072792

§ 371 Date: Oct. 22, 2024

(30) Foreign Application Priority Data
Feb. 8, 2023 (CN) 202310141644.1

(51) Int. Cl. H10K 50/16; C09K 11/06; H10K 50/18; H10K 85/60; H10K 101/30

(52) U.S. Cl. CPC H10K 50/16; C09K 11/06; H10K 50/181; H10K 85/615; H10K 85/654; H10K 85/656; H10K 85/6572; H10K 85/6574; H10K 85/6576; C09K 2211/1018; H10K 2101/30

(57) ABSTRACT

A compound, a light emitting device, and a displaying device, which relates to the technical field of displaying. The light emitting device has a high luminous efficiency and a long life. The light emitting device includes an electron transport layer, and the material of the electron transport layer includes a heteroaromatic ring group including at least one nitrogen atom, a first electron-withdrawing group, and at least one three-dimensional group having a rigidity. The triplet-state energy level T1 of the material of the electron transport layer satisfies T1≥2.3 eV. The absolute value of the energy value of the highest occupied molecular orbital HOMO of the material of the electron transport layer satisfies 6.0 eV≤|HOMO|≤7.0 eV. The absolute value of the energy value of the lowest unoccupied molecular orbital LUMO of the material of the electron transport layer satisfies 2.6 eV≤|LUMO|≤3.6 eV.

### CROSS REFERENCE TO RELEVANT APPLICATIONS

The present application claims the priority of the Chinese patent application filed on Feb. 8, 2023 before the Chinese Patent Office with the application number of 202310141644.1 and the title of “COMPOUND, LIGHT-EMITTING DEVICE, AND DISPLAY APPARATUS”, which is incorporated herein in its entirety by reference.

### TECHNICAL FIELD

The present application relates to the technical field of displaying, and particularly relates to a compound, a light emitting device, and a displaying device.

### BACKGROUND

With the development of science and technology, OLED (Organic Light Emitting Diode) displaying devices are used more and more widely. The OLED displaying devices comprise an electron transport layer. However, the conventional electron transport layers usually have problems such as a low luminous efficiency and a short life, which results in poor user experience. Therefore, it is urgently needed to provide a novel OLED displaying device, so as to solve the above problems.

### SUMMARY

In order to achieve the above object, the embodiments of the present application employ the following technical solutions:

In an aspect, there is provided a light emitting device, wherein the light emitting device comprises an electron transport layer, and a material of the electron transport layer comprises a heteroaromatic ring group including at least one nitrogen atom, a first electron-withdrawing group, and at least one three-dimensional group having a rigidity;
a triplet-state energy level T1 of a material of the electron transport layer satisfies T1≥2.3 eV;
an absolute value of an energy value of a highest occupied molecular orbital HOMO of the material of the electron transport layer satisfies 6.0 eV≤|HOMO|≤7.0 eV; and
an absolute value of an energy value of a lowest unoccupied molecular orbital LUMO of the material of the electron transport layer satisfies 2.6 eV≤|LUMO|≤3.6 eV.

Optionally, a general structural formula of the material of the electron transport layer is:

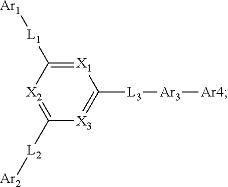

wherein X1 is C(Ra) or nitrogen, X2 is C(Rb) or nitrogen, X3 is C(Rc) or nitrogen, and at least one of X1, X2 and X3 is nitrogen, wherein each of Ra, Rb and Rc is independently any one of a group consisting of hydrogen, diplogen, fluorine, chlorine, an alkyl group whose carbon-atom quantity is 1-12, a halogenated alkyl group whose carbon-atom quantity is 1-12, an alkoxy group whose carbon-atom quantity is 1-12, a cycloalkyl group whose carbon-atom quantity is 3-10, C6-20 aryl, and a heteroaryl group whose carbon-atom quantity is 3-20;
each of L1, L2 and L3 is independently any one of a group consisting of a single bond, a substituted or unsubstituted arylene group whose carbon-atom quantity is 6-60, and a substituted or unsubstituted heteroarylene whose carbon-atom quantity is 1-60;
each of Ar1, Ar2 and Ar4 is independently any one of a group consisting of a substituted or unsubstituted alkyl group whose carbon-atom quantity is 1-12, a substituted or unsubstituted cycloalkyl group whose carbon-atom quantity is 3-10, a substituted or unsubstituted aralkyl group whose carbon-atom quantity is 7-30, a substituted or unsubstituted heterarylalkyl whose carbon-atom quantity is 2-60, a substituted or unsubstituted aryl whose carbon-atom quantity is 6-60, and a substituted or unsubstituted heteroaryl group whose carbon-atom quantity is 3-60, and at least one of Ar1, Ar2 and Ar4 is the three-dimensional group having a rigidity; and
Ar3 is the first electron-withdrawing group.

Optionally, in the material of the electron transport layer, a general structural formula of Ar3 is:

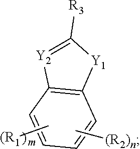

wherein Y1 is any one of carbon, oxygen, sulphur, selenium and N—R4;
Y2 is any one of carbon and nitrogen;
each of R1, R2 and R3 is independently any one of hydrogen, diplogen, halogen, amino, a nitrile group, nitro, C1-30 alkyl, C2-30 alkenyl, C2-30 alkynyl, C1-30 alkoxy, C6-30 aryloxy, and C6-50 aryl substituted by C6-30 aryl, or includes any one of at least one of heteroatoms selected from nitrogen, oxygen and sulphur and C2-50 heteroaryl unsubstituted or substituted by C6-30 aryloxy, and two of R1, R2 and R3 are directly connected to neighboring groups L3 and Ar4;
R4 is any one of a group consisting of hydrogen, diplogen, fluorine, chlorine, an alkyl group whose carbon-atom quantity is 1-12, a halogenated alkyl group whose carbon-atom quantity is 1-12, an alkoxy group whose carbon-atom quantity is 1-12, a cycloalkyl group whose carbon-atom quantity is 3-10, C6-20 aryl, and a heteroaryl group whose carbon-atom quantity is 3-20; and each of m and n is independently 1 or 2.

Optionally, a general structural formula of the material of the electron transport layer is any one of

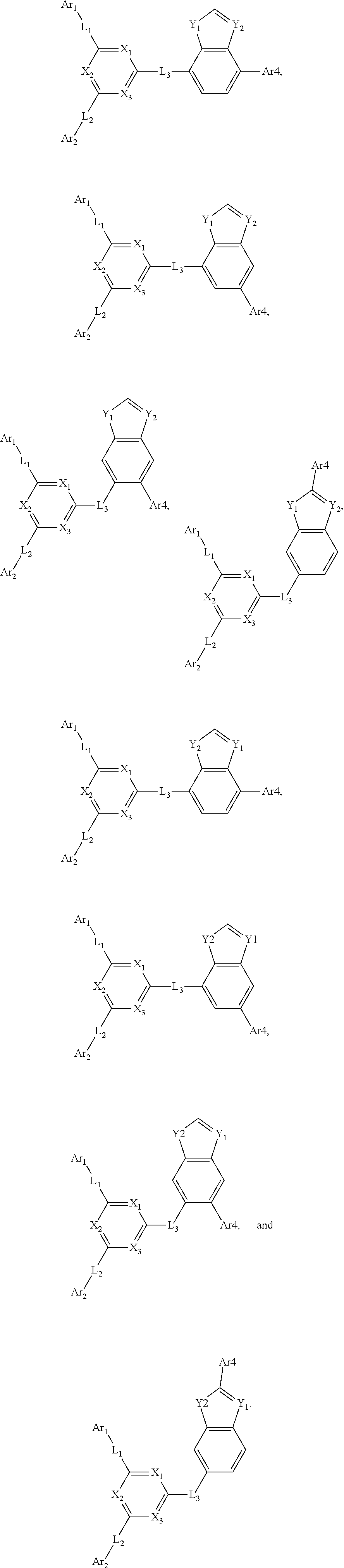

Optionally, Ar3 includes benzoxazole.

Optionally, a general structural formula of the three-dimensional group having a rigidity is any one of

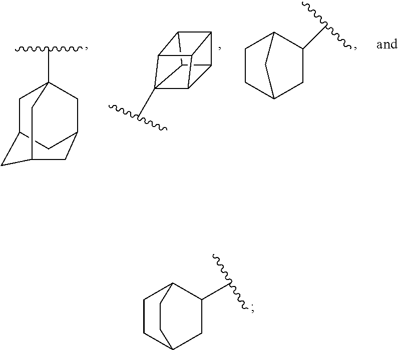

wherein

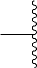

is a bond connected to a carbon atom.

Optionally, in the material of the electron transport layer, the heteroaromatic ring group including at least one nitrogen atom includes a triazine group.

Optionally, a chemical structural formula of a material of the electron transport layer having the triazine group includes any one of

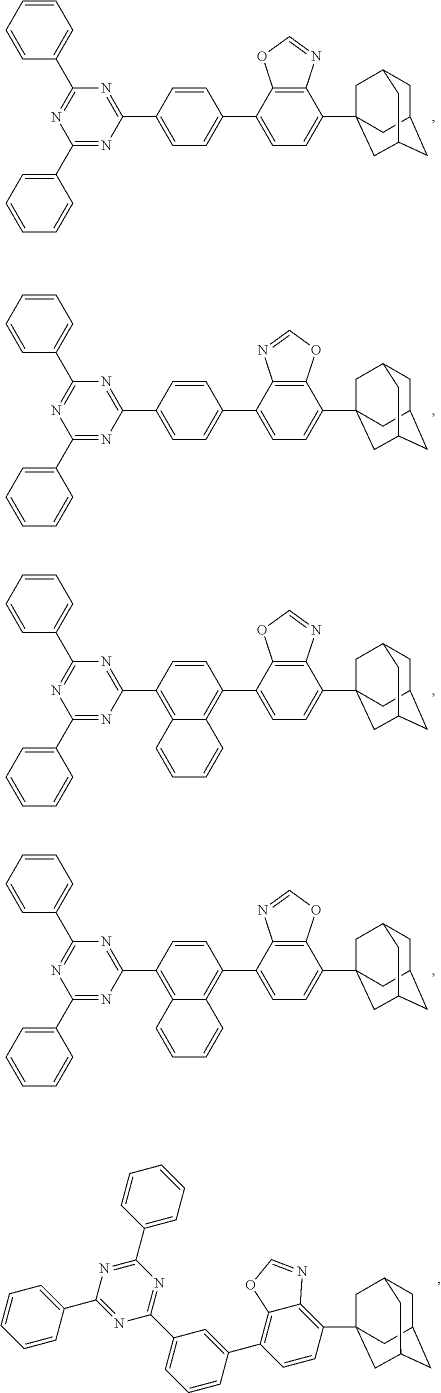 [SMILES: c1ccc(-c2nc(-c3ccccc3)nc(-c3ccc(-c4ccc(C56CC7CC(CC(C7)C5)C6)c5ncoc45)cc3)n2)cc1 ; c1ccc(-c2nc(-c3ccccc3)nc(-c3ccc(-c4ccc(C56CC7CC(CC(C7)C5)C6)c5ocnc45)cc3)n2)cc1 ; c1ccc(-c2nc(-c3ccccc3)nc(-c3ccc(-c4ccc(C56CC7CC(CC(C7)C5)C6)c5ncoc45)c4ccccc34)n2)cc1 ; c1ccc(-c2nc(-c3ccccc3)nc(-c3ccc(-c4ccc(C56CC7CC(CC(C7)C5)C6)c5ocnc45)c4ccccc34)n2)cc1 ; c1ccc(-c2nc(-c3ccccc3)nc(-c3cccc(-c4ccc(C56CC7CC(CC(C7)C5)C6)c5ncoc45)c3)n2)cc1]

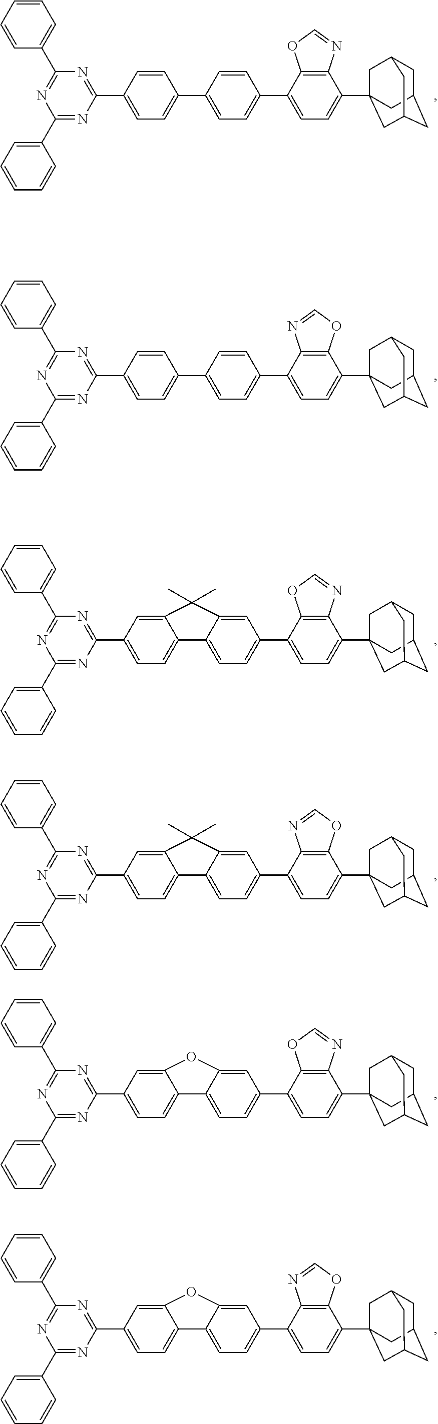

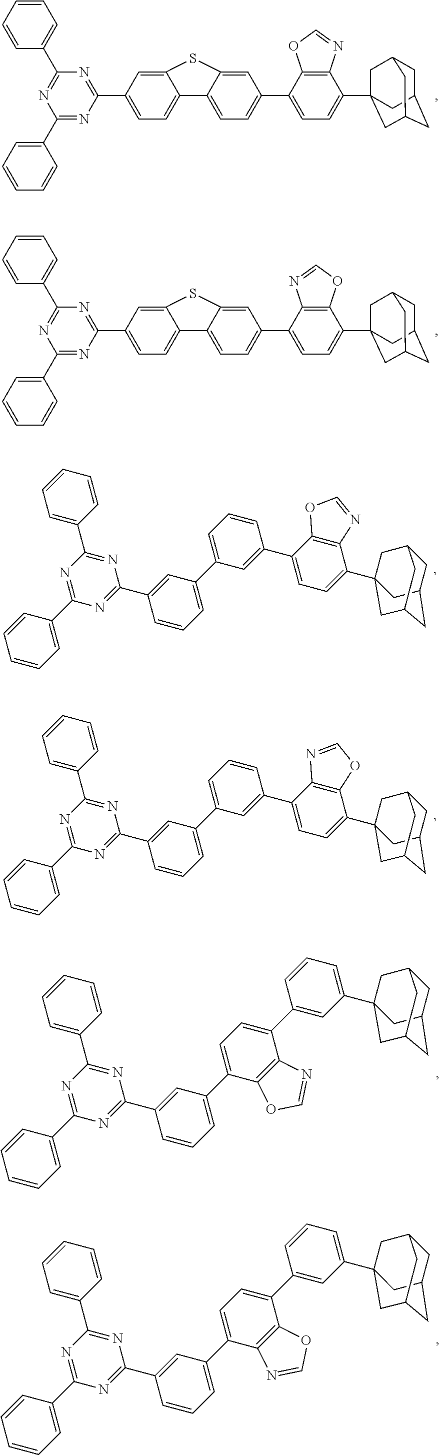

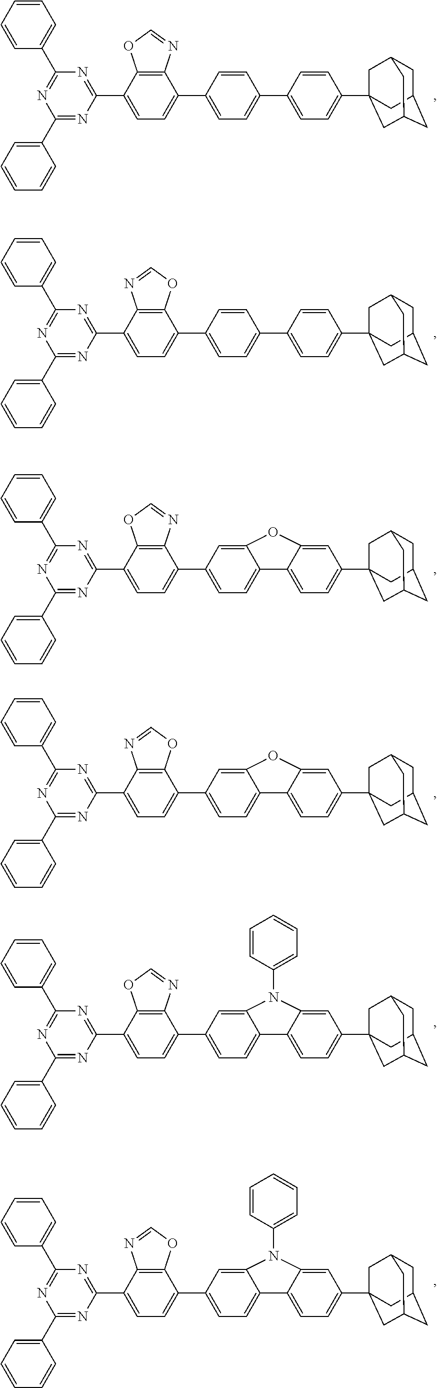

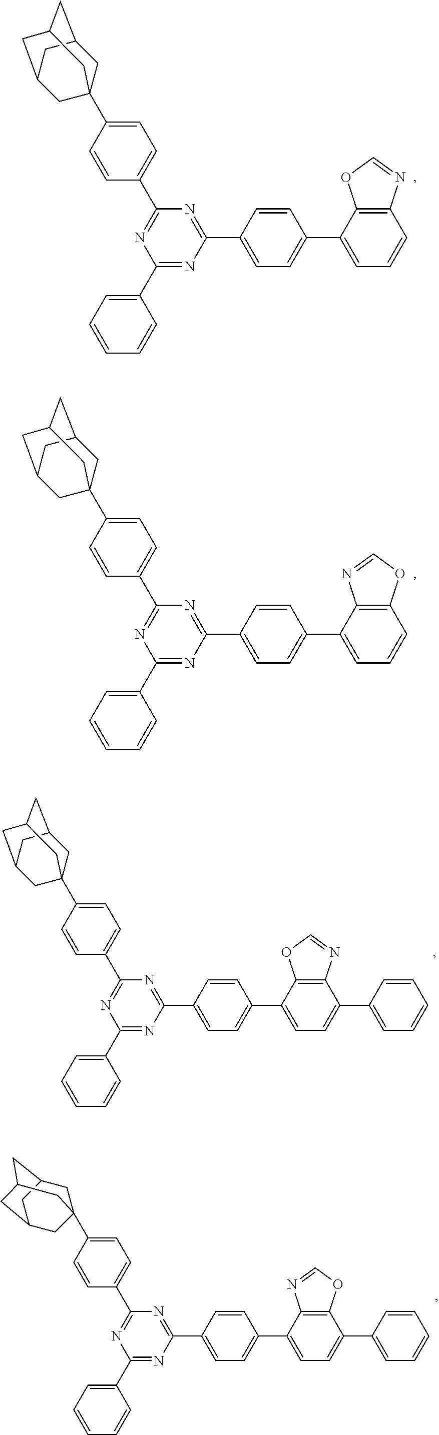 [SMILES: c1ccc(-c2nc(-c3ccc(-c4cccc5ncoc45)cc3)nc(-c3ccc(C45CC6CC(CC(C6)C4)C5)cc3)n2)cc1 ; c1ccc(-c2nc(-c3ccc(-c4cccc5ocnc45)cc3)nc(-c3ccc(C45CC6CC(CC(C6)C4)C5)cc3)n2)cc1 ; c1ccc(-c2nc(-c3ccc(-c4ccc(-c5ccccc5)c5ncoc45)cc3)nc(-c3ccc(C45CC6CC(CC(C6)C4)C5)cc3)n2)cc1 ; c1ccc(-c2nc(-c3ccc(-c4ccc(-c5ccccc5)c5ocnc45)cc3)nc(-c3ccc(C45CC6CC(CC(C6)C4)C5)cc3)n2)cc1]

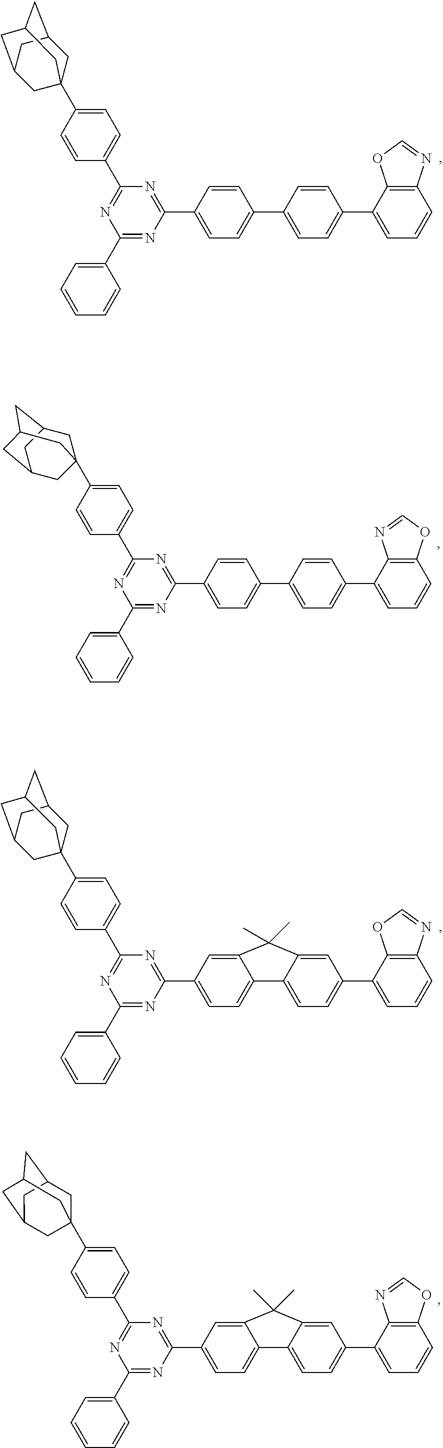 [SMILES: c1ccc(-c2nc(-c3ccc(-c4ccc(-c5cccc6ncoc56)cc4)cc3)nc(-c3ccc(C45CC6CC(CC(C6)C4)C5)cc3)n2)cc1 ; c1ccc(-c2nc(-c3ccc(-c4ccc(-c5cccc6ocnc56)cc4)cc3)nc(-c3ccc(C45CC6CC(CC(C6)C4)C5)cc3)n2)cc1 ; CC1(C)c2cc(-c3nc(-c4ccccc4)nc(-c4ccc(C56CC7CC(CC(C7)C5)C6)cc4)n3)ccc2-c2ccc(-c3cccc4ncoc34)cc21 ; CC1(C)c2cc(-c3nc(-c4ccccc4)nc(-c4ccc(C56CC7CC(CC(C7)C5)C6)cc4)n3)ccc2-c2ccc(-c3cccc4ocnc34)cc21]

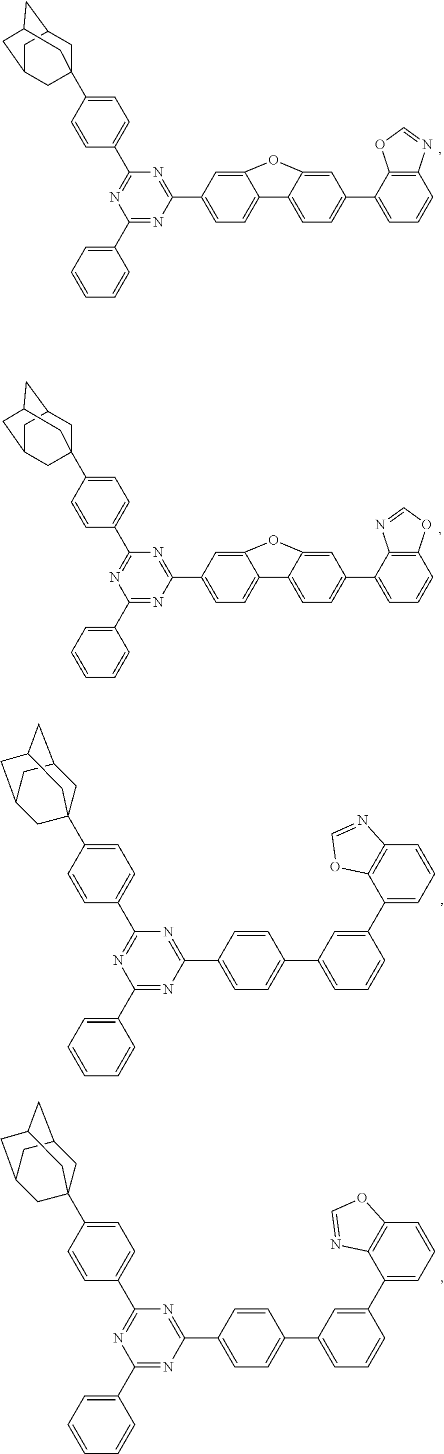 [SMILES: c1ccc(-c2nc(-c3ccc(C45CC6CC(CC(C6)C4)C5)cc3)nc(-c3ccc4c(c3)oc3cc(-c5cccc6ncoc56)ccc34)n2)cc1 ; c1ccc(-c2nc(-c3ccc(C45CC6CC(CC(C6)C4)C5)cc3)nc(-c3ccc4c(c3)oc3cc(-c5cccc6ocnc56)ccc34)n2)cc1 ; c1ccc(-c2nc(-c3ccc(-c4cccc(-c5cccc6ncoc56)c4)cc3)nc(-c3ccc(C45CC6CC(CC(C6)C4)C5)cc3)n2)cc1 ; c1ccc(-c2nc(-c3ccc(-c4cccc(-c5cccc6ocnc56)c4)cc3)nc(-c3ccc(C45CC6CC(CC(C6)C4)C5)cc3)n2)cc1]

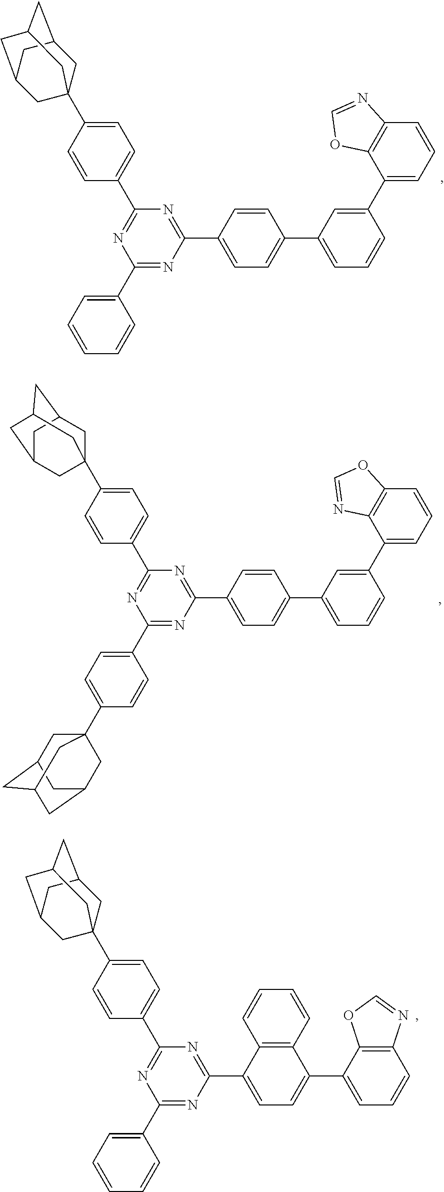

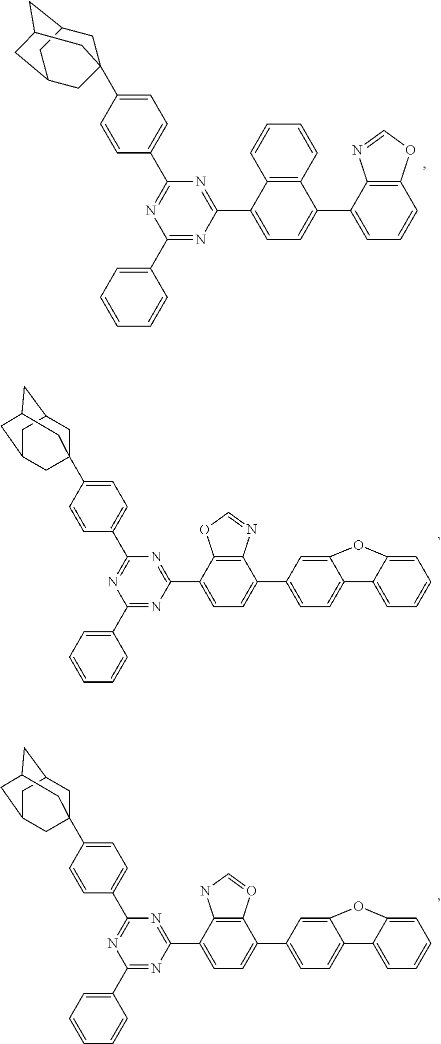

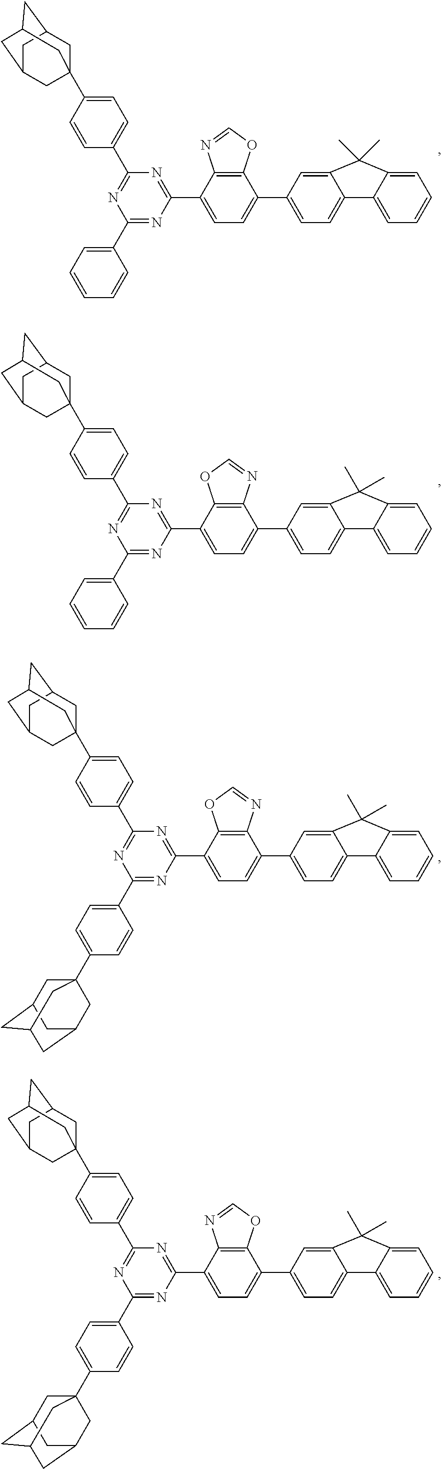

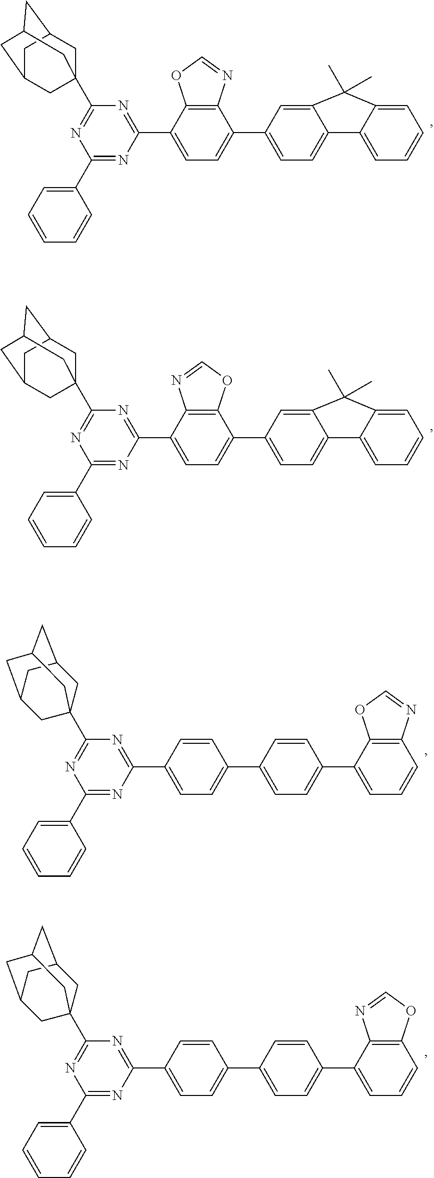 [SMILES: CC1(C)c2ccccc2-c2ccc(-c3ccc(-c4nc(-c5ccccc5)nc(C56CC7CC(CC(C7)C5)C6)n4)c4ocnc34)cc21 ; CC1(C)c2ccccc2-c2ccc(-c3ccc(-c4nc(-c5ccccc5)nc(C56CC7CC(CC(C7)C5)C6)n4)c4ncoc34)cc21 ; c1ccc(-c2nc(-c3ccc(-c4ccc(-c5cccc6ncoc56)cc4)cc3)nc(C34CC5CC(CC(C5)C3)C4)n2)cc1 ; c1ccc(-c2nc(-c3ccc(-c4ccc(-c5cccc6ocnc56)cc4)cc3)nc(C34CC5CC(CC(C5)C3)C4)n2)cc1]

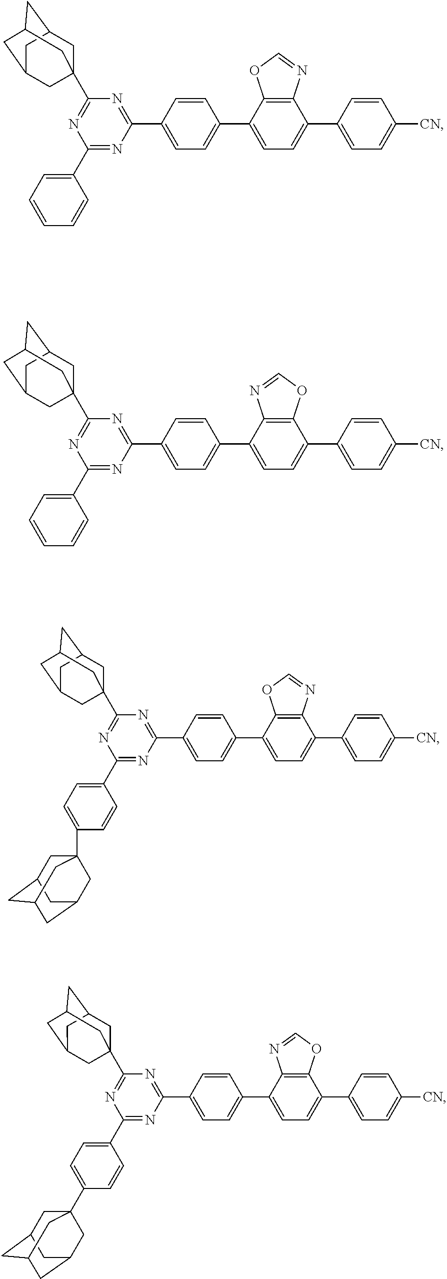 [SMILES: N#Cc1ccc(-c2ccc(-c3ccc(-c4nc(-c5ccccc5)nc(C56CC7CC(CC(C7)C5)C6)n4)cc3)c3ocnc23)cc1 ; N#Cc1ccc(-c2ccc(-c3ccc(-c4nc(-c5ccccc5)nc(C56CC7CC(CC(C7)C5)C6)n4)cc3)c3ncoc23)cc1 ; N#Cc1ccc(-c2ccc(-c3ccc(-c4nc(-c5ccc(C67CC8CC(CC(C8)C6)C7)cc5)nc(C56CC7CC(CC(C7)C5)C6)n4)cc3)c3ocnc23)cc1 ; N#Cc1ccc(-c2ccc(-c3ccc(-c4nc(-c5ccc(C67CC8CC(CC(C8)C6)C7)cc5)nc(C56CC7CC(CC(C7)C5)C6)n4)cc3)c3ncoc23)cc1]

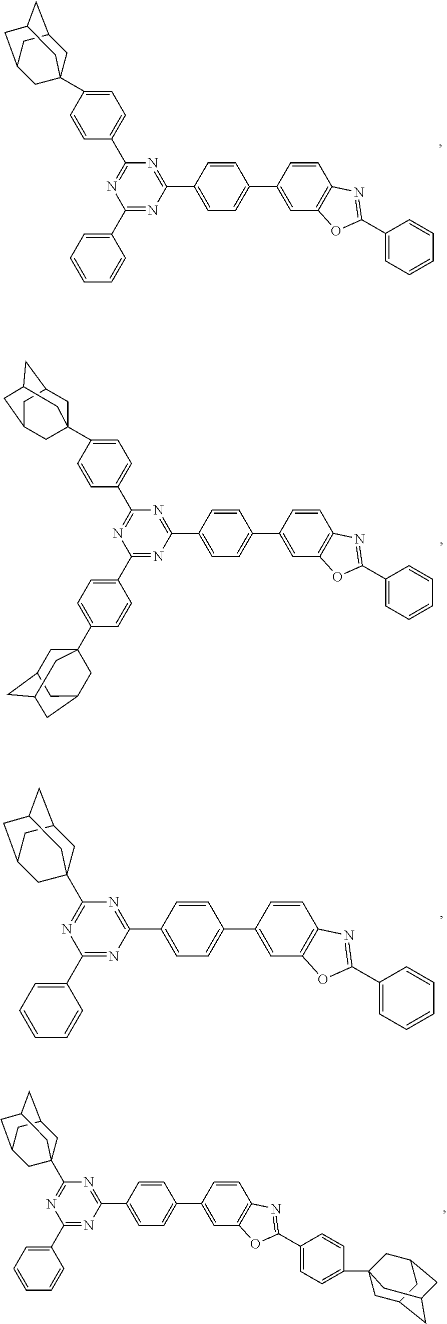 [SMILES: c1ccc(-c2nc(-c3ccc(-c4ccc5nc(-c6ccccc6)oc5c4)cc3)nc(-c3ccc(C45CC6CC(CC(C6)C4)C5)cc3)n2)cc1 ; c1ccc(-c2nc3ccc(-c4ccc(-c5nc(-c6ccc(C78CC9CC(CC(C9)C7)C8)cc6)nc(-c6ccc(C78CC9CC(CC(C9)C7)C8)cc6)n5)cc4)cc3o2)cc1 ; c1ccc(-c2nc(-c3ccc(-c4ccc5nc(-c6ccccc6)oc5c4)cc3)nc(C34CC5CC(CC(C5)C3)C4)n2)cc1 ; c1ccc(-c2nc(-c3ccc(-c4ccc5nc(-c6ccc(C78CC9CC(CC(C9)C7)C8)cc6)oc5c4)cc3)nc(C34CC5CC(CC(C5)C3)C4)n2)cc1]

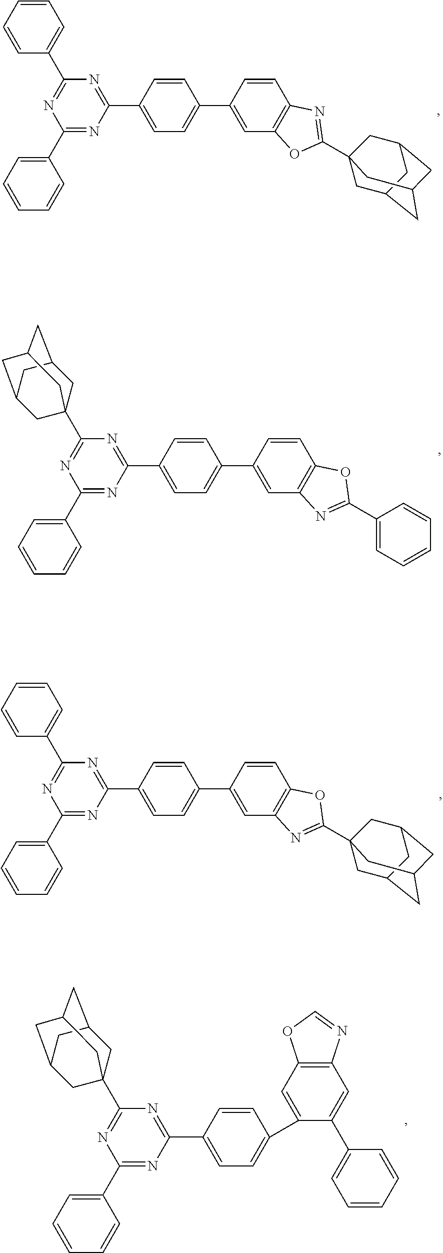 [SMILES: c1ccc(-c2nc(-c3ccccc3)nc(-c3ccc(-c4ccc5nc(C67CC8CC(CC(C8)C6)C7)oc5c4)cc3)n2)cc1 ; c1ccc(-c2nc(-c3ccc(-c4ccc5oc(-c6ccccc6)nc5c4)cc3)nc(C34CC5CC(CC(C5)C3)C4)n2)cc1 ; c1ccc(-c2nc(-c3ccccc3)nc(-c3ccc(-c4ccc5oc(C67CC8CC(CC(C8)C6)C7)nc5c4)cc3)n2)cc1 ; c1ccc(-c2nc(-c3ccc(-c4cc5ocnc5cc4-c4ccccc4)cc3)nc(C34CC5CC(CC(C5)C3)C4)n2)cc1]

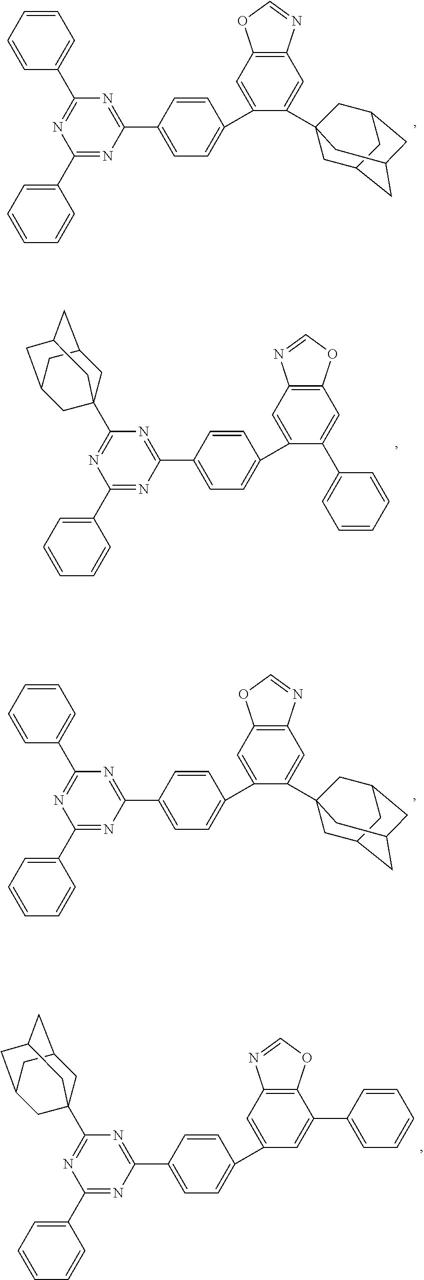 [SMILES: c1ccc(-c2nc(-c3ccccc3)nc(-c3ccc(-c4cc5ocnc5cc4C45CC6CC(CC(C6)C4)C5)cc3)n2)cc1 ; c1ccc(-c2nc(-c3ccc(-c4cc5ncoc5cc4-c4ccccc4)cc3)nc(C34CC5CC(CC(C5)C3)C4)n2)cc1 ; c1ccc(-c2nc(-c3ccccc3)nc(-c3ccc(-c4cc5ocnc5cc4C45CC6CC(CC(C6)C4)C5)cc3)n2)cc1 ; c1ccc(-c2nc(-c3ccc(-c4cc(-c5ccccc5)c5ocnc5c4)cc3)nc(C34CC5CC(CC(C5)C3)C4)n2)cc1]

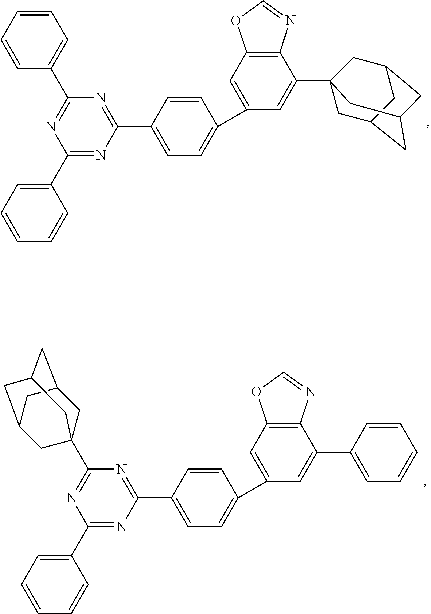 [SMILES: c1ccc(-c2nc(-c3ccccc3)nc(-c3ccc(-c4cc(C56CC7CC(CC(C7)C5)C6)c5ncoc5c4)cc3)n2)cc1 ; c1ccc(-c2nc(-c3ccc(-c4cc(-c5ccccc5)c5ncoc5c4)cc3)nc(C34CC5CC(CC(C5)C3)C4)n2)cc1]

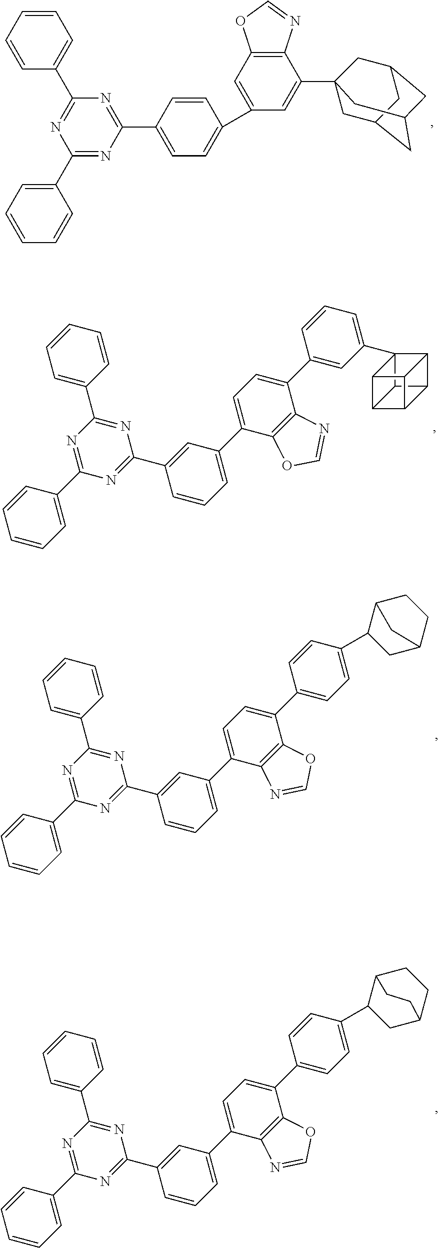 [SMILES: c1ccc(-c2nc(-c3ccccc3)nc(-c3ccc(-c4cc(C56CC7CC(CC(C7)C5)C6)c5ncoc5c4)cc3)n2)cc1 ; c1ccc(-c2nc(-c3ccccc3)nc(-c3cccc(-c4ccc(-c5cccc(C67C8C9C%10C8C6C%10C97)c5)c5ncoc45)c3)n2)cc1 ; c1ccc(-c2nc(-c3ccccc3)nc(-c3cccc(-c4ccc(-c5ccc(C6CC7CCC6C7)cc5)c5ocnc45)c3)n2)cc1 ; c1ccc(-c2nc(-c3ccccc3)nc(-c3cccc(-c4ccc(-c5ccc(C6CC7CCC6CC7)cc5)c5ocnc45)c3)n2)cc1]

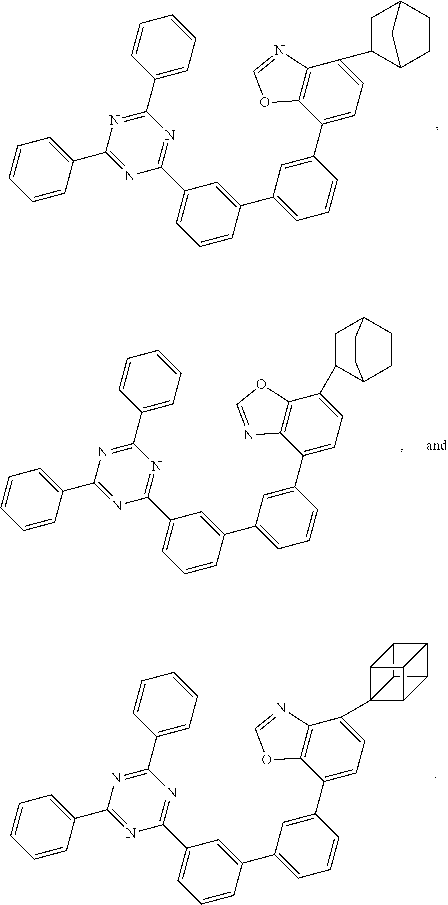 [SMILES: c1ccc(-c2nc(-c3ccccc3)nc(-c3cccc(-c4cccc(-c5ccc(C6CC7CCC6C7)c6ncoc56)c4)c3)n2)cc1 ; c1ccc(-c2nc(-c3ccccc3)nc(-c3cccc(-c4cccc(-c5ccc(C6CC7CCC6CC7)c6ocnc56)c4)c3)n2)cc1 ; c1ccc(-c2nc(-c3ccccc3)nc(-c3cccc(-c4cccc(-c5ccc(C67C8C9C%10C8C6C%10C97)c6ncoc56)c4)c3)n2)cc1]

Optionally, in the material of the electron transport layer, L3 includes a first benzene ring and a second benzene ring, and the first benzene ring is bonded to the second benzene ring; and

the first benzene ring is bonded to the heteroaromatic ring group including at least one nitrogen atom, and the heteroaromatic ring group including at least one nitrogen atom is located at a meta-position of the second benzene ring.

Optionally, a chemical structural formula of the material of the electron transport layer includes any one of

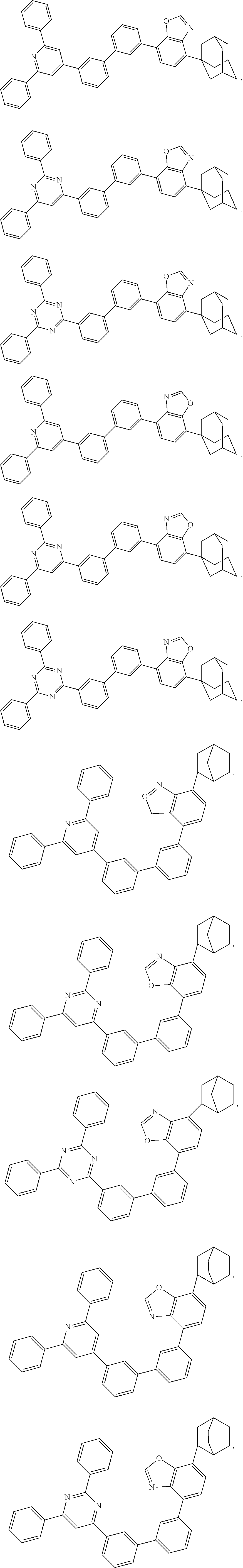

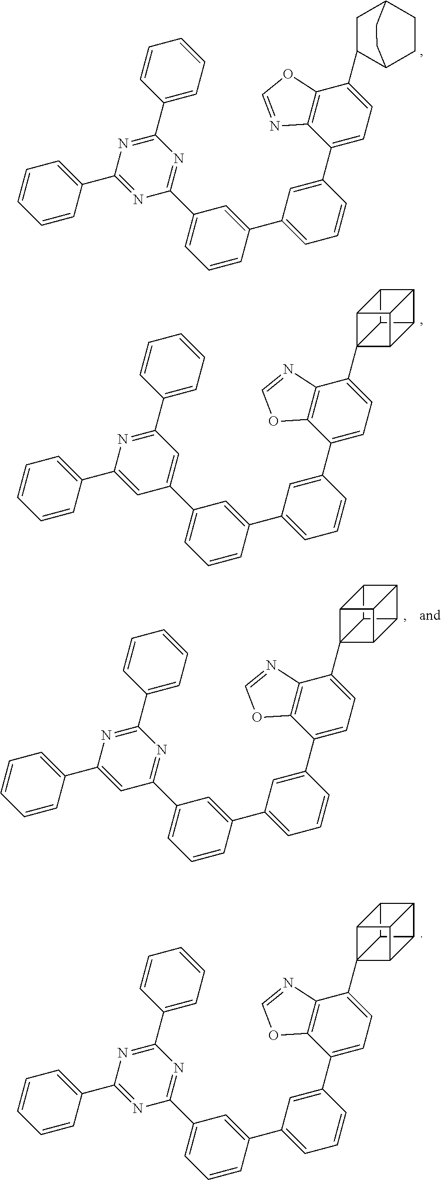 [SMILES: CCCC1CCCC(c2ccc(-c3cccc(-c4cccc(-c5nc(-c6ccccc6)nc(-c6ccccc6)n5)c4)c3)c3ncoc23)C1 ; c1ccc(-c2cc(-c3cccc(-c4cccc(-c5ccc(C67C8C9C%10C8C6C%10C97)c6ncoc56)c4)c3)cc(-c3ccccc3)n2)cc1 ; c1ccc(-c2cc(-c3cccc(-c4cccc(-c5ccc(C67C8C9C%10C8C6C%10C97)c6ncoc56)c4)c3)nc(-c3ccccc3)n2)cc1 ; c1ccc(-c2nc(-c3ccccc3)nc(-c3cccc(-c4cccc(-c5ccc(C67C8C9C%10C8C6C%10C97)c6ncoc56)c4)c3)n2)cc1]

Optionally, the material of the electron transport layer has cyano.

Optionally, a chemical structural formula of a material of the electron transport layer having the cyano includes any one of

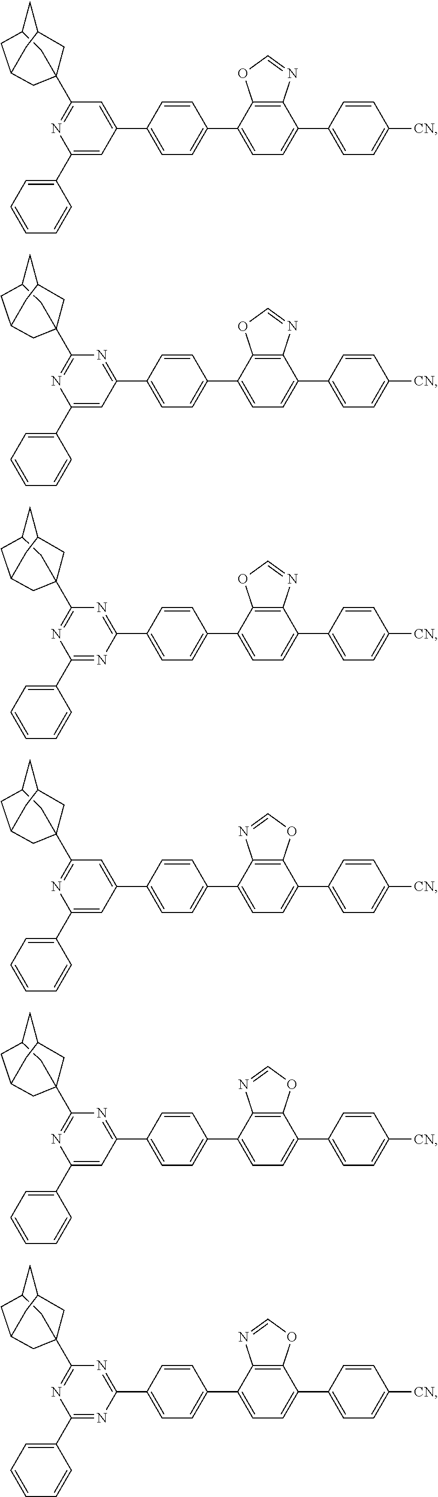

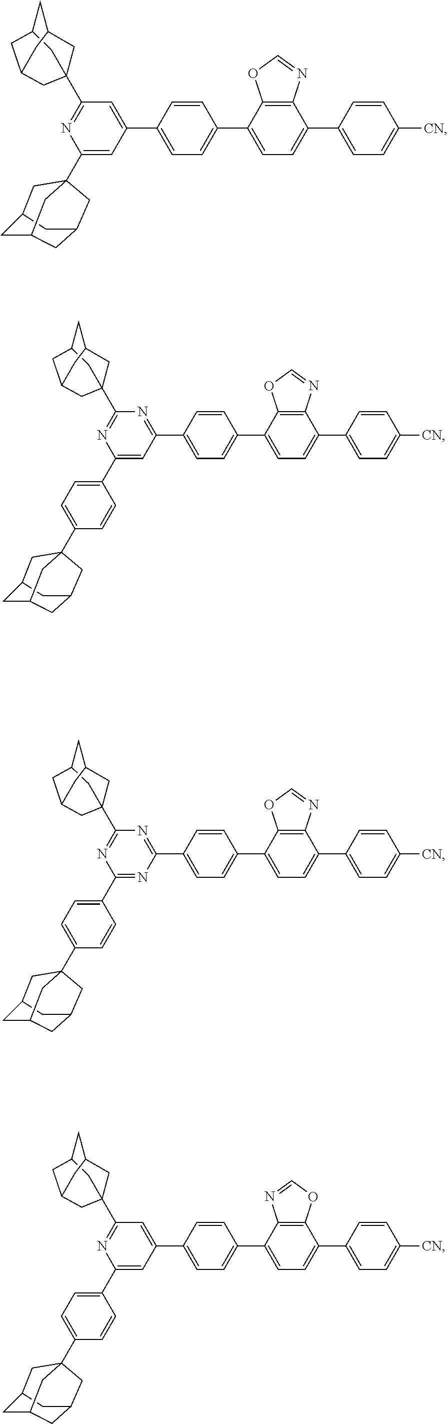 [SMILES: N#Cc1ccc(-c2ccc(-c3ccc(-c4cc(C56CC7CC(CC(C7)C5)C6)nc(C56CC7CC(CC(C7)C5)C6)c4)cc3)c3ocnc23)cc1 ; N#Cc1ccc(-c2ccc(-c3ccc(-c4cc(-c5ccc(C67CC8CC(CC(C8)C6)C7)cc5)nc(C56CC7CC(CC(C7)C5)C6)n4)cc3)c3ocnc23)cc1 ; N#Cc1ccc(-c2ccc(-c3ccc(-c4nc(-c5ccc(C67CC8CC(CC(C8)C6)C7)cc5)nc(C56CC7CC(CC(C7)C5)C6)n4)cc3)c3ocnc23)cc1 ; N#Cc1ccc(-c2ccc(-c3ccc(-c4cc(-c5ccc(C67CC8CC(CC(C8)C6)C7)cc5)nc(C56CC7CC(CC(C7)C5)C6)c4)cc3)c3ncoc23)cc1]

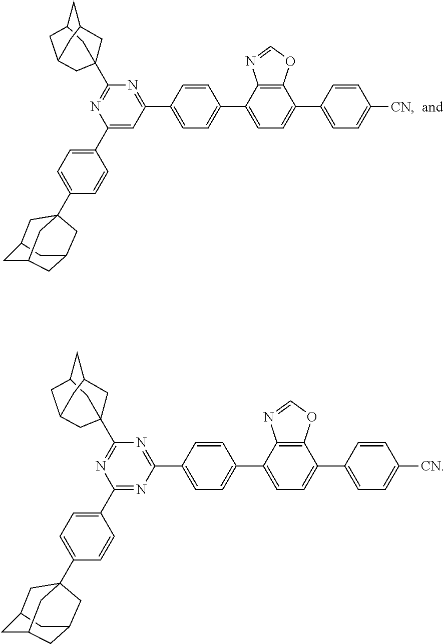

Optionally, in the material of the electron transport layer, the adamantane is connected to the heteroaromatic ring group including at least one nitrogen atom by a phenyl group, and is located at an ortho-position of the nitrogen atom in the heteroaromatic ring group.

Optionally, a chemical structural formula of the material of the electron transport layer includes any one of

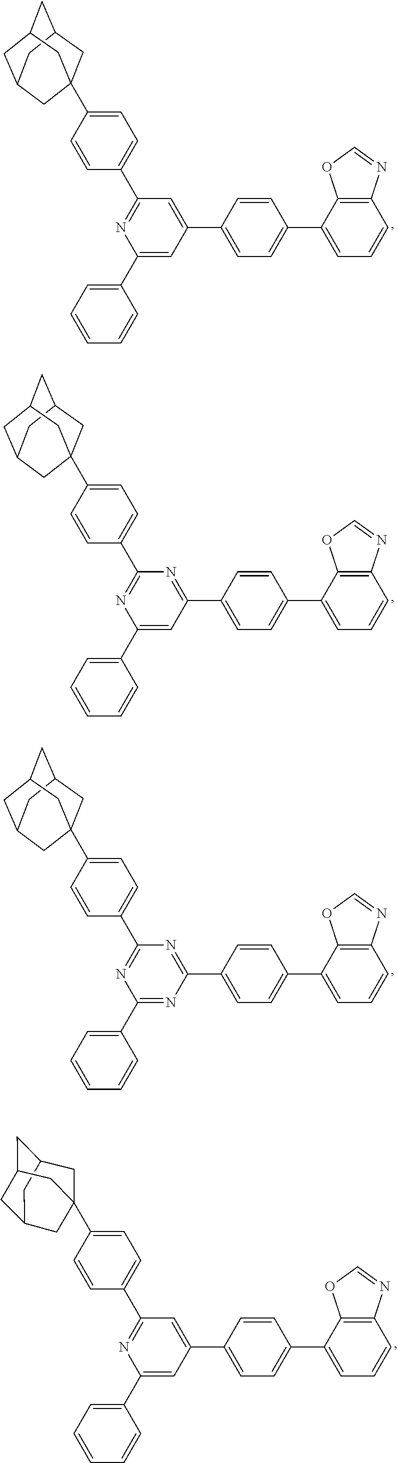

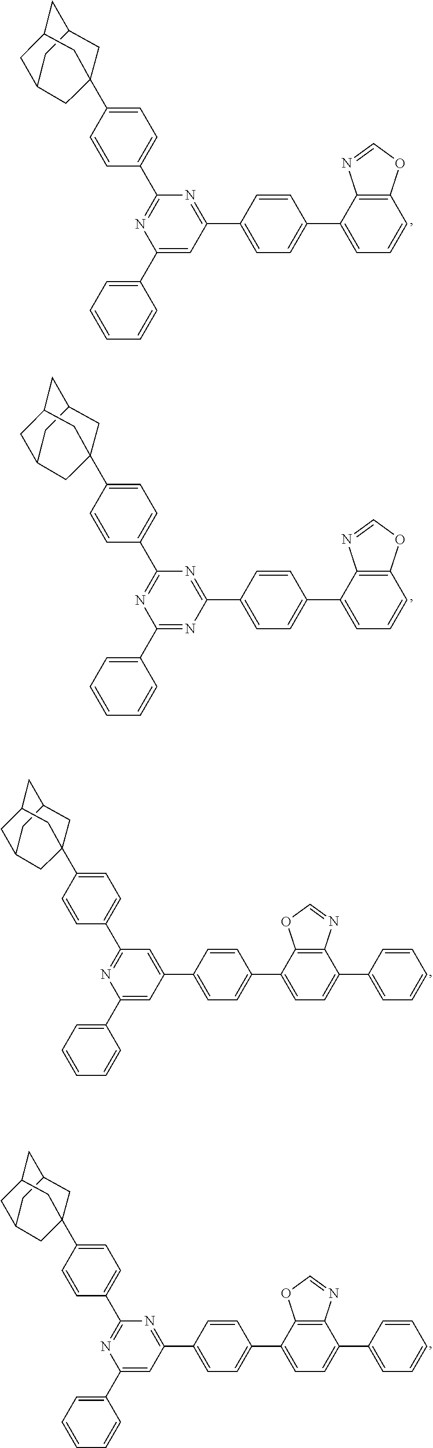

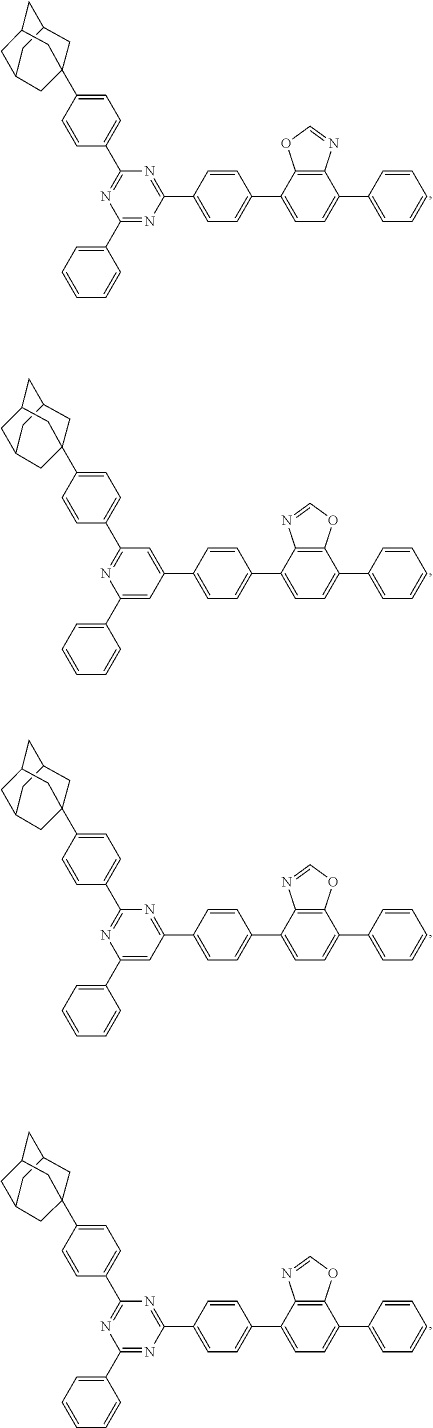

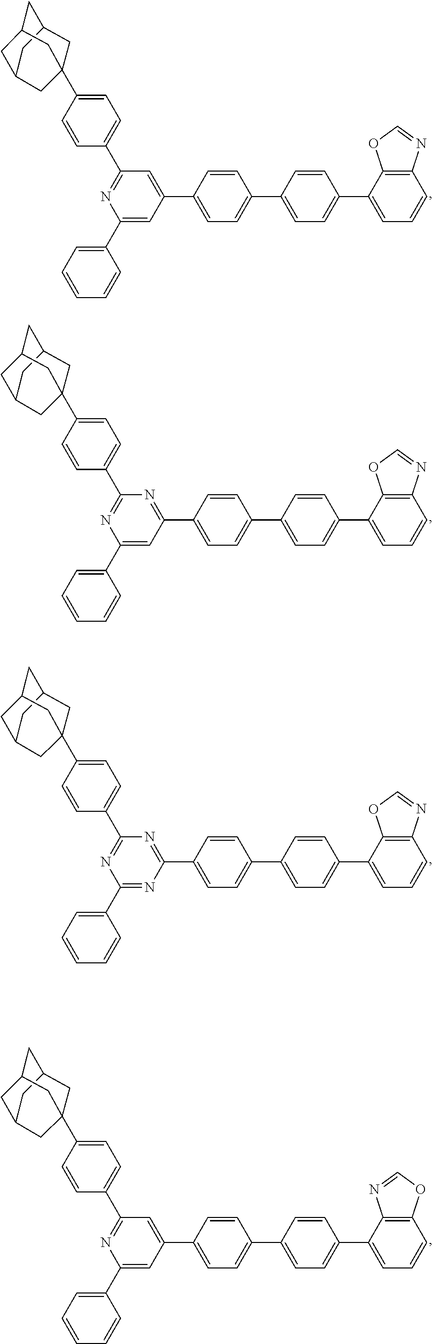

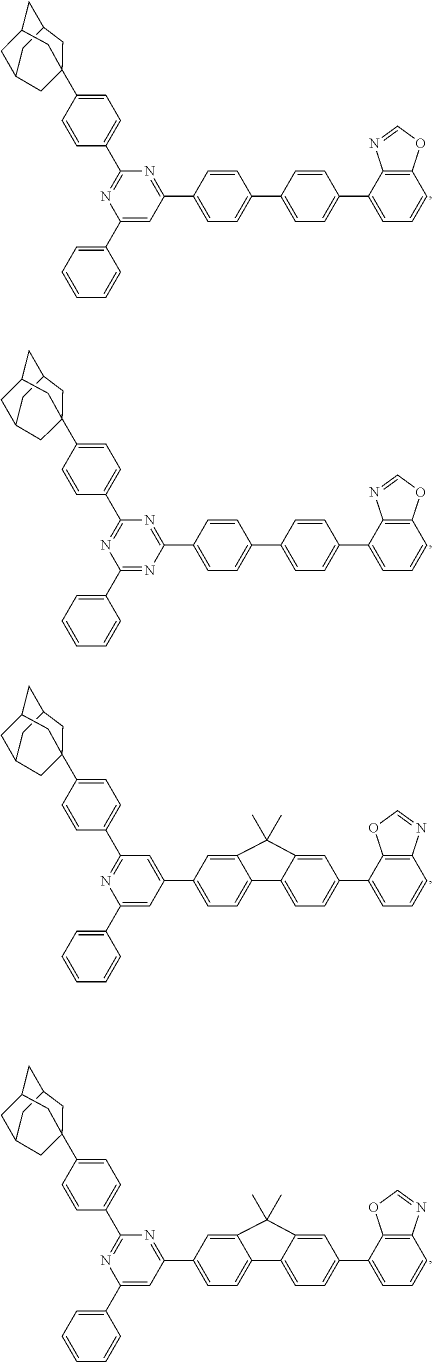

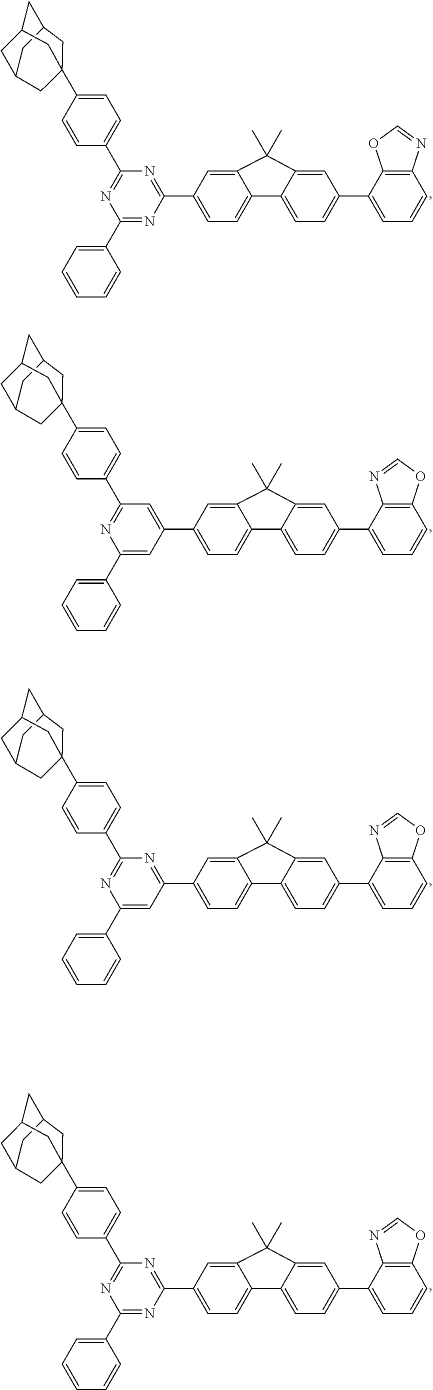

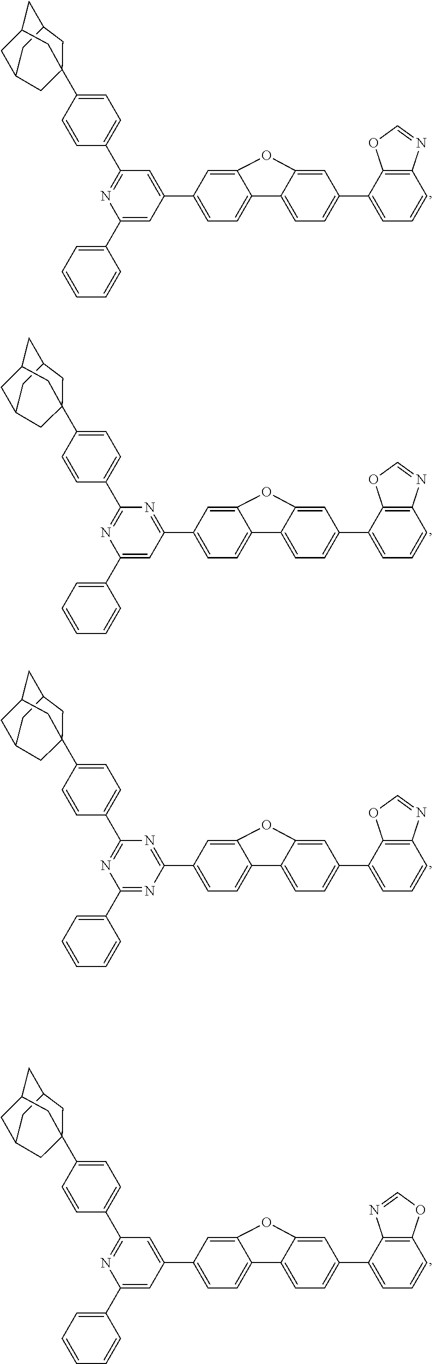

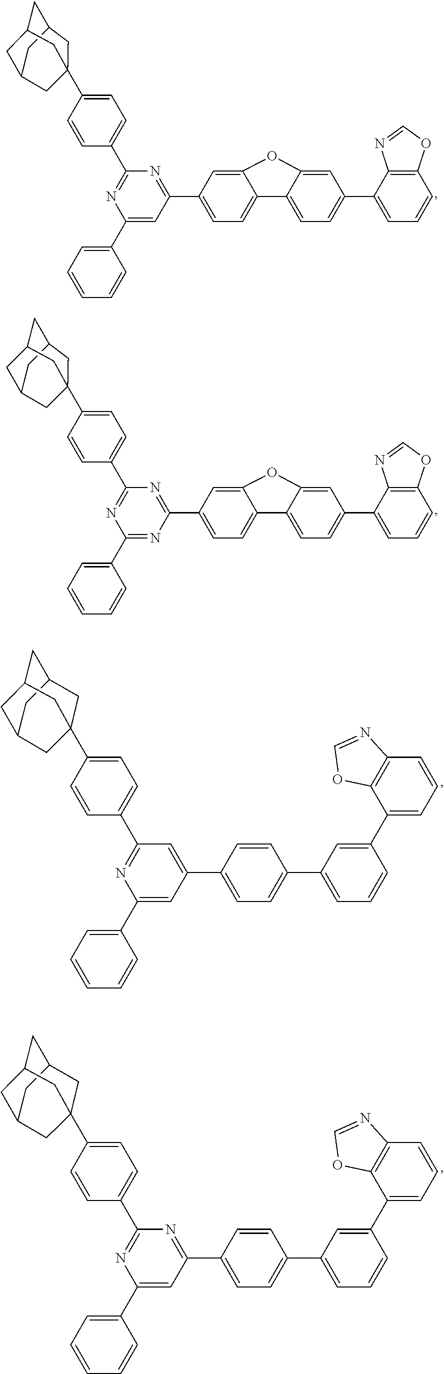

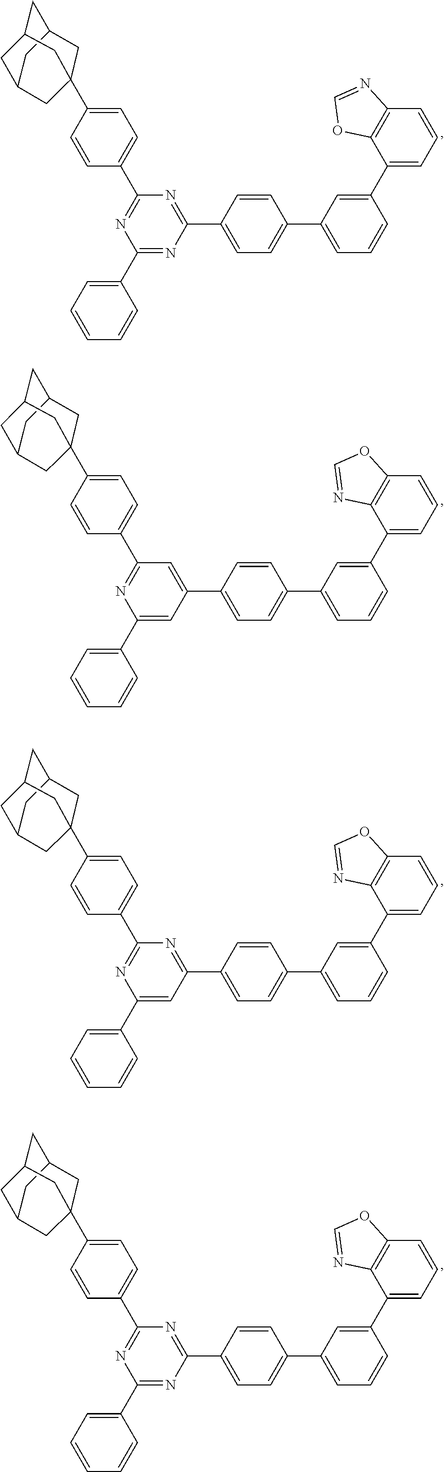

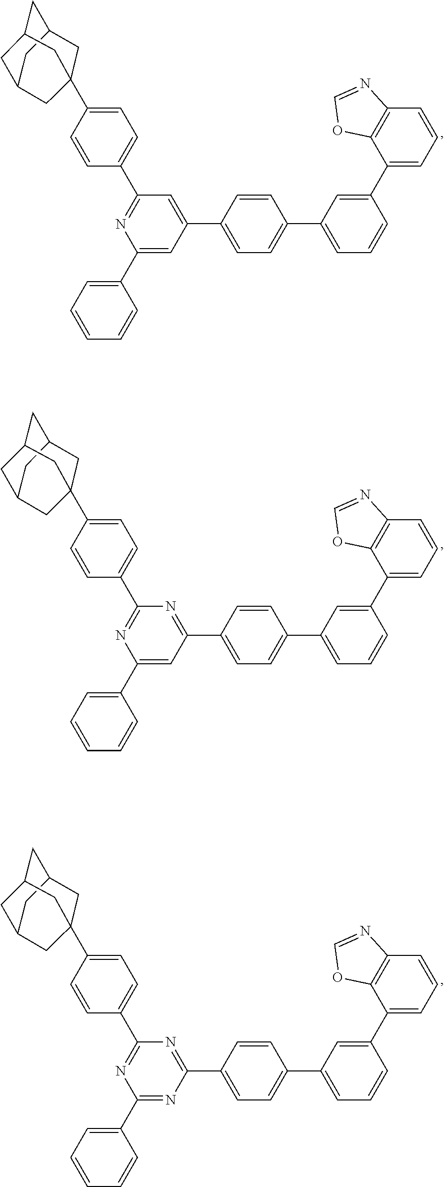 [SMILES: c1ccc(-c2cc(-c3ccc(-c4cccc(-c5cccc6ncoc56)c4)cc3)cc(-c3ccc(C45CC6CC(CC(C6)C4)C5)cc3)n2)cc1 ; "," ; c1ccc(-c2cc(-c3ccc(-c4cccc(-c5cccc6ncoc56)c4)cc3)nc(-c3ccc(C45CC6CC(CC(C6)C4)C5)cc3)n2)cc1 ; "," ; c1ccc(-c2nc(-c3ccc(-c4cccc(-c5cccc6ncoc56)c4)cc3)nc(-c3ccc(C45CC6CC(CC(C6)C4)C5)cc3)n2)cc1 ; ","]

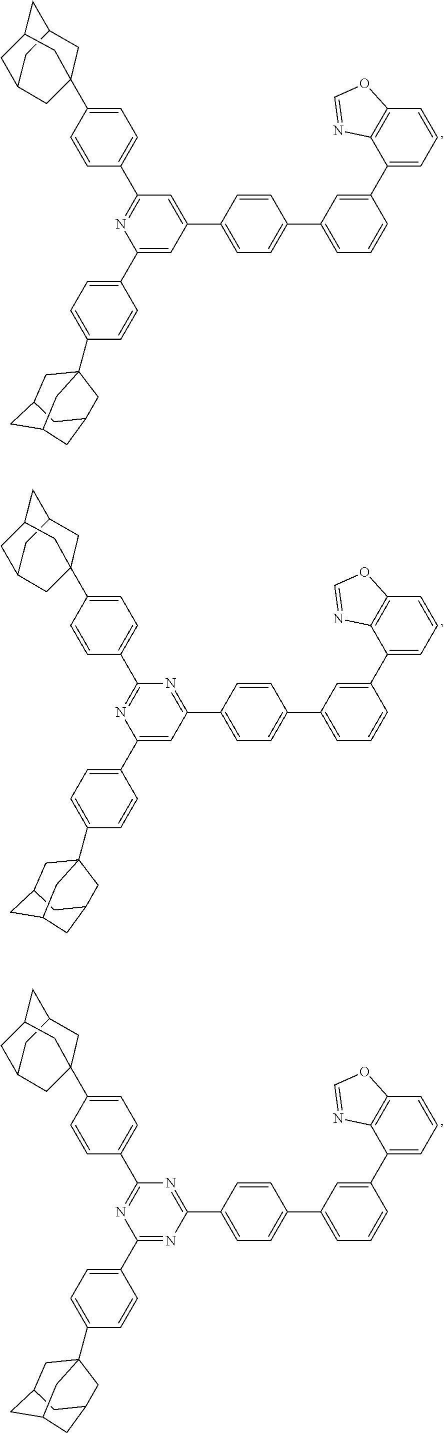 [SMILES: c1cc(-c2ccc(-c3cc(-c4ccc(C56CC7CC(CC(C7)C5)C6)cc4)nc(-c4ccc(C56CC7CC(CC(C7)C5)C6)cc4)c3)cc2)cc(-c2cccc3ocnc23)c1 ; "," ; c1cc(-c2ccc(-c3cc(-c4ccc(C56CC7CC(CC(C7)C5)C6)cc4)nc(-c4ccc(C56CC7CC(CC(C7)C5)C6)cc4)n3)cc2)cc(-c2cccc3ocnc23)c1 ; "," ; c1cc(-c2ccc(-c3nc(-c4ccc(C56CC7CC(CC(C7)C5)C6)cc4)nc(-c4ccc(C56CC7CC(CC(C7)C5)C6)cc4)n3)cc2)cc(-c2cccc3ocnc23)c1 ; ","]

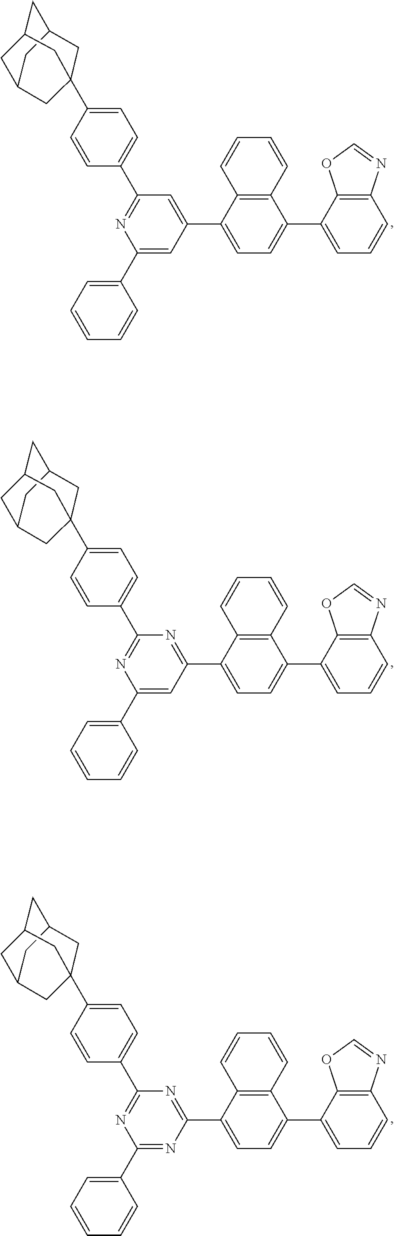 [SMILES: c1ccc(-c2cc(-c3ccc(-c4cccc5ncoc45)c4ccccc34)cc(-c3ccc(C45CC6CC(CC(C6)C4)C5)cc3)n2)cc1 ; "," ; c1ccc(-c2cc(-c3ccc(-c4cccc5ncoc45)c4ccccc34)nc(-c3ccc(C45CC6CC(CC(C6)C4)C5)cc3)n2)cc1 ; "," ; c1ccc(-c2nc(-c3ccc(C45CC6CC(CC(C6)C4)C5)cc3)nc(-c3ccc(-c4cccc5ncoc45)c4ccccc34)n2)cc1 ; ","]

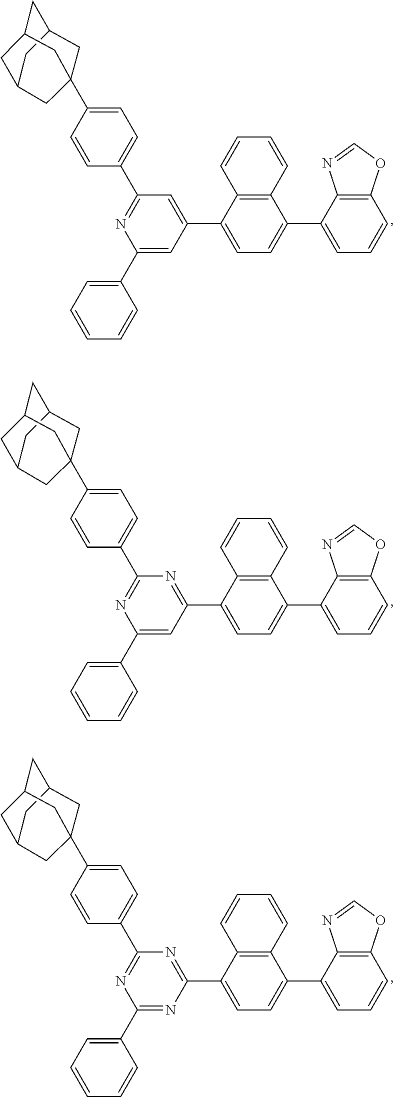 [SMILES: c1ccc(-c2cc(-c3ccc(-c4cccc5ocnc45)c4ccccc34)cc(-c3ccc(C45CC6CC(CC(C6)C4)C5)cc3)n2)cc1 ; "," ; c1ccc(-c2cc(-c3ccc(-c4cccc5ocnc45)c4ccccc34)nc(-c3ccc(C45CC6CC(CC(C6)C4)C5)cc3)n2)cc1 ; "," ; c1ccc(-c2nc(-c3ccc(C45CC6CC(CC(C6)C4)C5)cc3)nc(-c3ccc(-c4cccc5ocnc45)c4ccccc34)n2)cc1 ; ","]

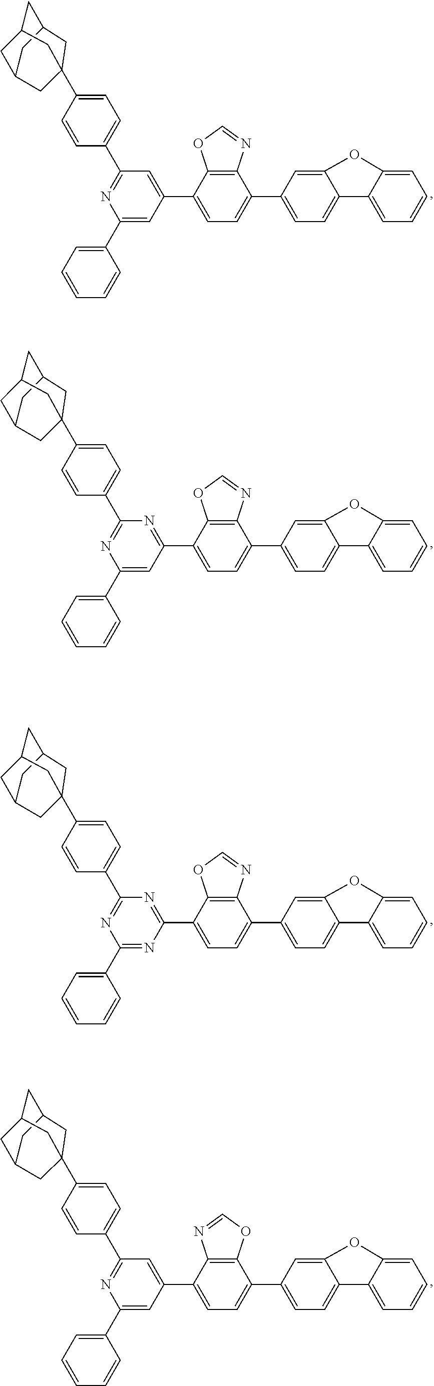

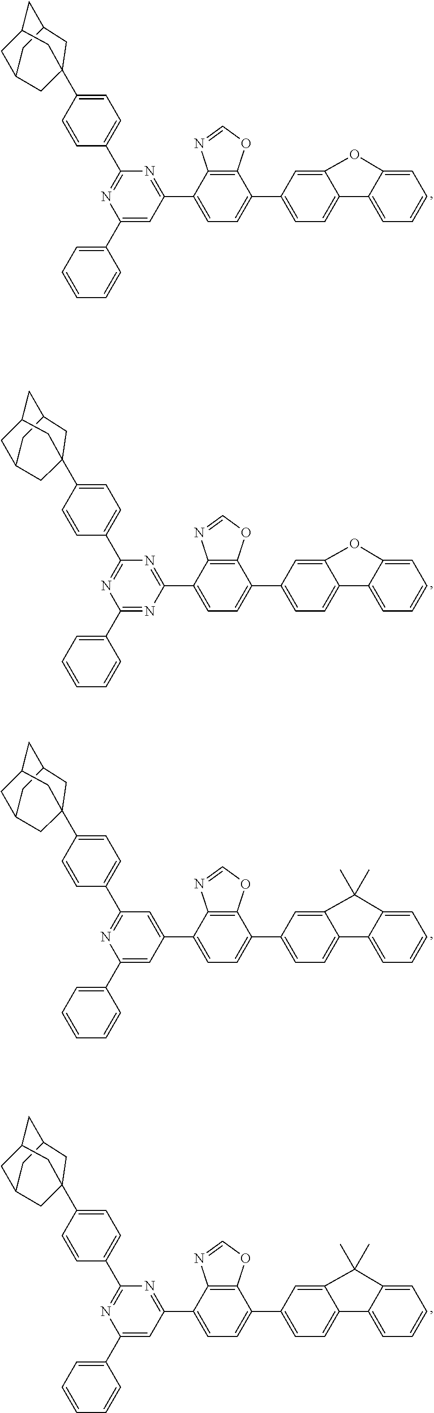

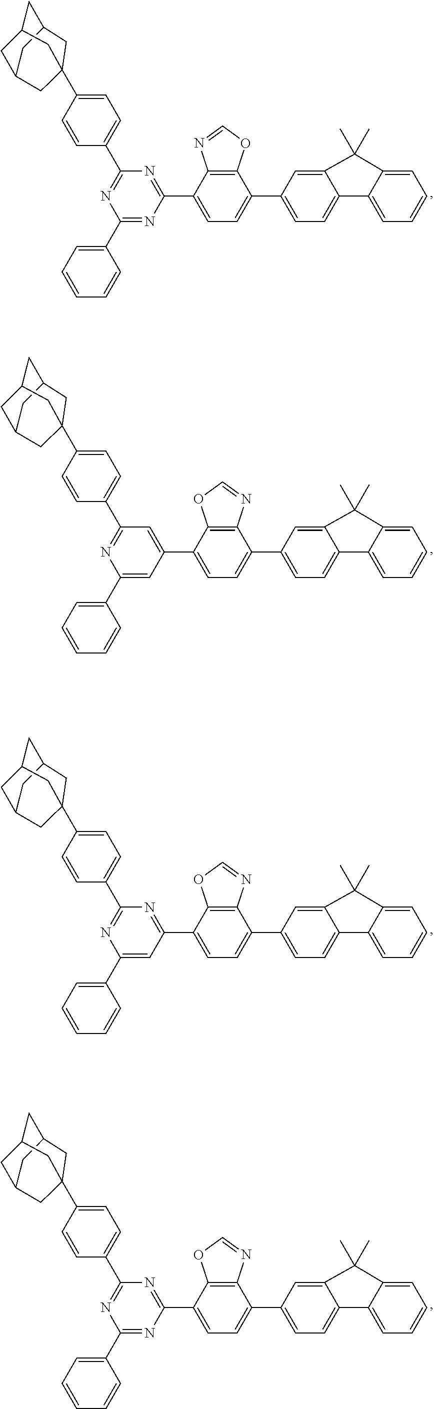

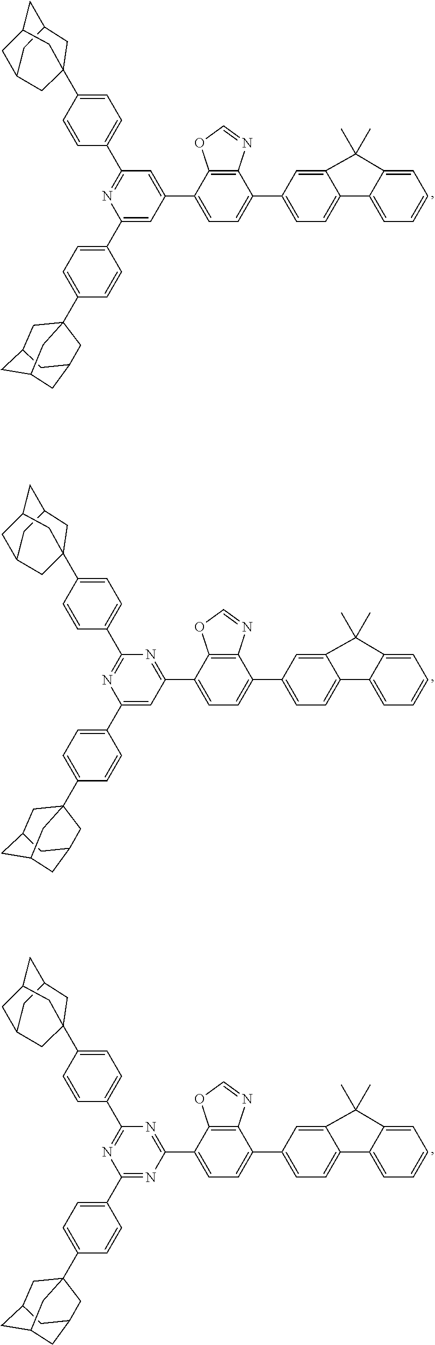 [SMILES: CC1(C)c2ccccc2-c2ccc(-c3ccc(-c4cc(-c5ccc(C67CC8CC(CC(C8)C6)C7)cc5)nc(-c5ccc(C67CC8CC(CC(C8)C6)C7)cc5)c4)c4ocnc34)cc21 ; "," ; CC1(C)c2ccccc2-c2ccc(-c3ccc(-c4cc(-c5ccc(C67CC8CC(CC(C8)C6)C7)cc5)nc(-c5ccc(C67CC8CC(CC(C8)C6)C7)cc5)n4)c4ocnc34)cc21 ; "," ; CC1(C)c2ccccc2-c2ccc(-c3ccc(-c4nc(-c5ccc(C67CC8CC(CC(C8)C6)C7)cc5)nc(-c5ccc(C67CC8CC(CC(C8)C6)C7)cc5)n4)c4ocnc34)cc21 ; ","]

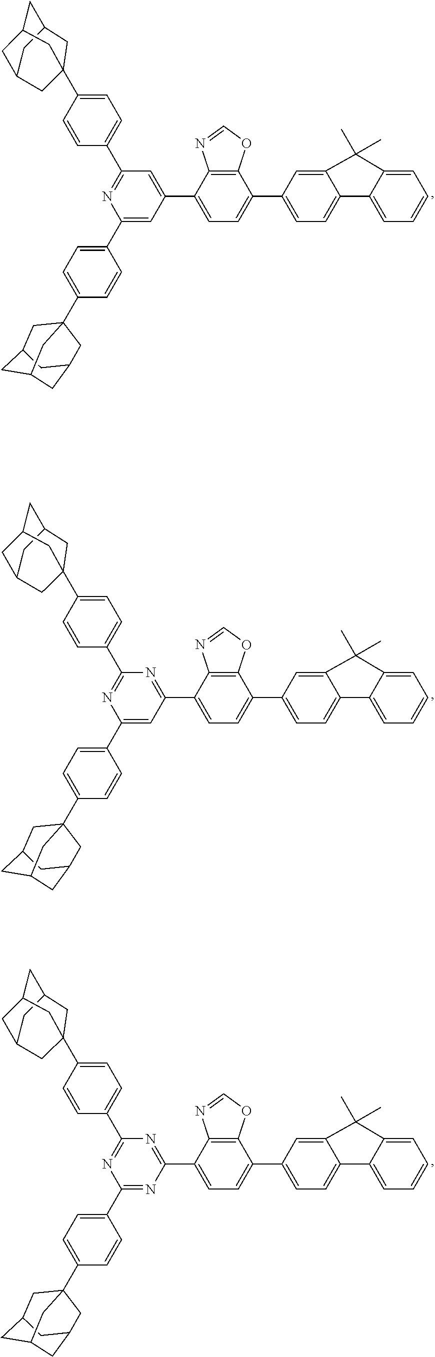 [SMILES: CC1(C)c2ccccc2-c2ccc(-c3ccc(-c4cc(-c5ccc(C67CC8CC(CC(C8)C6)C7)cc5)nc(-c5ccc(C67CC8CC(CC(C8)C6)C7)cc5)c4)c4ncoc34)cc21 ; "," ; CC1(C)c2ccccc2-c2ccc(-c3ccc(-c4cc(-c5ccc(C67CC8CC(CC(C8)C6)C7)cc5)nc(-c5ccc(C67CC8CC(CC(C8)C6)C7)cc5)n4)c4ncoc34)cc21 ; "," ; CC1(C)c2ccccc2-c2ccc(-c3ccc(-c4nc(-c5ccc(C67CC8CC(CC(C8)C6)C7)cc5)nc(-c5ccc(C67CC8CC(CC(C8)C6)C7)cc5)n4)c4ncoc34)cc21 ; ","]

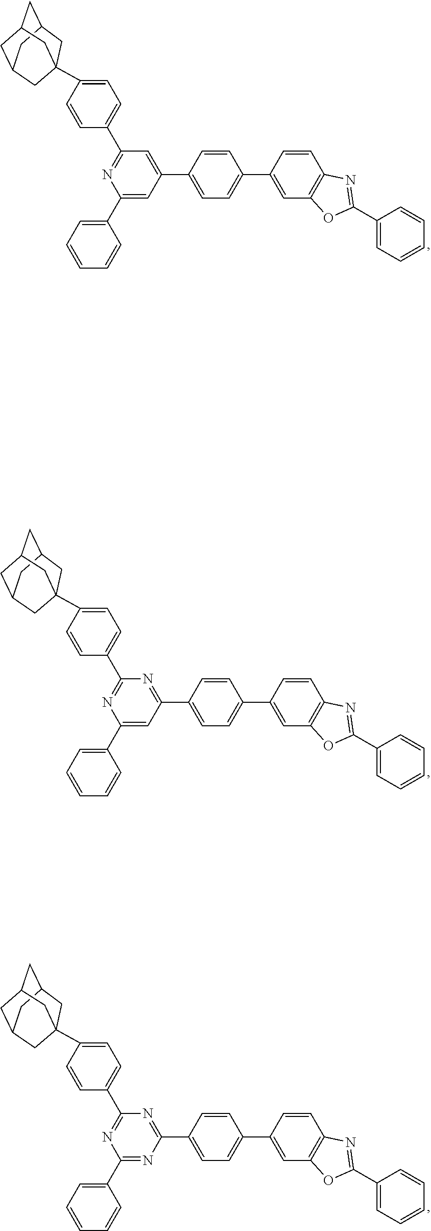 [SMILES: c1ccc(-c2cc(-c3ccc(-c4ccc5nc(-c6ccccc6)oc5c4)cc3)cc(-c3ccc(C45CC6CC(CC(C6)C4)C5)cc3)n2)cc1 ; "," ; c1ccc(-c2cc(-c3ccc(-c4ccc5nc(-c6ccccc6)oc5c4)cc3)nc(-c3ccc(C45CC6CC(CC(C6)C4)C5)cc3)n2)cc1 ; "," ; c1ccc(-c2nc(-c3ccc(-c4ccc5nc(-c6ccccc6)oc5c4)cc3)nc(-c3ccc(C45CC6CC(CC(C6)C4)C5)cc3)n2)cc1 ; ","]

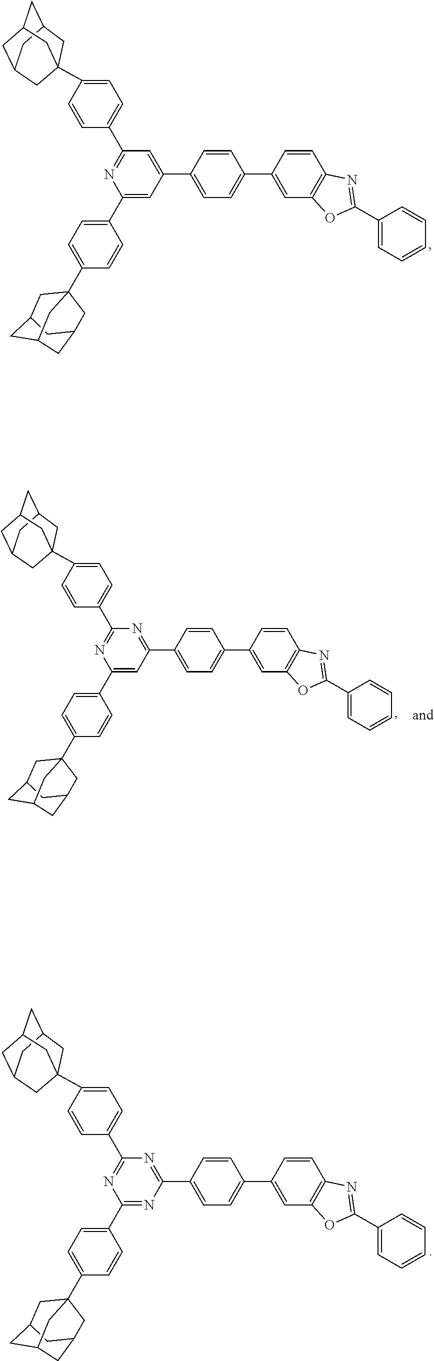

Optionally, a chemical structural formula of the material of the electron transport layer includes any one of

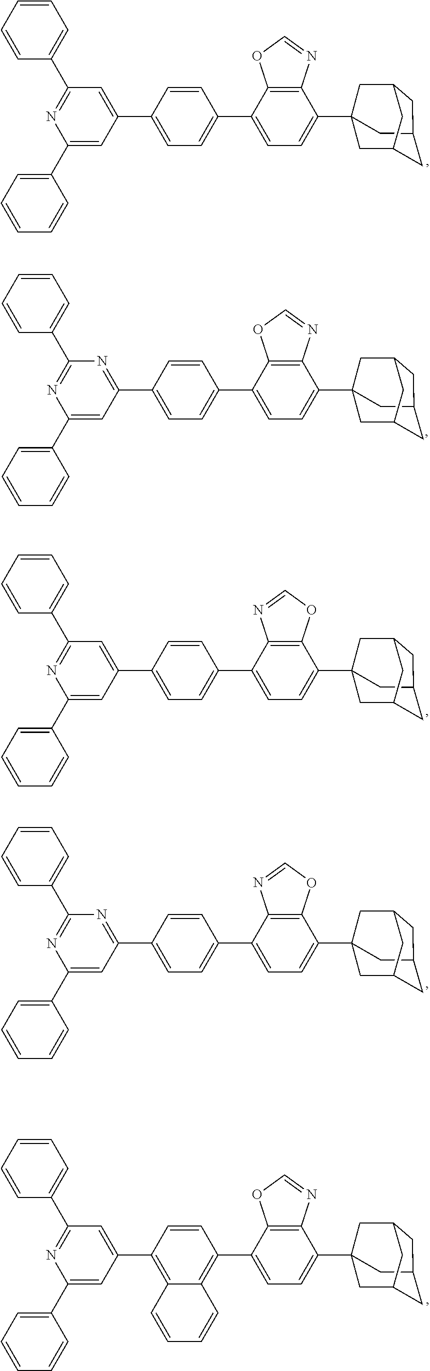

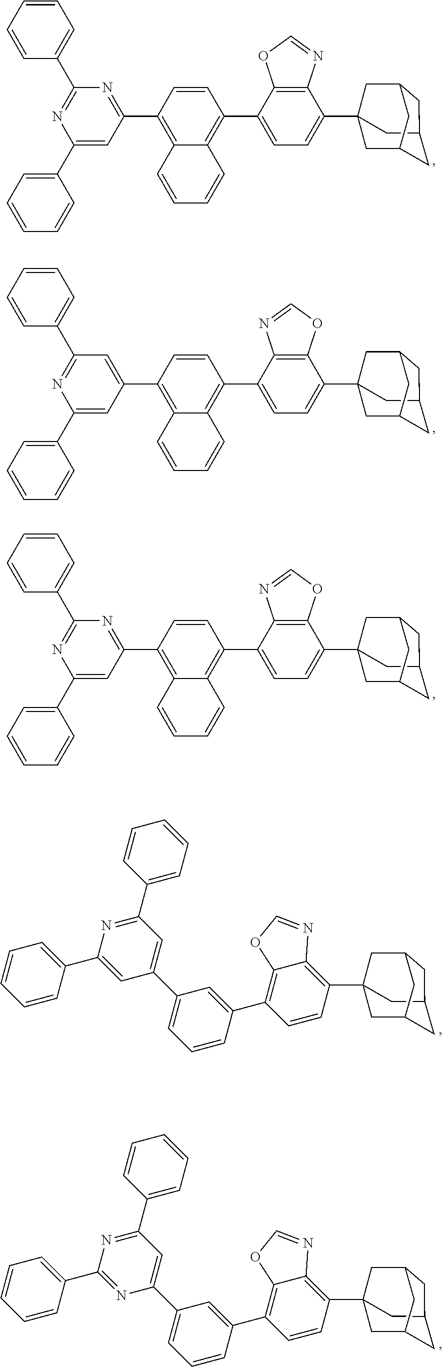

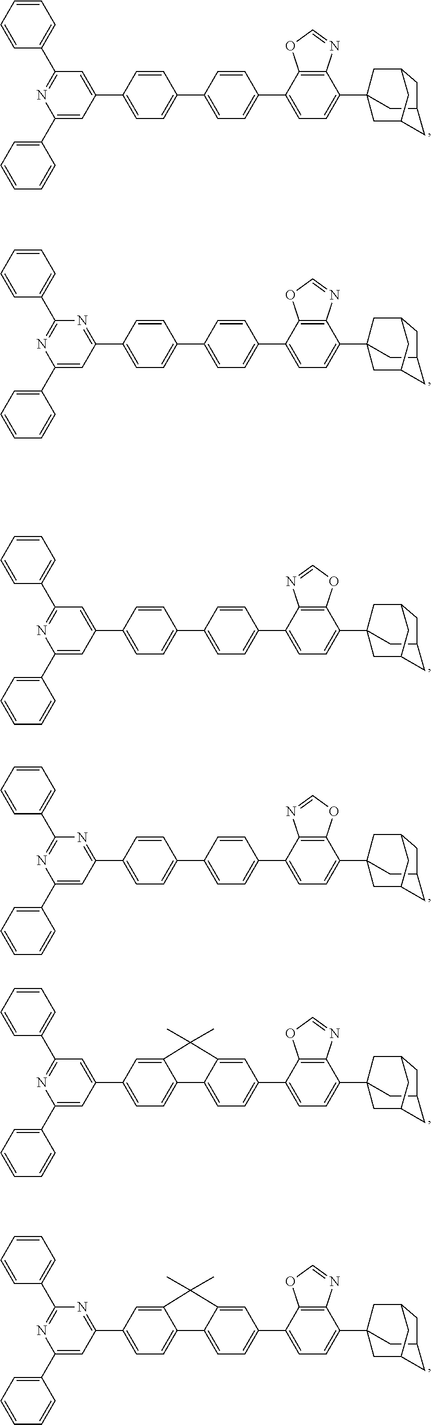

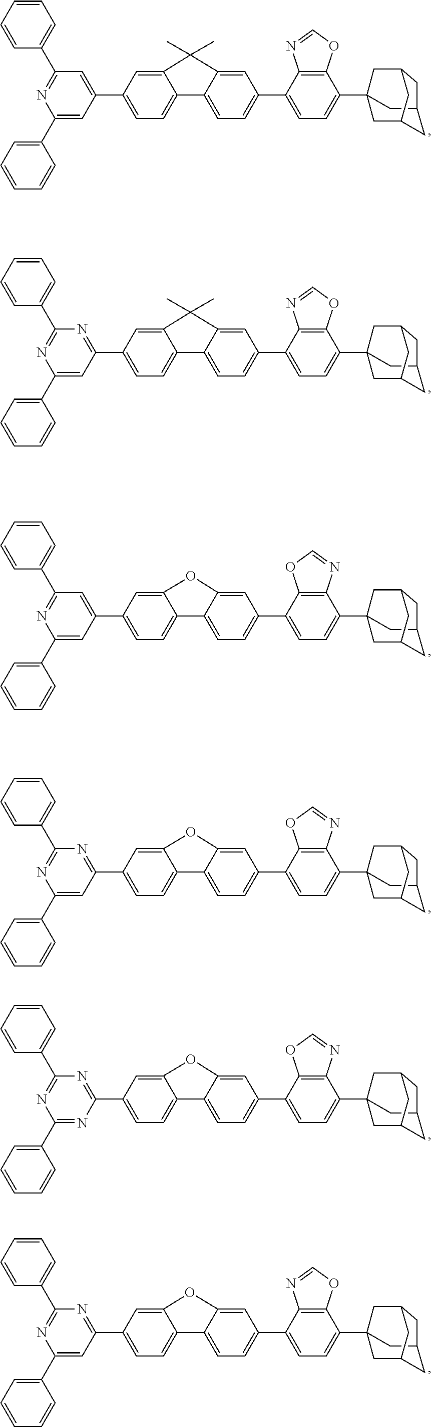

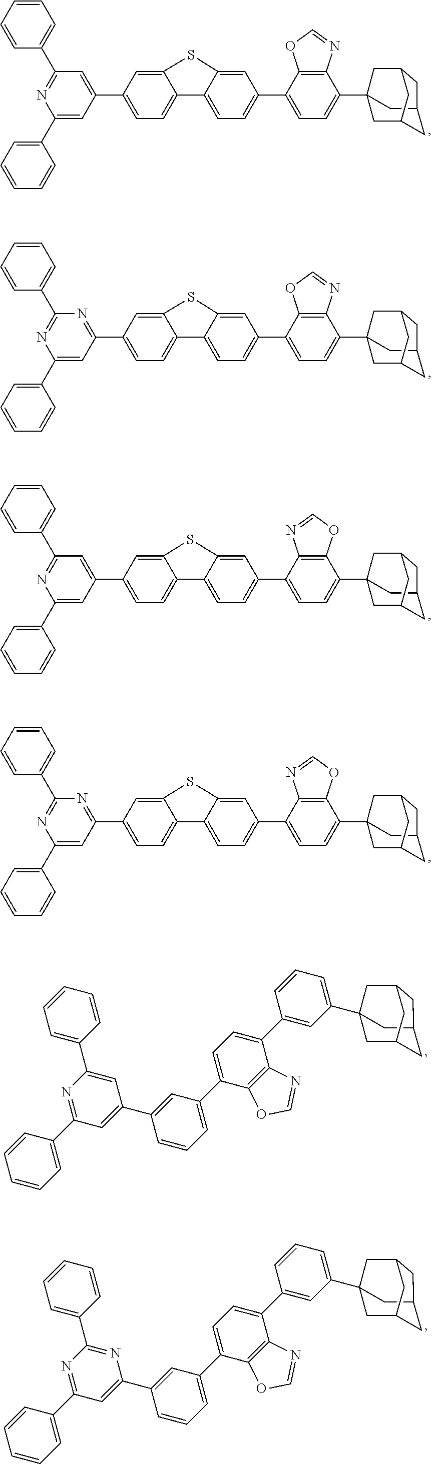

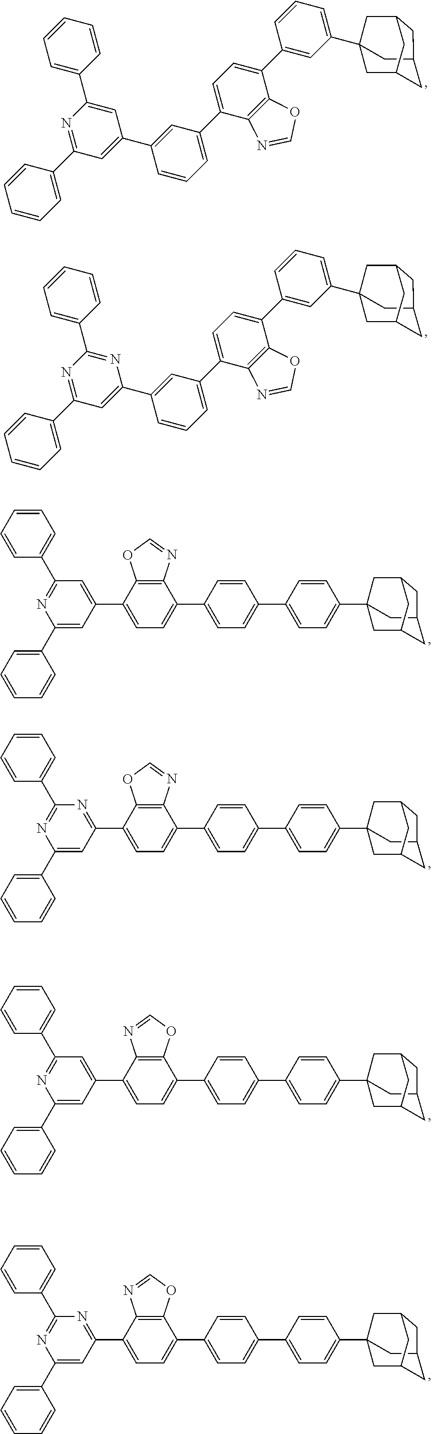

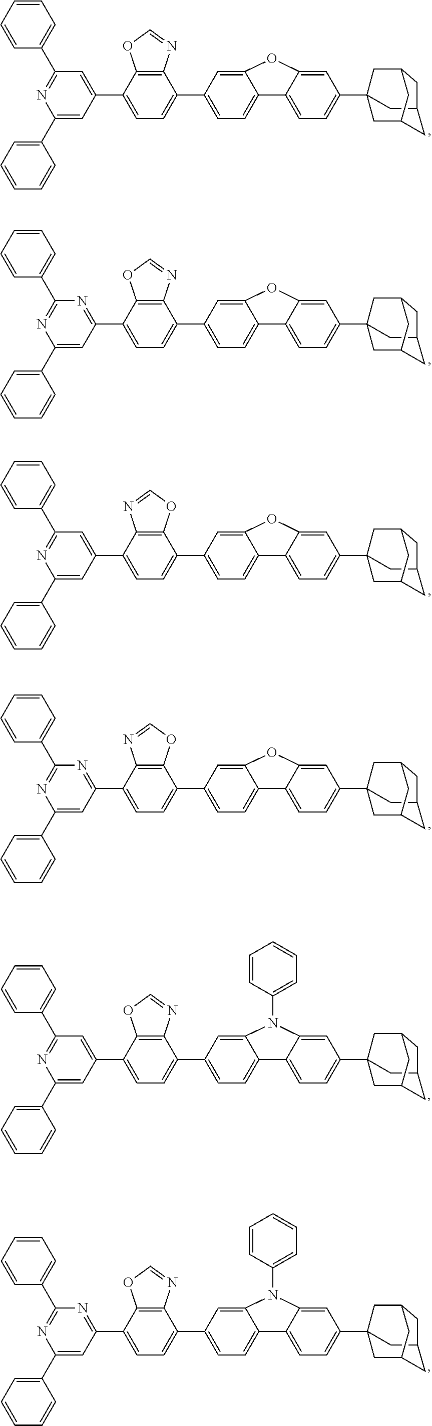

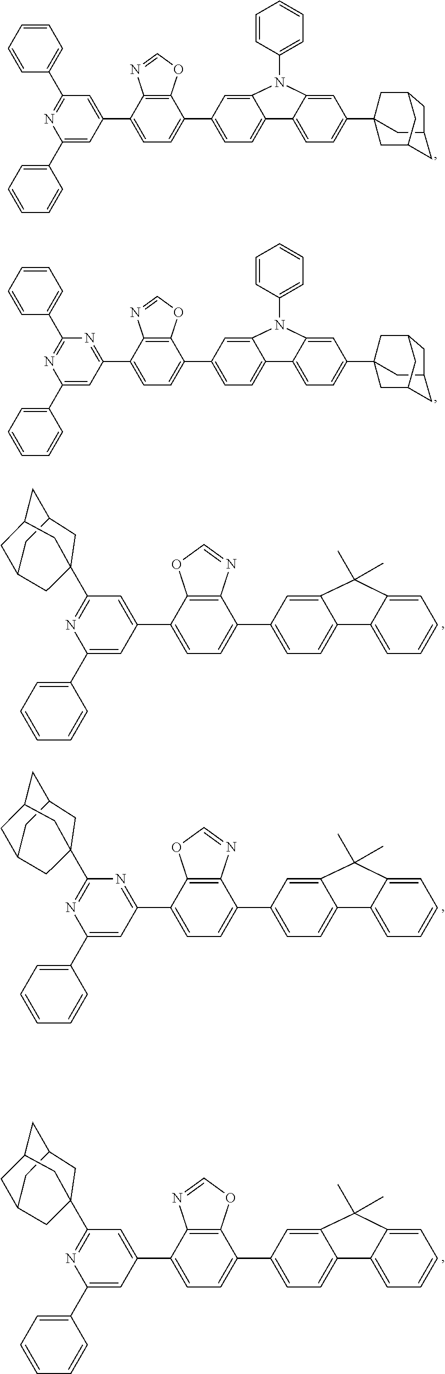

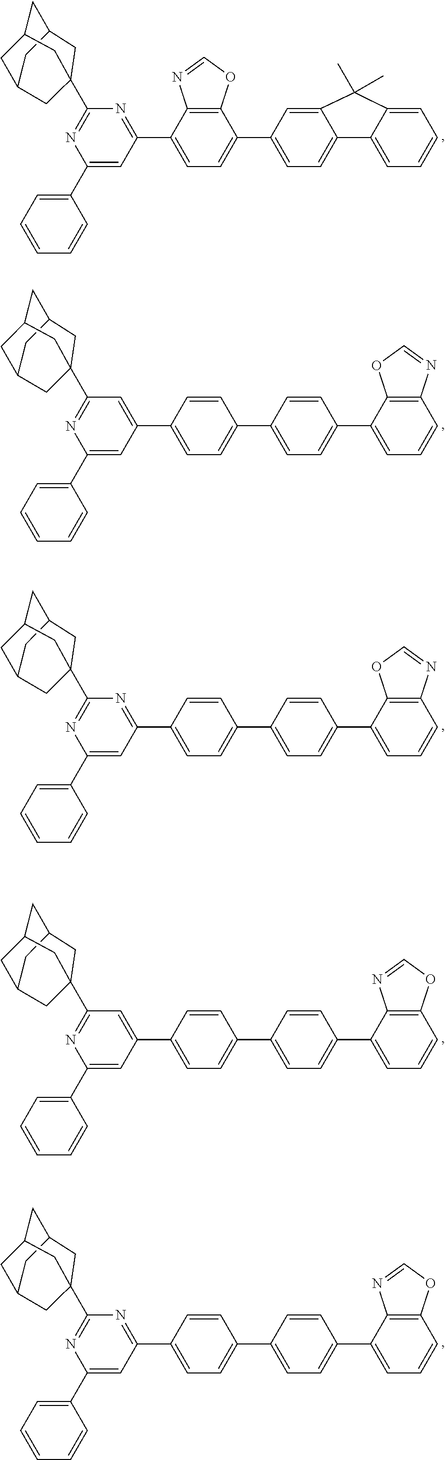

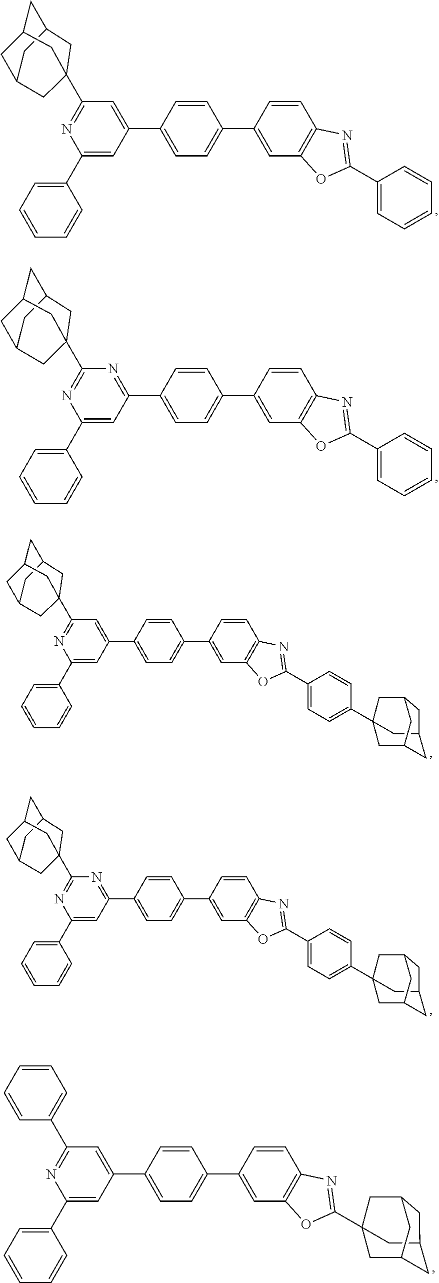

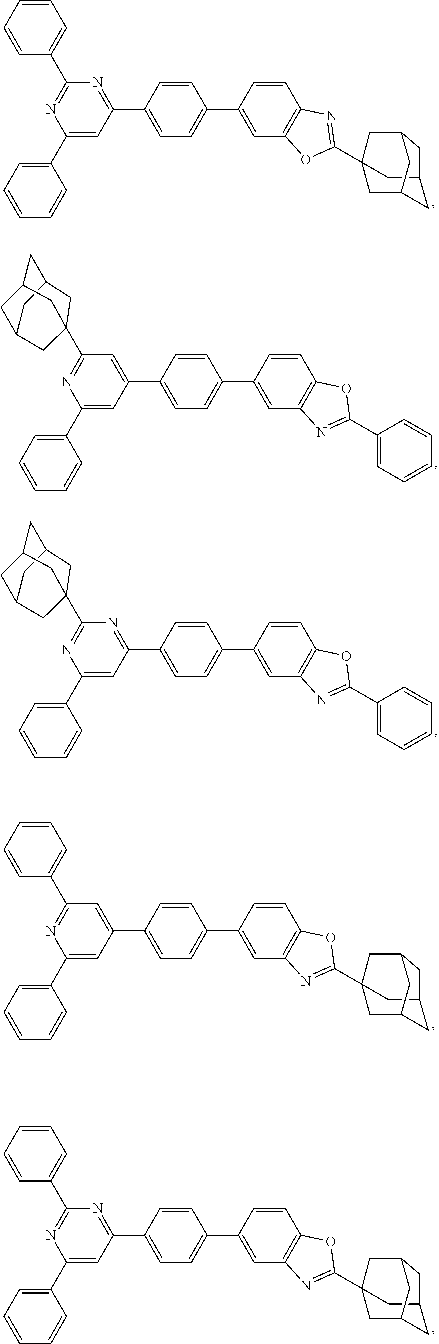

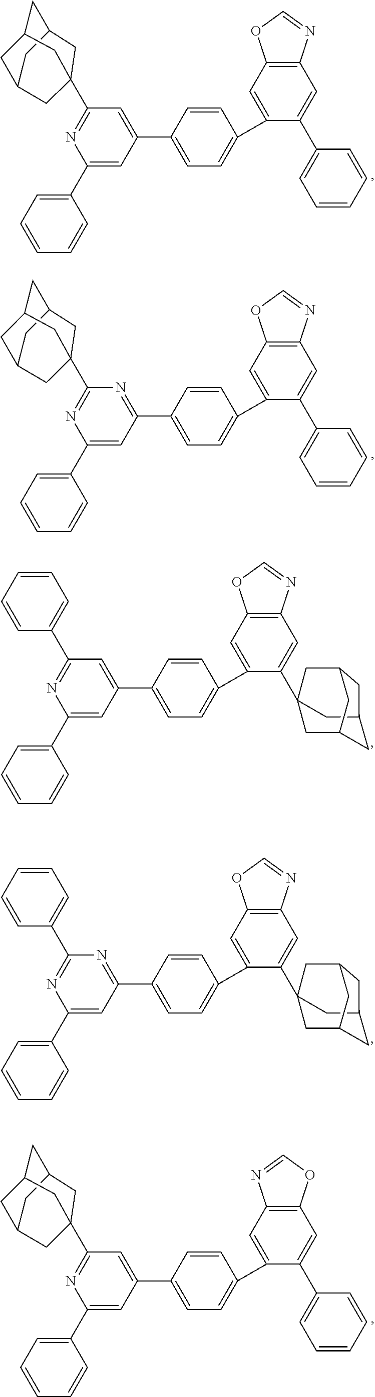

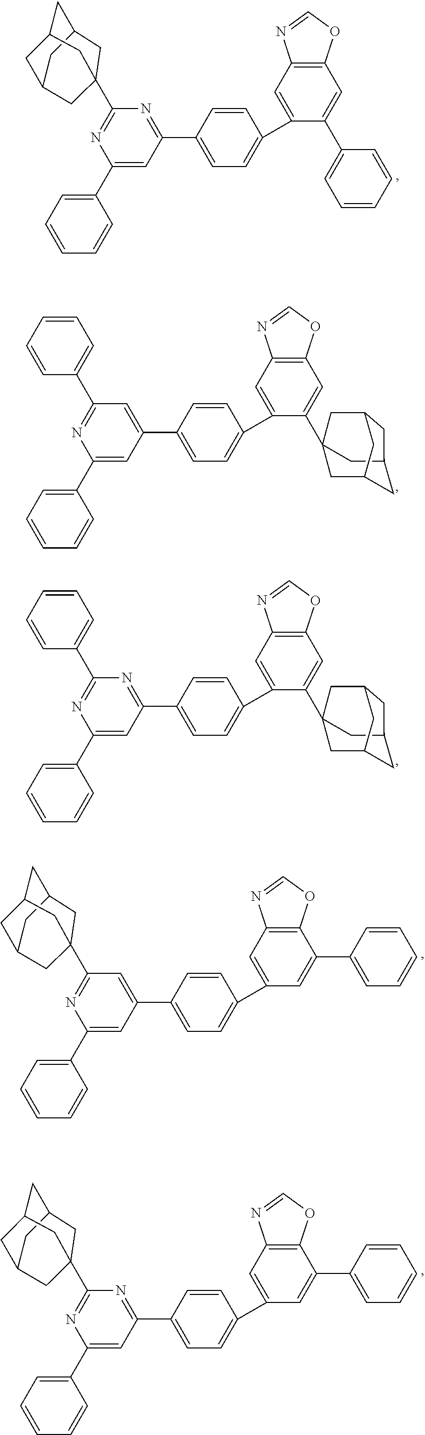

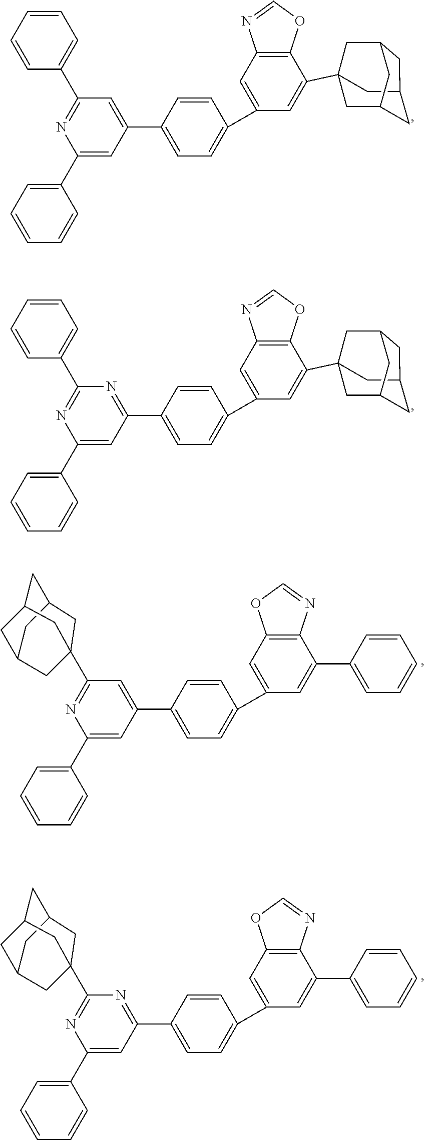

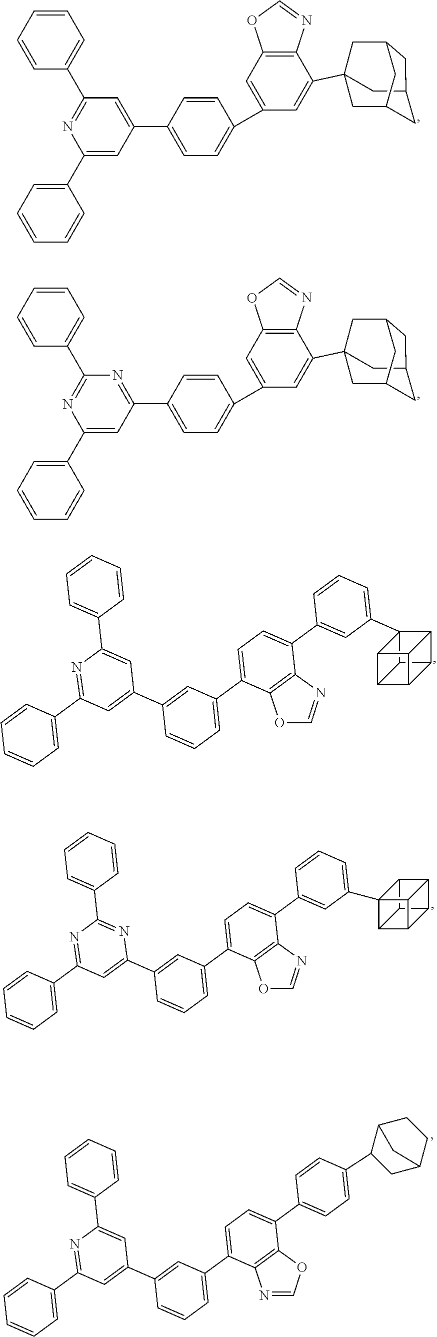

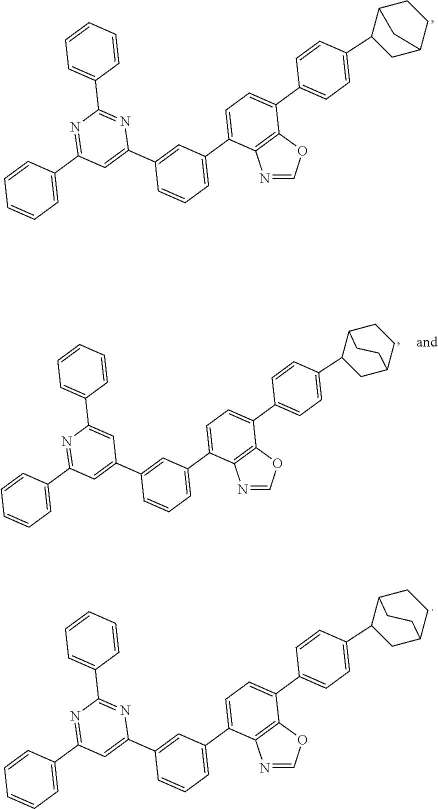

Optionally, the light emitting device further comprises an anode and a cathode, and the electron transport layer is located between the anode and the cathode;
the light emitting device further comprises a luminescent layer and an electron blocking layer, and the luminescent layer is located between the anode and the electron transport layer; and
the electron blocking layer is located between the anode and the luminescent layer.

In another aspect, there is provided a displaying device, wherein the displaying device comprises the light emitting device stated above.

In yet another aspect, there is provided a compound, wherein a general structural formula of the compound is:

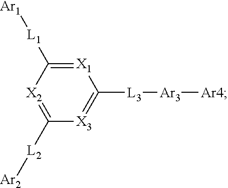

wherein X1 is C(Ra) or nitrogen, X2 is C(Rb) or nitrogen, X3 is C(Rc) or nitrogen, and at least one of X1, X2 and X3 is nitrogen, wherein each of Ra, Rb and Rc is independently any one of a group consisting of hydrogen, diplogen, fluorine, chlorine, an alkyl group whose carbon-atom quantity is 1-12, a halogenated alkyl group whose carbon-atom quantity is 1-12, an alkoxy group whose carbon-atom quantity is 1-12, a cycloalkyl group whose carbon-atom quantity is 3-10, C6-20 aryl, and a heteroaryl group whose carbon-atom quantity is 3-20;
each of L1, L2 and L3 is independently any one of a group consisting of a single bond, a substituted or unsubstituted arylene group whose carbon-atom quantity is 6-60, and a substituted or unsubstituted heteroarylene whose carbon-atom quantity is 1-60;
each of Ar1, Ar2 and Ar4 is independently any one of a group consisting of a substituted or unsubstituted alkyl group whose carbon-atom quantity is 1-12, a substituted or unsubstituted cycloalkyl group whose carbon-atom quantity is 3-10, a substituted or unsubstituted aralkyl group whose carbon-atom quantity is 7-30, a substituted or unsubstituted heterarylalkyl whose carbon-atom quantity is 2-60, a substituted or unsubstituted aryl whose carbon-atom quantity is 6-60, and a substituted or unsubstituted heteroaryl group whose carbon-atom quantity is 3-60, and at least one of Ar1, Ar2 and Ar4 is the three-dimensional group having a rigidity; and
a general structural formula of Ar3 is:

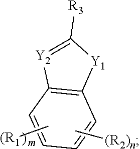

wherein Y1 is any one of carbon, oxygen, sulphur, selenium and N—R4;
Y2 is any one of carbon and nitrogen;
each of R1, R2 and R3 is independently any one of hydrogen, diplogen, halogen, amino, a nitrile group, nitro, C1-30 alkyl, C2-30 alkenyl, C2-30 alkynyl, C1-30 alkoxy, C6-30 aryloxy, and C6-50 aryl substituted by C6-30 aryl, or includes any one of at least one of heteroatoms selected from nitrogen, oxygen and sulphur and C2-50 heteroaryl unsubstituted or substituted by C6-30 aryloxy, and two of R1, R2 and R3 are directly connected to neighboring groups L3 and Ar4;
R4 is any one of a group consisting of hydrogen, diplogen, fluorine, chlorine, an alkyl group whose carbon-atom quantity is 1-12, a halogenated alkyl group whose carbon-atom quantity is 1-12, an alkoxy group whose carbon-atom quantity is 1-12, a cycloalkyl group whose carbon-atom quantity is 3-10, C6-20 aryl, and a heteroaryl group whose carbon-atom quantity is 3-20; and
each of m and n is independently 1 or 2.

Optionally, the general structural formula of the compound is any one of

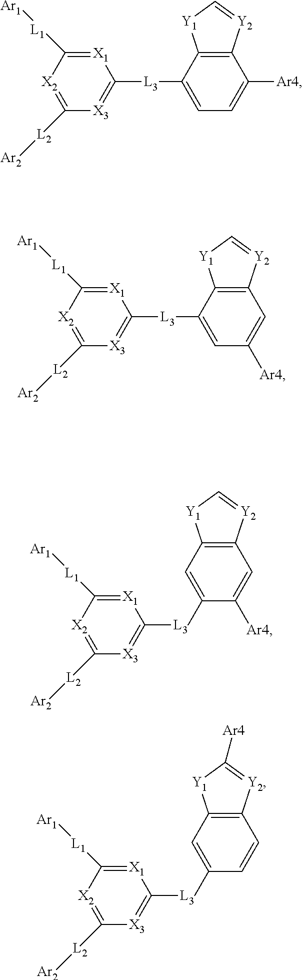

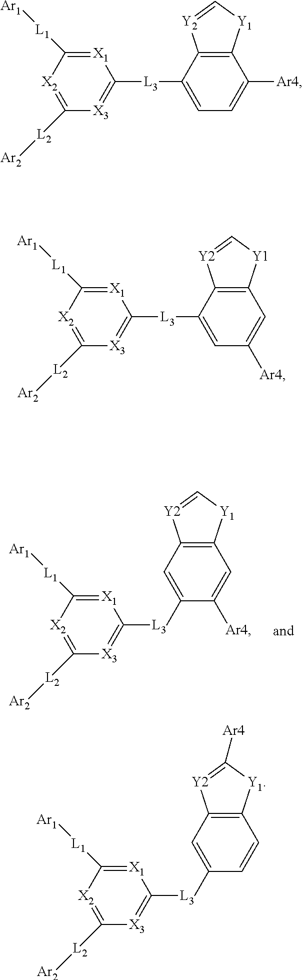

Optionally, a general structural formula of the three-dimensional group having a rigidity is any one of

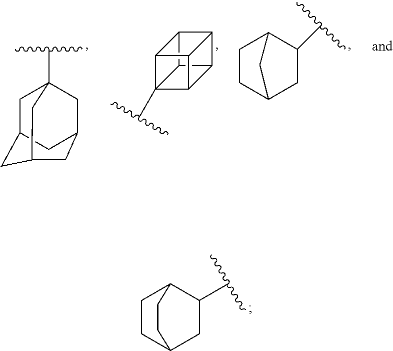

wherein

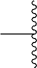

is a bond connected to a carbon atom.

Optionally, a chemical structural formula of the compound having a triazine group includes any one of

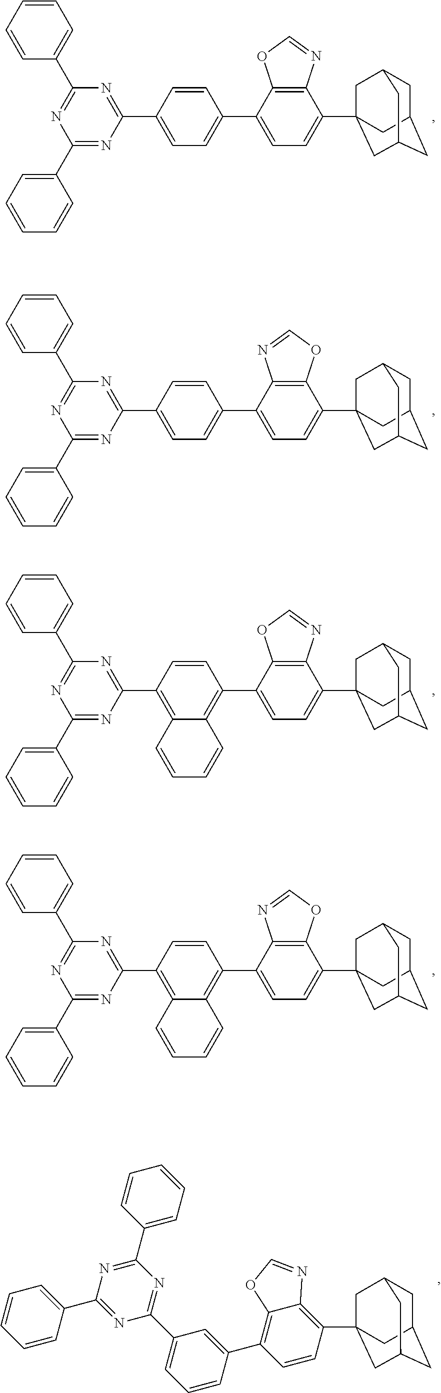 [SMILES: c1ccc(-c2nc(-c3ccccc3)nc(-c3ccc(-c4ccc(C56CC7CC(CC(C7)C5)C6)c5ncoc45)cc3)n2)cc1 ; c1ccc(-c2nc(-c3ccccc3)nc(-c3ccc(-c4ccc(C56CC7CC(CC(C7)C5)C6)c5ocnc45)cc3)n2)cc1 ; c1ccc(-c2nc(-c3ccccc3)nc(-c3ccc(-c4ccc(C56CC7CC(CC(C7)C5)C6)c5ncoc45)c4ccccc34)n2)cc1 ; c1ccc(-c2nc(-c3ccccc3)nc(-c3ccc(-c4ccc(C56CC7CC(CC(C7)C5)C6)c5ocnc45)c4ccccc34)n2)cc1 ; c1ccc(-c2nc(-c3ccccc3)nc(-c3cccc(-c4ccc(C56CC7CC(CC(C7)C5)C6)c5ncoc45)c3)n2)cc1]

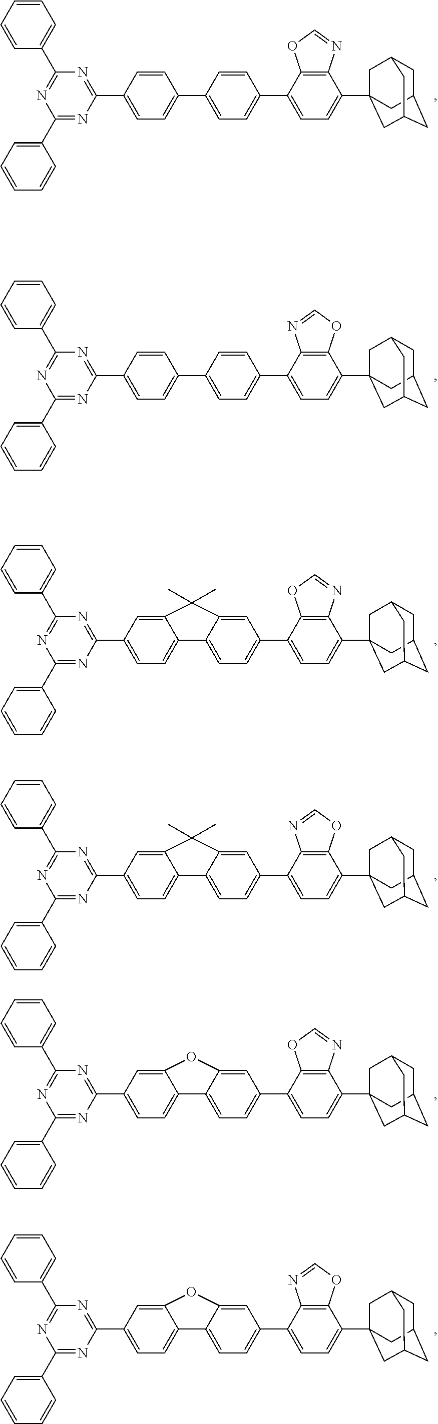

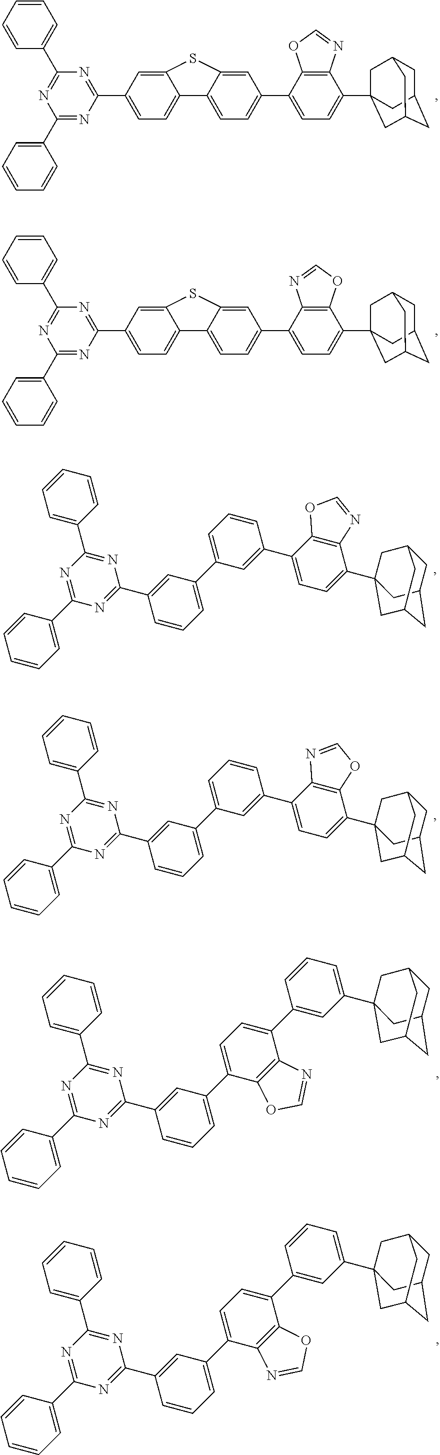

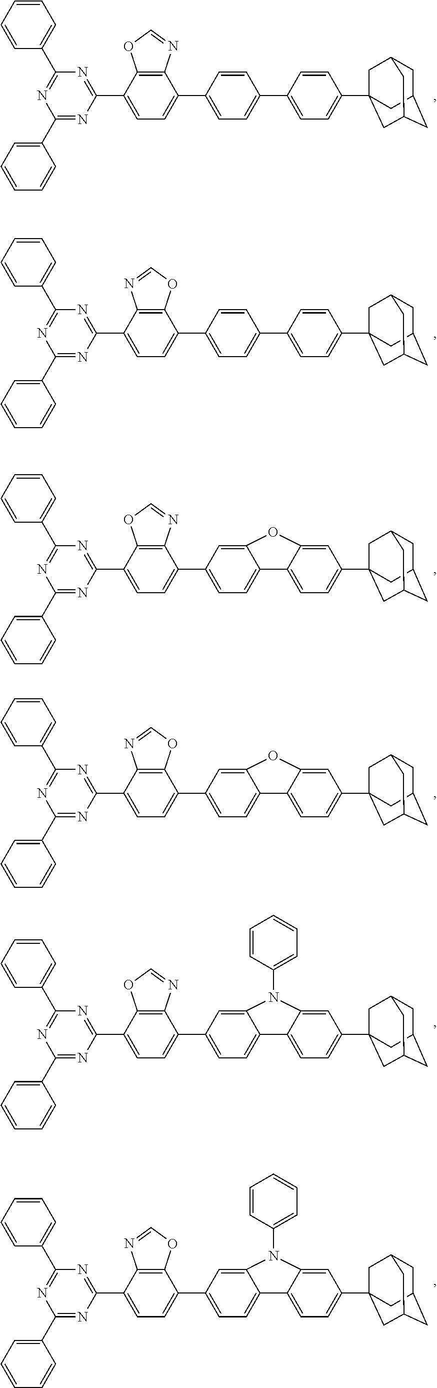

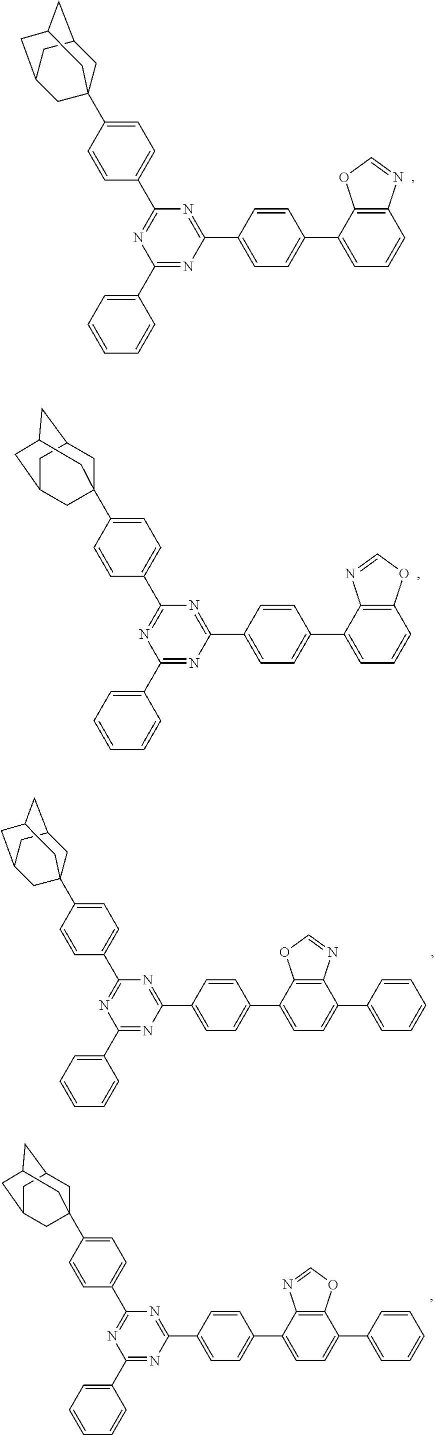 [SMILES: c1ccc(-c2nc(-c3ccc(-c4cccc5ncoc45)cc3)nc(-c3ccc(C45CC6CC(CC(C6)C4)C5)cc3)n2)cc1 ; c1ccc(-c2nc(-c3ccc(-c4cccc5ocnc45)cc3)nc(-c3ccc(C45CC6CC(CC(C6)C4)C5)cc3)n2)cc1 ; c1ccc(-c2nc(-c3ccc(-c4ccc(-c5ccccc5)c5ncoc45)cc3)nc(-c3ccc(C45CC6CC(CC(C6)C4)C5)cc3)n2)cc1 ; c1ccc(-c2nc(-c3ccc(-c4ccc(-c5ccccc5)c5ocnc45)cc3)nc(-c3ccc(C45CC6CC(CC(C6)C4)C5)cc3)n2)cc1]

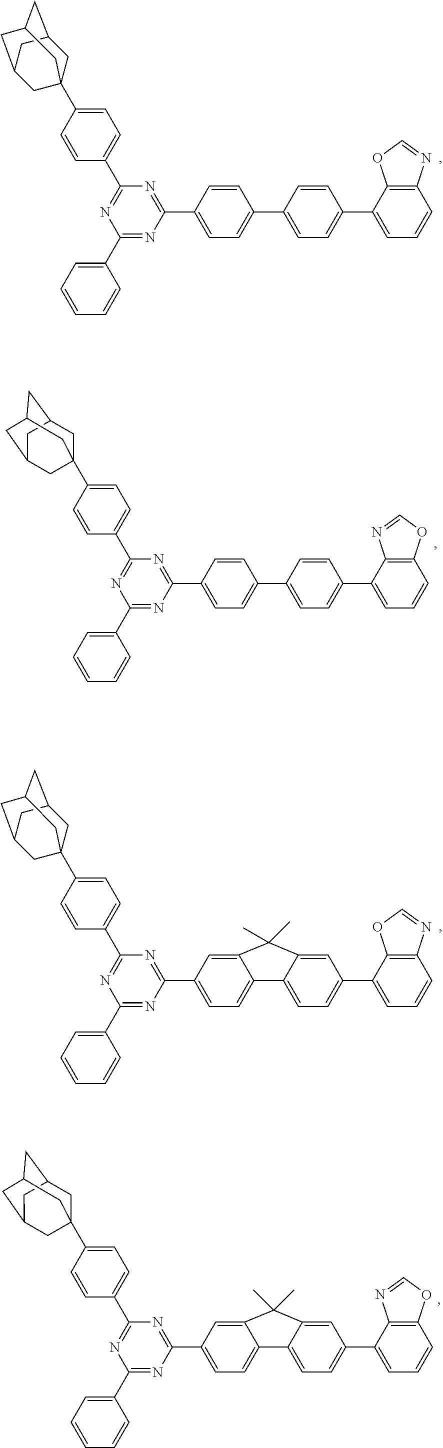 [SMILES: c1ccc(-c2nc(-c3ccc(-c4ccc(-c5cccc6ncoc56)cc4)cc3)nc(-c3ccc(C45CC6CC(CC(C6)C4)C5)cc3)n2)cc1 ; c1ccc(-c2nc(-c3ccc(-c4ccc(-c5cccc6ocnc56)cc4)cc3)nc(-c3ccc(C45CC6CC(CC(C6)C4)C5)cc3)n2)cc1 ; CC1(C)c2cc(-c3nc(-c4ccccc4)nc(-c4ccc(C56CC7CC(CC(C7)C5)C6)cc4)n3)ccc2-c2ccc(-c3cccc4ncoc34)cc21 ; CC1(C)c2cc(-c3nc(-c4ccccc4)nc(-c4ccc(C56CC7CC(CC(C7)C5)C6)cc4)n3)ccc2-c2ccc(-c3cccc4ocnc34)cc21]

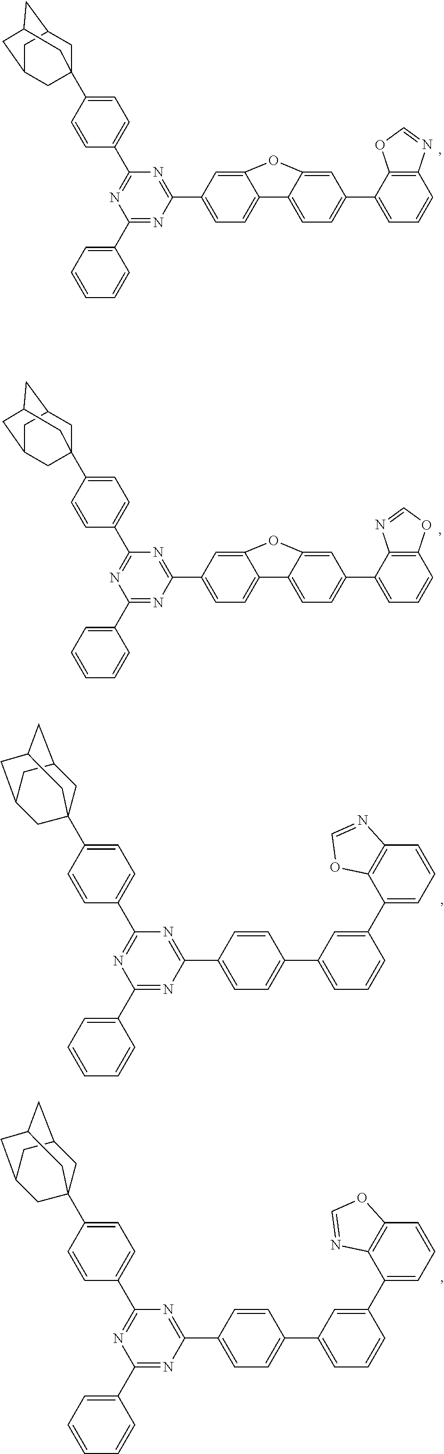 [SMILES: c1ccc(-c2nc(-c3ccc(C45CC6CC(CC(C6)C4)C5)cc3)nc(-c3ccc4c(c3)oc3cc(-c5cccc6ncoc56)ccc34)n2)cc1 ; c1ccc(-c2nc(-c3ccc(C45CC6CC(CC(C6)C4)C5)cc3)nc(-c3ccc4c(c3)oc3cc(-c5cccc6ocnc56)ccc34)n2)cc1 ; c1ccc(-c2nc(-c3ccc(-c4cccc(-c5cccc6ncoc56)c4)cc3)nc(-c3ccc(C45CC6CC(CC(C6)C4)C5)cc3)n2)cc1 ; c1ccc(-c2nc(-c3ccc(-c4cccc(-c5cccc6ocnc56)c4)cc3)nc(-c3ccc(C45CC6CC(CC(C6)C4)C5)cc3)n2)cc1]

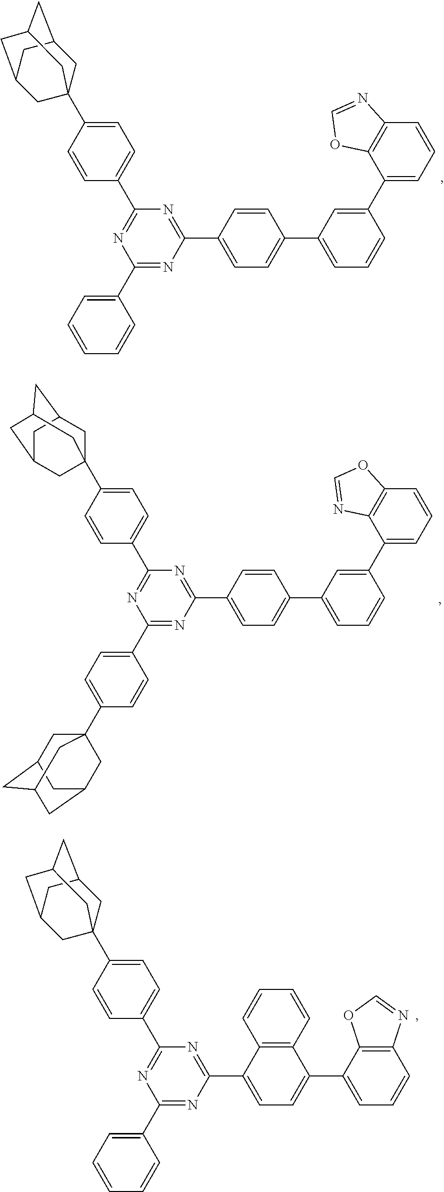

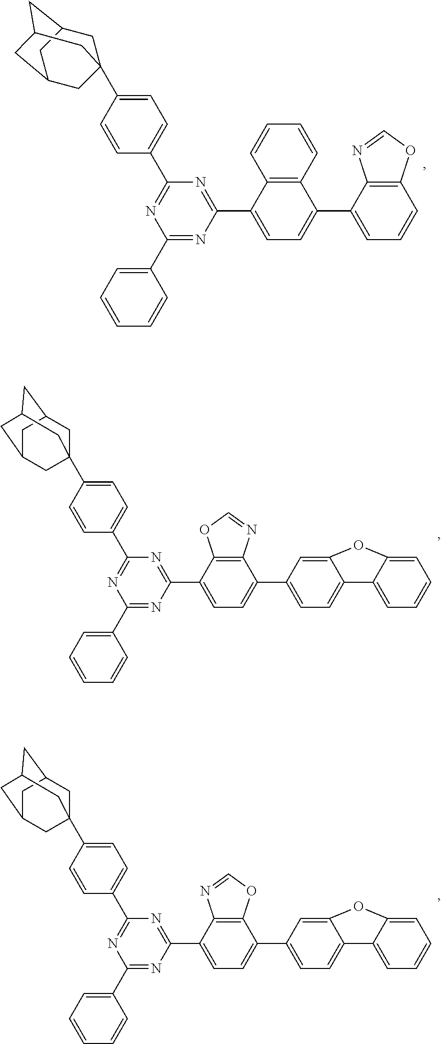 [SMILES: c1ccc(-c2nc(-c3ccc(C45CC6CC(CC(C6)C4)C5)cc3)nc(-c3ccc(-c4cccc5ocnc45)c4ccccc34)n2)cc1 ; c1ccc(-c2nc(-c3ccc(C45CC6CC(CC(C6)C4)C5)cc3)nc(-c3ccc(-c4ccc5c(c4)oc4ccccc45)c4ncoc34)n2)cc1 ; c1ccc(-c2nc(-c3ccc(C45CC6CC(CC(C6)C4)C5)cc3)nc(-c3ccc(-c4ccc5c(c4)oc4ccccc45)c4ocnc34)n2)cc1]

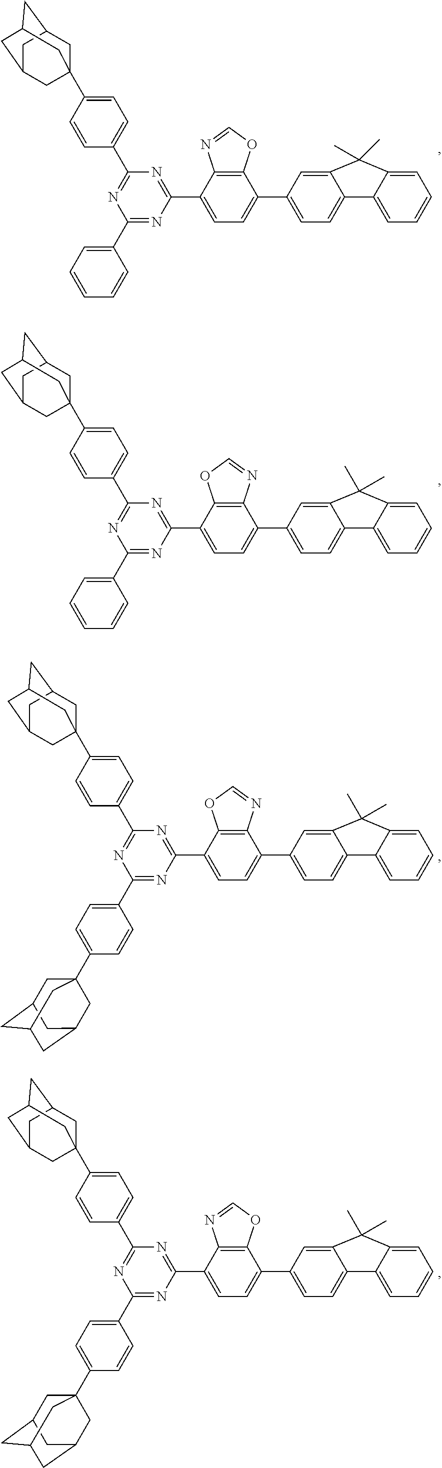

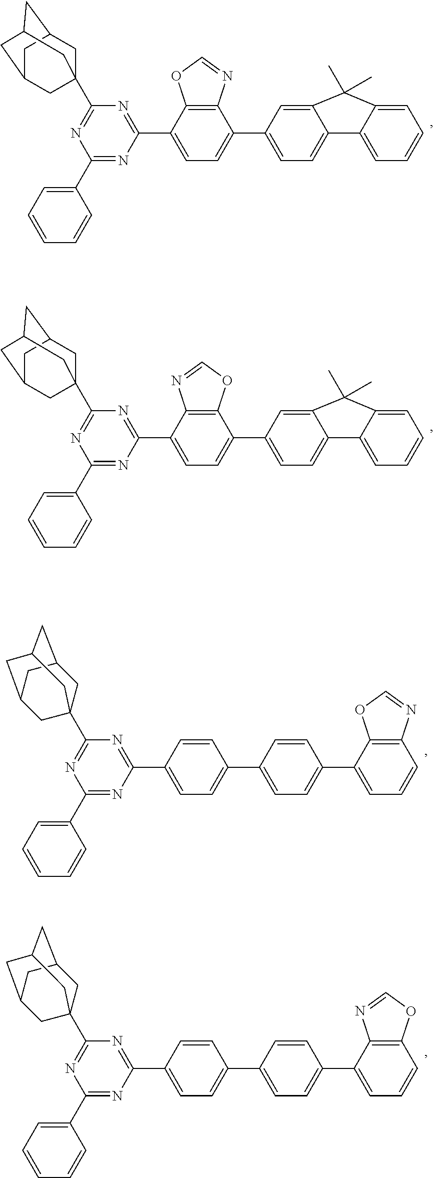 [SMILES: CC1(C)c2ccccc2-c2ccc(-c3ccc(-c4nc(-c5ccccc5)nc(C56CC7CC(CC(C7)C5)C6)n4)c4ocnc34)cc21 ; CC1(C)c2ccccc2-c2ccc(-c3ccc(-c4nc(-c5ccccc5)nc(C56CC7CC(CC(C7)C5)C6)n4)c4ncoc34)cc21 ; c1ccc(-c2nc(-c3ccc(-c4ccc(-c5cccc6ncoc56)cc4)cc3)nc(C34CC5CC(CC(C5)C3)C4)n2)cc1 ; c1ccc(-c2nc(-c3ccc(-c4ccc(-c5cccc6ocnc56)cc4)cc3)nc(C34CC5CC(CC(C5)C3)C4)n2)cc1]

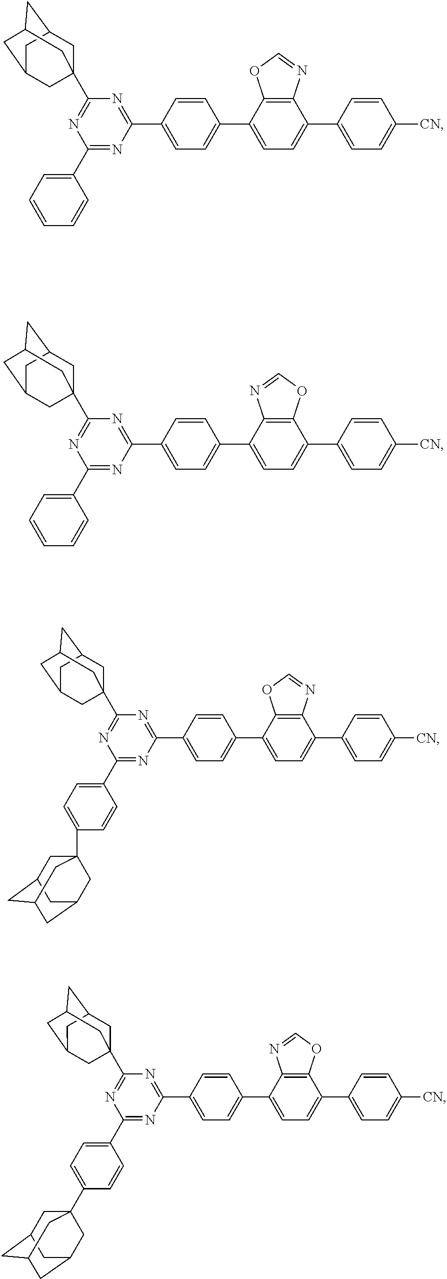 [SMILES: N#Cc1ccc(-c2ccc(-c3ccc(-c4nc(-c5ccccc5)nc(C56CC7CC(CC(C7)C5)C6)n4)cc3)c3ocnc23)cc1 ; N#Cc1ccc(-c2ccc(-c3ccc(-c4nc(-c5ccccc5)nc(C56CC7CC(CC(C7)C5)C6)n4)cc3)c3ncoc23)cc1 ; N#Cc1ccc(-c2ccc(-c3ccc(-c4nc(-c5ccc(C67CC8CC(CC(C8)C6)C7)cc5)nc(C56CC7CC(CC(C7)C5)C6)n4)cc3)c3ocnc23)cc1 ; N#Cc1ccc(-c2ccc(-c3ccc(-c4nc(-c5ccc(C67CC8CC(CC(C8)C6)C7)cc5)nc(C56CC7CC(CC(C7)C5)C6)n4)cc3)c3ncoc23)cc1]

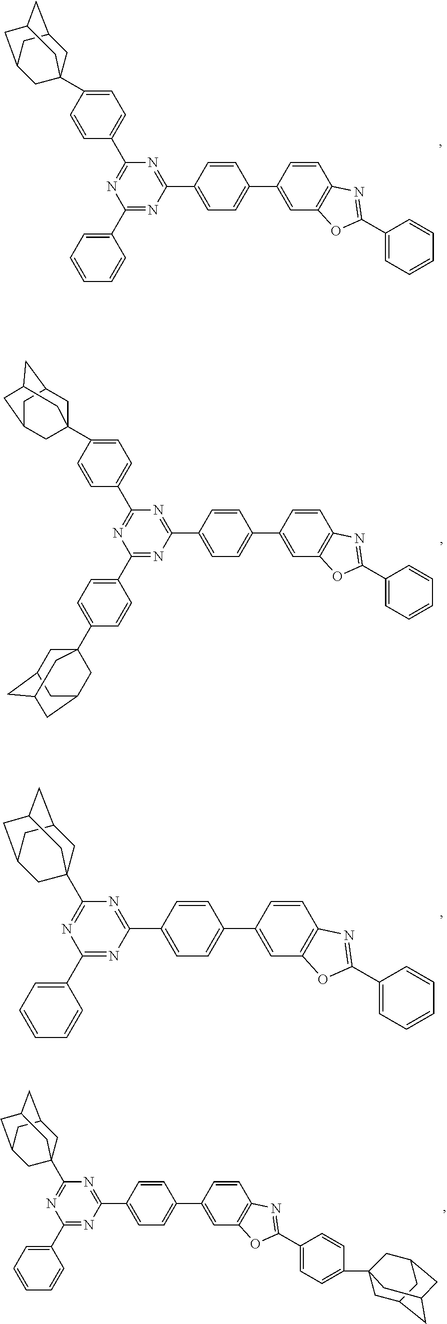 [SMILES: c1ccc(-c2nc(-c3ccc(-c4ccc5nc(-c6ccccc6)oc5c4)cc3)nc(-c3ccc(C45CC6CC(CC(C6)C4)C5)cc3)n2)cc1 ; c1ccc(-c2nc3ccc(-c4ccc(-c5nc(-c6ccc(C78CC9CC(CC(C9)C7)C8)cc6)nc(-c6ccc(C78CC9CC(CC(C9)C7)C8)cc6)n5)cc4)cc3o2)cc1 ; c1ccc(-c2nc(-c3ccc(-c4ccc5nc(-c6ccccc6)oc5c4)cc3)nc(C34CC5CC(CC(C5)C3)C4)n2)cc1 ; c1ccc(-c2nc(-c3ccc(-c4ccc5nc(-c6ccc(C78CC9CC(CC(C9)C7)C8)cc6)oc5c4)cc3)nc(C34CC5CC(CC(C5)C3)C4)n2)cc1]

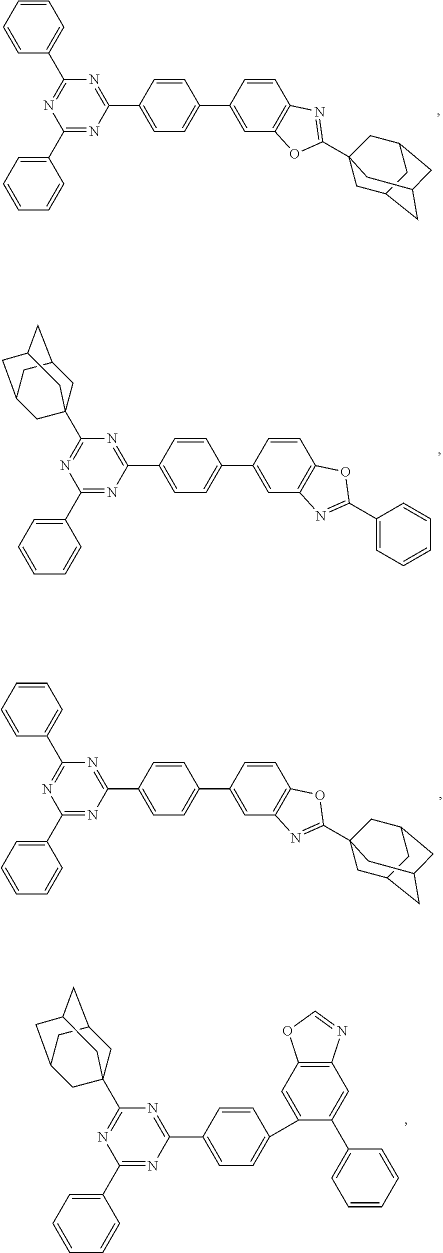 [SMILES: c1ccc(-c2nc(-c3ccccc3)nc(-c3ccc(-c4ccc5nc(C67CC8CC(CC(C8)C6)C7)oc5c4)cc3)n2)cc1 ; c1ccc(-c2nc(-c3ccc(-c4ccc5oc(-c6ccccc6)nc5c4)cc3)nc(C34CC5CC(CC(C5)C3)C4)n2)cc1 ; c1ccc(-c2nc(-c3ccccc3)nc(-c3ccc(-c4ccc5oc(C67CC8CC(CC(C8)C6)C7)nc5c4)cc3)n2)cc1 ; c1ccc(-c2nc(-c3ccc(-c4cc5ocnc5cc4-c4ccccc4)cc3)nc(C34CC5CC(CC(C5)C3)C4)n2)cc1]

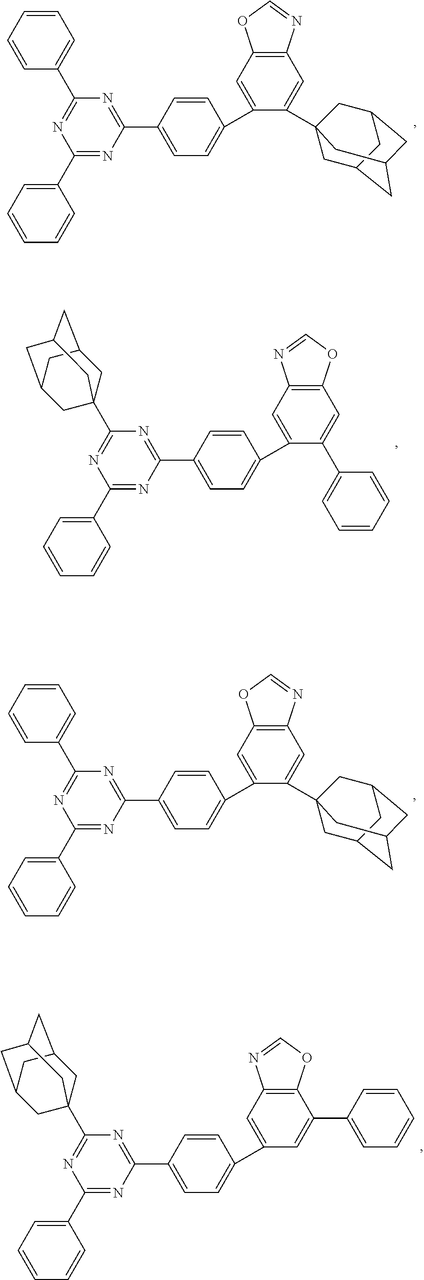 [SMILES: c1ccc(-c2nc(-c3ccccc3)nc(-c3ccc(-c4cc5ocnc5cc4C45CC6CC(CC(C6)C4)C5)cc3)n2)cc1 ; c1ccc(-c2nc(-c3ccc(-c4cc5ncoc5cc4-c4ccccc4)cc3)nc(C34CC5CC(CC(C5)C3)C4)n2)cc1 ; c1ccc(-c2nc(-c3ccccc3)nc(-c3ccc(-c4cc5ocnc5cc4C45CC6CC(CC(C6)C4)C5)cc3)n2)cc1 ; c1ccc(-c2nc(-c3ccc(-c4cc(-c5ccccc5)c5ocnc5c4)cc3)nc(C34CC5CC(CC(C5)C3)C4)n2)cc1]

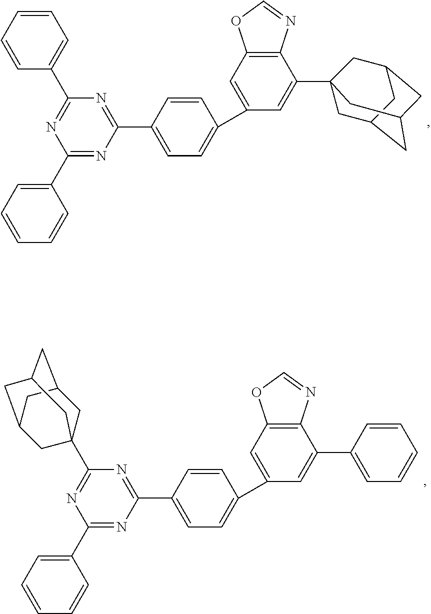 [SMILES: c1ccc(-c2nc(-c3ccccc3)nc(-c3ccc(-c4cc(C56CC7CC(CC(C7)C5)C6)c5ncoc5c4)cc3)n2)cc1 ; c1ccc(-c2nc(-c3ccc(-c4cc(-c5ccccc5)c5ncoc5c4)cc3)nc(C34CC5CC(CC(C5)C3)C4)n2)cc1]

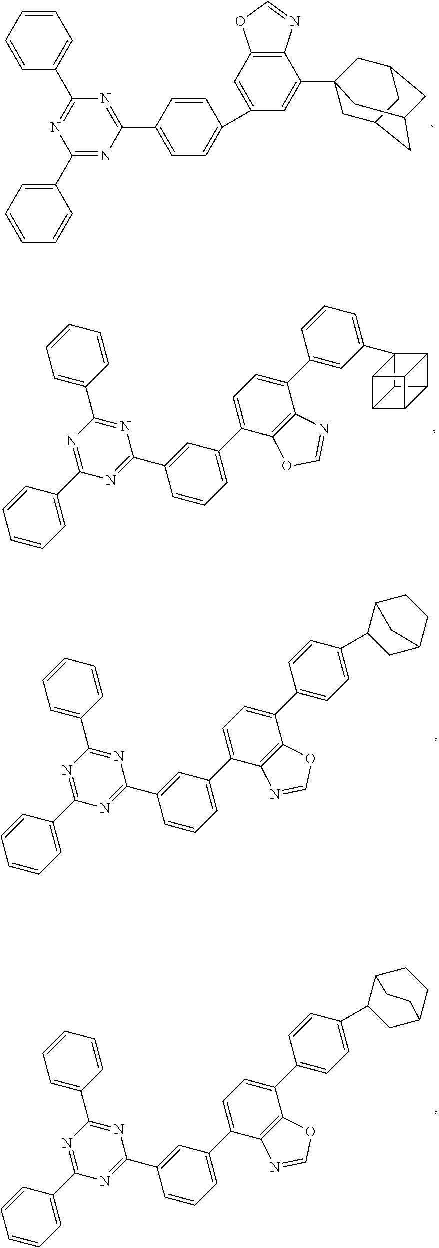 [SMILES: c1ccc(-c2nc(-c3ccccc3)nc(-c3ccc(-c4cc(C56CC7CC(CC(C7)C5)C6)c5ncoc5c4)cc3)n2)cc1 ; c1ccc(-c2nc(-c3ccccc3)nc(-c3cccc(-c4ccc(-c5cccc(C67C8C9C%10C8C6C%10C97)c5)c5ncoc45)c3)n2)cc1 ; c1ccc(-c2nc(-c3ccccc3)nc(-c3cccc(-c4ccc(-c5ccc(C6CC7CCC6C7)cc5)c5ocnc45)c3)n2)cc1 ; c1ccc(-c2nc(-c3ccccc3)nc(-c3cccc(-c4ccc(-c5ccc(C6CC7CCC6CC7)cc5)c5ocnc45)c3)n2)cc1]

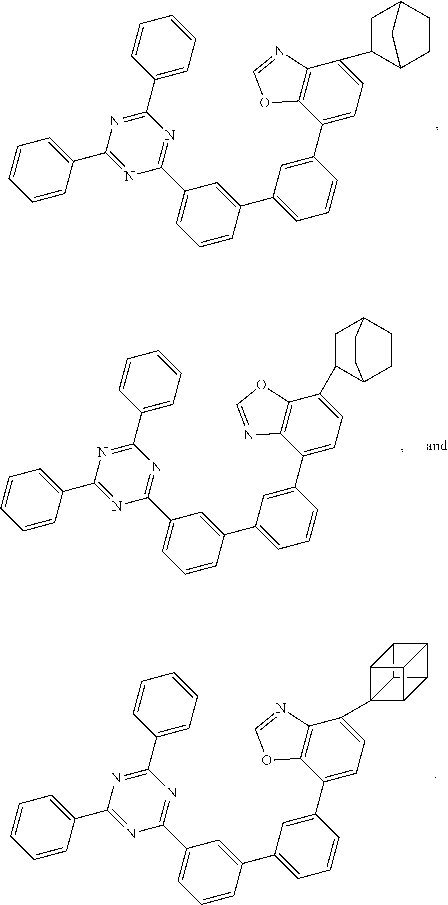 [SMILES: c1ccc(-c2nc(-c3ccccc3)nc(-c3cccc(-c4cccc(-c5ccc(C6CC7CCC6C7)c6ncoc56)c4)c3)n2)cc1 ; c1ccc(-c2nc(-c3ccccc3)nc(-c3cccc(-c4cccc(-c5ccc(C6CC7CCC6CC7)c6ocnc56)c4)c3)n2)cc1 ; c1ccc(-c2nc(-c3ccccc3)nc(-c3cccc(-c4cccc(-c5ccc(C67C8C9C%10C8C6C%10C97)c6ncoc56)c4)c3)n2)cc1]

Optionally, a chemical structural formula of the compound includes any one of

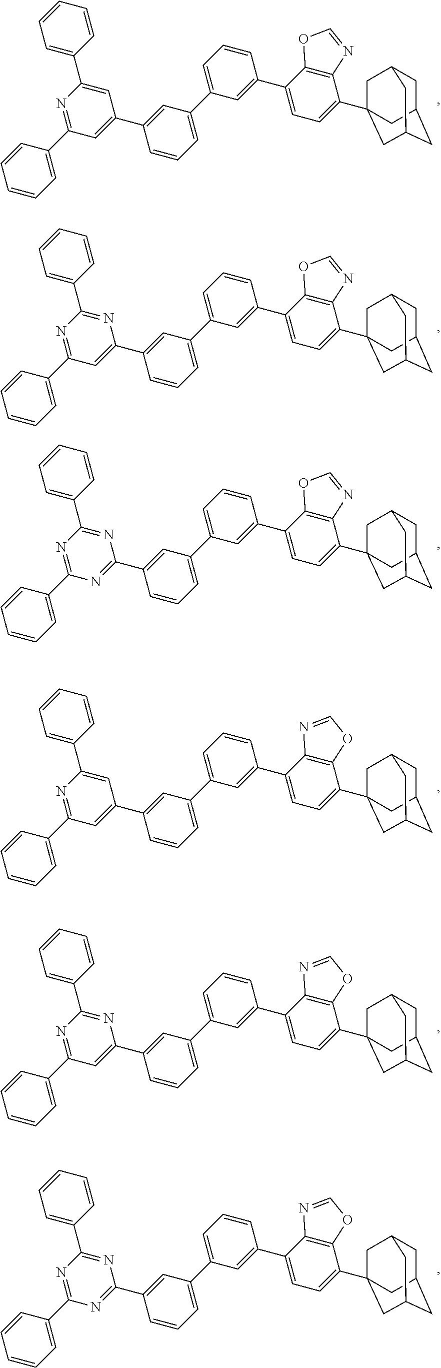

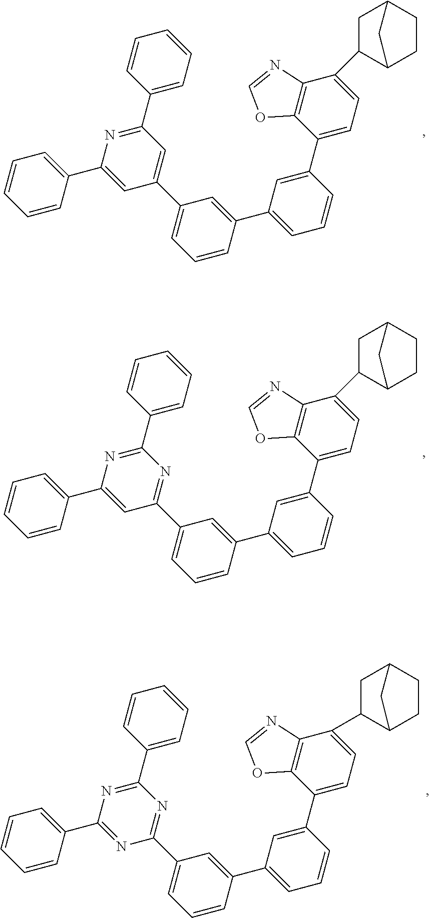 [SMILES: c1ccc(-c2cc(-c3cccc(-c4cccc(-c5ccc(C6CC7CCC6C7)c6ncoc56)c4)c3)cc(-c3ccccc3)n2)cc1 ; c1ccc(-c2cc(-c3cccc(-c4cccc(-c5ccc(C6CC7CCC6C7)c6ncoc56)c4)c3)nc(-c3ccccc3)n2)cc1 ; c1ccc(-c2nc(-c3ccccc3)nc(-c3cccc(-c4cccc(-c5ccc(C6CC7CCC6C7)c6ncoc56)c4)c3)n2)cc1]

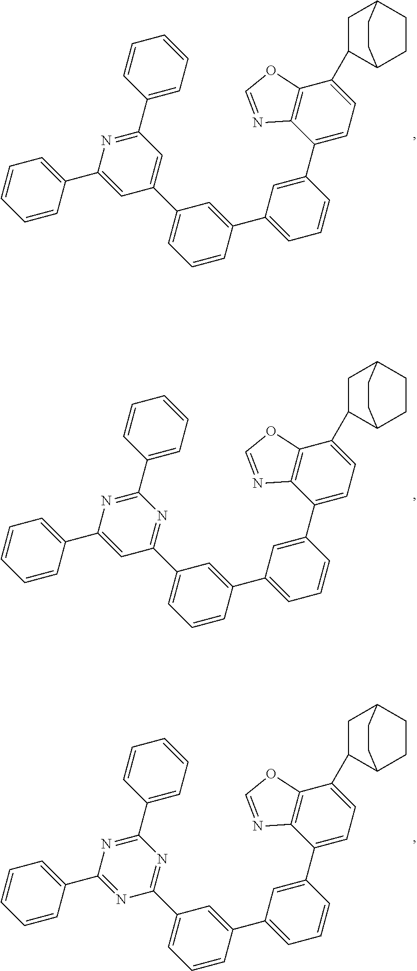 [SMILES: c1ccc(-c2cc(-c3cccc(-c4cccc(-c5ccc(C6CC7CCC6CC7)c6ocnc56)c4)c3)cc(-c3ccccc3)n2)cc1 ; c1ccc(-c2cc(-c3cccc(-c4cccc(-c5ccc(C6CC7CCC6CC7)c6ocnc56)c4)c3)nc(-c3ccccc3)n2)cc1 ; c1ccc(-c2nc(-c3ccccc3)nc(-c3cccc(-c4cccc(-c5ccc(C6CC7CCC6CC7)c6ocnc56)c4)c3)n2)cc1]

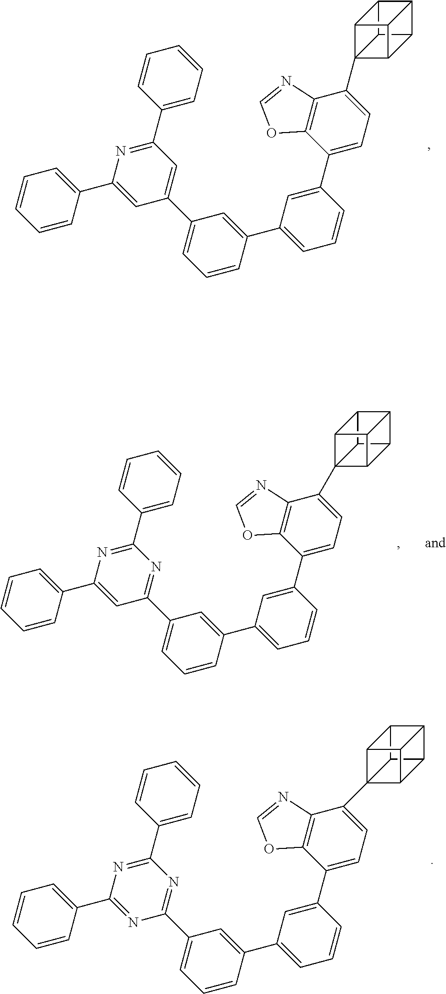 [SMILES: c1ccc(-c2cc(-c3cccc(-c4cccc(-c5ccc(C67C8C9C%10C8C6C%10C97)c6ncoc56)c4)c3)cc(-c3ccccc3)n2)cc1 ; c1ccc(-c2cc(-c3cccc(-c4cccc(-c5ccc(C67C8C9C%10C8C6C%10C97)c6ncoc56)c4)c3)nc(-c3ccccc3)n2)cc1 ; c1ccc(-c2nc(-c3ccccc3)nc(-c3cccc(-c4cccc(-c5ccc(C67C8C9C%10C8C6C%10C97)c6ncoc56)c4)c3)n2)cc1]

Optionally, a chemical structural formula of the compound having cyano includes any one of

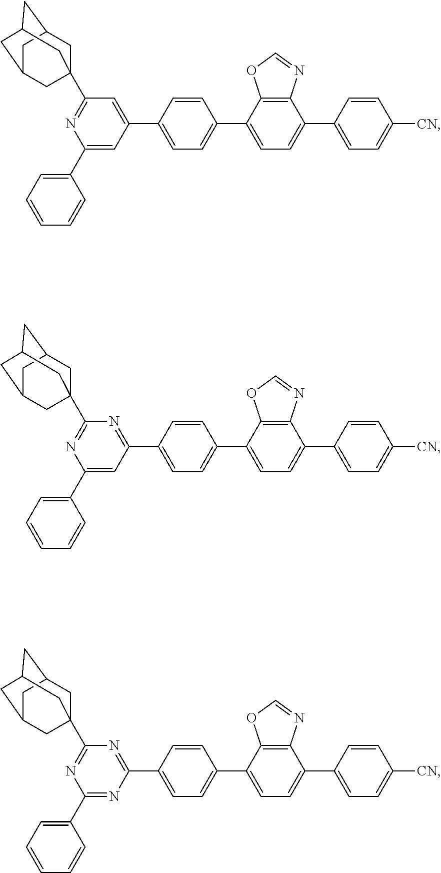 [SMILES: N#Cc1ccc(-c2ccc(-c3ccc(-c4cc(-c5ccccc5)nc(C56CC7CC(CC(C7)C5)C6)c4)cc3)c3ocnc23)cc1 ; N#Cc1ccc(-c2ccc(-c3ccc(-c4cc(-c5ccccc5)nc(C56CC7CC(CC(C7)C5)C6)n4)cc3)c3ocnc23)cc1 ; N#Cc1ccc(-c2ccc(-c3ccc(-c4nc(-c5ccccc5)nc(C56CC7CC(CC(C7)C5)C6)n4)cc3)c3ocnc23)cc1]

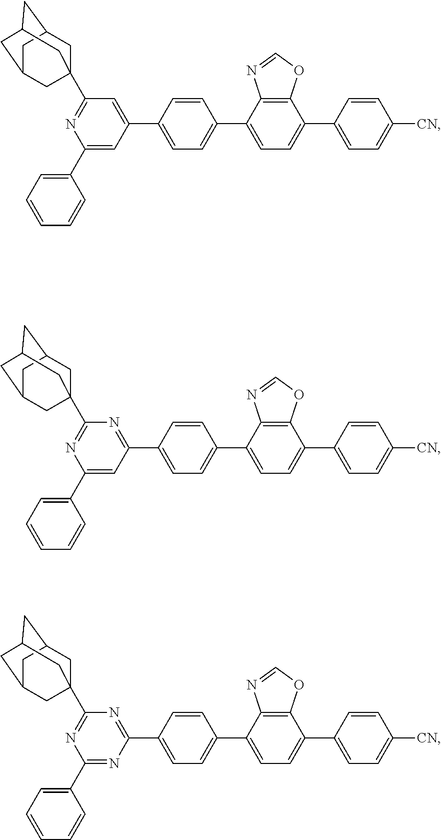 [SMILES: N#Cc1ccc(-c2ccc(-c3ccc(-c4cc(-c5ccccc5)nc(C56CC7CC(CC(C7)C5)C6)c4)cc3)c3ncoc23)cc1 ; N#Cc1ccc(-c2ccc(-c3ccc(-c4cc(-c5ccccc5)nc(C56CC7CC(CC(C7)C5)C6)n4)cc3)c3ncoc23)cc1 ; N#Cc1ccc(-c2ccc(-c3ccc(-c4nc(-c5ccccc5)nc(C56CC7CC(CC(C7)C5)C6)n4)cc3)c3ncoc23)cc1]

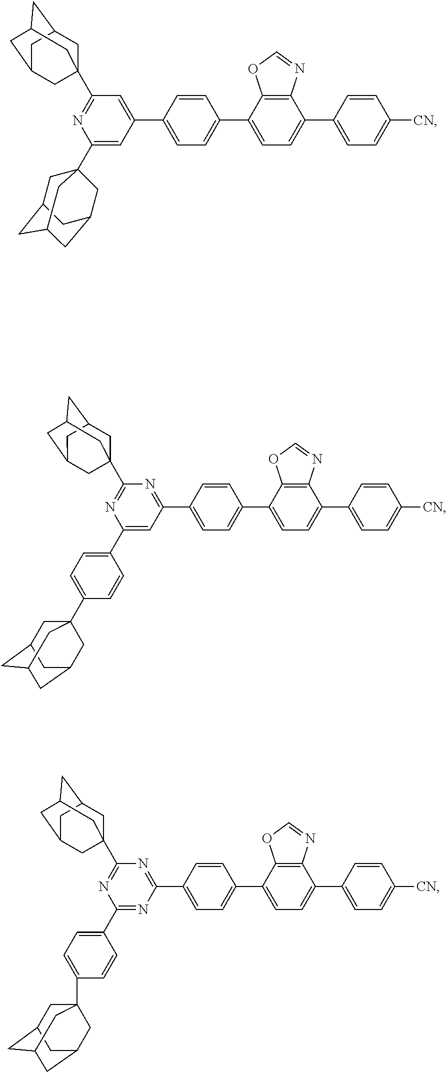 [SMILES: N#Cc1ccc(-c2ccc(-c3ccc(-c4cc(C56CC7CC(CC(C7)C5)C6)nc(C56CC7CC(CC(C7)C5)C6)c4)cc3)c3ocnc23)cc1 ; N#Cc1ccc(-c2ccc(-c3ccc(-c4cc(-c5ccc(C67CC8CC(CC(C8)C6)C7)cc5)nc(C56CC7CC(CC(C7)C5)C6)n4)cc3)c3ocnc23)cc1 ; N#Cc1ccc(-c2ccc(-c3ccc(-c4nc(-c5ccc(C67CC8CC(CC(C8)C6)C7)cc5)nc(C56CC7CC(CC(C7)C5)C6)n4)cc3)c3ocnc23)cc1]

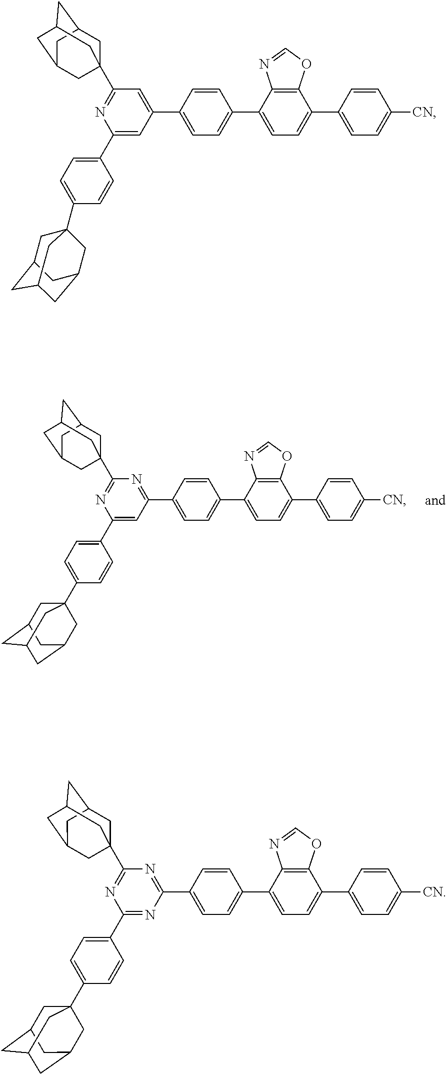 [SMILES: N#Cc1ccc(-c2ccc(-c3ccc(-c4cc(-c5ccc(C67CC8CC(CC(C8)C6)C7)cc5)nc(C56CC7CC(CC(C7)C5)C6)c4)cc3)c3ncoc23)cc1 ; N#Cc1ccc(-c2ccc(-c3ccc(-c4cc(-c5ccc(C67CC8CC(CC(C8)C6)C7)cc5)nc(C56CC7CC(CC(C7)C5)C6)n4)cc3)c3ncoc23)cc1 ; N#Cc1ccc(-c2ccc(-c3ccc(-c4nc(-c5ccc(C67CC8CC(CC(C8)C6)C7)cc5)nc(C56CC7CC(CC(C7)C5)C6)n4)cc3)c3ncoc23)cc1]

Optionally, a chemical structural formula of the compound includes any one of

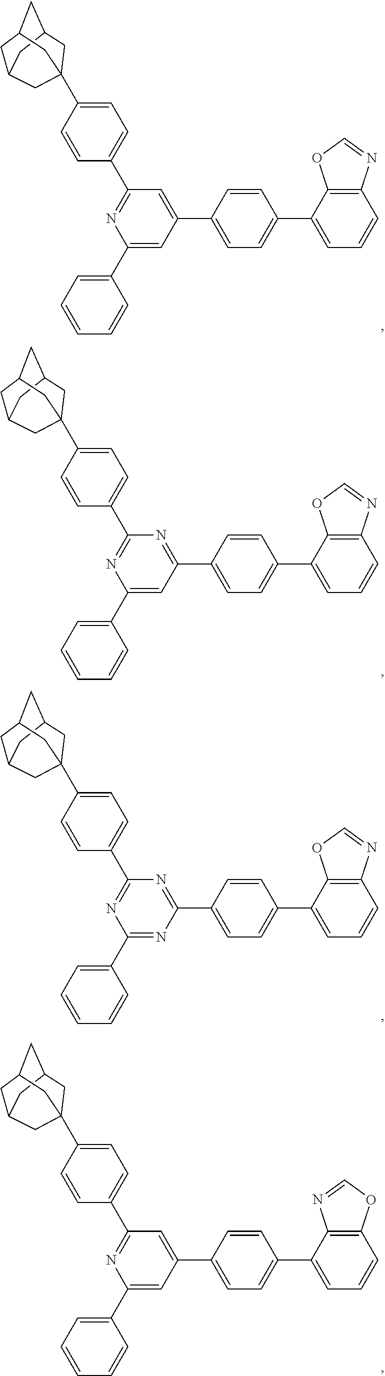 [SMILES: c1ccc(-c2cc(-c3ccc(-c4cccc5ncoc45)cc3)cc(-c3ccc(C45CC6CC(CC(C6)C4)C5)cc3)n2)cc1 ; c1ccc(-c2cc(-c3ccc(-c4cccc5ncoc45)cc3)nc(-c3ccc(C45CC6CC(CC(C6)C4)C5)cc3)n2)cc1 ; c1ccc(-c2nc(-c3ccc(-c4cccc5ncoc45)cc3)nc(-c3ccc(C45CC6CC(CC(C6)C4)C5)cc3)n2)cc1 ; c1ccc(-c2cc(-c3ccc(-c4cccc5ocnc45)cc3)cc(-c3ccc(C45CC6CC(CC(C6)C4)C5)cc3)n2)cc1]

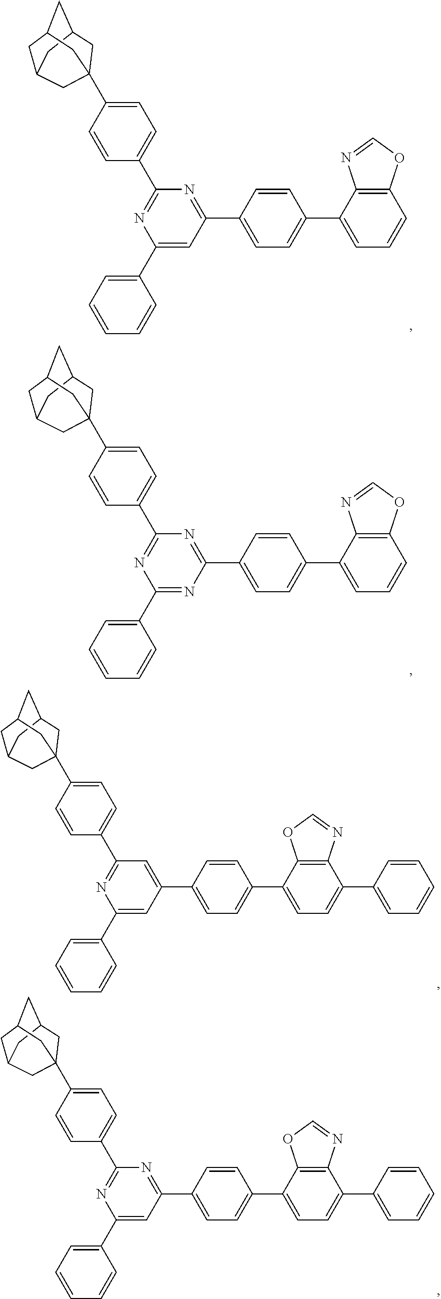

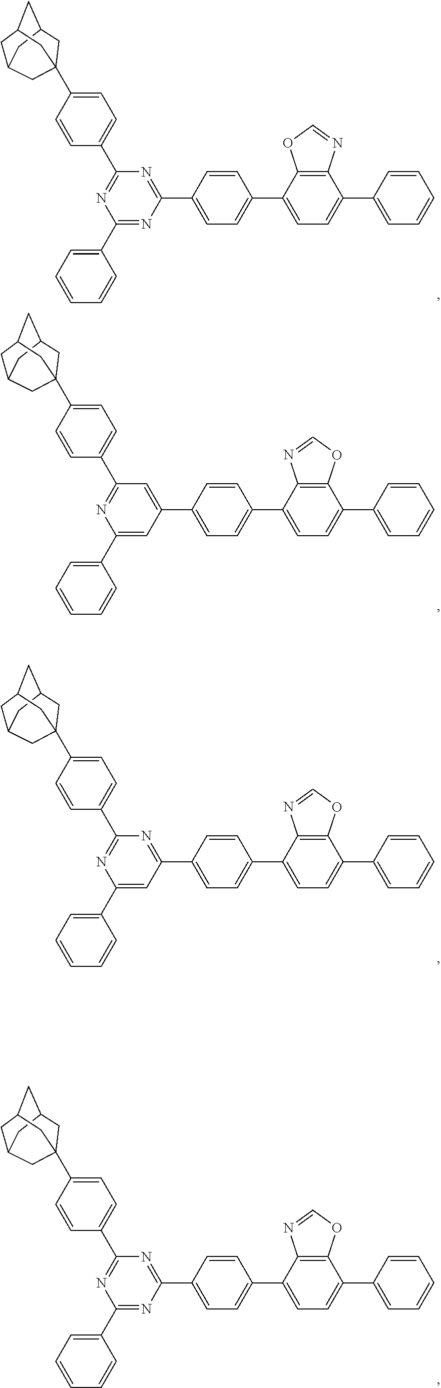 [SMILES: c1ccc(-c2nc(-c3ccc(-c4ccc(-c5ccccc5)c5ncoc45)cc3)nc(-c3ccc(C45CC6CC(CC(C6)C4)C5)cc3)n2)cc1 ; c1ccc(-c2cc(-c3ccc(-c4ccc(-c5ccccc5)c5ocnc45)cc3)cc(-c3ccc(C45CC6CC(CC(C6)C4)C5)cc3)n2)cc1 ; c1ccc(-c2cc(-c3ccc(-c4ccc(-c5ccccc5)c5ocnc45)cc3)nc(-c3ccc(C45CC6CC(CC(C6)C4)C5)cc3)n2)cc1 ; c1ccc(-c2nc(-c3ccc(-c4ccc(-c5ccccc5)c5ocnc45)cc3)nc(-c3ccc(C45CC6CC(CC(C6)C4)C5)cc3)n2)cc1]

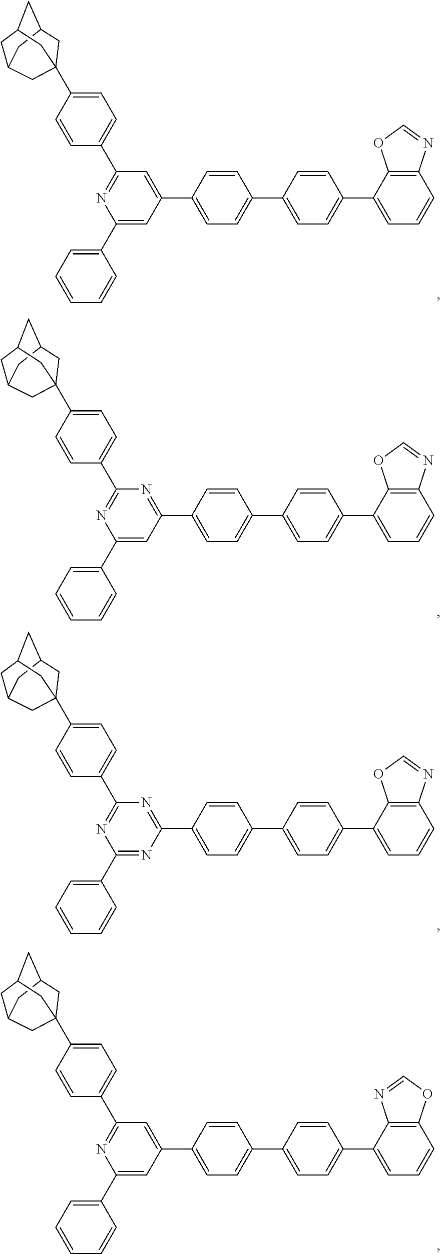 [SMILES: c1ccc(-c2cc(-c3ccc(-c4ccc(-c5cccc6ncoc56)cc4)cc3)cc(-c3ccc(C45CC6CC(CC(C6)C4)C5)cc3)n2)cc1 ; c1ccc(-c2cc(-c3ccc(-c4ccc(-c5cccc6ncoc56)cc4)cc3)nc(-c3ccc(C45CC6CC(CC(C6)C4)C5)cc3)n2)cc1 ; c1ccc(-c2nc(-c3ccc(-c4ccc(-c5cccc6ncoc56)cc4)cc3)nc(-c3ccc(C45CC6CC(CC(C6)C4)C5)cc3)n2)cc1 ; c1ccc(-c2cc(-c3ccc(-c4ccc(-c5cccc6ocnc56)cc4)cc3)cc(-c3ccc(C45CC6CC(CC(C6)C4)C5)cc3)n2)cc1]

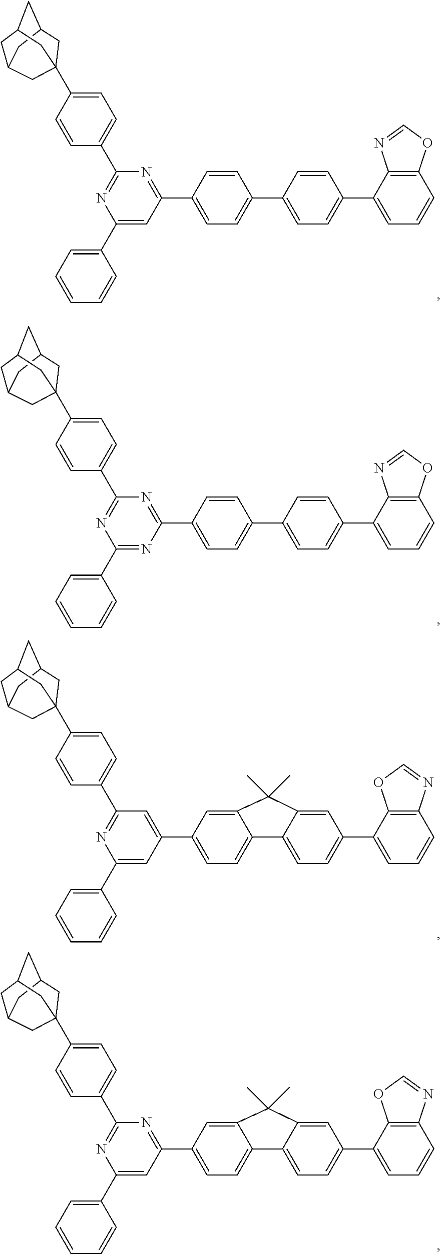 [SMILES: c1ccc(-c2cc(-c3ccc(-c4ccc(-c5cccc6ocnc56)cc4)cc3)nc(-c3ccc(C45CC6CC(CC(C6)C4)C5)cc3)n2)cc1 ; c1ccc(-c2nc(-c3ccc(-c4ccc(-c5cccc6ocnc56)cc4)cc3)nc(-c3ccc(C45CC6CC(CC(C6)C4)C5)cc3)n2)cc1 ; CC1(C)c2cc(-c3cc(-c4ccccc4)nc(-c4ccc(C56CC7CC(CC(C7)C5)C6)cc4)c3)ccc2-c2ccc(-c3cccc4ncoc34)cc21 ; CC1(C)c2cc(-c3cc(-c4ccccc4)nc(-c4ccc(C56CC7CC(CC(C7)C5)C6)cc4)n3)ccc2-c2ccc(-c3cccc4ncoc34)cc21]

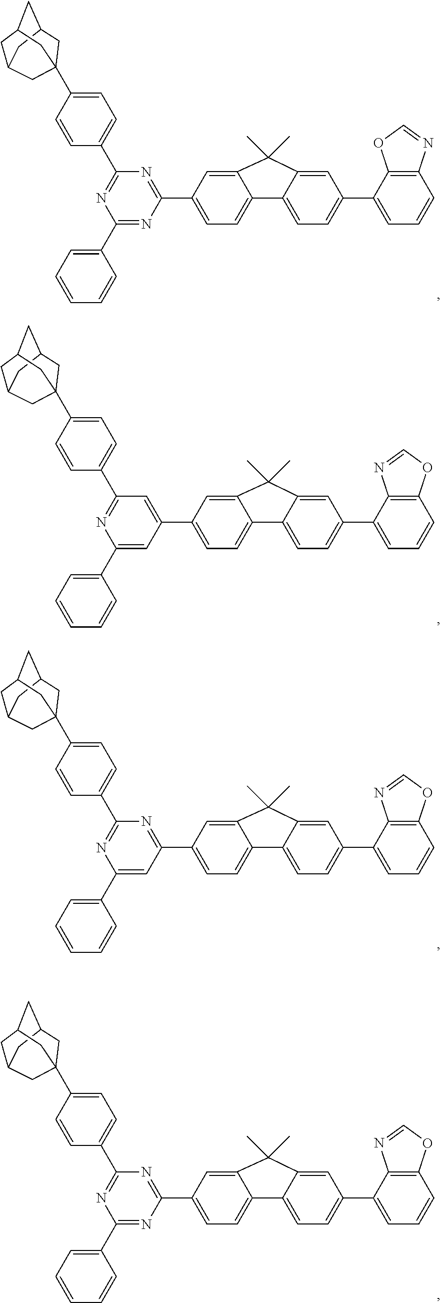 [SMILES: CC1(C)c2cc(-c3nc(-c4ccccc4)nc(-c4ccc(C56CC7CC(CC(C7)C5)C6)cc4)n3)ccc2-c2ccc(-c3cccc4ncoc34)cc21 ; CC1(C)c2cc(-c3cc(-c4ccccc4)nc(-c4ccc(C56CC7CC(CC(C7)C5)C6)cc4)c3)ccc2-c2ccc(-c3cccc4ocnc34)cc21 ; CC1(C)c2cc(-c3cc(-c4ccccc4)nc(-c4ccc(C56CC7CC(CC(C7)C5)C6)cc4)n3)ccc2-c2ccc(-c3cccc4ocnc34)cc21 ; CC1(C)c2cc(-c3nc(-c4ccccc4)nc(-c4ccc(C56CC7CC(CC(C7)C5)C6)cc4)n3)ccc2-c2ccc(-c3cccc4ocnc34)cc21]

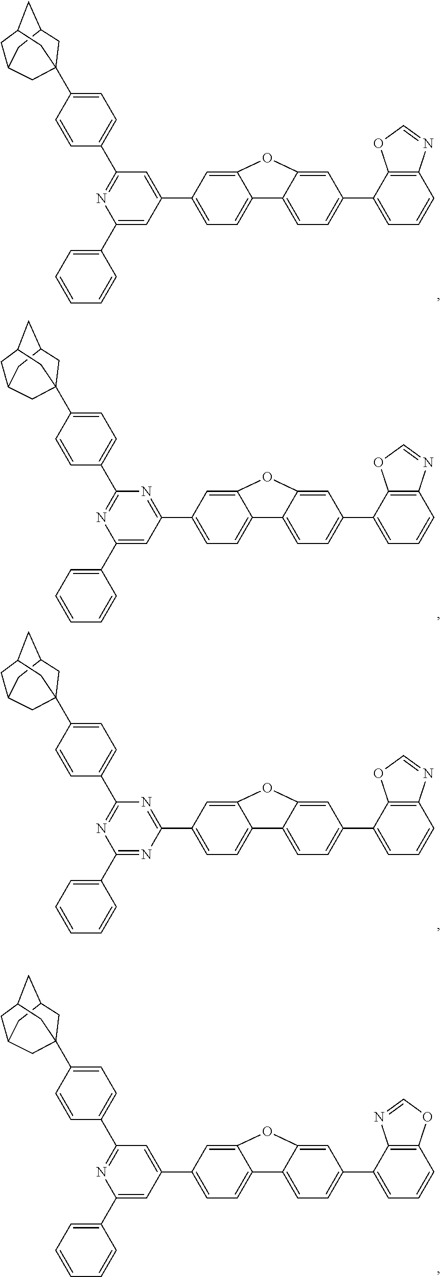 [SMILES: c1ccc(-c2cc(-c3ccc4c(c3)oc3cc(-c5cccc6ncoc56)ccc34)cc(-c3ccc(C45CC6CC(CC(C6)C4)C5)cc3)n2)cc1 ; c1ccc(-c2cc(-c3ccc4c(c3)oc3cc(-c5cccc6ncoc56)ccc34)nc(-c3ccc(C45CC6CC(CC(C6)C4)C5)cc3)n2)cc1 ; c1ccc(-c2nc(-c3ccc(C45CC6CC(CC(C6)C4)C5)cc3)nc(-c3ccc4c(c3)oc3cc(-c5cccc6ncoc56)ccc34)n2)cc1 ; c1ccc(-c2cc(-c3ccc4c(c3)oc3cc(-c5cccc6ocnc56)ccc34)cc(-c3ccc(C45CC6CC(CC(C6)C4)C5)cc3)n2)cc1]

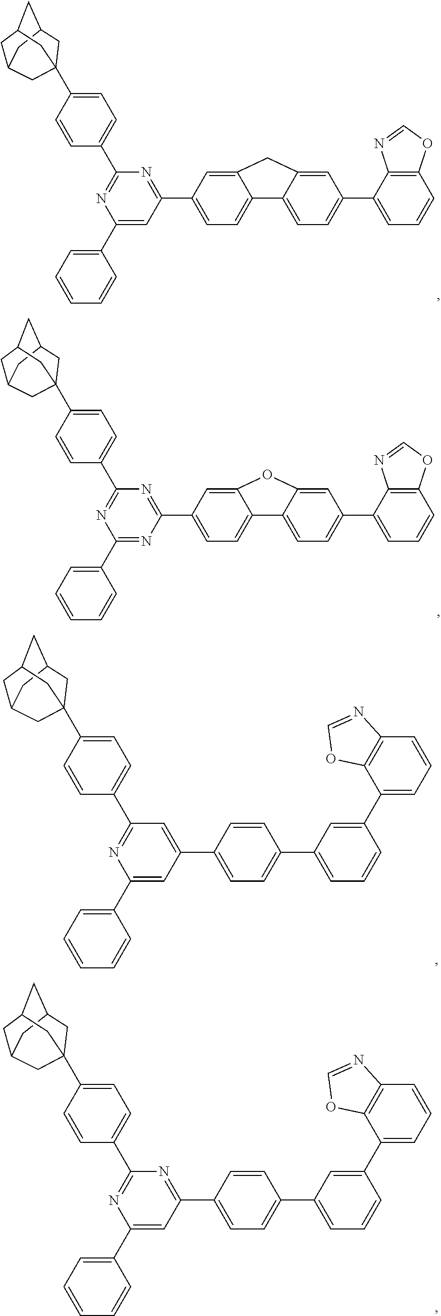

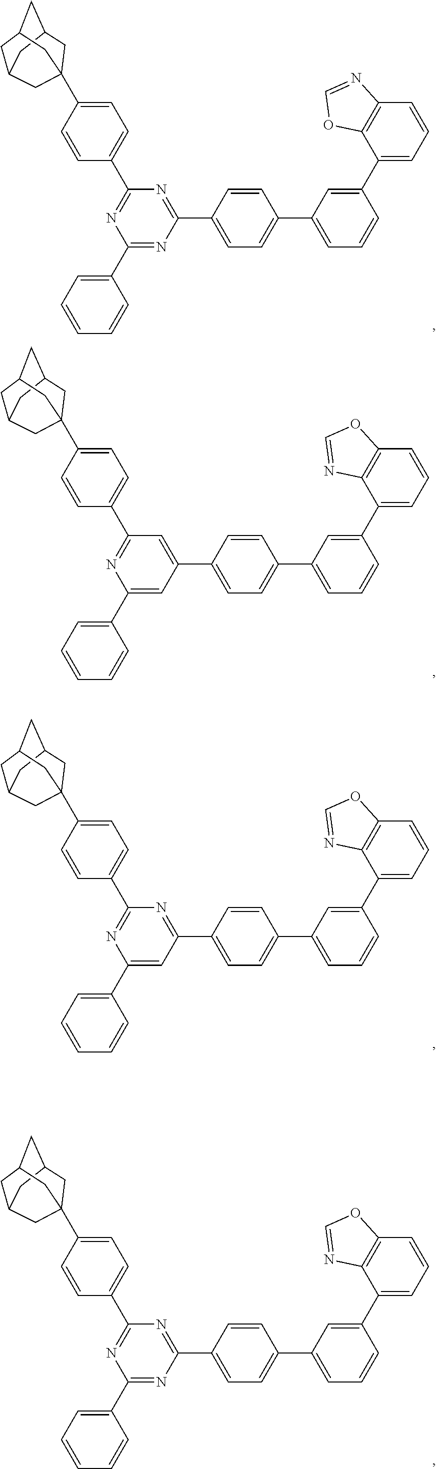 [SMILES: c1ccc(-c2nc(-c3ccc(-c4cccc(-c5cccc6ncoc56)c4)cc3)nc(-c3ccc(C45CC6CC(CC(C6)C4)C5)cc3)n2)cc1 ; c1ccc(-c2cc(-c3ccc(-c4cccc(-c5cccc6ocnc56)c4)cc3)cc(-c3ccc(C45CC6CC(CC(C6)C4)C5)cc3)n2)cc1 ; c1ccc(-c2cc(-c3ccc(-c4cccc(-c5cccc6ocnc56)c4)cc3)nc(-c3ccc(C45CC6CC(CC(C6)C4)C5)cc3)n2)cc1 ; c1ccc(-c2nc(-c3ccc(-c4cccc(-c5cccc6ocnc56)c4)cc3)nc(-c3ccc(C45CC6CC(CC(C6)C4)C5)cc3)n2)cc1]

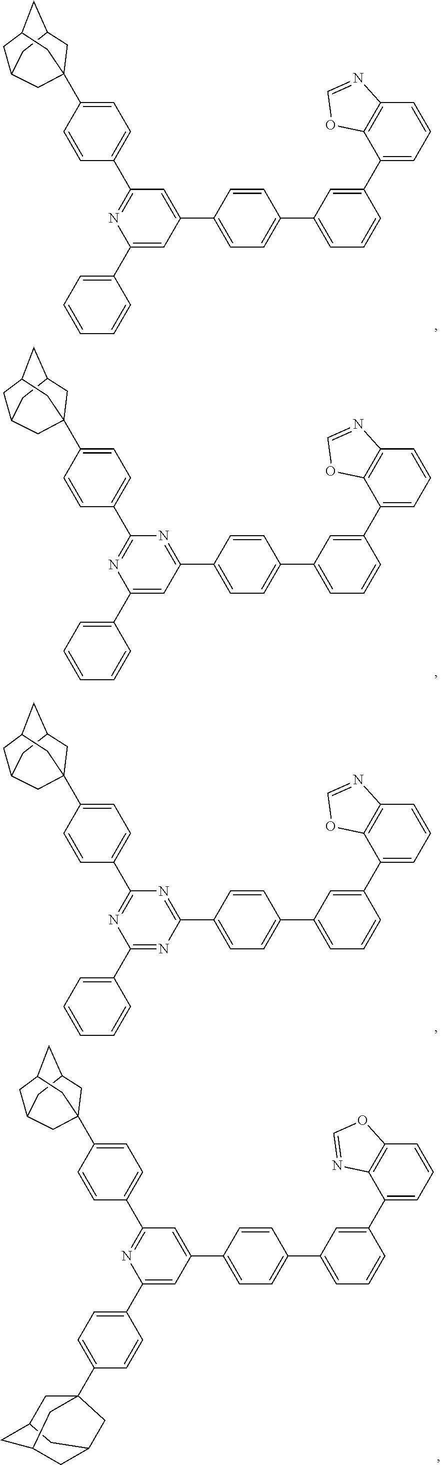

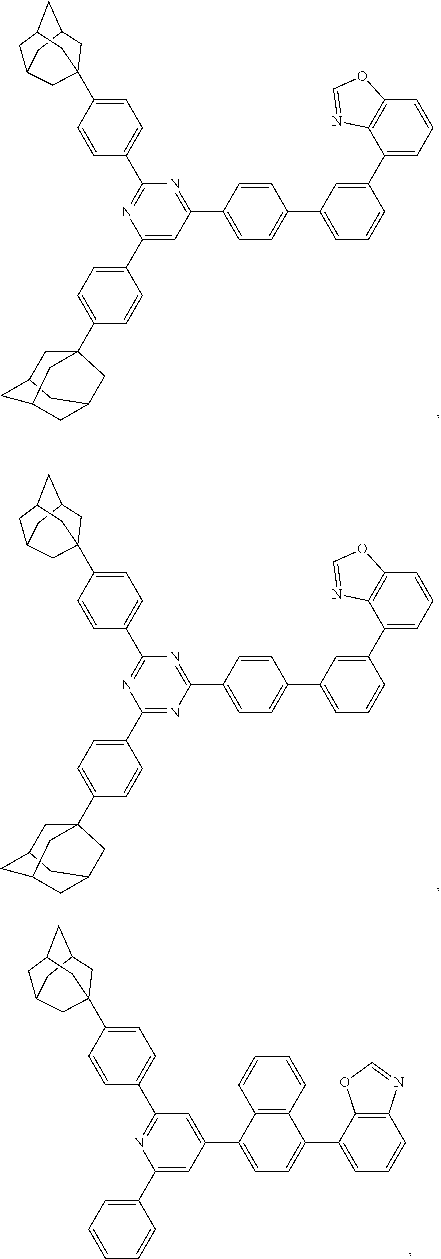

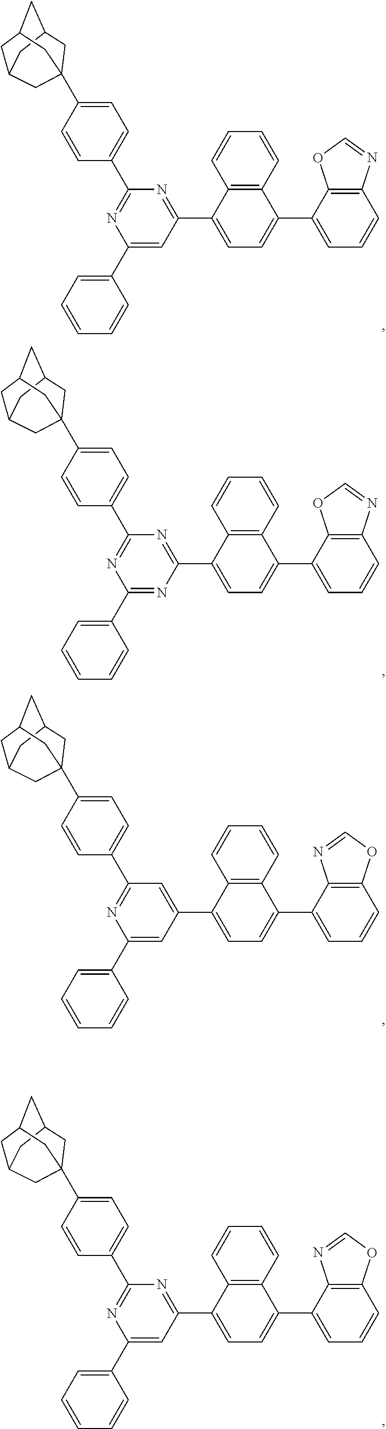 [SMILES: c1ccc(-c2cc(-c3ccc(-c4cccc5ncoc45)c4ccccc34)nc(-c3ccc(C45CC6CC(CC(C6)C4)C5)cc3)n2)cc1 ; c1ccc(-c2nc(-c3ccc(C45CC6CC(CC(C6)C4)C5)cc3)nc(-c3ccc(-c4cccc5ncoc45)c4ccccc34)n2)cc1 ; c1ccc(-c2cc(-c3ccc(-c4cccc5ocnc45)c4ccccc34)cc(-c3ccc(C45CC6CC(CC(C6)C4)C5)cc3)n2)cc1 ; c1ccc(-c2cc(-c3ccc(-c4cccc5ocnc45)c4ccccc34)nc(-c3ccc(C45CC6CC(CC(C6)C4)C5)cc3)n2)cc1]

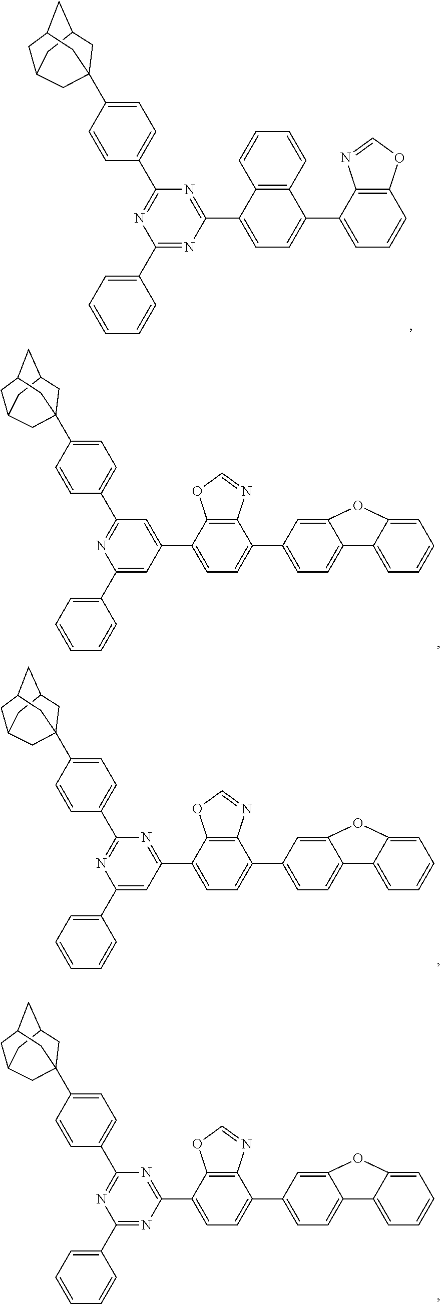 [SMILES: c1ccc(-c2nc(-c3ccc(C45CC6CC(CC(C6)C4)C5)cc3)nc(-c3ccc(-c4cccc5ocnc45)c4ccccc34)n2)cc1 ; c1ccc(-c2cc(-c3ccc(-c4ccc5c(c4)oc4ccccc45)c4ncoc34)cc(-c3ccc(C45CC6CC(CC(C6)C4)C5)cc3)n2)cc1 ; c1ccc(-c2cc(-c3ccc(-c4ccc5c(c4)oc4ccccc45)c4ncoc34)nc(-c3ccc(C45CC6CC(CC(C6)C4)C5)cc3)n2)cc1 ; c1ccc(-c2nc(-c3ccc(C45CC6CC(CC(C6)C4)C5)cc3)nc(-c3ccc(-c4ccc5c(c4)oc4ccccc45)c4ncoc34)n2)cc1]

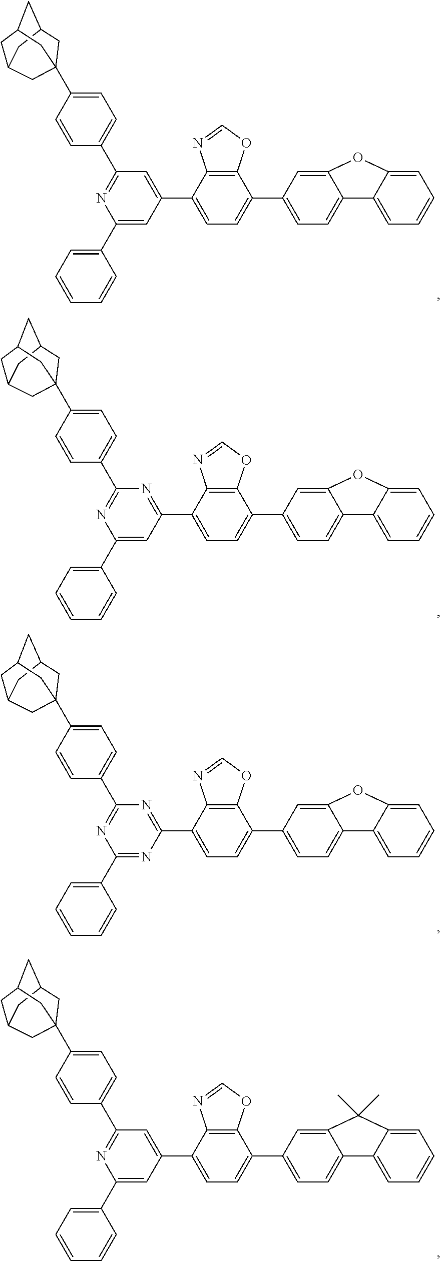 [SMILES: c1ccc(-c2cc(-c3ccc(-c4ccc5c(c4)oc4ccccc45)c4ocnc34)cc(-c3ccc(C45CC6CC(CC(C6)C4)C5)cc3)n2)cc1 ; c1ccc(-c2cc(-c3ccc(-c4ccc5c(c4)oc4ccccc45)c4ocnc34)nc(-c3ccc(C45CC6CC(CC(C6)C4)C5)cc3)n2)cc1 ; c1ccc(-c2nc(-c3ccc(C45CC6CC(CC(C6)C4)C5)cc3)nc(-c3ccc(-c4ccc5c(c4)oc4ccccc45)c4ocnc34)n2)cc1 ; CC1(C)c2ccccc2-c2ccc(-c3ccc(-c4cc(-c5ccccc5)nc(-c5ccc(C67CC8CC(CC(C8)C6)C7)cc5)c4)c4ncoc34)cc21]

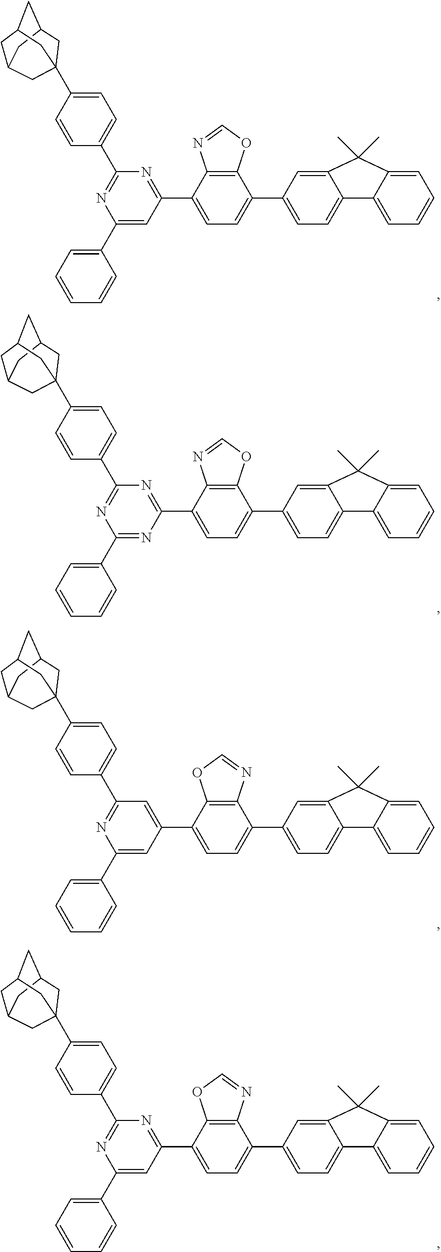 [SMILES: CC1(C)c2ccccc2-c2ccc(-c3ccc(-c4cc(-c5ccccc5)nc(-c5ccc(C67CC8CC(CC(C8)C6)C7)cc5)n4)c4ncoc34)cc21 ; CC1(C)c2ccccc2-c2ccc(-c3ccc(-c4nc(-c5ccccc5)nc(-c5ccc(C67CC8CC(CC(C8)C6)C7)cc5)n4)c4ncoc34)cc21 ; CC1(C)c2ccccc2-c2ccc(-c3ccc(-c4cc(-c5ccccc5)nc(-c5ccc(C67CC8CC(CC(C8)C6)C7)cc5)c4)c4ocnc34)cc21 ; CC1(C)c2ccccc2-c2ccc(-c3ccc(-c4cc(-c5ccccc5)nc(-c5ccc(C67CC8CC(CC(C8)C6)C7)cc5)n4)c4ocnc34)cc21]

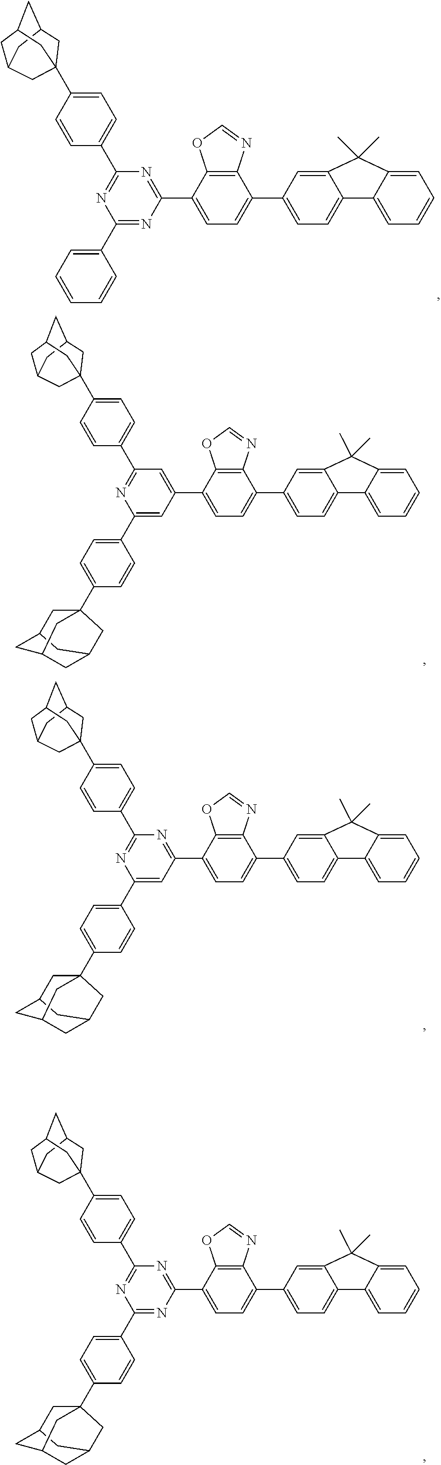

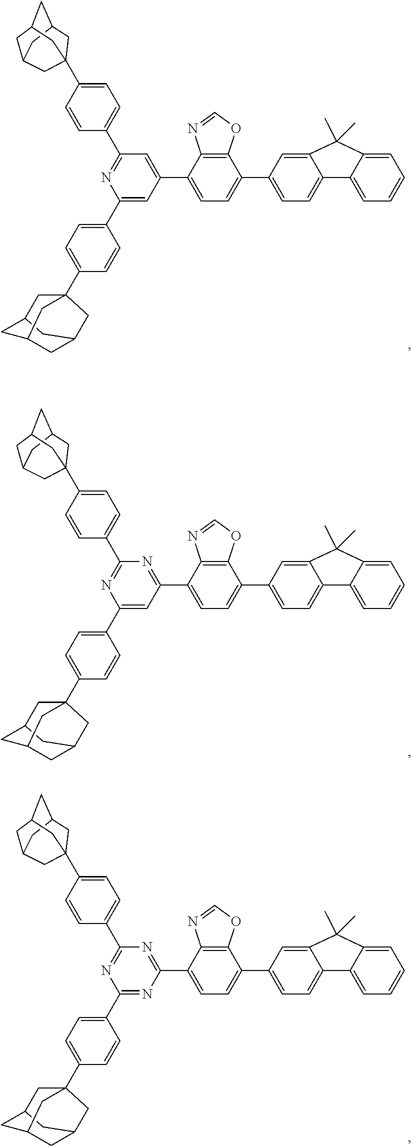

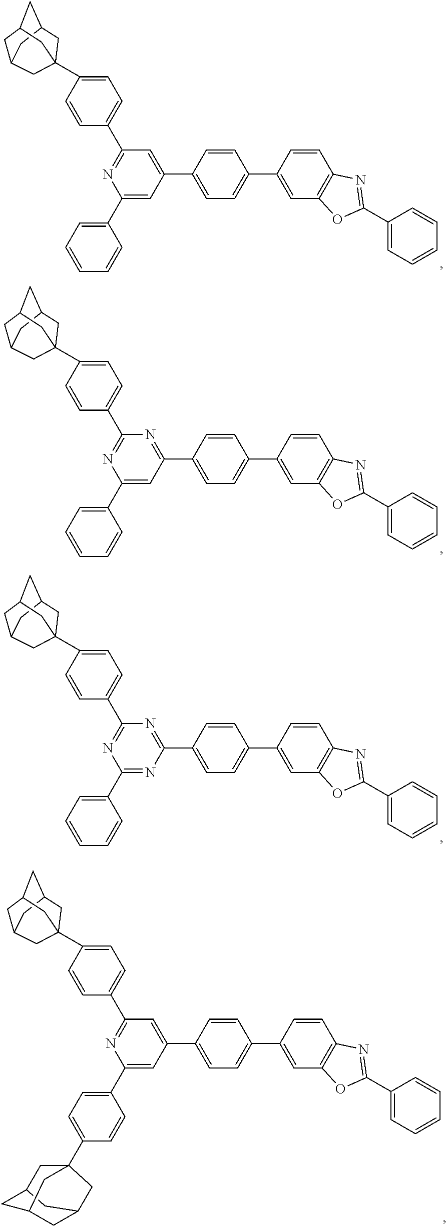 [SMILES: c1ccc(-c2cc(-c3ccc(-c4ccc5nc(-c6ccccc6)oc5c4)cc3)cc(-c3ccc(C45CC6CC(CC(C6)C4)C5)cc3)n2)cc1 ; c1ccc(-c2cc(-c3ccc(-c4ccc5nc(-c6ccccc6)oc5c4)cc3)nc(-c3ccc(C45CC6CC(CC(C6)C4)C5)cc3)n2)cc1 ; c1ccc(-c2nc(-c3ccc(-c4ccc5nc(-c6ccccc6)oc5c4)cc3)nc(-c3ccc(C45CC6CC(CC(C6)C4)C5)cc3)n2)cc1 ; c1ccc(-c2nc3ccc(-c4ccc(-c5cc(-c6ccc(C78CC9CC(CC(C9)C7)C8)cc6)nc(-c6ccc(C78CC9CC(CC(C9)C7)C8)cc6)c5)cc4)cc3o2)cc1]

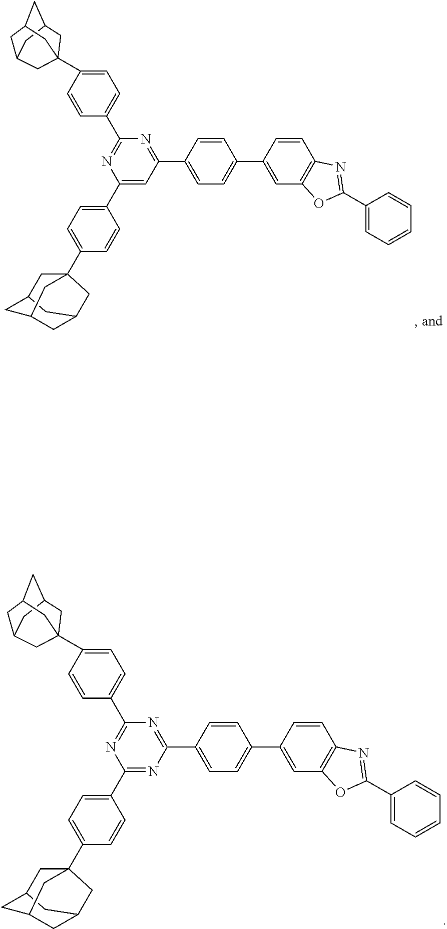 [SMILES: c1ccc(-c2nc3ccc(-c4ccc(-c5cc(-c6ccc(C78CC9CC(CC(C9)C7)C8)cc6)nc(-c6ccc(C78CC9CC(CC(C9)C7)C8)cc6)n5)cc4)cc3o2)cc1 ; c1ccc(-c2nc3ccc(-c4ccc(-c5nc(-c6ccc(C78CC9CC(CC(C9)C7)C8)cc6)nc(-c6ccc(C78CC9CC(CC(C9)C7)C8)cc6)n5)cc4)cc3o2)cc1]

Optionally, a chemical structural formula of the compound includes any one of

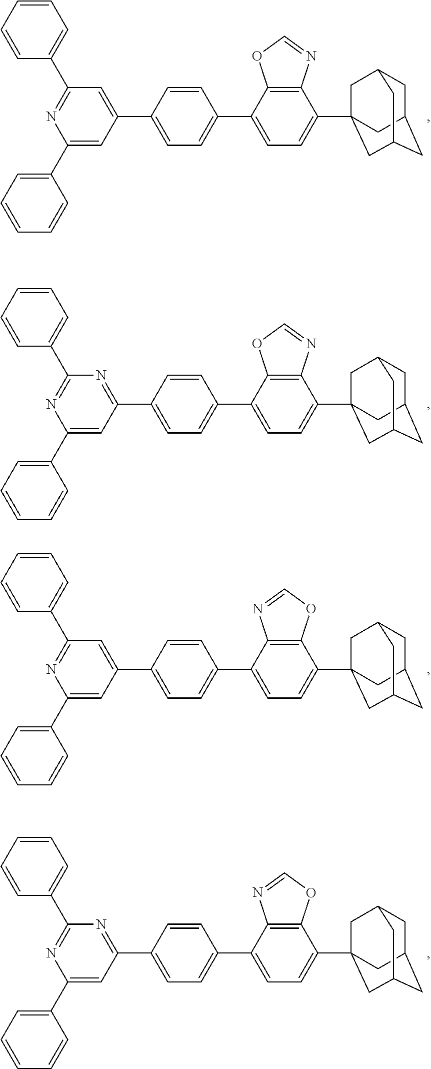

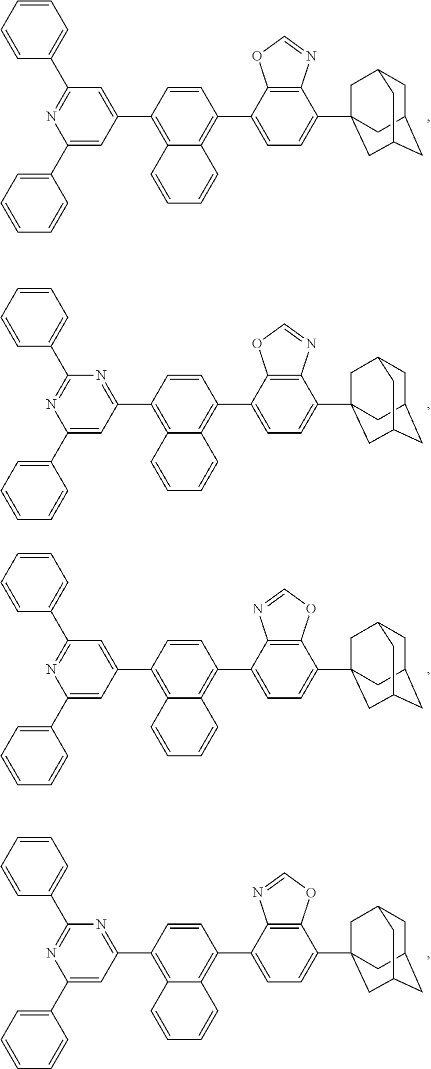

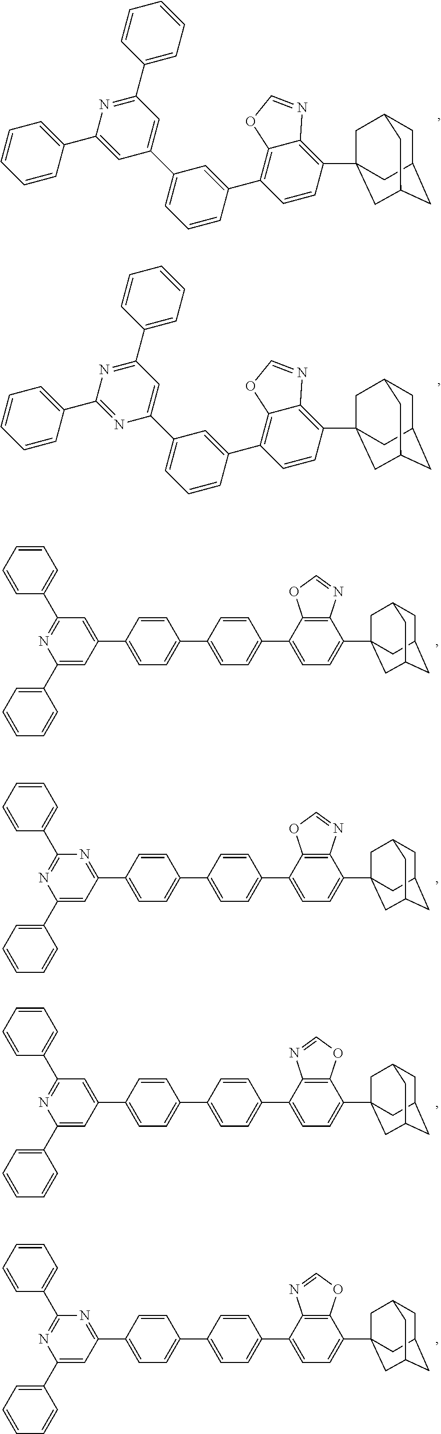

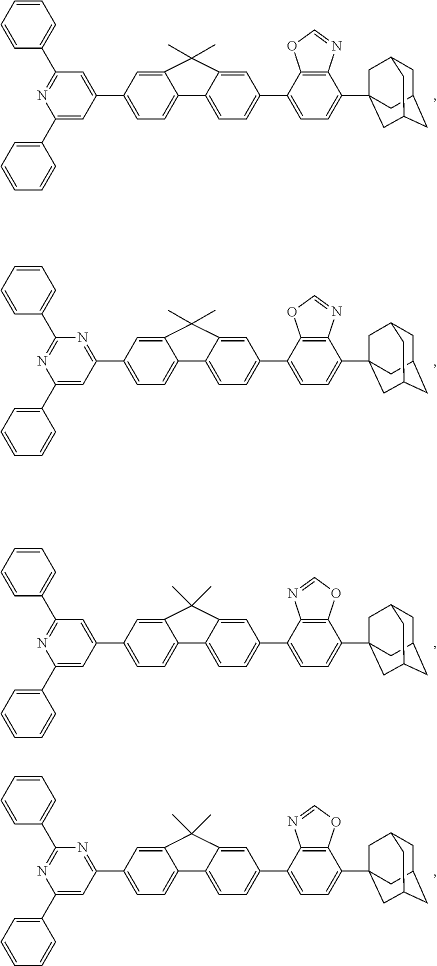 [SMILES: CC1(C)c2cc(-c3cc(-c4ccccc4)nc(-c4ccccc4)c3)ccc2-c2ccc(-c3ccc(C45CC6CC(CC(C6)C4)C5)c4ncoc34)cc21 ; CC1(C)c2cc(-c3cc(-c4ccccc4)nc(-c4ccccc4)n3)ccc2-c2ccc(-c3ccc(C45CC6CC(CC(C6)C4)C5)c4ncoc34)cc21 ; CC1(C)c2cc(-c3cc(-c4ccccc4)nc(-c4ccccc4)c3)ccc2-c2ccc(-c3ccc(C45CC6CC(CC(C6)C4)C5)c4ocnc34)cc21 ; CC1(C)c2cc(-c3cc(-c4ccccc4)nc(-c4ccccc4)n3)ccc2-c2ccc(-c3ccc(C45CC6CC(CC(C6)C4)C5)c4ocnc34)cc21]

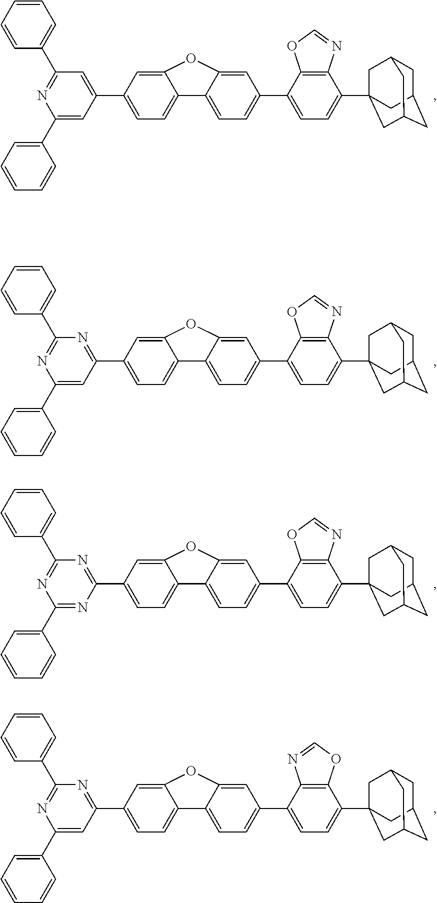

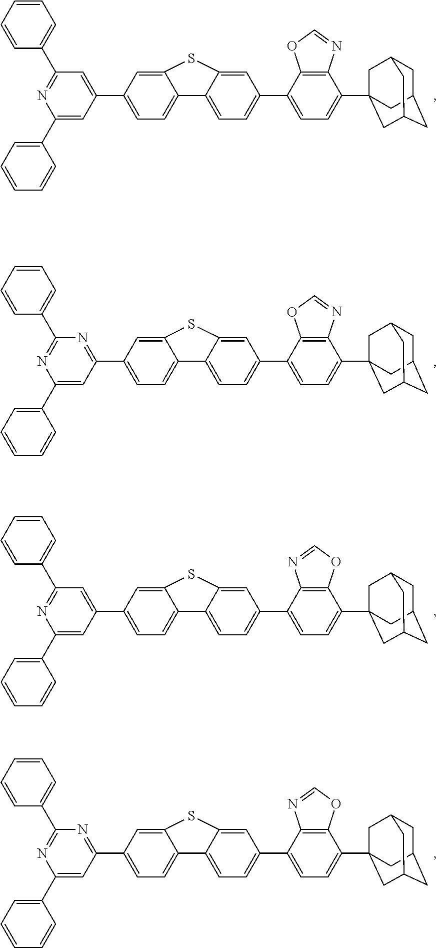 [SMILES: c1ccc(-c2cc(-c3ccc4c(c3)sc3cc(-c5ccc(C67CC8CC(CC(C8)C6)C7)c6ncoc56)ccc34)cc(-c3ccccc3)n2)cc1 ; c1ccc(-c2cc(-c3ccc4c(c3)sc3cc(-c5ccc(C67CC8CC(CC(C8)C6)C7)c6ncoc56)ccc34)nc(-c3ccccc3)n2)cc1 ; c1ccc(-c2cc(-c3ccc4c(c3)sc3cc(-c5ccc(C67CC8CC(CC(C8)C6)C7)c6ocnc56)ccc34)cc(-c3ccccc3)n2)cc1 ; c1ccc(-c2cc(-c3ccc4c(c3)sc3cc(-c5ccc(C67CC8CC(CC(C8)C6)C7)c6ocnc56)ccc34)nc(-c3ccccc3)n2)cc1]

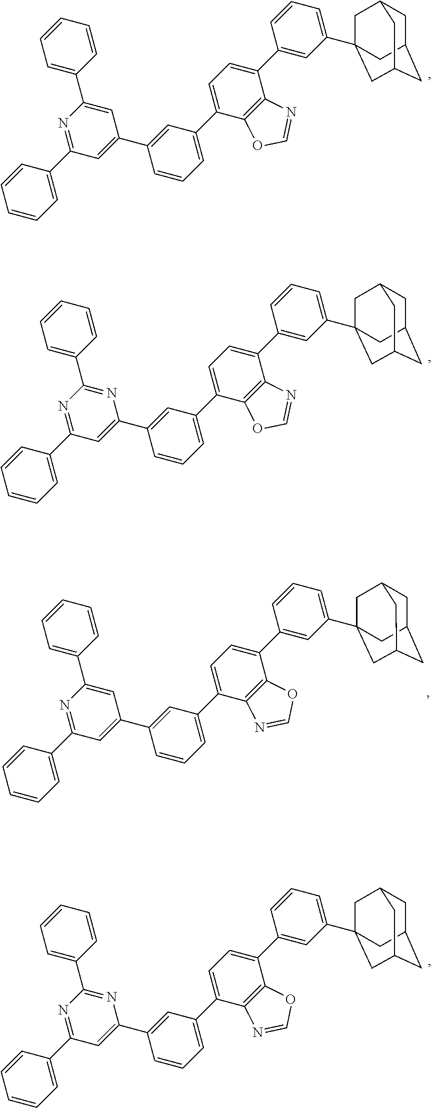

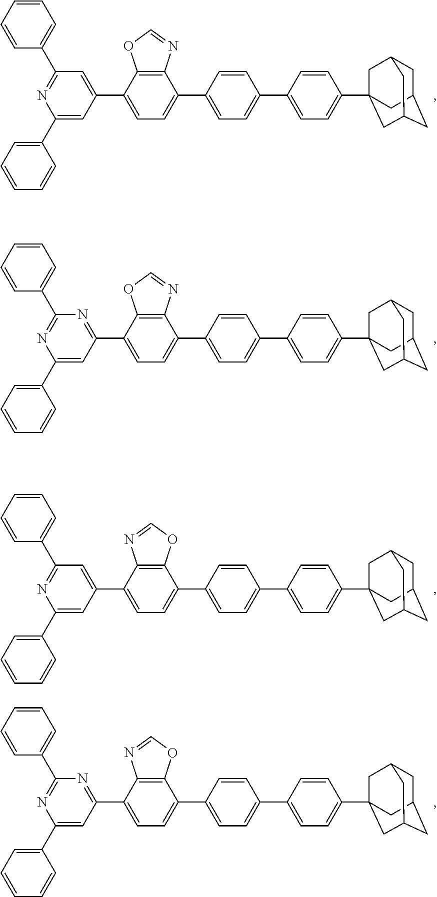

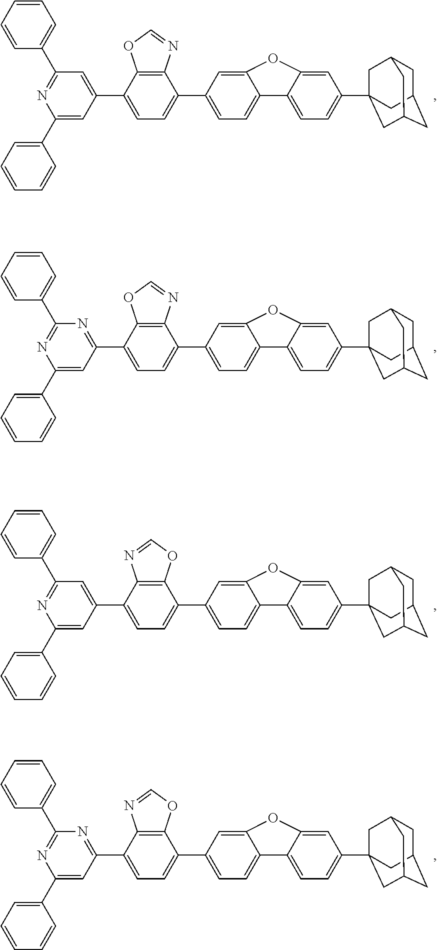 [SMILES: c1ccc(-c2cc(-c3ccc(-c4ccc5c(c4)oc4cc(C67CC8CC(CC(C8)C6)C7)ccc45)c4ncoc34)cc(-c3ccccc3)n2)cc1 ; c1ccc(-c2cc(-c3ccc(-c4ccc5c(c4)oc4cc(C67CC8CC(CC(C8)C6)C7)ccc45)c4ncoc34)nc(-c3ccccc3)n2)cc1 ; c1ccc(-c2cc(-c3ccc(-c4ccc5c(c4)oc4cc(C67CC8CC(CC(C8)C6)C7)ccc45)c4ocnc34)cc(-c3ccccc3)n2)cc1 ; c1ccc(-c2cc(-c3ccc(-c4ccc5c(c4)oc4cc(C67CC8CC(CC(C8)C6)C7)ccc45)c4ocnc34)nc(-c3ccccc3)n2)cc1]

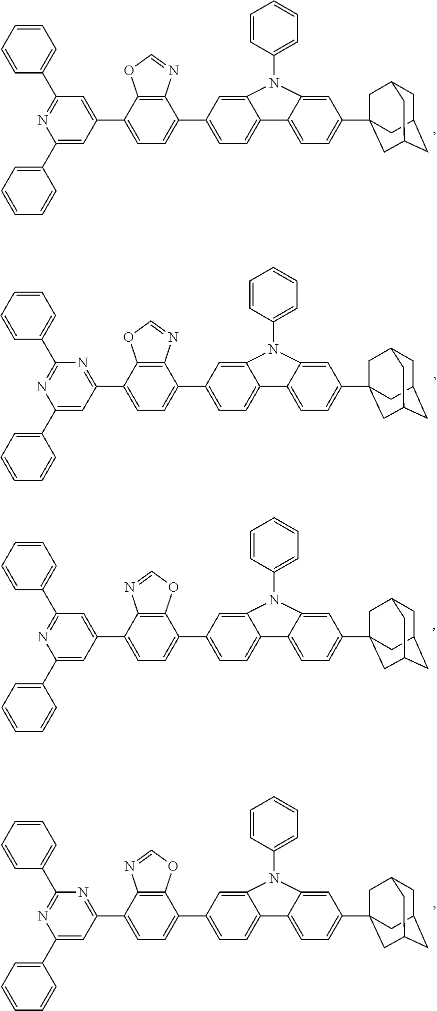 [SMILES: c1ccc(-c2cc(-c3ccc(-c4ccc5c6ccc(C78CC9CC(CC(C9)C7)C8)cc6n(-c6ccccc6)c5c4)c4ncoc34)cc(-c3ccccc3)n2)cc1 ; c1ccc(-c2cc(-c3ccc(-c4ccc5c6ccc(C78CC9CC(CC(C9)C7)C8)cc6n(-c6ccccc6)c5c4)c4ncoc34)nc(-c3ccccc3)n2)cc1 ; c1ccc(-c2cc(-c3ccc(-c4ccc5c6ccc(C78CC9CC(CC(C9)C7)C8)cc6n(-c6ccccc6)c5c4)c4ocnc34)cc(-c3ccccc3)n2)cc1 ; c1ccc(-c2cc(-c3ccc(-c4ccc5c6ccc(C78CC9CC(CC(C9)C7)C8)cc6n(-c6ccccc6)c5c4)c4ocnc34)nc(-c3ccccc3)n2)cc1]

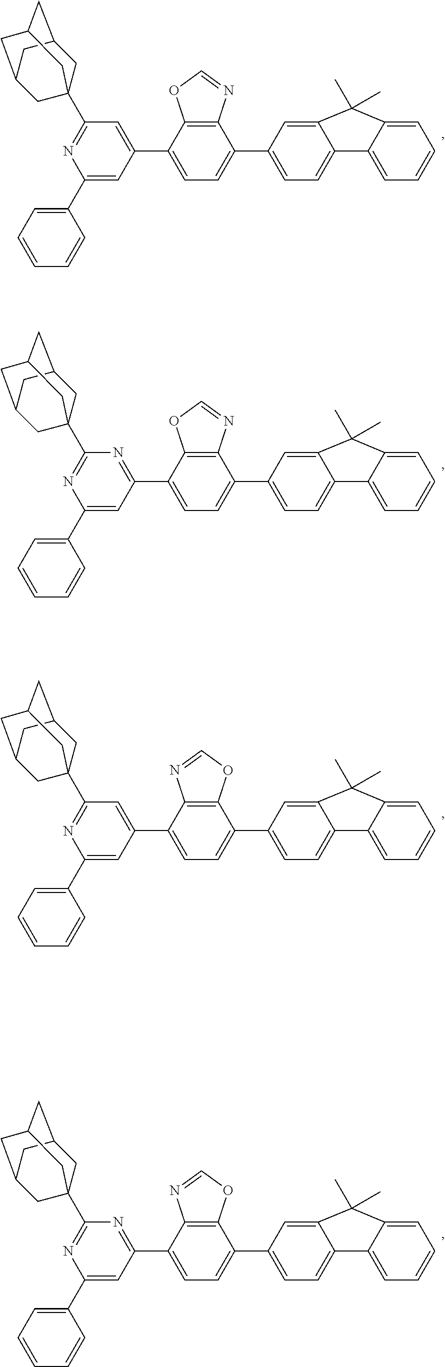 [SMILES: CC1(C)c2ccccc2-c2ccc(-c3ccc(-c4cc(-c5ccccc5)nc(C56CC7CC(CC(C7)C5)C6)c4)c4ocnc34)cc21 ; CC1(C)c2ccccc2-c2ccc(-c3ccc(-c4cc(-c5ccccc5)nc(C56CC7CC(CC(C7)C5)C6)n4)c4ocnc34)cc21 ; CC1(C)c2ccccc2-c2ccc(-c3ccc(-c4cc(-c5ccccc5)nc(C56CC7CC(CC(C7)C5)C6)c4)c4ncoc34)cc21 ; CC1(C)c2ccccc2-c2ccc(-c3ccc(-c4cc(-c5ccccc5)nc(C56CC7CC(CC(C7)C5)C6)n4)c4ncoc34)cc21]

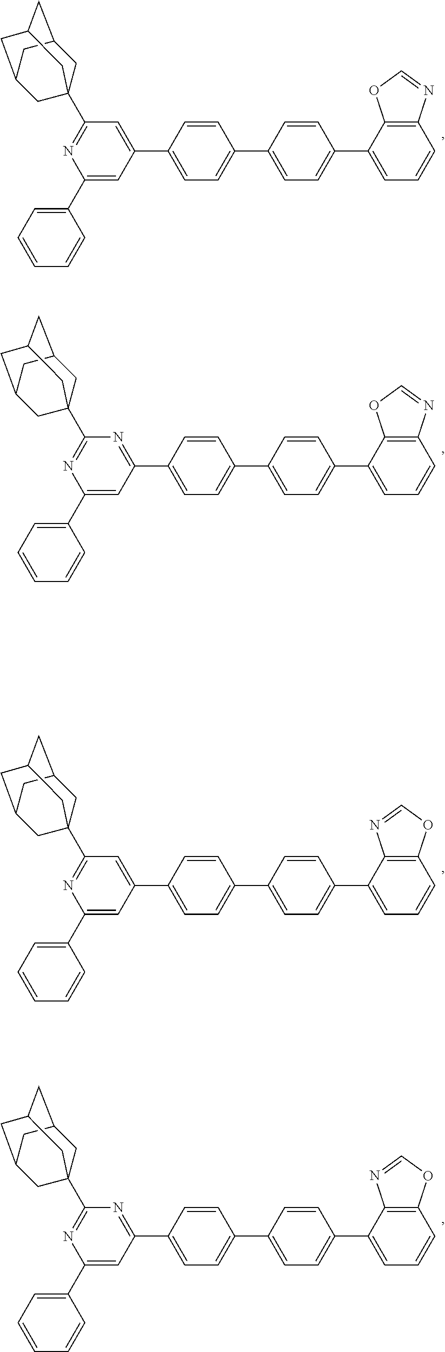 [SMILES: c1ccc(-c2cc(-c3ccc(-c4ccc(-c5cccc6ncoc56)cc4)cc3)cc(C34CC5CC(CC(C5)C3)C4)n2)cc1 ; c1ccc(-c2cc(-c3ccc(-c4ccc(-c5cccc6ncoc56)cc4)cc3)nc(C34CC5CC(CC(C5)C3)C4)n2)cc1 ; c1ccc(-c2cc(-c3ccc(-c4ccc(-c5cccc6ocnc56)cc4)cc3)cc(C34CC5CC(CC(C5)C3)C4)n2)cc1 ; c1ccc(-c2cc(-c3ccc(-c4ccc(-c5cccc6ocnc56)cc4)cc3)nc(C34CC5CC(CC(C5)C3)C4)n2)cc1]

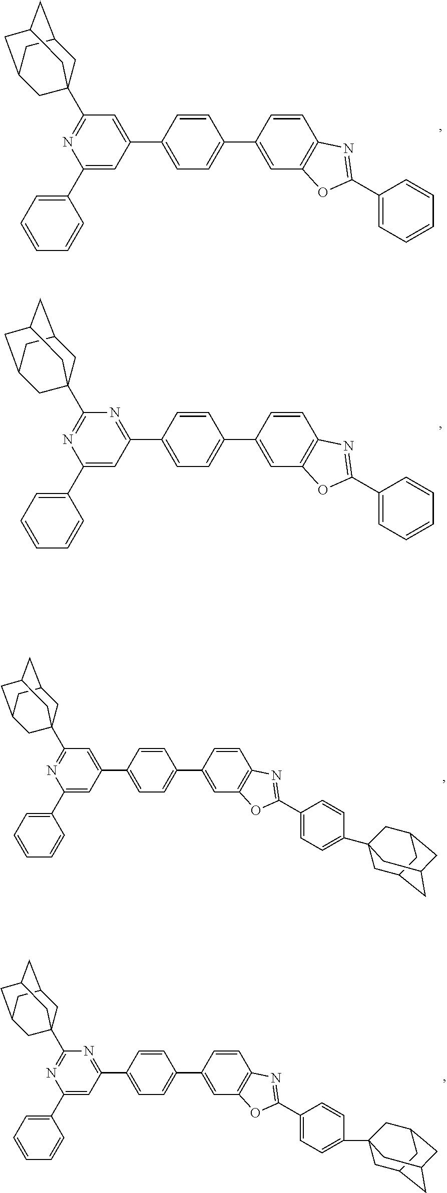 [SMILES: c1ccc(-c2cc(-c3ccc(-c4ccc5nc(-c6ccccc6)oc5c4)cc3)cc(C34CC5CC(CC(C5)C3)C4)n2)cc1 ; c1ccc(-c2cc(-c3ccc(-c4ccc5nc(-c6ccccc6)oc5c4)cc3)nc(C34CC5CC(CC(C5)C3)C4)n2)cc1 ; c1ccc(-c2cc(-c3ccc(-c4ccc5nc(-c6ccc(C78CC9CC(CC(C9)C7)C8)cc6)oc5c4)cc3)cc(C34CC5CC(CC(C5)C3)C4)n2)cc1 ; c1ccc(-c2cc(-c3ccc(-c4ccc5nc(-c6ccc(C78CC9CC(CC(C9)C7)C8)cc6)oc5c4)cc3)nc(C34CC5CC(CC(C5)C3)C4)n2)cc1]

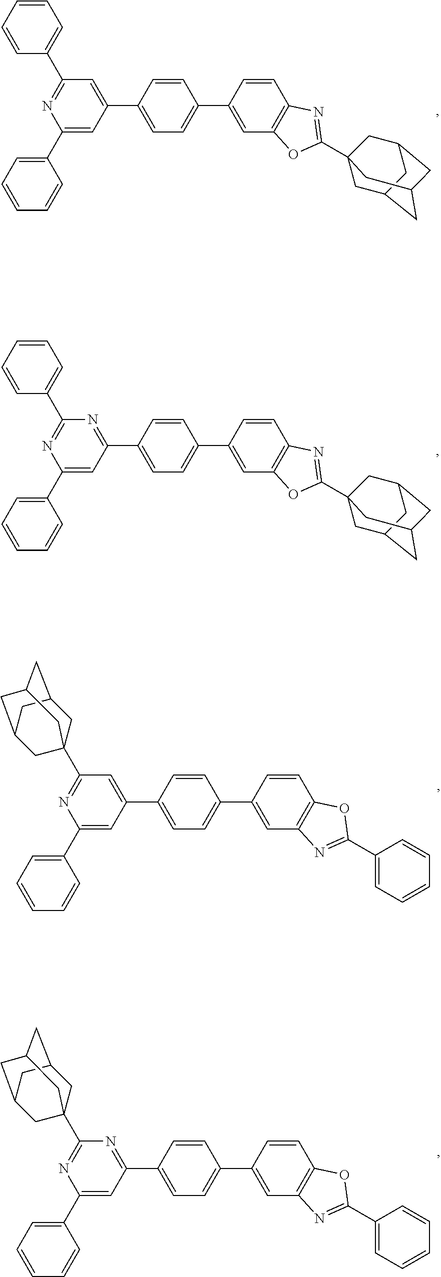 [SMILES: c1ccc(-c2cc(-c3ccc(-c4ccc5nc(C67CC8CC(CC(C8)C6)C7)oc5c4)cc3)cc(-c3ccccc3)n2)cc1 ; c1ccc(-c2cc(-c3ccc(-c4ccc5nc(C67CC8CC(CC(C8)C6)C7)oc5c4)cc3)nc(-c3ccccc3)n2)cc1 ; c1ccc(-c2cc(-c3ccc(-c4ccc5oc(-c6ccccc6)nc5c4)cc3)cc(C34CC5CC(CC(C5)C3)C4)n2)cc1 ; c1ccc(-c2cc(-c3ccc(-c4ccc5oc(-c6ccccc6)nc5c4)cc3)nc(C34CC5CC(CC(C5)C3)C4)n2)cc1]

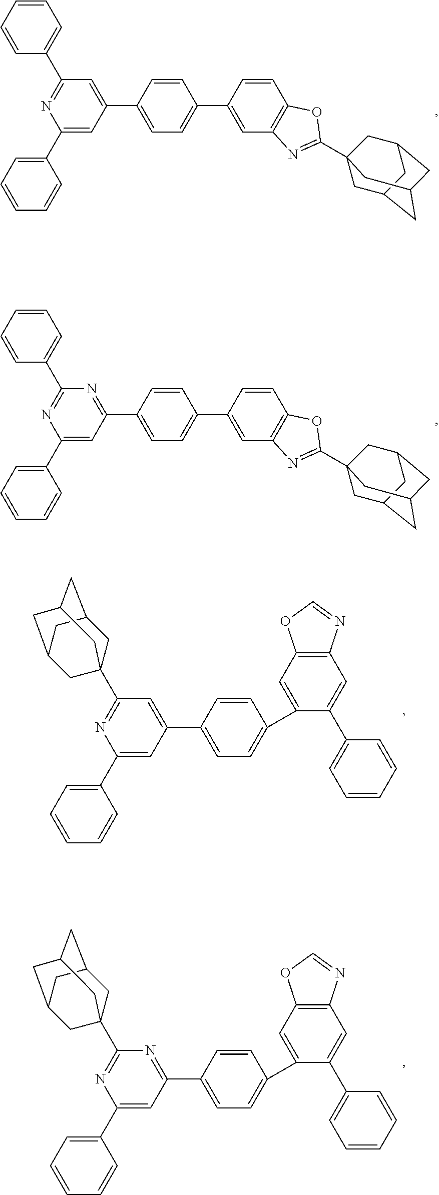 [SMILES: c1ccc(-c2cc(-c3ccc(-c4ccc5oc(C67CC8CC(CC(C8)C6)C7)nc5c4)cc3)cc(-c3ccccc3)n2)cc1 ; c1ccc(-c2cc(-c3ccc(-c4ccc5oc(C67CC8CC(CC(C8)C6)C7)nc5c4)cc3)nc(-c3ccccc3)n2)cc1 ; c1ccc(-c2cc(-c3ccc(-c4cc5ocnc5cc4-c4ccccc4)cc3)cc(C34CC5CC(CC(C5)C3)C4)n2)cc1 ; c1ccc(-c2cc(-c3ccc(-c4cc5ocnc5cc4-c4ccccc4)cc3)nc(C34CC5CC(CC(C5)C3)C4)n2)cc1]

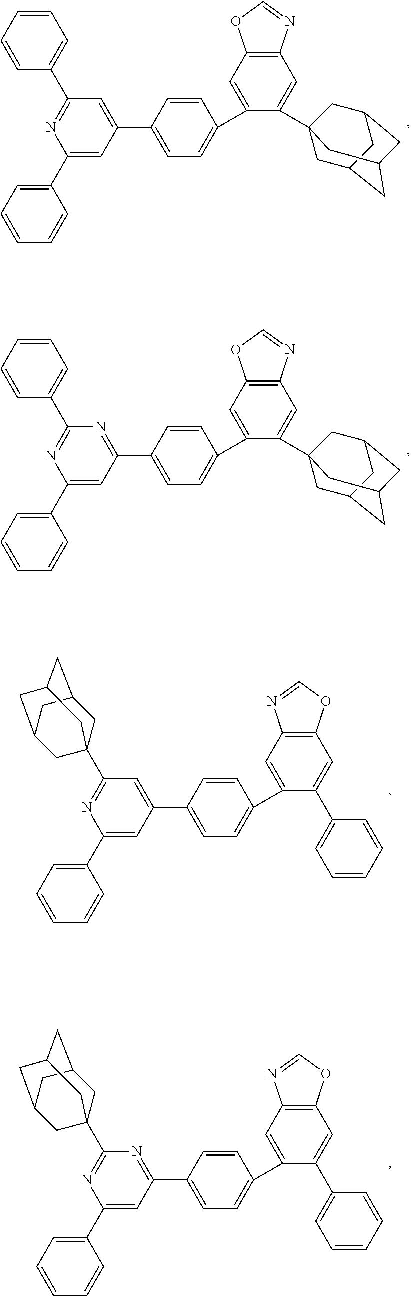 [SMILES: c1ccc(-c2cc(-c3ccc(-c4cc5ocnc5cc4C45CC6CC(CC(C6)C4)C5)cc3)cc(-c3ccccc3)n2)cc1 ; c1ccc(-c2cc(-c3ccc(-c4cc5ocnc5cc4C45CC6CC(CC(C6)C4)C5)cc3)nc(-c3ccccc3)n2)cc1 ; c1ccc(-c2cc(-c3ccc(-c4cc5ncoc5cc4-c4ccccc4)cc3)cc(C34CC5CC(CC(C5)C3)C4)n2)cc1 ; c1ccc(-c2cc(-c3ccc(-c4cc5ncoc5cc4-c4ccccc4)cc3)nc(C34CC5CC(CC(C5)C3)C4)n2)cc1]

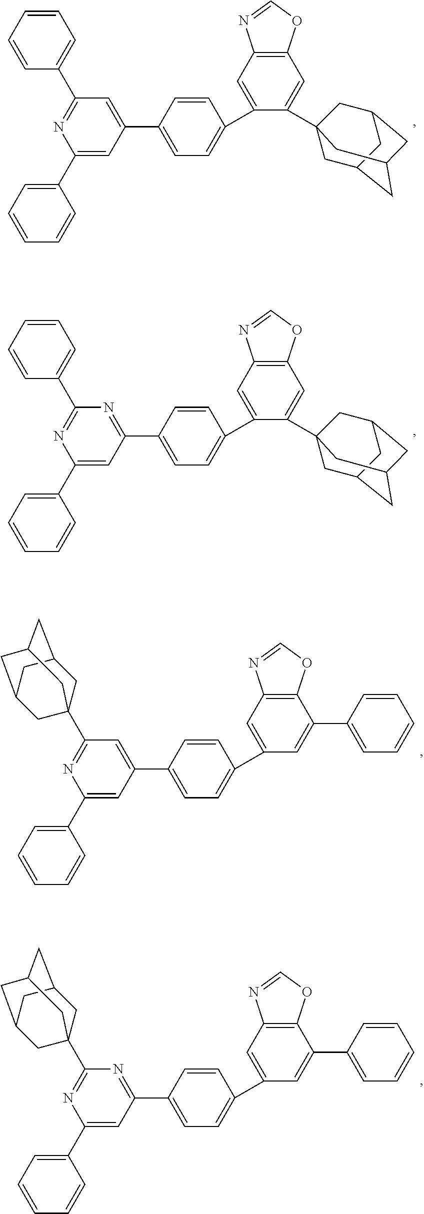 [SMILES: c1ccc(-c2cc(-c3ccc(-c4cc5ncoc5cc4C45CC6CC(CC(C6)C4)C5)cc3)cc(-c3ccccc3)n2)cc1 ; c1ccc(-c2cc(-c3ccc(-c4cc5ncoc5cc4C45CC6CC(CC(C6)C4)C5)cc3)nc(-c3ccccc3)n2)cc1 ; c1ccc(-c2cc(-c3ccc(-c4cc(-c5ccccc5)c5ocnc5c4)cc3)cc(C34CC5CC(CC(C5)C3)C4)n2)cc1 ; c1ccc(-c2cc(-c3ccc(-c4cc(-c5ccccc5)c5ocnc5c4)cc3)nc(C34CC5CC(CC(C5)C3)C4)n2)cc1]

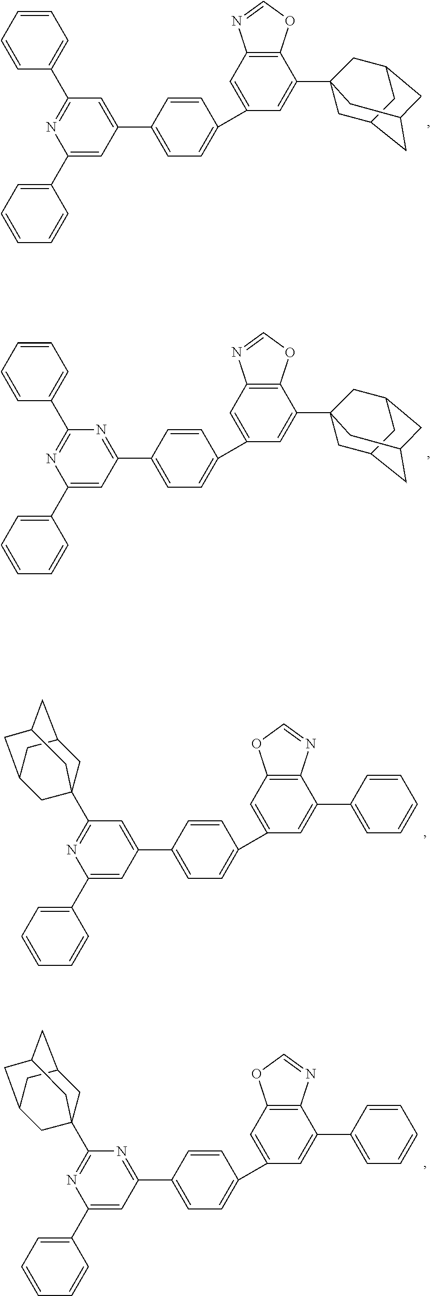 [SMILES: c1ccc(-c2cc(-c3ccc(-c4cc(C56CC7CC(CC(C7)C5)C6)c5ocnc5c4)cc3)cc(-c3ccccc3)n2)cc1 ; c1ccc(-c2cc(-c3ccc(-c4cc(C56CC7CC(CC(C7)C5)C6)c5ocnc5c4)cc3)nc(-c3ccccc3)n2)cc1 ; c1ccc(-c2cc(-c3ccc(-c4cc(-c5ccccc5)c5ncoc5c4)cc3)cc(C34CC5CC(CC(C5)C3)C4)n2)cc1 ; c1ccc(-c2cc(-c3ccc(-c4cc(-c5ccccc5)c5ncoc5c4)cc3)nc(C34CC5CC(CC(C5)C3)C4)n2)cc1]

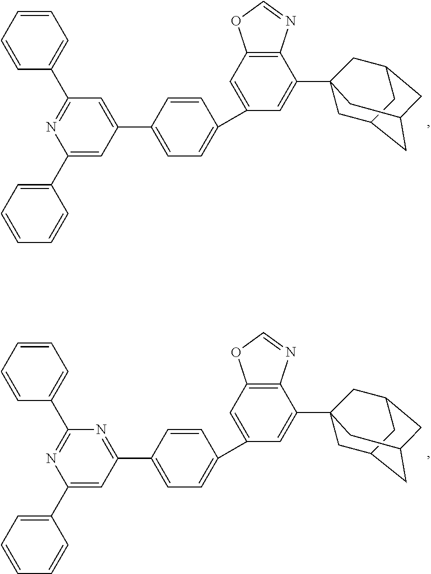 [SMILES: c1ccc(-c2cc(-c3ccc(-c4cc(C56CC7CC(CC(C7)C5)C6)c5ncoc5c4)cc3)cc(-c3ccccc3)n2)cc1 ; c1ccc(-c2cc(-c3ccc(-c4cc(C56CC7CC(CC(C7)C5)C6)c5ncoc5c4)cc3)nc(-c3ccccc3)n2)cc1]

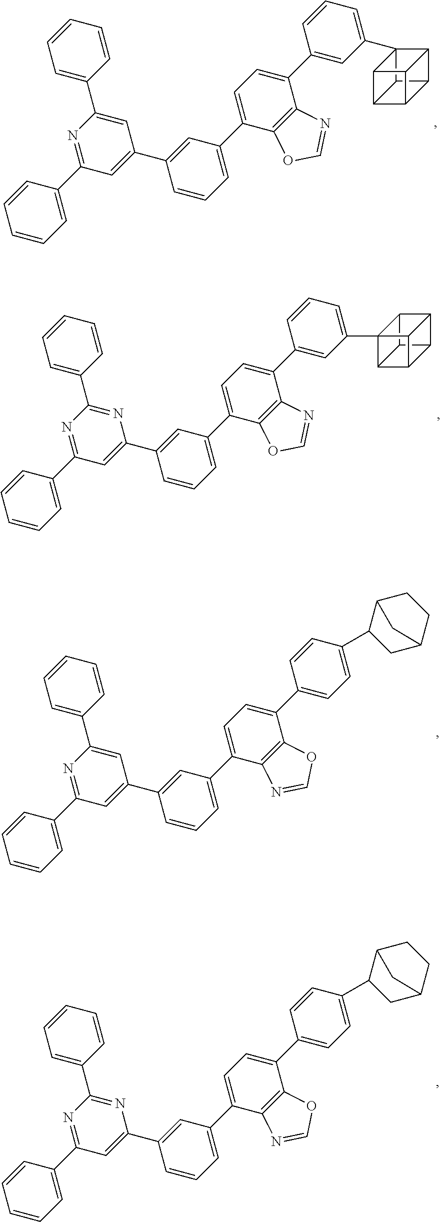 [SMILES: c1ccc(-c2cc(-c3cccc(-c4ccc(-c5cccc(C67C8C9C%10C8C6C%10C97)c5)c5ncoc45)c3)cc(-c3ccccc3)n2)cc1 ; c1ccc(-c2cc(-c3cccc(-c4ccc(-c5cccc(C67C8C9C%10C8C6C%10C97)c5)c5ncoc45)c3)nc(-c3ccccc3)n2)cc1 ; c1ccc(-c2cc(-c3cccc(-c4ccc(-c5ccc(C6CC7CCC6C7)cc5)c5ocnc45)c3)cc(-c3ccccc3)n2)cc1 ; c1ccc(-c2cc(-c3cccc(-c4ccc(-c5ccc(C6CC7CCC6C7)cc5)c5ocnc45)c3)nc(-c3ccccc3)n2)cc1]

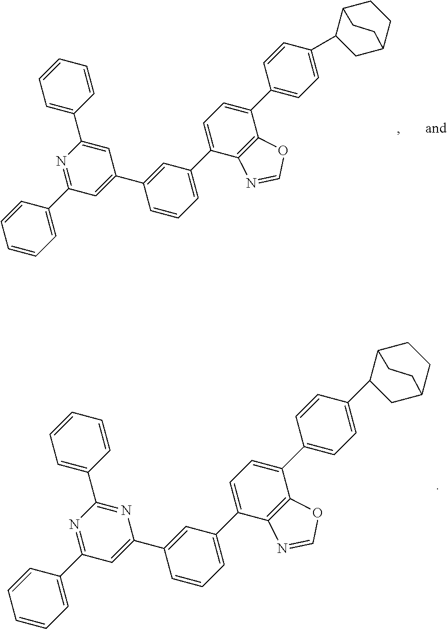 [SMILES: c1ccc(-c2cc(-c3cccc(-c4ccc(-c5ccc(C6CC7CCC6CC7)cc5)c5ocnc45)c3)cc(-c3ccccc3)n2)cc1 ; c1ccc(-c2cc(-c3cccc(-c4ccc(-c5ccc(C6CC7CCC6CC7)cc5)c5ocnc45)c3)nc(-c3ccccc3)n2)cc1]

The above description is merely a summary of the technical solutions of the present application. In order to more clearly know the elements of the present application to enable the implementation according to the contents of the description, and in order to make the above and other purposes, features and advantages of the present application more apparent and understandable, the particular embodiments of the present application are provided below.

### BRIEF DESCRIPTION OF THE DRAWINGS

In order to more clearly illustrate the technical solutions of the embodiments of the present application or the prior art, the figures that are required to describe the embodiments or the prior art will be briefly described below. Apparently, the figures that are described below are merely embodiments of the present application, and a person skilled in the art can obtain other figures according to these figures without paying creative work.

FIG. 1 is a schematic structural diagram of a light emitting device according to an embodiment of the present application;

FIG. 2 is a schematic structural diagram of another light emitting device according to an embodiment of the present application; and

FIG. 3 is a schematic structural diagram of a displaying device according to an embodiment of the present application.

### DETAILED DESCRIPTION

The technical solutions of the embodiments of the present application will be clearly and completely described below with reference to the drawings of the embodiments of the present application. Apparently, the described embodiments are merely certain embodiments of the present application, rather than all of the embodiments. All of the other embodiments that a person skilled in the art obtains on the basis of the embodiments of the present application without paying creative work fall within the protection scope of the present application.

In the embodiments of the present application, terms such as “first” and “second” are used to distinguish identical items or similar items that have substantially the same functions and effects, merely in order to clearly describe the technical solutions of the embodiments of the present application, and should not be construed as indicating or implying the degrees of importance or implicitly indicating the quantity of the specified technical features.

In the embodiments of the present application, the meaning of “at least one” is “one or more”, unless explicitly and particularly defined otherwise.

In the embodiments of the present application, the terms that indicate orientation or position relations, such as “upper”, are based on the orientation or position relations shown in the drawings, and are merely for conveniently describing the present application and simplifying the description, rather than indicating or implying that the device or element must have the specific orientation and be constructed and operated according to the specific orientation. Therefore, they should not be construed as a limitation on the present application.

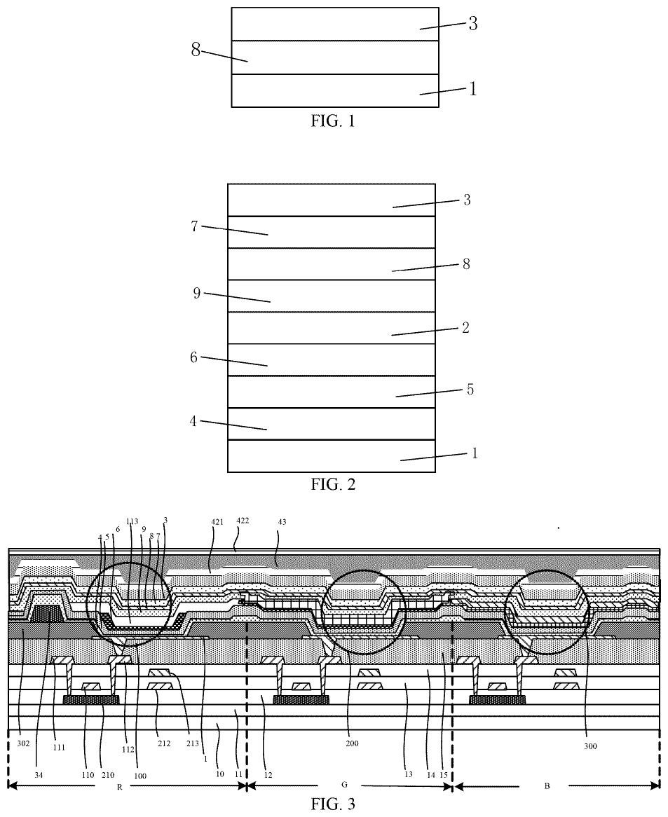

An embodiment of the present application provides a light emitting device. Referring to FIG. 1, the light emitting device comprises an electron transport layer, and the material of the electron transport layer comprises a heteroaromatic ring group including at least one nitrogen atom, a first electron-withdrawing group, and at least one three-dimensional group having a rigidity. The triplet-state energy level T1 of the material of the electron transport layer satisfies T1≥2.3 eV. The absolute value of the energy value of the highest occupied molecular orbital HOMO of the material of the electron transport layer satisfies 6.0 eV≤|HOMO|≤7.0 eV. The absolute value of the energy value of the lowest unoccupied molecular orbital LUMO of the material of the electron transport layer satisfies 2.6 eV≤|LUMO|≤3.6 eV.

The fabricating process of the light emitting device is not particularly limited herein. As an example, the film layers of the light emitting device may be fabricated by vacuum vapor deposition.

The type of the light emitting device is not particularly limited herein. As an example, the light emitting device may include a top-emission-type light emitting device or a bottom-emission-type light emitting device.

The first electron-withdrawing group is not particularly limited. As an example, the first electron-withdrawing group may include a group having a strong electron-withdrawing capability, i.e., a strong electron-withdrawing group.

The triplet-state energy level T1 of the material of the electron transport layer is not particularly limited. As an example, the triplet-state energy level T1 of the material of the electron transport layer may be 2.3 eV, 2.4 eV, 2.6 eV, 2.9 eV, 3 eV and so on.

The highest occupied molecular orbital (HOMO) refers to the molecular orbital of the highest energy among all of the molecular orbitals occupied by electrons. The energy value of the highest occupied molecular orbital is also referred to as the HOMO value.

6.0 eV≤|HOMO|≤7.0 eV. The |HOMO| is not particularly limited herein. As an example, the |HOMO| may be 6.0 eV, 6.2 eV, 6.3 eV, 6.4 eV, 6.5 eV, 6.6 eV, 6.8 eV, 7.0 eV and so on.

The lowest unoccupied molecular orbital (LUMO) refers to the molecular orbital of the lowest energy among all of the molecular orbitals not occupied by electrons. The energy value of the lowest unoccupied molecular orbital is also referred to as the LUMO value.

2.6 eV≤|LUMO|≤3.6 eV. The |LUMO| is not particularly limited herein. As an example, the |LUMO| may be 2.6 eV, 2.8 eV, 2.9 eV, 3.0 eV, 3.1 eV, 3.3 eV, 3.4 eV, 3.6 eV and so on.

The embodiments of the present application provide a light emitting device, wherein the light emitting device comprises an electron transport layer. In an aspect, the material of the electron transport layer comprises the first electron-withdrawing group, and the first electron-withdrawing group is a strong electron-withdrawing group, and has deep HOMO energy level and LUMO energy level. The low LUMO energy level can effectively reduce the injection barrier between the material and the neighboring functional layer, which facilitates the electron injection, and accordingly can reduce the lightening voltage of the light emitting device. The low HOMO energy level can effectively block holes, to increase the efficiency of the recombination of the holes and the electrons. Moreover, because the first electron-withdrawing group has a high T1, the material of the electron transport layer also has a high T1, whereby the material of the electron transport layer can prevent triplet excitons from diffusing toward the electron transport layer, thereby increasing the efficiency of the light emitting device. Additionally, the first electron-withdrawing group can interact with the neighboring groups, so that the material of the electron transport layer has a stable geometrical configuration, whereby it does not easily deform by the effect of an external electric field, and has a lower reorganization energy and a higher electron mobility. In another aspect, the material of the electron transport layer comprises at least one three-dimensional group, which can effectively inhibit material crystallization. Moreover, the three-dimensional group has a good rigidity, so that the material has a high glass-transition temperature Tg. The high Tg enables the material not to easily be cracked in the vapor deposition, which facilitates to improve the stability, the film-forming property, the durability, the heat resistance, and so on, of the material, thereby increasing the efficiency and the life of the light emitting device. Accordingly, a stable and high-efficiency material of the electron transport layer can be obtained, and further a light emitting device that has a good stability, a low voltage, a high luminous efficiency and a long life is obtained.

Optionally, the general structural formula of the material of the electron transport layer is:

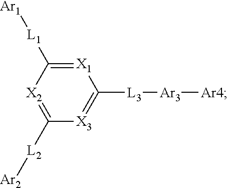

wherein X1 is C(Ra) or nitrogen, X2 is C(Rb) or nitrogen, X3 is C(Rc) or nitrogen, and at least one of X1, X2 and X3 is nitrogen, wherein each of Ra, Rb and Rc is independently any one of a group consisting of hydrogen, diplogen, fluorine, chlorine, an alkyl group whose carbon-atom quantity is 1-12, a halogenated alkyl group whose carbon-atom quantity is 1-12, an alkoxy group whose carbon-atom quantity is 1-12, a cycloalkyl group whose carbon-atom quantity is 3-10, C6-20 aryl, and a heteroaryl group whose carbon-atom quantity is 3-20; each of L1, L2 and L3 is independently any one of a group consisting of a single bond, a substituted or unsubstituted arylene group whose carbon-atom quantity is 6-60, and a substituted or unsubstituted heteroarylene whose carbon-atom quantity is 1-60; each of Ar1, Ar2 and Ar4 is independently any one of a group consisting of a substituted or unsubstituted alkyl group whose carbon-atom quantity is 1-12, a substituted or unsubstituted cycloalkyl group whose carbon-atom quantity is 3-10, a substituted or unsubstituted aralkyl group whose carbon-atom quantity is 7-30, a substituted or unsubstituted heterarylalkyl whose carbon-atom quantity is 2-60, a substituted or unsubstituted aryl whose carbon-atom quantity is 6-60, and a substituted or unsubstituted heteroaryl group whose carbon-atom quantity is 3-60, and at least one of Ar1, Ar2 and Ar4 is the three-dimensional group having a rigidity; and Ar3 is the first electron-withdrawing group.

The embodiments of the present application provide a light emitting device, wherein the light emitting device comprises an electron transport layer. The groups of the material of the electron transport layer are connected in a particular mode, which can effectively destroy or reduce the conjugation among the molecules, so as to further increase the T1 of the material, to further increase the utilization ratio of the excitons, thereby increasing the luminous efficiency and the life of the light emitting device.

Optionally, in the material of the electron transport layer, the general structural formula of Ar3 is:

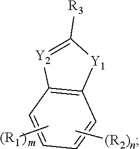

wherein Y1 is any one of carbon, oxygen, sulphur, selenium and N—R4; Y2 is any one of carbon and nitrogen; each of R1, R2 and R3 is independently any one of hydrogen, diplogen, halogen, amino, a nitrile group, nitro, C1-30 alkyl, C2-30 alkenyl, C2-30 alkynyl, C1-30 alkoxy, C6-30 aryloxy, and C6-50 aryl substituted by C6-30 aryl, or includes any one of at least one of heteroatoms selected from nitrogen, oxygen and sulphur and C2-50 heteroaryl unsubstituted or substituted by C6-30 aryloxy, and two of R1, R2 and R3 are directly connected to neighboring groups L3 and Ar4; R4 is any one of a group consisting of hydrogen, diplogen, fluorine, chlorine, an alkyl group whose carbon-atom quantity is 1-12, a halogenated alkyl group whose carbon-atom quantity is 1-12, an alkoxy group whose carbon-atom quantity is 1-12, a cycloalkyl group whose carbon-atom quantity is 3-10, C6-20 aryl, and a heteroaryl group whose carbon-atom quantity is 3-20; and each of m and n is independently 1 or 2.

Optionally, the general structural formula of the material of the electron transport layer is any one of

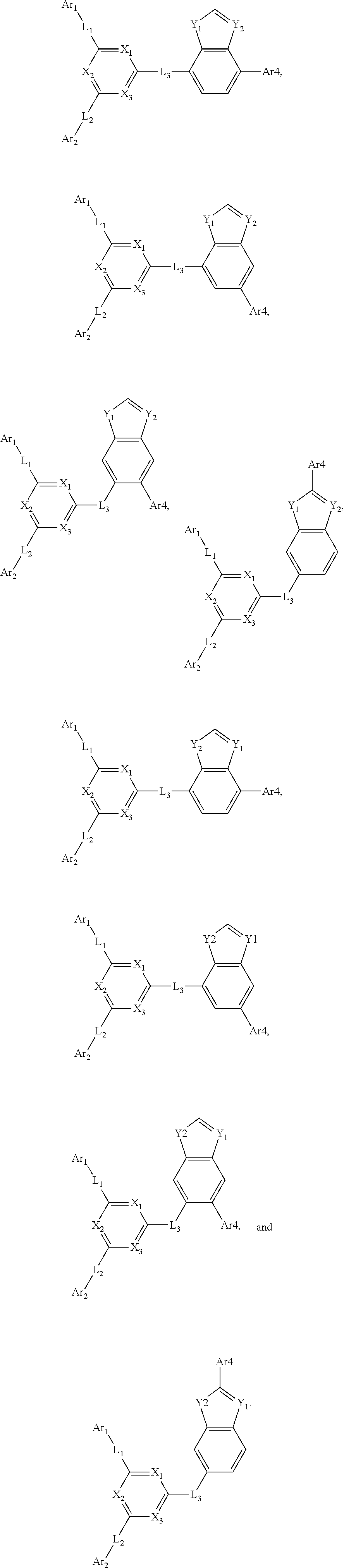

Optionally, Ar3 includes benzoxazole.

Optionally, the general structural formula of the three-dimensional group having a rigidity is any one of

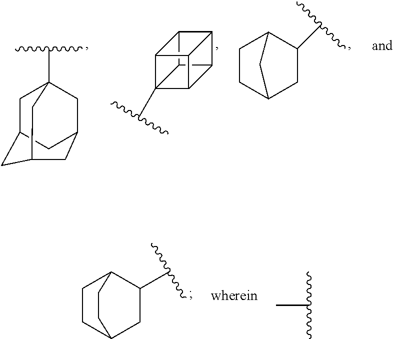

is a bond connected to a carbon atom.

Optionally, in the material of the electron transport layer, the heteroaromatic ring group including at least one nitrogen atom includes a triazine group. Therefore, the material of the electron transport layer has a stronger electron-withdrawing capability, and a higher electron mobility.

Optionally, the chemical structural formula of the material of the electron transport layer having the triazine group includes any one of

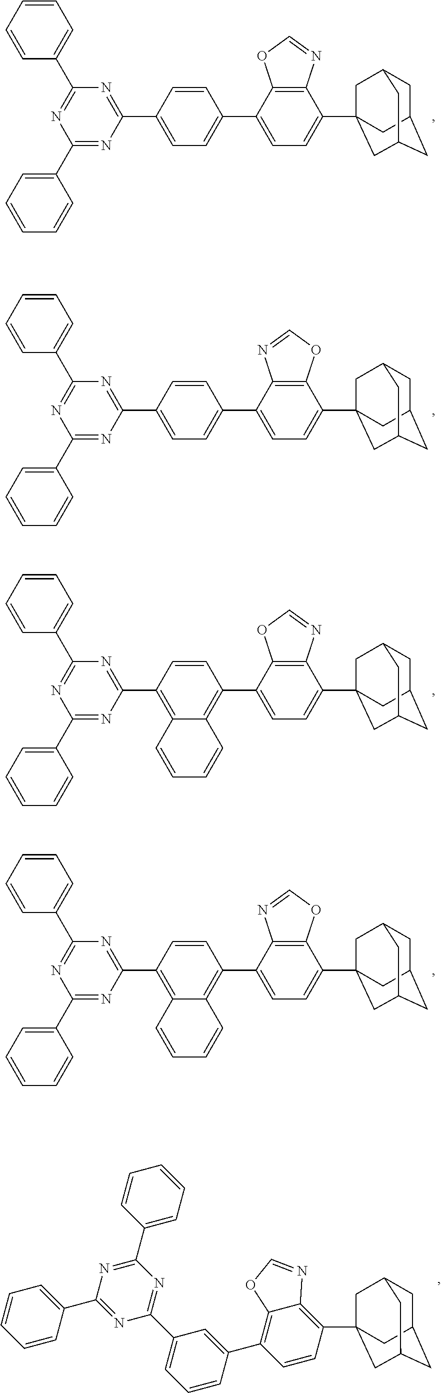 [SMILES: c1ccc(-c2nc(-c3ccccc3)nc(-c3ccc(-c4ccc(C56CC7CC(CC(C7)C5)C6)c5ncoc45)cc3)n2)cc1 ; c1ccc(-c2nc(-c3ccccc3)nc(-c3ccc(-c4ccc(C56CC7CC(CC(C7)C5)C6)c5ocnc45)cc3)n2)cc1 ; c1ccc(-c2nc(-c3ccccc3)nc(-c3ccc(-c4ccc(C56CC7CC(CC(C7)C5)C6)c5ncoc45)c4ccccc34)n2)cc1 ; c1ccc(-c2nc(-c3ccccc3)nc(-c3ccc(-c4ccc(C56CC7CC(CC(C7)C5)C6)c5ocnc45)c4ccccc34)n2)cc1 ; c1ccc(-c2nc(-c3ccccc3)nc(-c3cccc(-c4ccc(C56CC7CC(CC(C7)C5)C6)c5ncoc45)c3)n2)cc1]

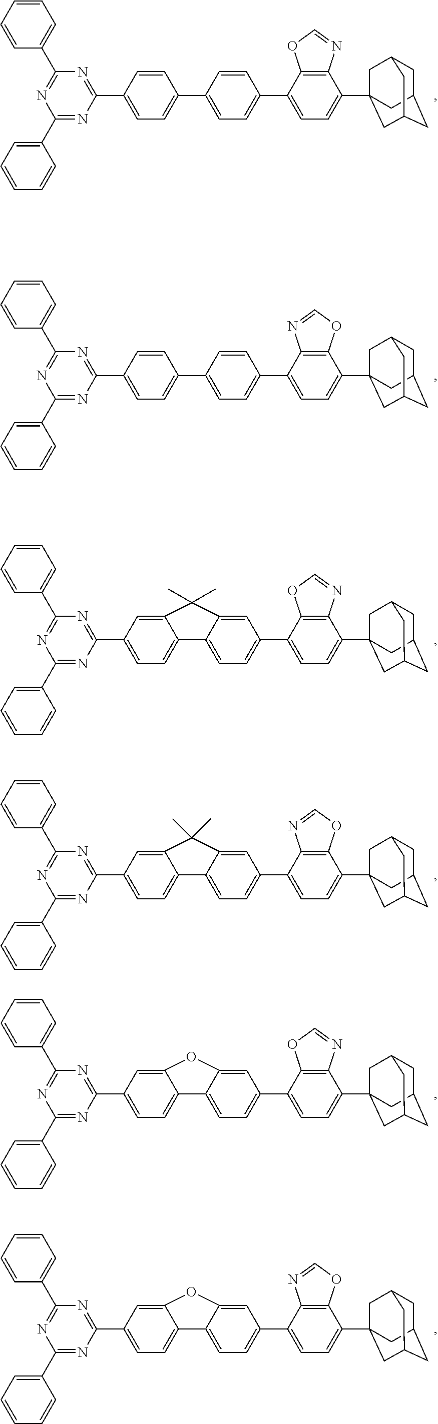

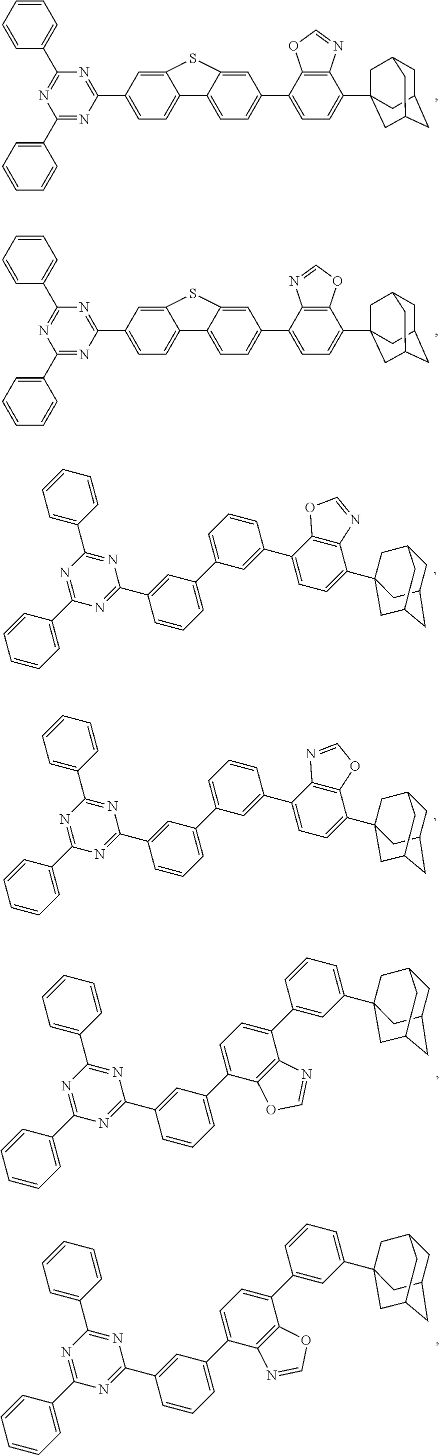

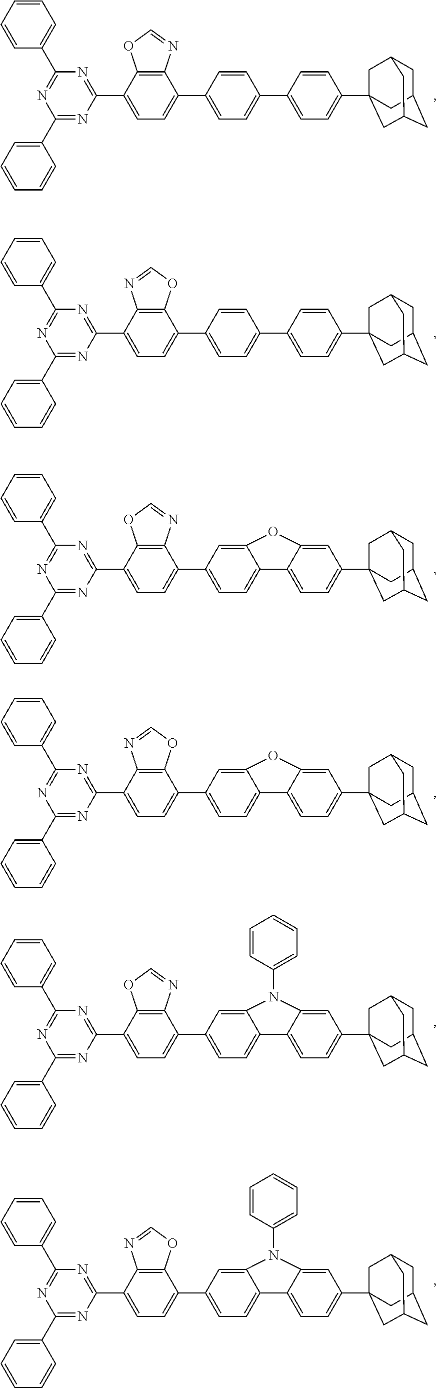

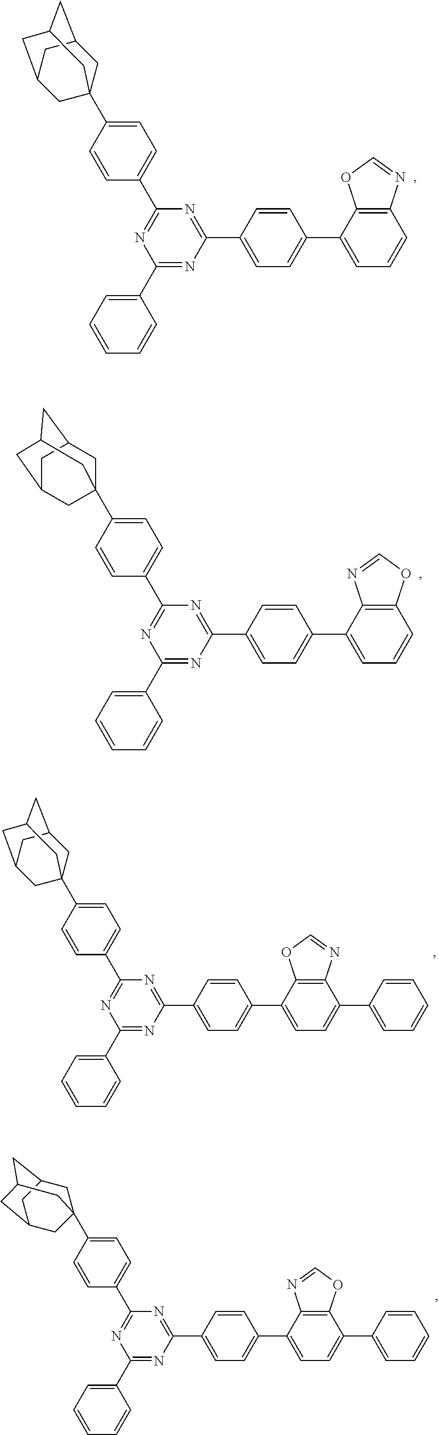 [SMILES: c1ccc(-c2nc(-c3ccc(-c4cccc5ncoc45)cc3)nc(-c3ccc(C45CC6CC(CC(C6)C4)C5)cc3)n2)cc1 ; c1ccc(-c2nc(-c3ccc(-c4cccc5ocnc45)cc3)nc(-c3ccc(C45CC6CC(CC(C6)C4)C5)cc3)n2)cc1 ; c1ccc(-c2nc(-c3ccc(-c4ccc(-c5ccccc5)c5ncoc45)cc3)nc(-c3ccc(C45CC6CC(CC(C6)C4)C5)cc3)n2)cc1 ; c1ccc(-c2nc(-c3ccc(-c4ccc(-c5ccccc5)c5ocnc45)cc3)nc(-c3ccc(C45CC6CC(CC(C6)C4)C5)cc3)n2)cc1]

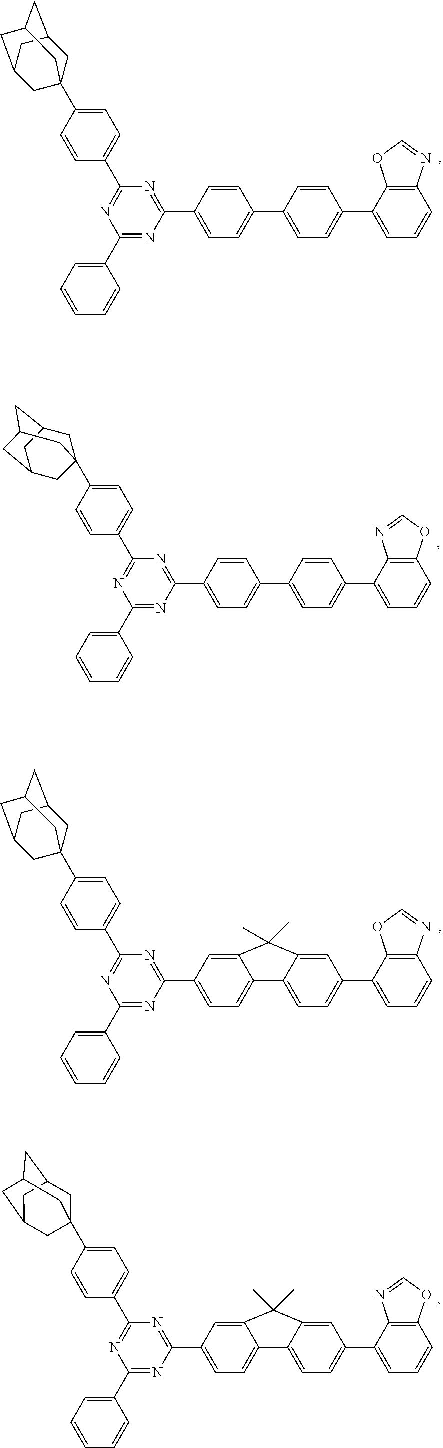 [SMILES: c1ccc(-c2nc(-c3ccc(-c4ccc(-c5cccc6ncoc56)cc4)cc3)nc(-c3ccc(C45CC6CC(CC(C6)C4)C5)cc3)n2)cc1 ; c1ccc(-c2nc(-c3ccc(-c4ccc(-c5cccc6ocnc56)cc4)cc3)nc(-c3ccc(C45CC6CC(CC(C6)C4)C5)cc3)n2)cc1 ; CC1(C)c2cc(-c3nc(-c4ccccc4)nc(-c4ccc(C56CC7CC(CC(C7)C5)C6)cc4)n3)ccc2-c2ccc(-c3cccc4ncoc34)cc21 ; CC1(C)c2cc(-c3nc(-c4ccccc4)nc(-c4ccc(C56CC7CC(CC(C7)C5)C6)cc4)n3)ccc2-c2ccc(-c3cccc4ocnc34)cc21]

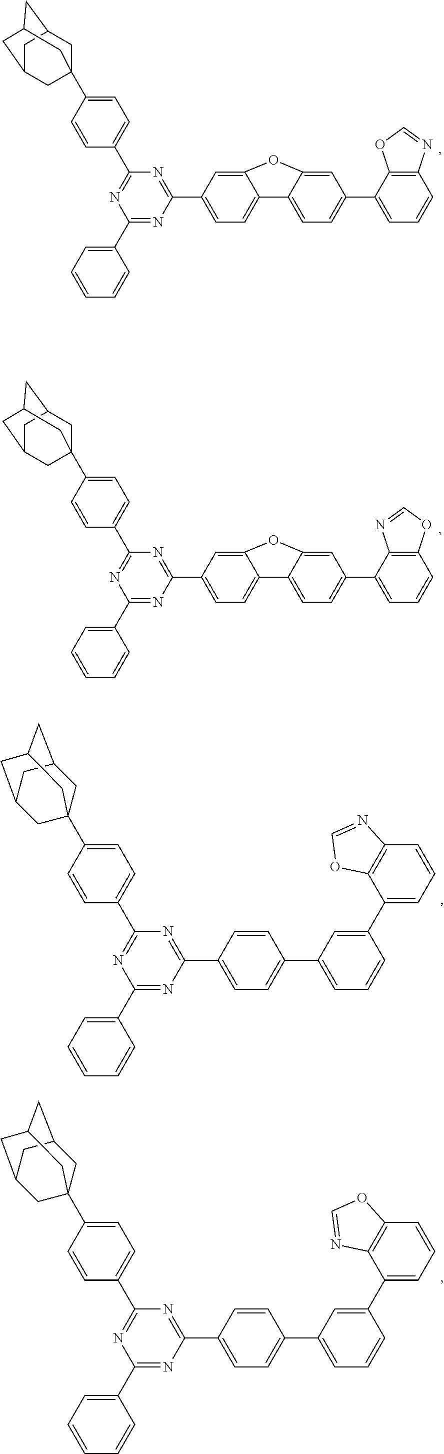 [SMILES: c1ccc(-c2nc(-c3ccc(C45CC6CC(CC(C6)C4)C5)cc3)nc(-c3ccc4c(c3)oc3cc(-c5cccc6ncoc56)ccc34)n2)cc1 ; c1ccc(-c2nc(-c3ccc(C45CC6CC(CC(C6)C4)C5)cc3)nc(-c3ccc4c(c3)oc3cc(-c5cccc6ocnc56)ccc34)n2)cc1 ; c1ccc(-c2nc(-c3ccc(-c4cccc(-c5cccc6ncoc56)c4)cc3)nc(-c3ccc(C45CC6CC(CC(C6)C4)C5)cc3)n2)cc1 ; c1ccc(-c2nc(-c3ccc(-c4cccc(-c5cccc6ocnc56)c4)cc3)nc(-c3ccc(C45CC6CC(CC(C6)C4)C5)cc3)n2)cc1]

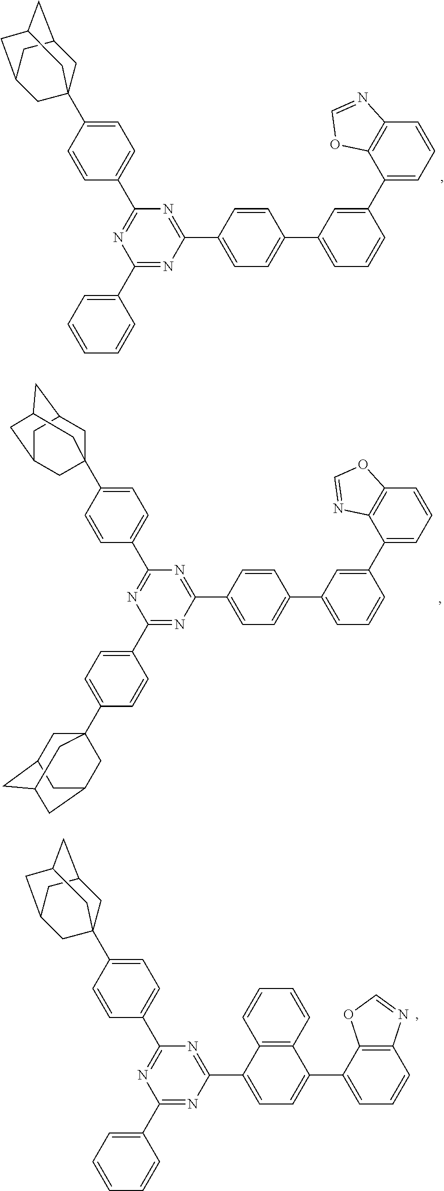

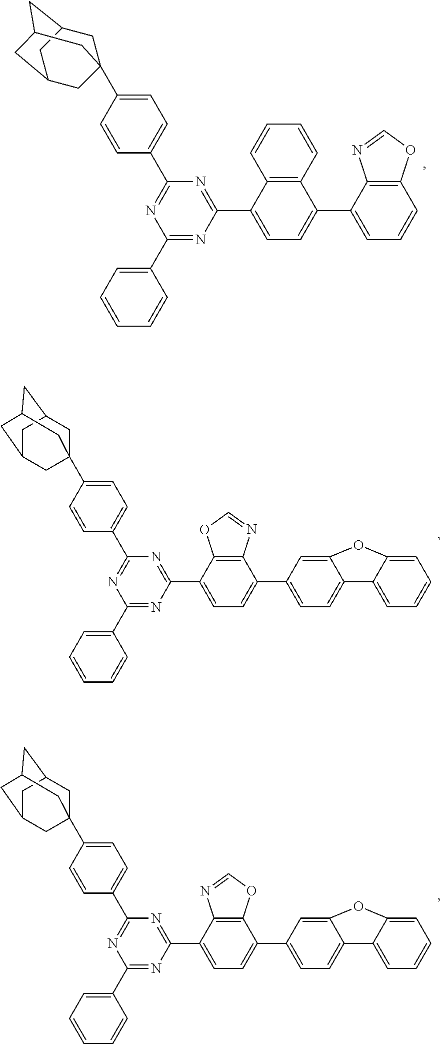 [SMILES: c1ccc(-c2nc(-c3ccc(C45CC6CC(CC(C6)C4)C5)cc3)nc(-c3ccc(-c4cccc5ocnc45)c4ccccc34)n2)cc1 ; c1ccc(-c2nc(-c3ccc(C45CC6CC(CC(C6)C4)C5)cc3)nc(-c3ccc(-c4ccc5c(c4)oc4ccccc45)c4ncoc34)n2)cc1 ; c1ccc(-c2nc(-c3ccc(C45CC6CC(CC(C6)C4)C5)cc3)nc(-c3ccc(-c4ccc5c(c4)oc4ccccc45)c4ocnc34)n2)cc1]

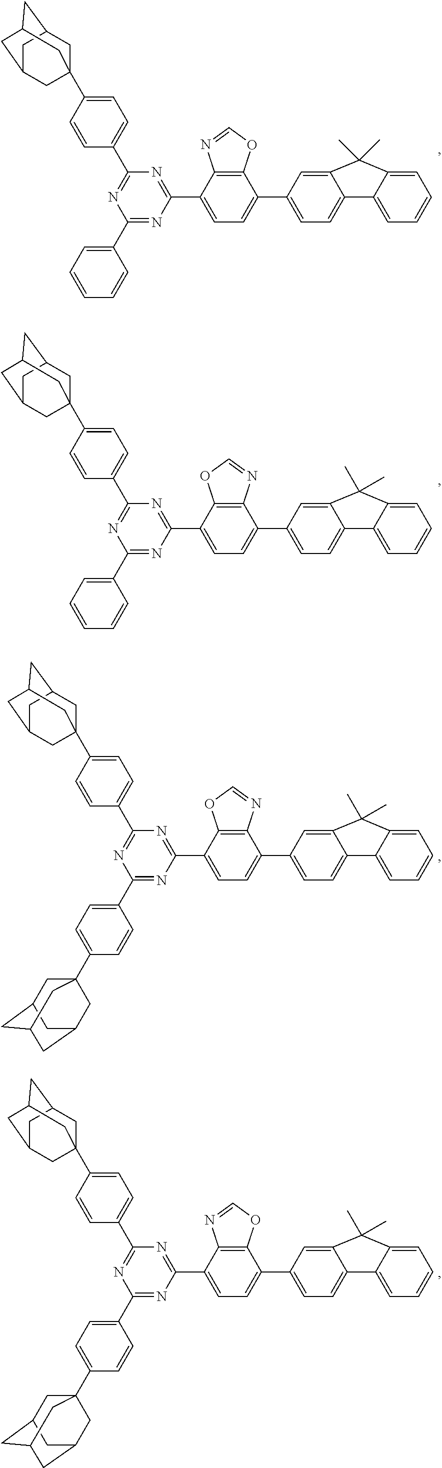

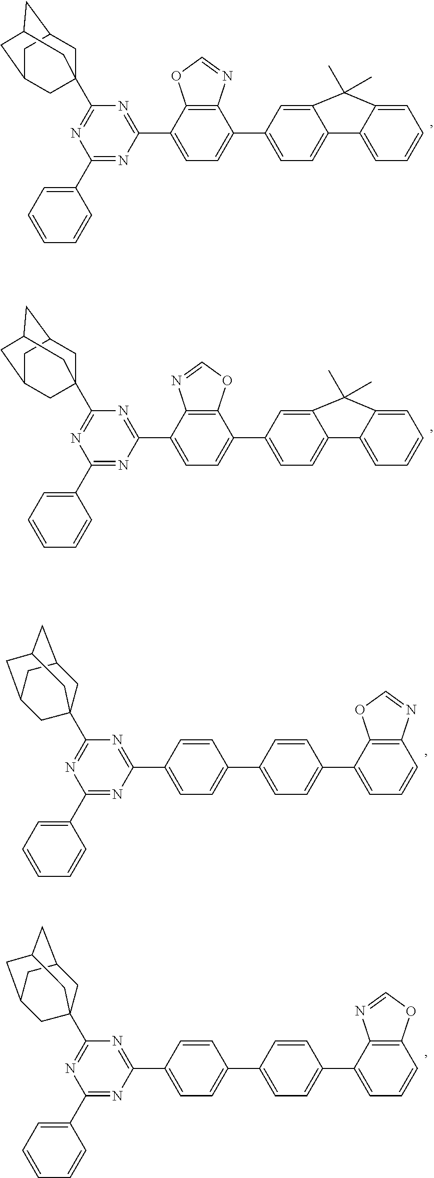 [SMILES: CC1(C)c2ccccc2-c2ccc(-c3ccc(-c4nc(-c5ccccc5)nc(C56CC7CC(CC(C7)C5)C6)n4)c4ocnc34)cc21 ; CC1(C)c2ccccc2-c2ccc(-c3ccc(-c4nc(-c5ccccc5)nc(C56CC7CC(CC(C7)C5)C6)n4)c4ncoc34)cc21 ; c1ccc(-c2nc(-c3ccc(-c4ccc(-c5cccc6ncoc56)cc4)cc3)nc(C34CC5CC(CC(C5)C3)C4)n2)cc1 ; c1ccc(-c2nc(-c3ccc(-c4ccc(-c5cccc6ocnc56)cc4)cc3)nc(C34CC5CC(CC(C5)C3)C4)n2)cc1]

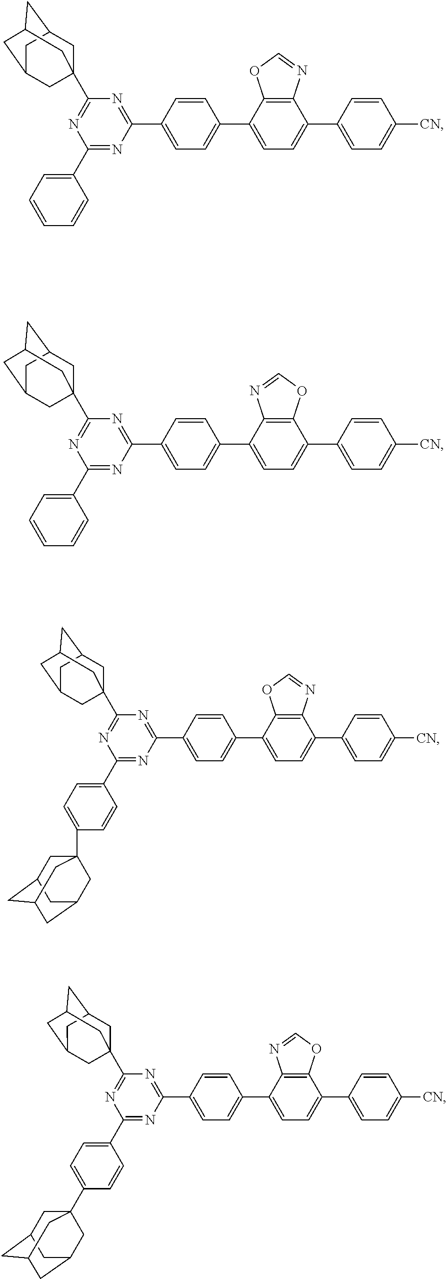 [SMILES: N#Cc1ccc(-c2ccc(-c3ccc(-c4nc(-c5ccccc5)nc(C56CC7CC(CC(C7)C5)C6)n4)cc3)c3ocnc23)cc1 ; N#Cc1ccc(-c2ccc(-c3ccc(-c4nc(-c5ccccc5)nc(C56CC7CC(CC(C7)C5)C6)n4)cc3)c3ncoc23)cc1 ; N#Cc1ccc(-c2ccc(-c3ccc(-c4nc(-c5ccc(C67CC8CC(CC(C8)C6)C7)cc5)nc(C56CC7CC(CC(C7)C5)C6)n4)cc3)c3ocnc23)cc1 ; N#Cc1ccc(-c2ccc(-c3ccc(-c4nc(-c5ccc(C67CC8CC(CC(C8)C6)C7)cc5)nc(C56CC7CC(CC(C7)C5)C6)n4)cc3)c3ncoc23)cc1]

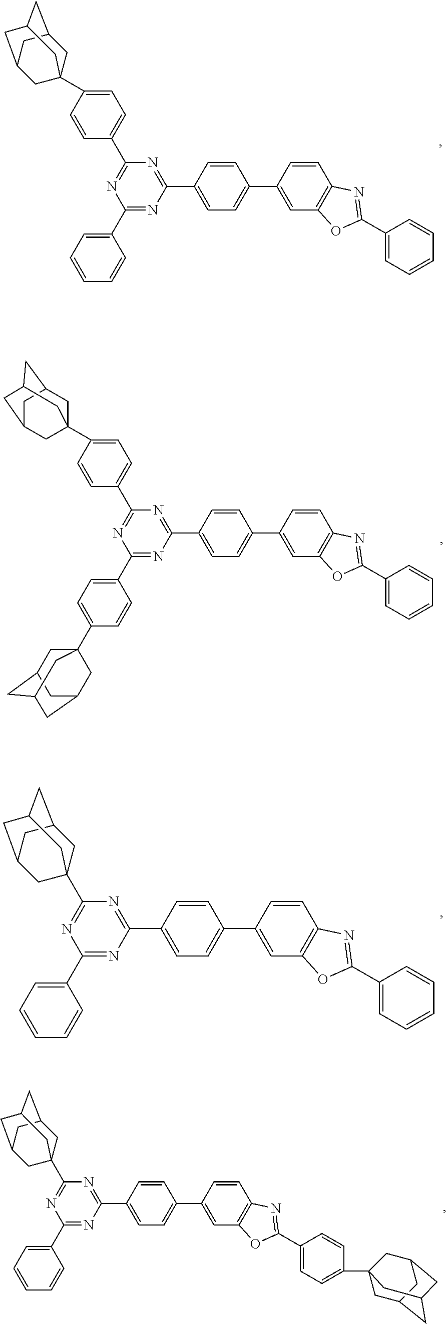 [SMILES: c1ccc(-c2nc(-c3ccc(-c4ccc5nc(-c6ccccc6)oc5c4)cc3)nc(-c3ccc(C45CC6CC(CC(C6)C4)C5)cc3)n2)cc1 ; c1ccc(-c2nc3ccc(-c4ccc(-c5nc(-c6ccc(C78CC9CC(CC(C9)C7)C8)cc6)nc(-c6ccc(C78CC9CC(CC(C9)C7)C8)cc6)n5)cc4)cc3o2)cc1 ; c1ccc(-c2nc(-c3ccc(-c4ccc5nc(-c6ccccc6)oc5c4)cc3)nc(C34CC5CC(CC(C5)C3)C4)n2)cc1 ; c1ccc(-c2nc(-c3ccc(-c4ccc5nc(-c6ccc(C78CC9CC(CC(C9)C7)C8)cc6)oc5c4)cc3)nc(C34CC5CC(CC(C5)C3)C4)n2)cc1]

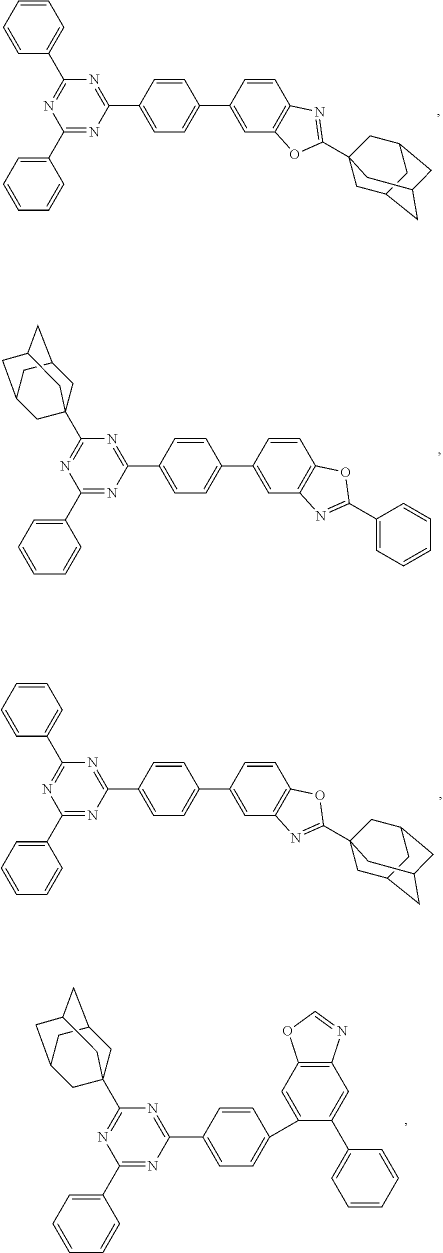 [SMILES: c1ccc(-c2nc(-c3ccccc3)nc(-c3ccc(-c4ccc5nc(C67CC8CC(CC(C8)C6)C7)oc5c4)cc3)n2)cc1 ; c1ccc(-c2nc(-c3ccc(-c4ccc5oc(-c6ccccc6)nc5c4)cc3)nc(C34CC5CC(CC(C5)C3)C4)n2)cc1 ; c1ccc(-c2nc(-c3ccccc3)nc(-c3ccc(-c4ccc5oc(C67CC8CC(CC(C8)C6)C7)nc5c4)cc3)n2)cc1 ; c1ccc(-c2nc(-c3ccc(-c4cc5ocnc5cc4-c4ccccc4)cc3)nc(C34CC5CC(CC(C5)C3)C4)n2)cc1]

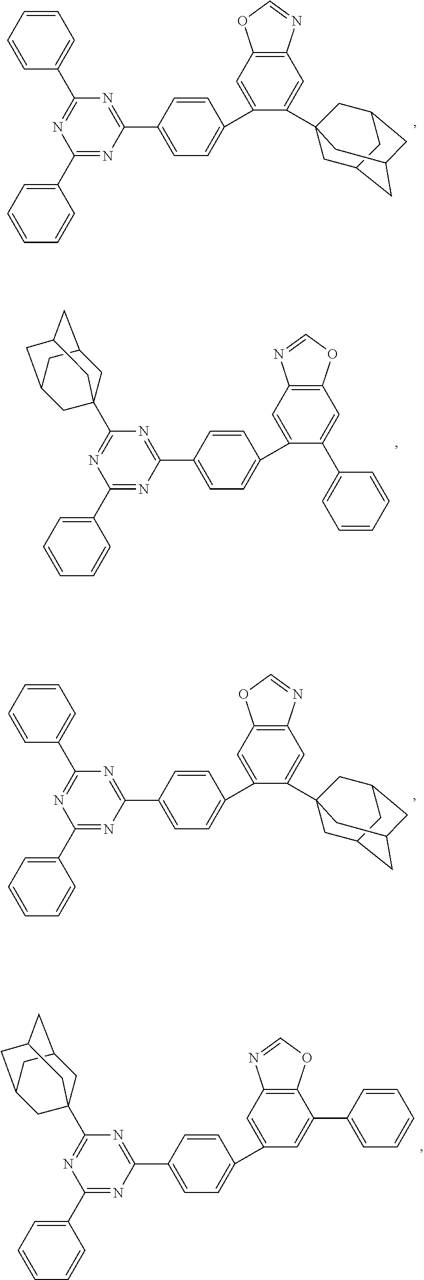 [SMILES: c1ccc(-c2nc(-c3ccccc3)nc(-c3ccc(-c4cc5ocnc5cc4C45CC6CC(CC(C6)C4)C5)cc3)n2)cc1 ; c1ccc(-c2nc(-c3ccc(-c4cc5ncoc5cc4-c4ccccc4)cc3)nc(C34CC5CC(CC(C5)C3)C4)n2)cc1 ; c1ccc(-c2nc(-c3ccccc3)nc(-c3ccc(-c4cc5ocnc5cc4C45CC6CC(CC(C6)C4)C5)cc3)n2)cc1 ; c1ccc(-c2nc(-c3ccc(-c4cc(-c5ccccc5)c5ocnc5c4)cc3)nc(C34CC5CC(CC(C5)C3)C4)n2)cc1]

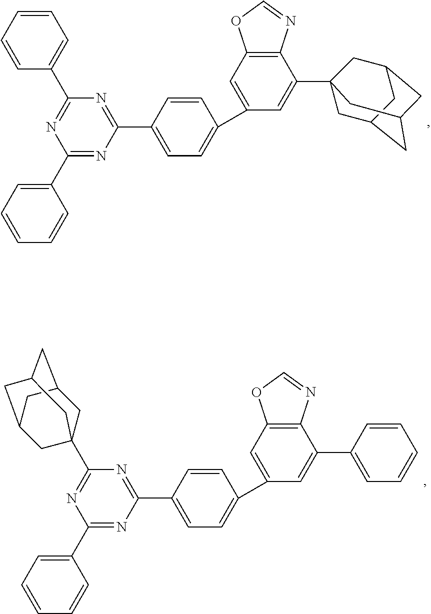 [SMILES: c1ccc(-c2nc(-c3ccccc3)nc(-c3ccc(-c4cc(C56CC7CC(CC(C7)C5)C6)c5ncoc5c4)cc3)n2)cc1 ; c1ccc(-c2nc(-c3ccc(-c4cc(-c5ccccc5)c5ncoc5c4)cc3)nc(C34CC5CC(CC(C5)C3)C4)n2)cc1]

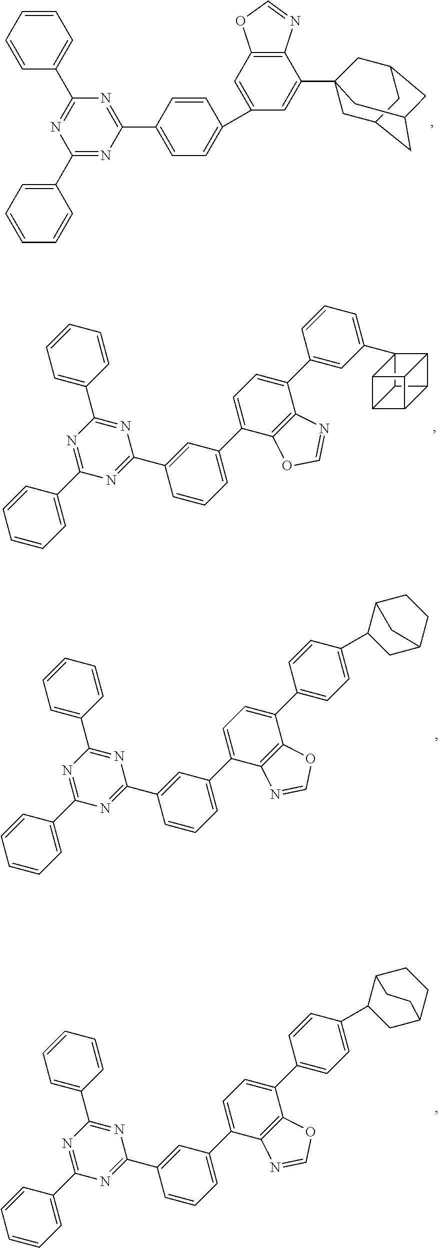 [SMILES: c1ccc(-c2nc(-c3ccccc3)nc(-c3ccc(-c4cc(C56CC7CC(CC(C7)C5)C6)c5ncoc5c4)cc3)n2)cc1 ; c1ccc(-c2nc(-c3ccccc3)nc(-c3cccc(-c4ccc(-c5cccc(C67C8C9C%10C8C6C%10C97)c5)c5ncoc45)c3)n2)cc1 ; c1ccc(-c2nc(-c3ccccc3)nc(-c3cccc(-c4ccc(-c5ccc(C6CC7CCC6C7)cc5)c5ocnc45)c3)n2)cc1 ; c1ccc(-c2nc(-c3ccccc3)nc(-c3cccc(-c4ccc(-c5ccc(C6CC7CCC6CC7)cc5)c5ocnc45)c3)n2)cc1]

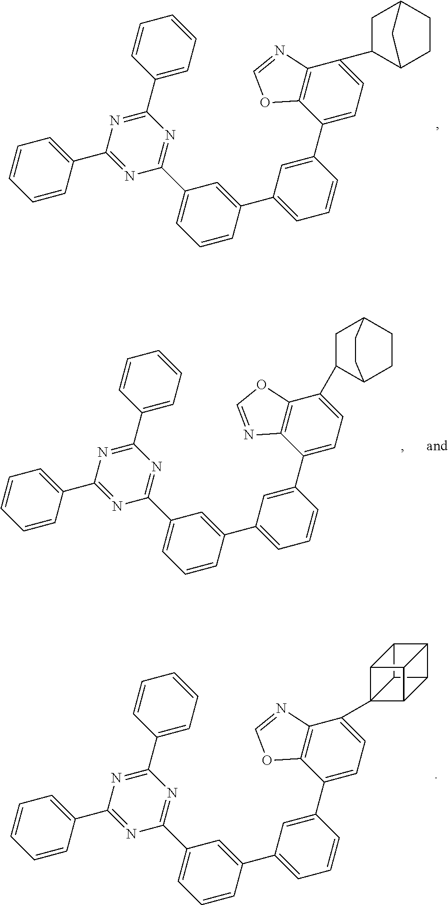 [SMILES: c1ccc(-c2nc(-c3ccccc3)nc(-c3cccc(-c4cccc(-c5ccc(C6CC7CCC6C7)c6ncoc56)c4)c3)n2)cc1 ; c1ccc(-c2nc(-c3ccccc3)nc(-c3cccc(-c4cccc(-c5ccc(C6CC7CCC6CC7)c6ocnc56)c4)c3)n2)cc1 ; c1ccc(-c2nc(-c3ccccc3)nc(-c3cccc(-c4cccc(-c5ccc(C67C8C9C%10C8C6C%10C97)c6ncoc56)c4)c3)n2)cc1]

Optionally, in the material of the electron transport layer, L3 includes a first benzene ring and a second benzene ring, and the first benzene ring is bonded to the second benzene ring; and the first benzene ring is bonded to the heteroaromatic ring group including at least one nitrogen atom, and the heteroaromatic ring group including at least one nitrogen atom is located at the meta-position of the second benzene ring. In this case, the two phenyl groups are connected at the meta-position, with a large torsion angle, which can effectively destroy or reduce the conjugation among the molecules, and can further increase the T1 of the material. The high T1 can further increase the utilization ratio of the excitons, thereby increasing the luminous efficiency and the life of the light emitting device.

Optionally, the chemical structural formula of the material of the electron transport layer includes any one of

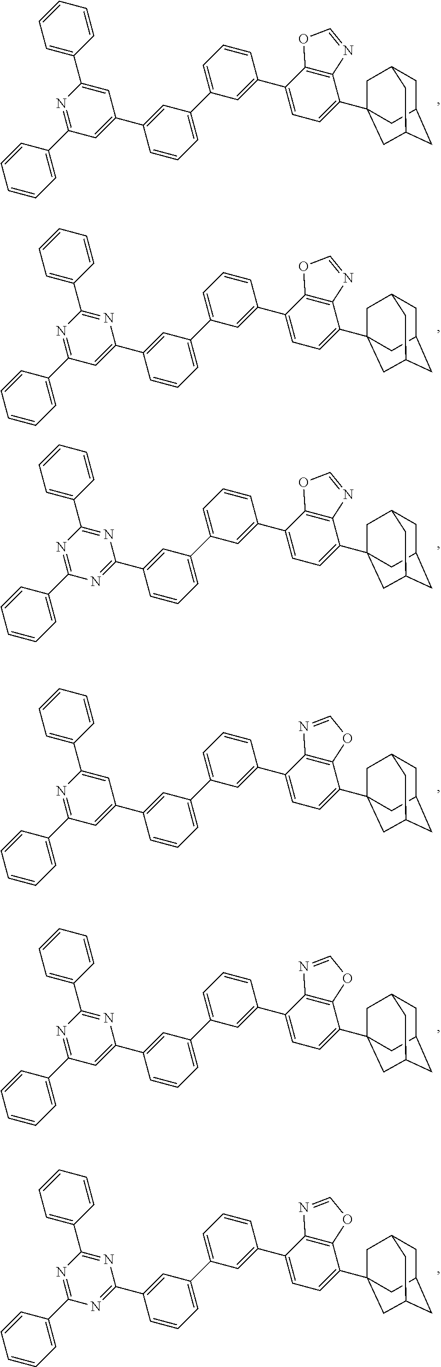

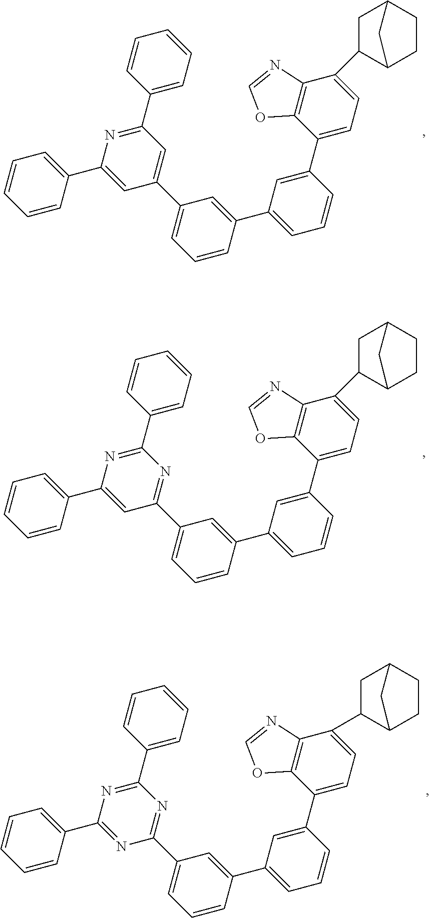 [SMILES: c1ccc(-c2cc(-c3cccc(-c4cccc(-c5ccc(C6CC7CCC6C7)c6ncoc56)c4)c3)cc(-c3ccccc3)n2)cc1 ; c1ccc(-c2cc(-c3cccc(-c4cccc(-c5ccc(C6CC7CCC6C7)c6ncoc56)c4)c3)nc(-c3ccccc3)n2)cc1 ; c1ccc(-c2nc(-c3ccccc3)nc(-c3cccc(-c4cccc(-c5ccc(C6CC7CCC6C7)c6ncoc56)c4)c3)n2)cc1]

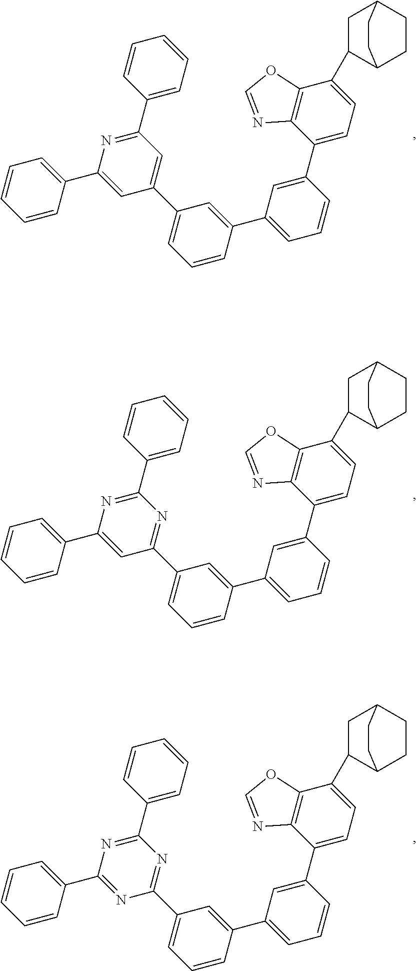 [SMILES: c1ccc(-c2cc(-c3cccc(-c4cccc(-c5ccc(C6CC7CCC6CC7)c6ocnc56)c4)c3)cc(-c3ccccc3)n2)cc1 ; c1ccc(-c2cc(-c3cccc(-c4cccc(-c5ccc(C6CC7CCC6CC7)c6ocnc56)c4)c3)nc(-c3ccccc3)n2)cc1 ; c1ccc(-c2nc(-c3ccccc3)nc(-c3cccc(-c4cccc(-c5ccc(C6CC7CCC6CC7)c6ocnc56)c4)c3)n2)cc1]

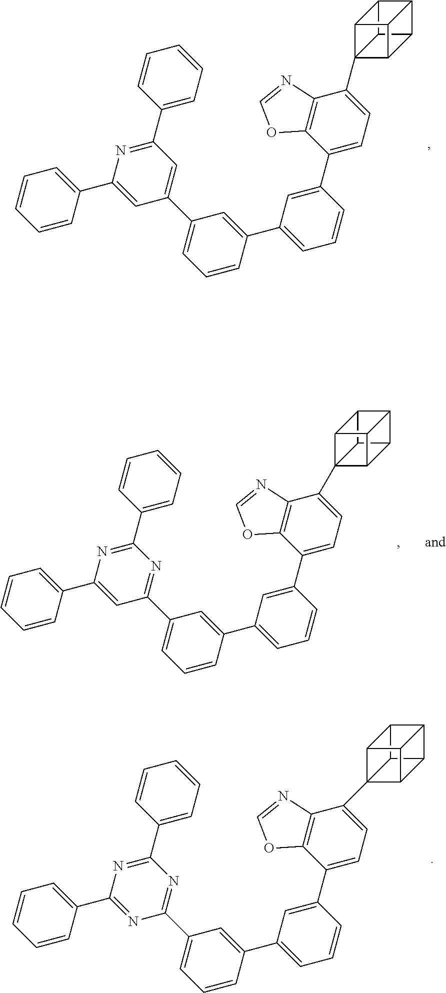 [SMILES: c1ccc(-c2cc(-c3cccc(-c4cccc(-c5ccc(C67C8C9C%10C8C6C%10C97)c6ncoc56)c4)c3)cc(-c3ccccc3)n2)cc1 ; c1ccc(-c2cc(-c3cccc(-c4cccc(-c5ccc(C67C8C9C%10C8C6C%10C97)c6ncoc56)c4)c3)nc(-c3ccccc3)n2)cc1 ; c1ccc(-c2nc(-c3ccccc3)nc(-c3cccc(-c4cccc(-c5ccc(C67C8C9C%10C8C6C%10C97)c6ncoc56)c4)c3)n2)cc1]

Optionally, the material of the electron transport layer has cyano. In this case, the material of the electron transport layer has a strong electron-withdrawing capability, and a high electron mobility.

Optionally, the chemical structural formula of the material of the electron transport layer having the cyano includes any one of

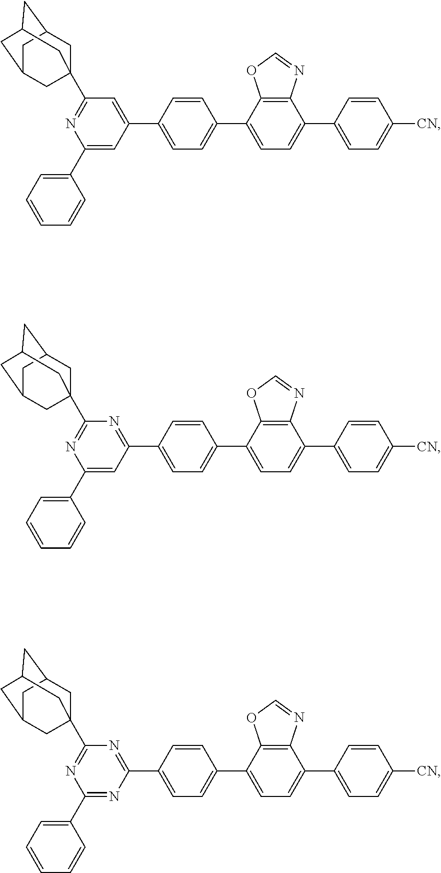 [SMILES: N#Cc1ccc(-c2ccc(-c3ccc(-c4cc(-c5ccccc5)nc(C56CC7CC(CC(C7)C5)C6)c4)cc3)c3ocnc23)cc1 ; N#Cc1ccc(-c2ccc(-c3ccc(-c4cc(-c5ccccc5)nc(C56CC7CC(CC(C7)C5)C6)n4)cc3)c3ocnc23)cc1 ; N#Cc1ccc(-c2ccc(-c3ccc(-c4nc(-c5ccccc5)nc(C56CC7CC(CC(C7)C5)C6)n4)cc3)c3ocnc23)cc1]

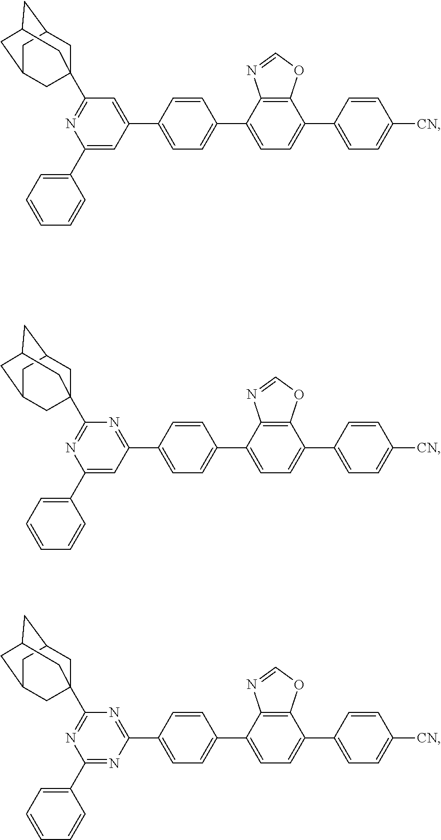 [SMILES: N#Cc1ccc(-c2ccc(-c3ccc(-c4cc(-c5ccccc5)nc(C56CC7CC(CC(C7)C5)C6)c4)cc3)c3ncoc23)cc1 ; N#Cc1ccc(-c2ccc(-c3ccc(-c4cc(-c5ccccc5)nc(C56CC7CC(CC(C7)C5)C6)n4)cc3)c3ncoc23)cc1 ; N#Cc1ccc(-c2ccc(-c3ccc(-c4nc(-c5ccccc5)nc(C56CC7CC(CC(C7)C5)C6)n4)cc3)c3ncoc23)cc1]

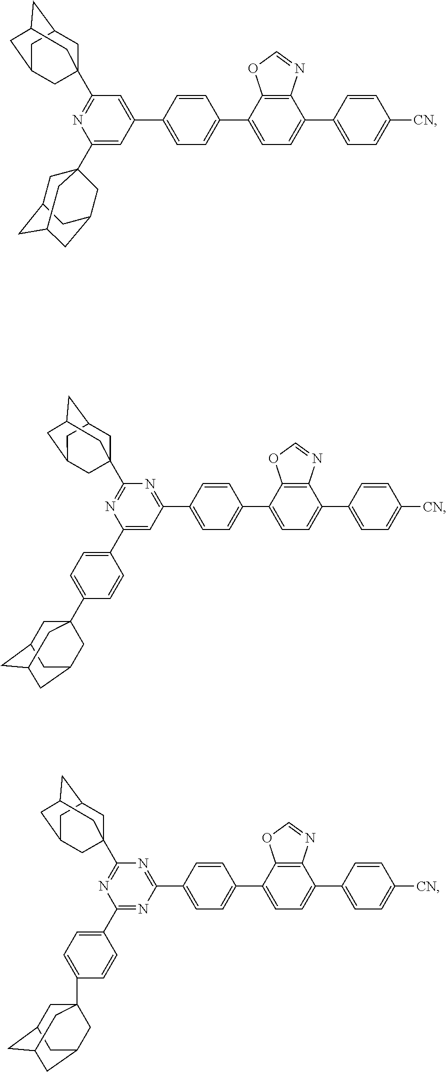 [SMILES: N#Cc1ccc(-c2ccc(-c3ccc(-c4cc(C56CC7CC(CC(C7)C5)C6)nc(C56CC7CC(CC(C7)C5)C6)c4)cc3)c3ocnc23)cc1 ; N#Cc1ccc(-c2ccc(-c3ccc(-c4cc(-c5ccc(C67CC8CC(CC(C8)C6)C7)cc5)nc(C56CC7CC(CC(C7)C5)C6)n4)cc3)c3ocnc23)cc1 ; N#Cc1ccc(-c2ccc(-c3ccc(-c4nc(-c5ccc(C67CC8CC(CC(C8)C6)C7)cc5)nc(C56CC7CC(CC(C7)C5)C6)n4)cc3)c3ocnc23)cc1]

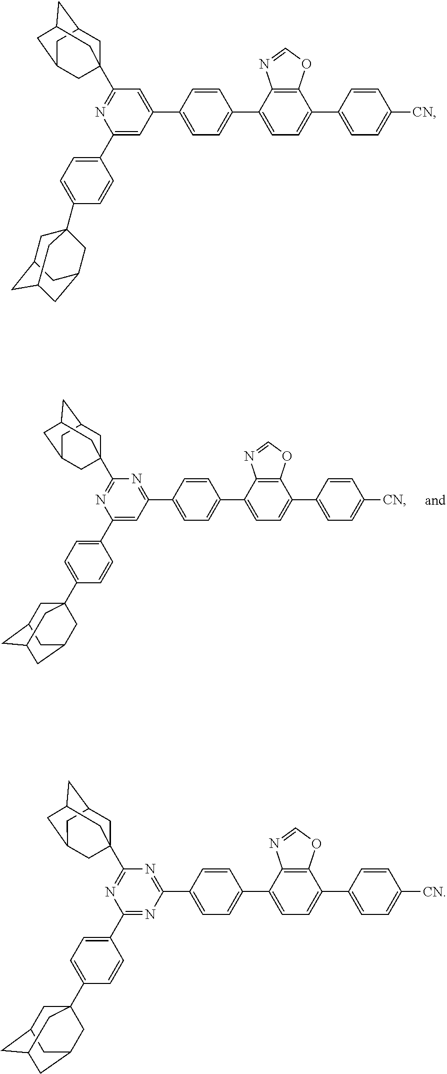 [SMILES: N#Cc1ccc(-c2ccc(-c3ccc(-c4cc(-c5ccc(C67CC8CC(CC(C8)C6)C7)cc5)nc(C56CC7CC(CC(C7)C5)C6)c4)cc3)c3ncoc23)cc1 ; N#Cc1ccc(-c2ccc(-c3ccc(-c4cc(-c5ccc(C67CC8CC(CC(C8)C6)C7)cc5)nc(C56CC7CC(CC(C7)C5)C6)n4)cc3)c3ncoc23)cc1 ; N#Cc1ccc(-c2ccc(-c3ccc(-c4nc(-c5ccc(C67CC8CC(CC(C8)C6)C7)cc5)nc(C56CC7CC(CC(C7)C5)C6)n4)cc3)c3ncoc23)cc1]

Optionally, in the material of the electron transport layer, the adamantane is connected to the heteroaromatic ring group including at least one nitrogen atom by a phenyl group, and is located at the ortho-position of the nitrogen atom in the heteroaromatic ring group. If the adamantane is directly connected to the heteroaromatic ring group including at least one nitrogen atom, that destroys the conjugation of the strong electron-withdrawing group. Accordingly, the adamantane is connected to the heteroaromatic ring group including at least one nitrogen atom by a phenyl group, and is located at the ortho-position of the nitrogen atom in the heteroaromatic ring group, which does not destroy the conjugation of the strong electron-withdrawing group.

Optionally, the chemical structural formula of the material of the electron transport layer includes any one of

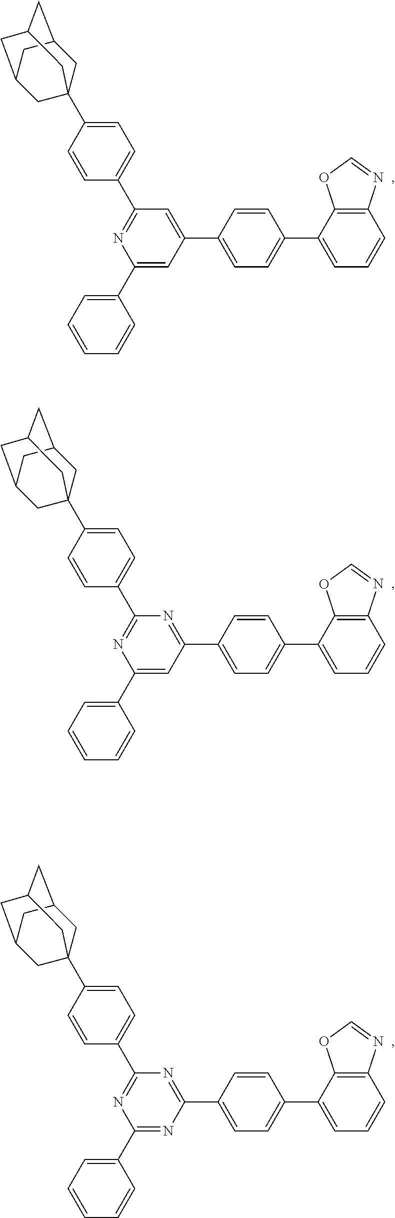 [SMILES: c1ccc(-c2cc(-c3ccc(-c4cccc5ncoc45)cc3)cc(-c3ccc(C45CC6CC(CC(C6)C4)C5)cc3)n2)cc1 ; c1ccc(-c2cc(-c3ccc(-c4cccc5ncoc45)cc3)nc(-c3ccc(C45CC6CC(CC(C6)C4)C5)cc3)n2)cc1 ; c1ccc(-c2nc(-c3ccc(-c4cccc5ncoc45)cc3)nc(-c3ccc(C45CC6CC(CC(C6)C4)C5)cc3)n2)cc1]

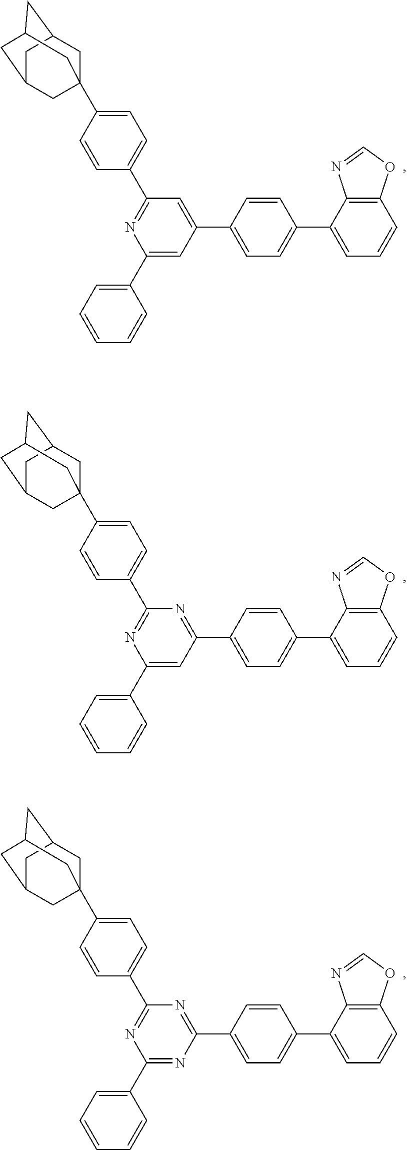 [SMILES: c1ccc(-c2cc(-c3ccc(-c4cccc5ocnc45)cc3)cc(-c3ccc(C45CC6CC(CC(C6)C4)C5)cc3)n2)cc1 ; c1ccc(-c2cc(-c3ccc(-c4cccc5ocnc45)cc3)nc(-c3ccc(C45CC6CC(CC(C6)C4)C5)cc3)n2)cc1 ; c1ccc(-c2nc(-c3ccc(-c4cccc5ocnc45)cc3)nc(-c3ccc(C45CC6CC(CC(C6)C4)C5)cc3)n2)cc1]

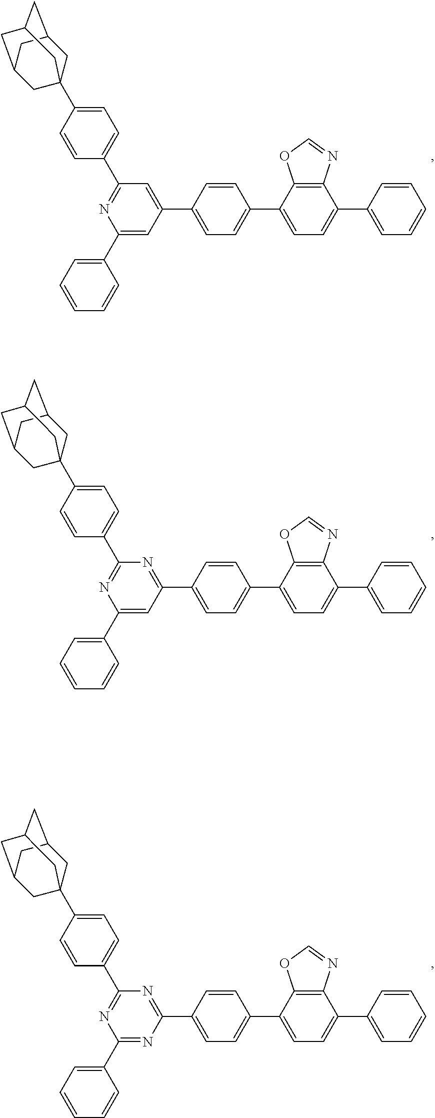 [SMILES: c1ccc(-c2cc(-c3ccc(-c4ccc(-c5ccccc5)c5ncoc45)cc3)cc(-c3ccc(C45CC6CC(CC(C6)C4)C5)cc3)n2)cc1 ; c1ccc(-c2cc(-c3ccc(-c4ccc(-c5ccccc5)c5ncoc45)cc3)nc(-c3ccc(C45CC6CC(CC(C6)C4)C5)cc3)n2)cc1 ; c1ccc(-c2nc(-c3ccc(-c4ccc(-c5ccccc5)c5ncoc45)cc3)nc(-c3ccc(C45CC6CC(CC(C6)C4)C5)cc3)n2)cc1]

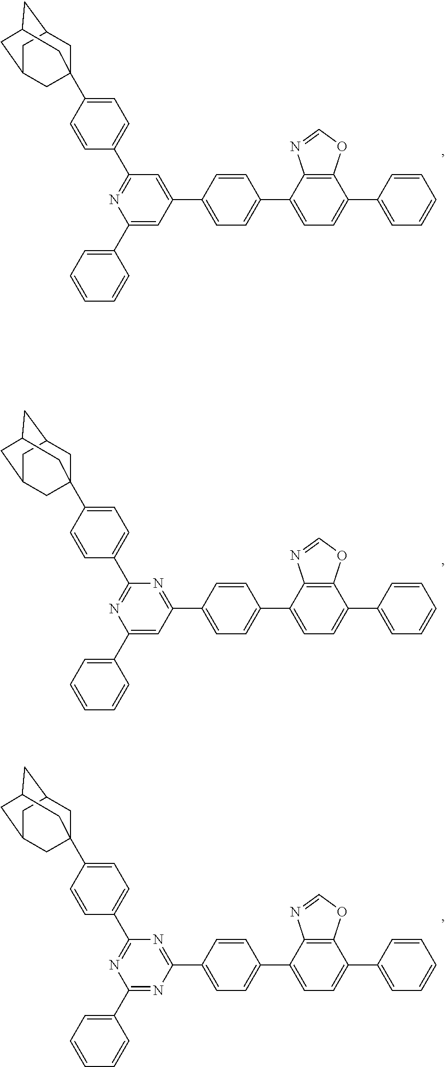 [SMILES: c1ccc(-c2cc(-c3ccc(-c4ccc(-c5ccccc5)c5ocnc45)cc3)cc(-c3ccc(C45CC6CC(CC(C6)C4)C5)cc3)n2)cc1 ; c1ccc(-c2cc(-c3ccc(-c4ccc(-c5ccccc5)c5ocnc45)cc3)nc(-c3ccc(C45CC6CC(CC(C6)C4)C5)cc3)n2)cc1 ; c1ccc(-c2nc(-c3ccc(-c4ccc(-c5ccccc5)c5ocnc45)cc3)nc(-c3ccc(C45CC6CC(CC(C6)C4)C5)cc3)n2)cc1]

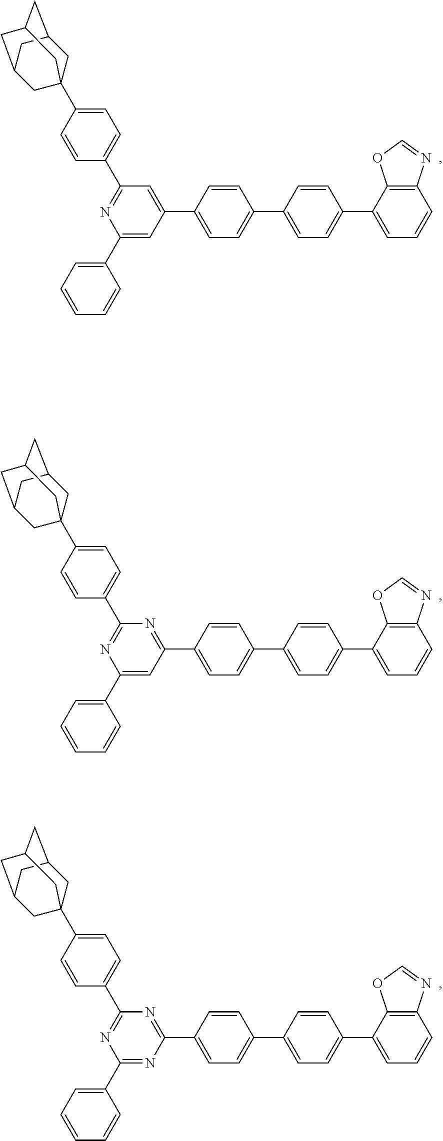 [SMILES: c1ccc(-c2cc(-c3ccc(-c4ccc(-c5cccc6ncoc56)cc4)cc3)cc(-c3ccc(C45CC6CC(CC(C6)C4)C5)cc3)n2)cc1 ; c1ccc(-c2cc(-c3ccc(-c4ccc(-c5cccc6ncoc56)cc4)cc3)nc(-c3ccc(C45CC6CC(CC(C6)C4)C5)cc3)n2)cc1 ; c1ccc(-c2nc(-c3ccc(-c4ccc(-c5cccc6ncoc56)cc4)cc3)nc(-c3ccc(C45CC6CC(CC(C6)C4)C5)cc3)n2)cc1]

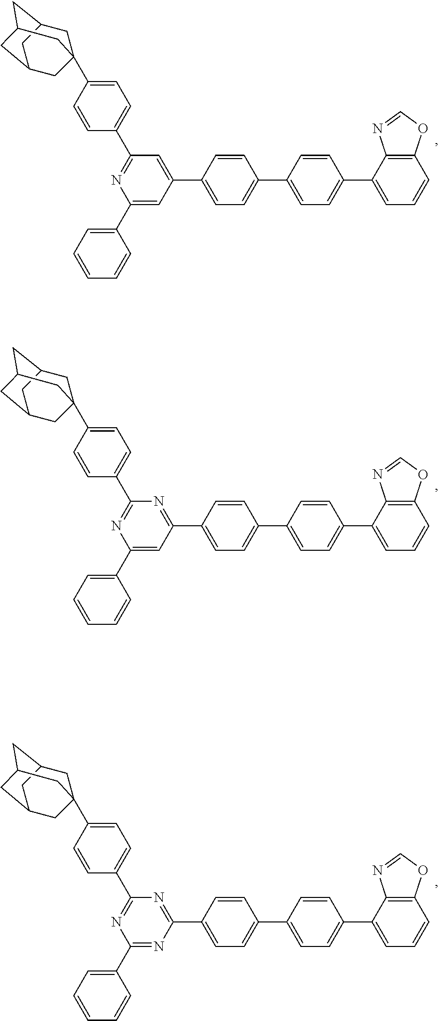 [SMILES: c1ccc(-c2cc(-c3ccc(-c4ccc(-c5cccc6ocnc56)cc4)cc3)cc(-c3ccc(C45CC6CC(CC(C6)C4)C5)cc3)n2)cc1 ; c1ccc(-c2cc(-c3ccc(-c4ccc(-c5cccc6ocnc56)cc4)cc3)nc(-c3ccc(C45CC6CC(CC(C6)C4)C5)cc3)n2)cc1 ; c1ccc(-c2nc(-c3ccc(-c4ccc(-c5cccc6ocnc56)cc4)cc3)nc(-c3ccc(C45CC6CC(CC(C6)C4)C5)cc3)n2)cc1]

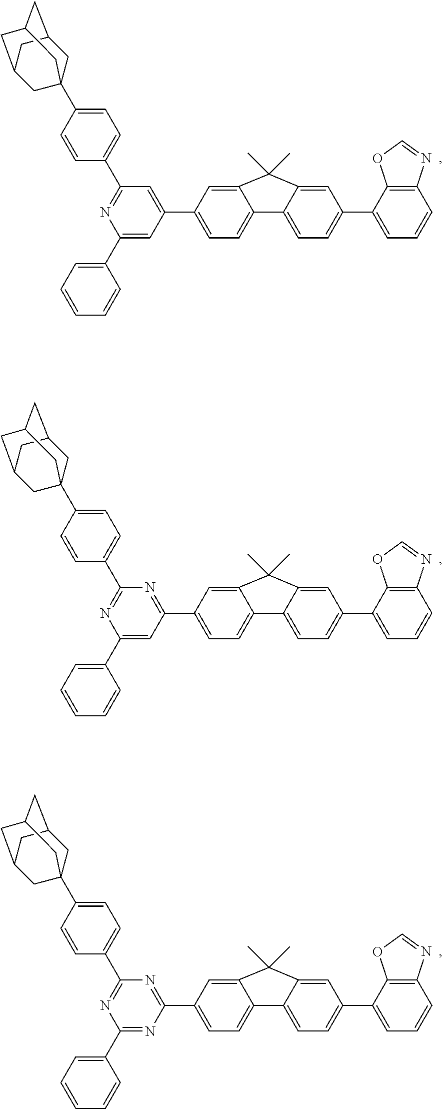 [SMILES: CC1(C)c2cc(-c3cc(-c4ccccc4)nc(-c4ccc(C56CC7CC(CC(C7)C5)C6)cc4)c3)ccc2-c2ccc(-c3cccc4ncoc34)cc21 ; CC1(C)c2cc(-c3cc(-c4ccccc4)nc(-c4ccc(C56CC7CC(CC(C7)C5)C6)cc4)n3)ccc2-c2ccc(-c3cccc4ncoc34)cc21 ; CC1(C)c2cc(-c3nc(-c4ccccc4)nc(-c4ccc(C56CC7CC(CC(C7)C5)C6)cc4)n3)ccc2-c2ccc(-c3cccc4ncoc34)cc21]

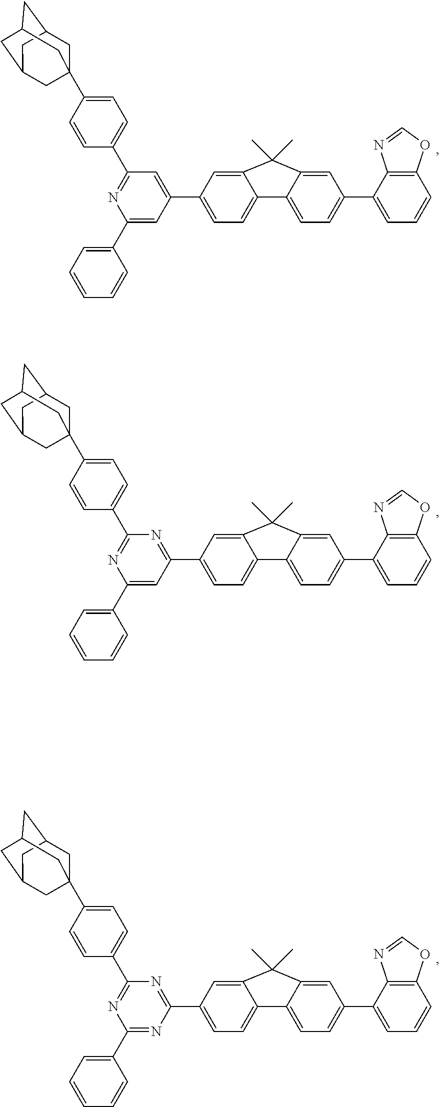 [SMILES: CC1(C)c2cc(-c3cc(-c4ccccc4)nc(-c4ccc(C56CC7CC(CC(C7)C5)C6)cc4)c3)ccc2-c2ccc(-c3cccc4ocnc34)cc21 ; CC1(C)c2cc(-c3cc(-c4ccccc4)nc(-c4ccc(C56CC7CC(CC(C7)C5)C6)cc4)n3)ccc2-c2ccc(-c3cccc4ocnc34)cc21 ; CC1(C)c2cc(-c3nc(-c4ccccc4)nc(-c4ccc(C56CC7CC(CC(C7)C5)C6)cc4)n3)ccc2-c2ccc(-c3cccc4ocnc34)cc21]

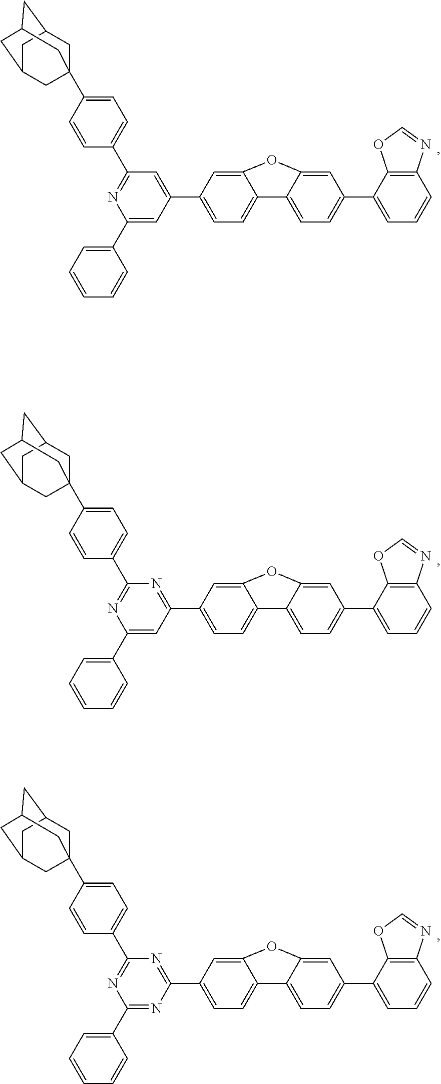 [SMILES: c1ccc(-c2cc(-c3ccc4c(c3)oc3cc(-c5cccc6ncoc56)ccc34)cc(-c3ccc(C45CC6CC(CC(C6)C4)C5)cc3)n2)cc1 ; c1ccc(-c2cc(-c3ccc4c(c3)oc3cc(-c5cccc6ncoc56)ccc34)nc(-c3ccc(C45CC6CC(CC(C6)C4)C5)cc3)n2)cc1 ; c1ccc(-c2nc(-c3ccc(C45CC6CC(CC(C6)C4)C5)cc3)nc(-c3ccc4c(c3)oc3cc(-c5cccc6ncoc56)ccc34)n2)cc1]

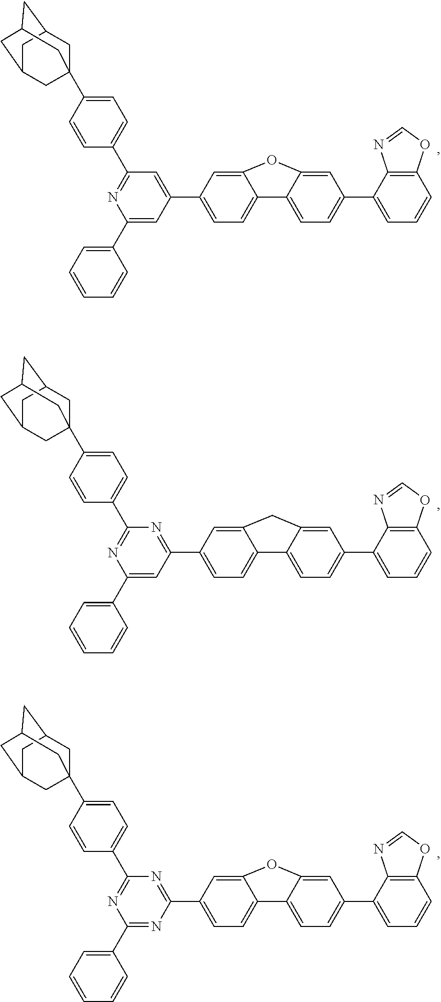 [SMILES: c1ccc(-c2cc(-c3ccc4c(c3)oc3cc(-c5cccc6ocnc56)ccc34)cc(-c3ccc(C45CC6CC(CC(C6)C4)C5)cc3)n2)cc1 ; c1ccc(-c2cc(-c3ccc4c(c3)Cc3cc(-c5cccc6ocnc56)ccc3-4)nc(-c3ccc(C45CC6CC(CC(C6)C4)C5)cc3)n2)cc1 ; c1ccc(-c2nc(-c3ccc(C45CC6CC(CC(C6)C4)C5)cc3)nc(-c3ccc4c(c3)oc3cc(-c5cccc6ocnc56)ccc34)n2)cc1]

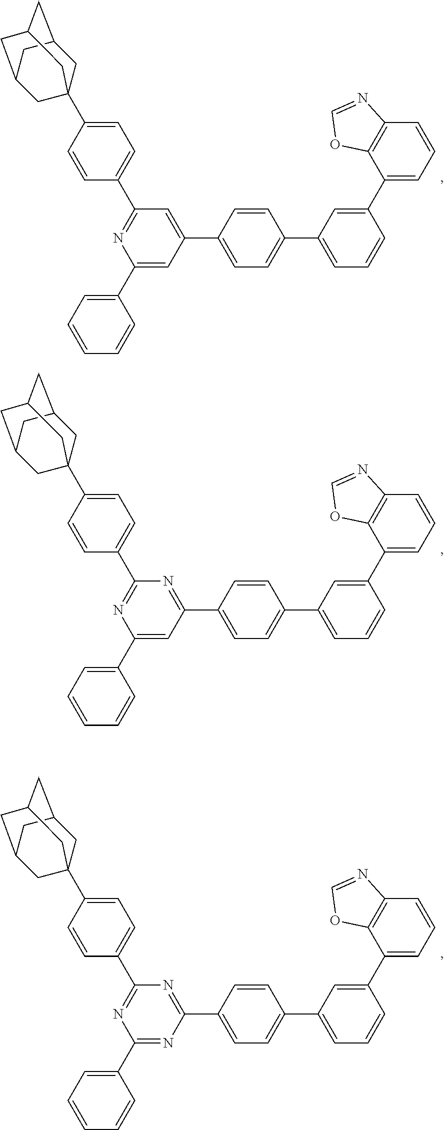 [SMILES: c1ccc(-c2cc(-c3ccc(-c4cccc(-c5cccc6ncoc56)c4)cc3)cc(-c3ccc(C45CC6CC(CC(C6)C4)C5)cc3)n2)cc1 ; c1ccc(-c2cc(-c3ccc(-c4cccc(-c5cccc6ncoc56)c4)cc3)nc(-c3ccc(C45CC6CC(CC(C6)C4)C5)cc3)n2)cc1 ; c1ccc(-c2nc(-c3ccc(-c4cccc(-c5cccc6ncoc56)c4)cc3)nc(-c3ccc(C45CC6CC(CC(C6)C4)C5)cc3)n2)cc1]

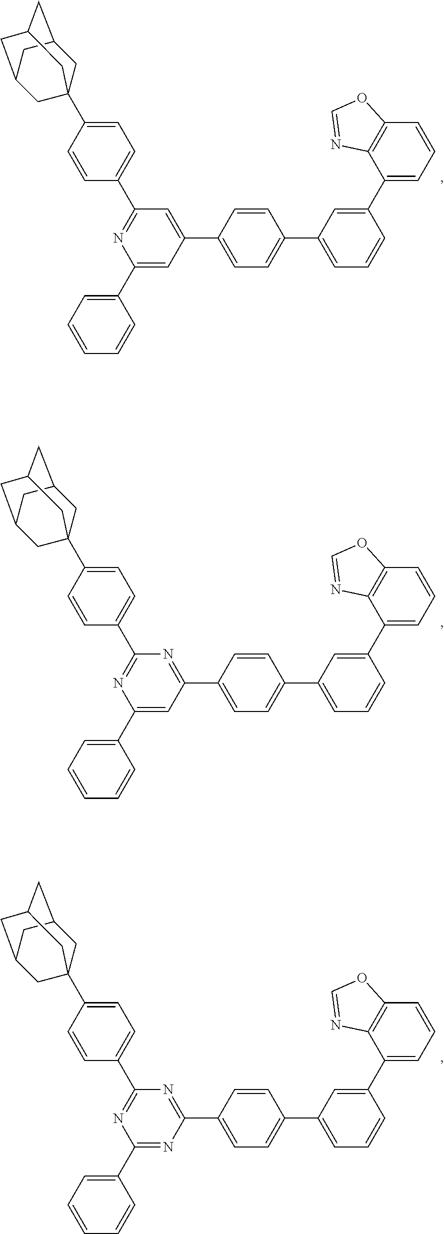 [SMILES: c1ccc(-c2cc(-c3ccc(-c4cccc(-c5cccc6ocnc56)c4)cc3)cc(-c3ccc(C45CC6CC(CC(C6)C4)C5)cc3)n2)cc1 ; c1ccc(-c2cc(-c3ccc(-c4cccc(-c5cccc6ocnc56)c4)cc3)nc(-c3ccc(C45CC6CC(CC(C6)C4)C5)cc3)n2)cc1 ; c1ccc(-c2nc(-c3ccc(-c4cccc(-c5cccc6ocnc56)c4)cc3)nc(-c3ccc(C45CC6CC(CC(C6)C4)C5)cc3)n2)cc1]

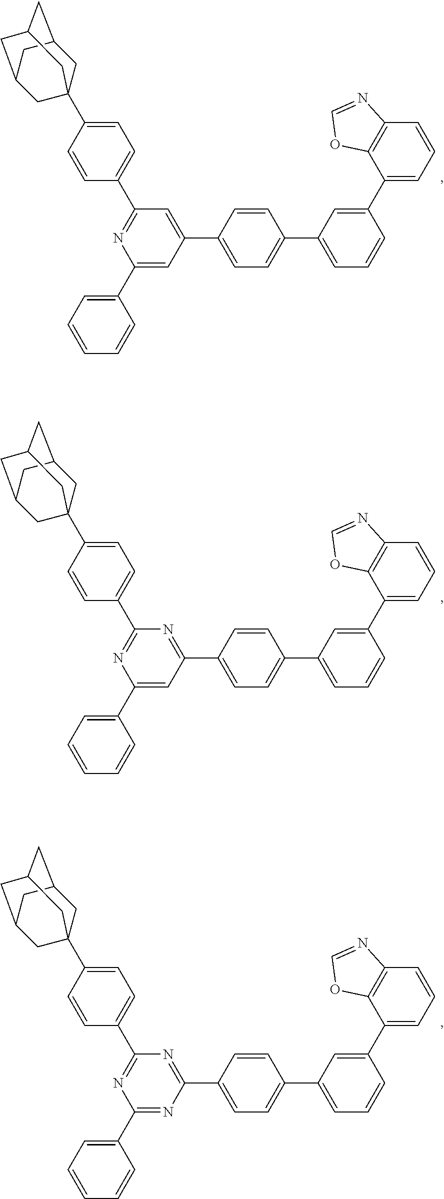 [SMILES: c1ccc(-c2cc(-c3ccc(-c4cccc(-c5cccc6ncoc56)c4)cc3)cc(-c3ccc(C45CC6CC(CC(C6)C4)C5)cc3)n2)cc1 ; c1ccc(-c2cc(-c3ccc(-c4cccc(-c5cccc6ncoc56)c4)cc3)nc(-c3ccc(C45CC6CC(CC(C6)C4)C5)cc3)n2)cc1 ; c1ccc(-c2nc(-c3ccc(-c4cccc(-c5cccc6ncoc56)c4)cc3)nc(-c3ccc(C45CC6CC(CC(C6)C4)C5)cc3)n2)cc1]

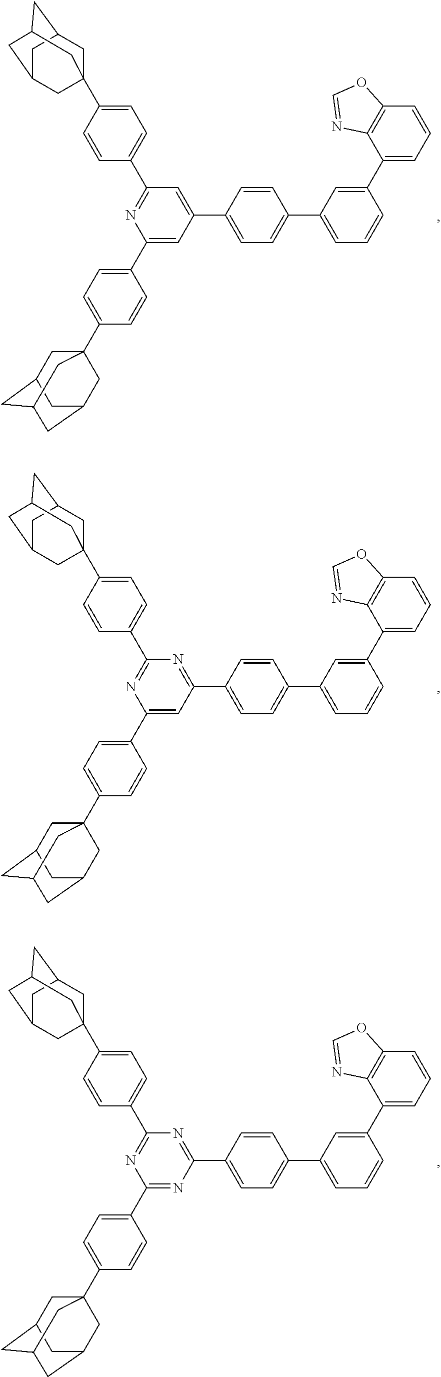 [SMILES: c1cc(-c2ccc(-c3cc(-c4ccc(C56CC7CC(CC(C7)C5)C6)cc4)nc(-c4ccc(C56CC7CC(CC(C7)C5)C6)cc4)c3)cc2)cc(-c2cccc3ocnc23)c1 ; c1cc(-c2ccc(-c3cc(-c4ccc(C56CC7CC(CC(C7)C5)C6)cc4)nc(-c4ccc(C56CC7CC(CC(C7)C5)C6)cc4)n3)cc2)cc(-c2cccc3ocnc23)c1 ; c1cc(-c2ccc(-c3nc(-c4ccc(C56CC7CC(CC(C7)C5)C6)cc4)nc(-c4ccc(C56CC7CC(CC(C7)C5)C6)cc4)n3)cc2)cc(-c2cccc3ocnc23)c1]

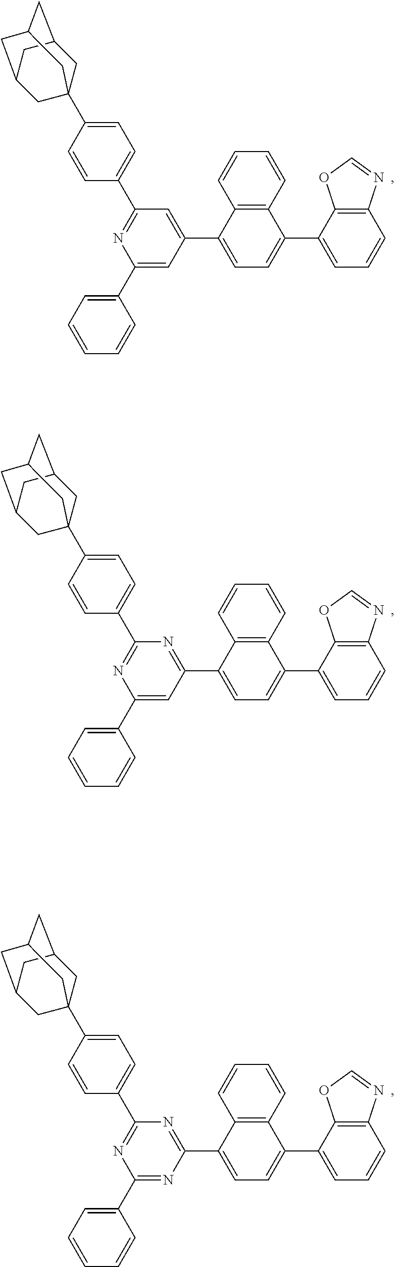 [SMILES: c1ccc(-c2cc(-c3ccc(-c4cccc5ncoc45)c4ccccc34)cc(-c3ccc(C45CC6CC(CC(C6)C4)C5)cc3)n2)cc1 ; c1ccc(-c2cc(-c3ccc(-c4cccc5ncoc45)c4ccccc34)nc(-c3ccc(C45CC6CC(CC(C6)C4)C5)cc3)n2)cc1 ; c1ccc(-c2nc(-c3ccc(C45CC6CC(CC(C6)C4)C5)cc3)nc(-c3ccc(-c4cccc5ncoc45)c4ccccc34)n2)cc1]

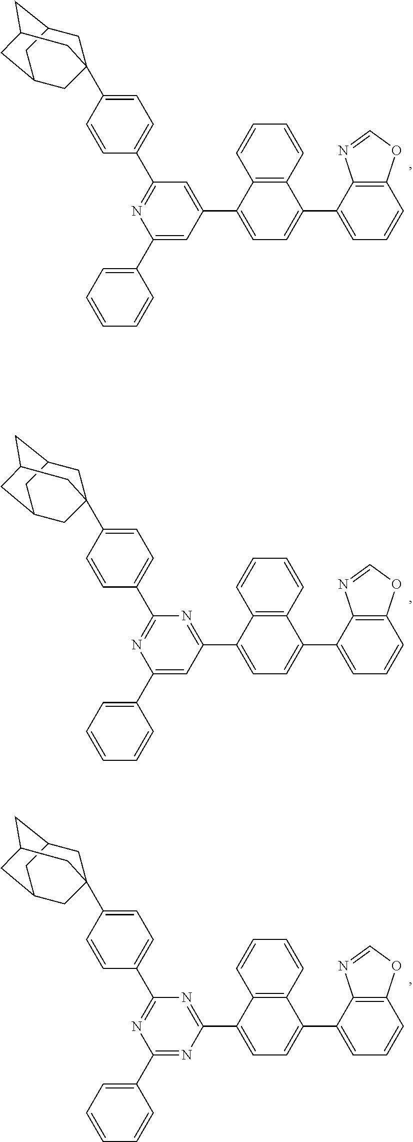 [SMILES: c1ccc(-c2cc(-c3ccc(-c4cccc5ocnc45)c4ccccc34)cc(-c3ccc(C45CC6CC(CC(C6)C4)C5)cc3)n2)cc1 ; c1ccc(-c2cc(-c3ccc(-c4cccc5ocnc45)c4ccccc34)nc(-c3ccc(C45CC6CC(CC(C6)C4)C5)cc3)n2)cc1 ; c1ccc(-c2nc(-c3ccc(C45CC6CC(CC(C6)C4)C5)cc3)nc(-c3ccc(-c4cccc5ocnc45)c4ccccc34)n2)cc1]

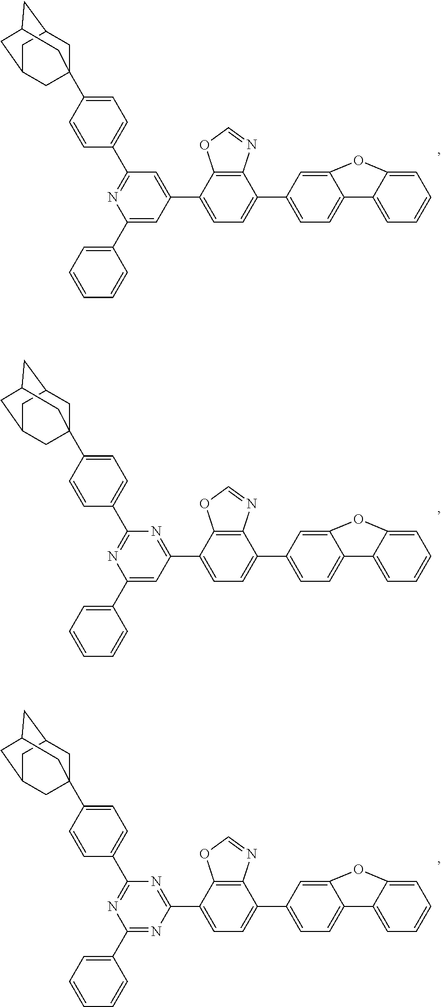 [SMILES: c1ccc(-c2cc(-c3ccc(-c4ccc5c(c4)oc4ccccc45)c4ncoc34)cc(-c3ccc(C45CC6CC(CC(C6)C4)C5)cc3)n2)cc1 ; c1ccc(-c2cc(-c3ccc(-c4ccc5c(c4)oc4ccccc45)c4ncoc34)nc(-c3ccc(C45CC6CC(CC(C6)C4)C5)cc3)n2)cc1 ; c1ccc(-c2nc(-c3ccc(C45CC6CC(CC(C6)C4)C5)cc3)nc(-c3ccc(-c4ccc5c(c4)oc4ccccc45)c4ncoc34)n2)cc1]

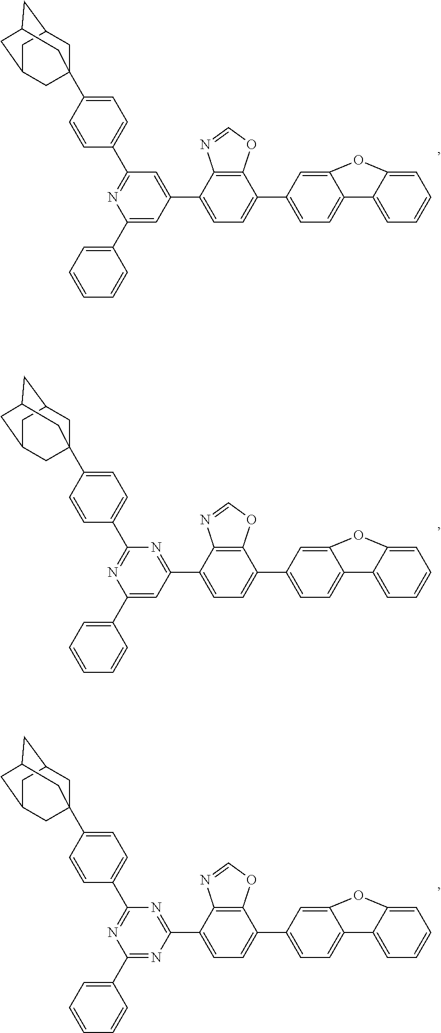 [SMILES: c1ccc(-c2cc(-c3ccc(-c4ccc5c(c4)oc4ccccc45)c4ocnc34)cc(-c3ccc(C45CC6CC(CC(C6)C4)C5)cc3)n2)cc1 ; c1ccc(-c2cc(-c3ccc(-c4ccc5c(c4)oc4ccccc45)c4ocnc34)nc(-c3ccc(C45CC6CC(CC(C6)C4)C5)cc3)n2)cc1 ; c1ccc(-c2nc(-c3ccc(C45CC6CC(CC(C6)C4)C5)cc3)nc(-c3ccc(-c4ccc5c(c4)oc4ccccc45)c4ocnc34)n2)cc1]

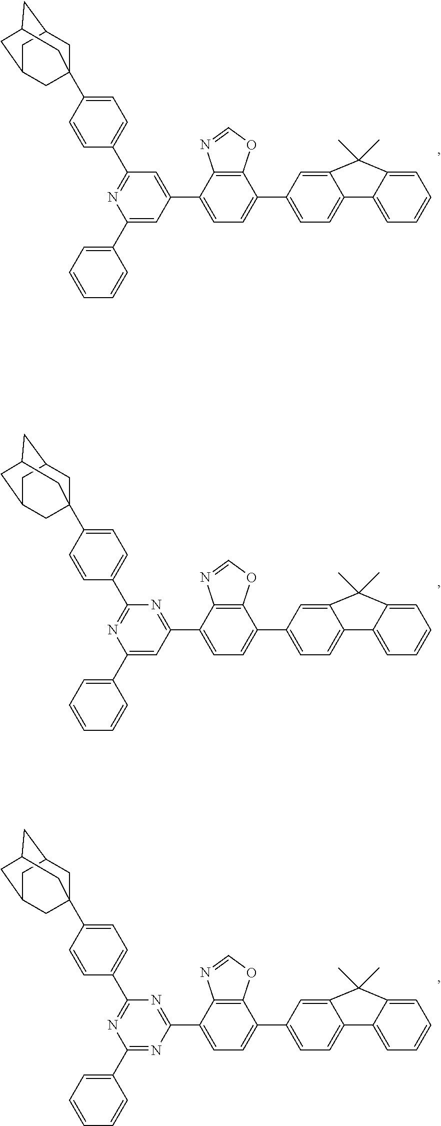 [SMILES: CC1(C)c2ccccc2-c2ccc(-c3ccc(-c4cc(-c5ccccc5)nc(-c5ccc(C67CC8CC(CC(C8)C6)C7)cc5)c4)c4ncoc34)cc21 ; CC1(C)c2ccccc2-c2ccc(-c3ccc(-c4cc(-c5ccccc5)nc(-c5ccc(C67CC8CC(CC(C8)C6)C7)cc5)n4)c4ncoc34)cc21 ; CC1(C)c2ccccc2-c2ccc(-c3ccc(-c4nc(-c5ccccc5)nc(-c5ccc(C67CC8CC(CC(C8)C6)C7)cc5)n4)c4ncoc34)cc21]

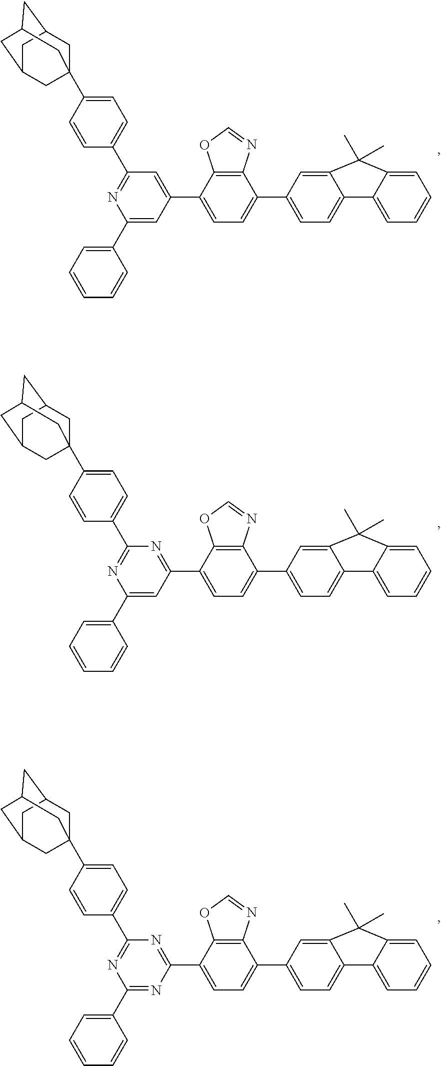 [SMILES: CC1(C)c2ccccc2-c2ccc(-c3ccc(-c4cc(-c5ccccc5)nc(-c5ccc(C67CC8CC(CC(C8)C6)C7)cc5)c4)c4ocnc34)cc21 ; CC1(C)c2ccccc2-c2ccc(-c3ccc(-c4cc(-c5ccccc5)nc(-c5ccc(C67CC8CC(CC(C8)C6)C7)cc5)n4)c4ocnc34)cc21 ; CC1(C)c2ccccc2-c2ccc(-c3ccc(-c4nc(-c5ccccc5)nc(-c5ccc(C67CC8CC(CC(C8)C6)C7)cc5)n4)c4ocnc34)cc21]

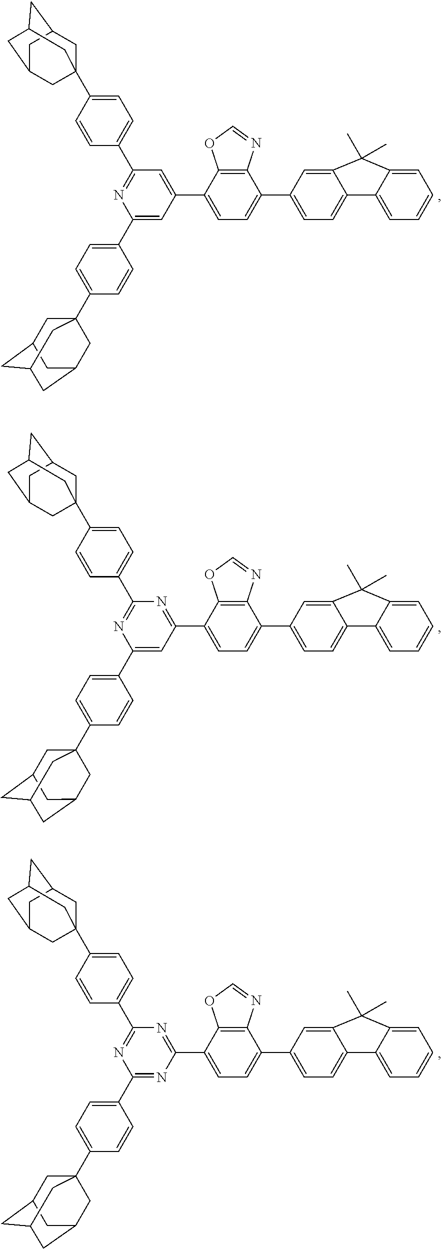

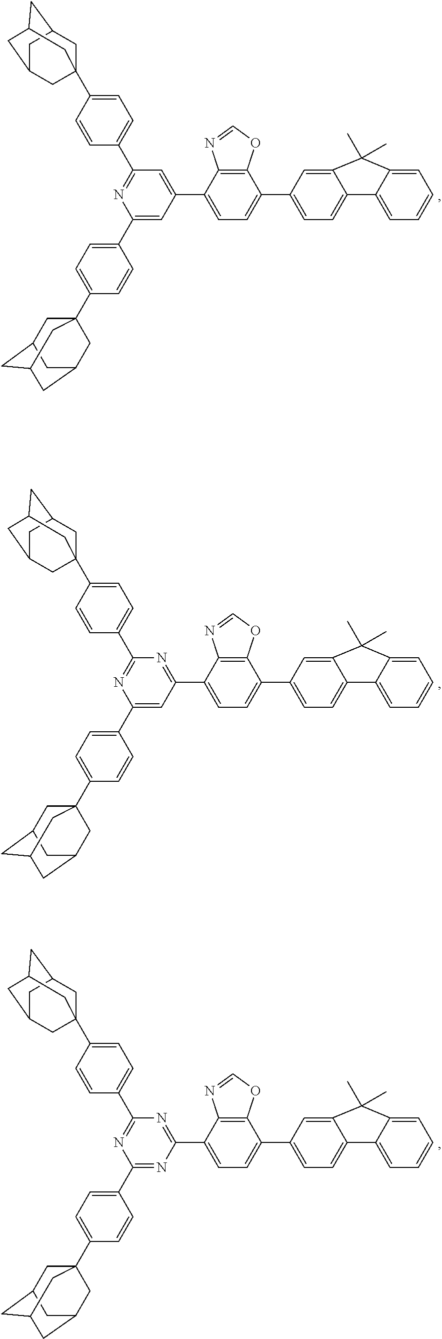 [SMILES: CC1(C)c2ccccc2-c2ccc(-c3ccc(-c4cc(-c5ccc(C67CC8CC(CC(C8)C6)C7)cc5)nc(-c5ccc(C67CC8CC(CC(C8)C6)C7)cc5)c4)c4ncoc34)cc21 ; CC1(C)c2ccccc2-c2ccc(-c3ccc(-c4cc(-c5ccc(C67CC8CC(CC(C8)C6)C7)cc5)nc(-c5ccc(C67CC8CC(CC(C8)C6)C7)cc5)n4)c4ncoc34)cc21 ; CC1(C)c2ccccc2-c2ccc(-c3ccc(-c4nc(-c5ccc(C67CC8CC(CC(C8)C6)C7)cc5)nc(-c5ccc(C67CC8CC(CC(C8)C6)C7)cc5)n4)c4ncoc34)cc21]

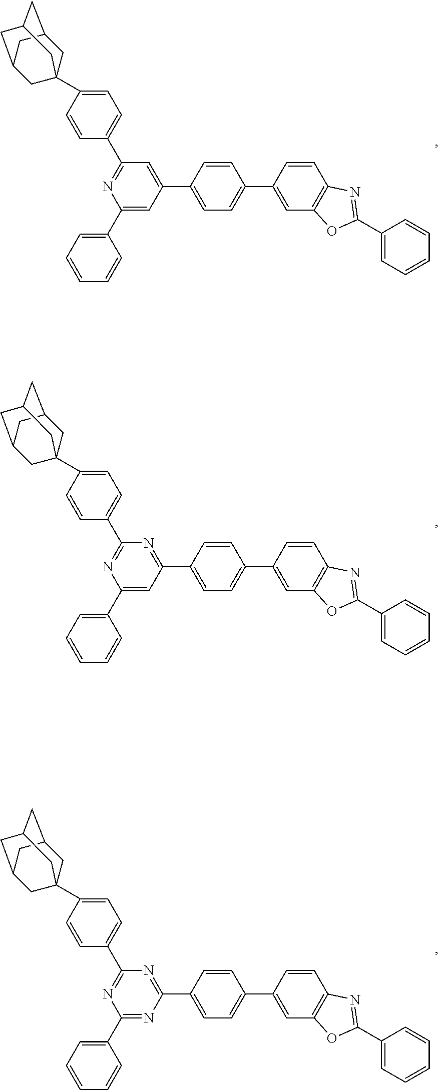 [SMILES: c1ccc(-c2cc(-c3ccc(-c4ccc5nc(-c6ccccc6)oc5c4)cc3)cc(-c3ccc(C45CC6CC(CC(C6)C4)C5)cc3)n2)cc1 ; c1ccc(-c2cc(-c3ccc(-c4ccc5nc(-c6ccccc6)oc5c4)cc3)nc(-c3ccc(C45CC6CC(CC(C6)C4)C5)cc3)n2)cc1 ; c1ccc(-c2nc(-c3ccc(-c4ccc5nc(-c6ccccc6)oc5c4)cc3)nc(-c3ccc(C45CC6CC(CC(C6)C4)C5)cc3)n2)cc1]

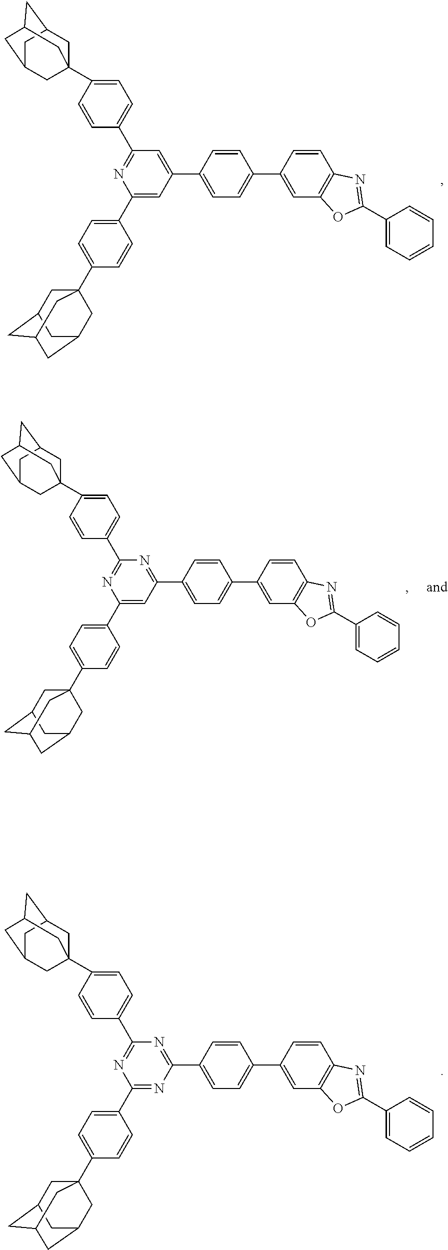 [SMILES: c1ccc(-c2nc3ccc(-c4ccc(-c5cc(-c6ccc(C78CC9CC(CC(C9)C7)C8)cc6)nc(-c6ccc(C78CC9CC(CC(C9)C7)C8)cc6)c5)cc4)cc3o2)cc1 ; c1ccc(-c2nc3ccc(-c4ccc(-c5cc(-c6ccc(C78CC9CC(CC(C9)C7)C8)cc6)nc(-c6ccc(C78CC9CC(CC(C9)C7)C8)cc6)n5)cc4)cc3o2)cc1 ; c1ccc(-c2nc3ccc(-c4ccc(-c5nc(-c6ccc(C78CC9CC(CC(C9)C7)C8)cc6)nc(-c6ccc(C78CC9CC(CC(C9)C7)C8)cc6)n5)cc4)cc3o2)cc1]

Optionally, the chemical structural formula of the material of the electron transport layer includes any one of

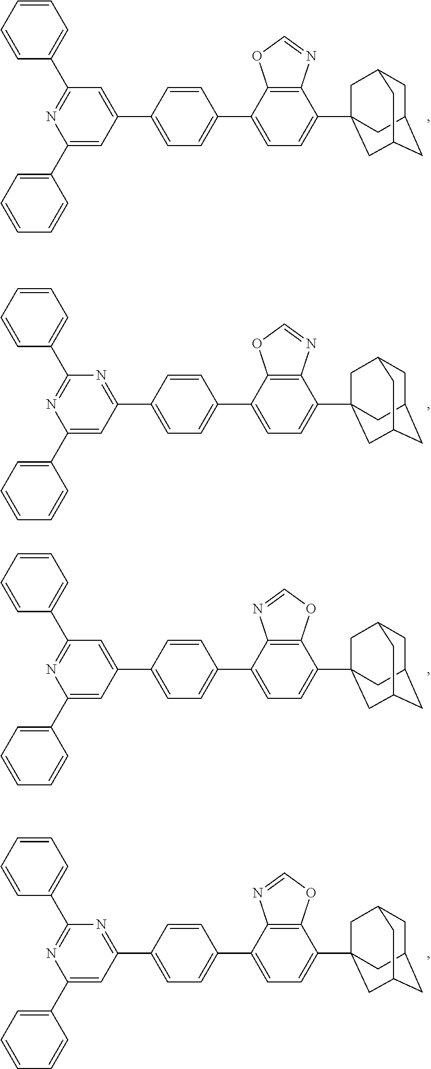

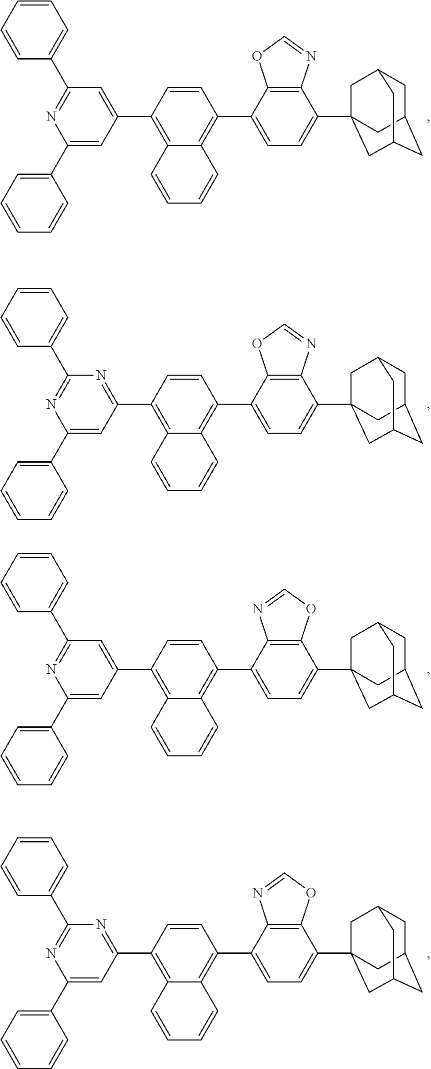

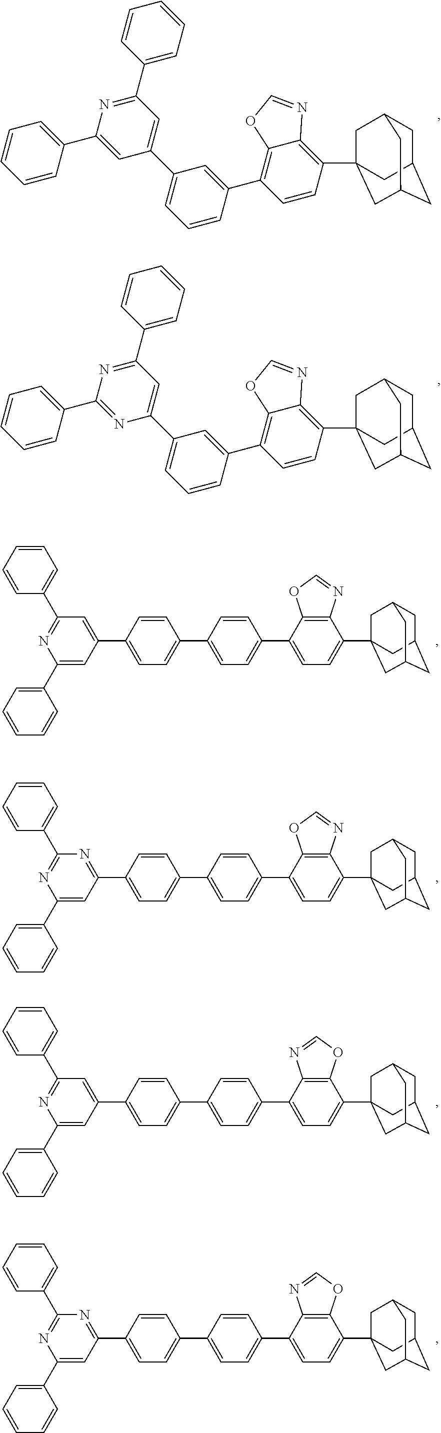

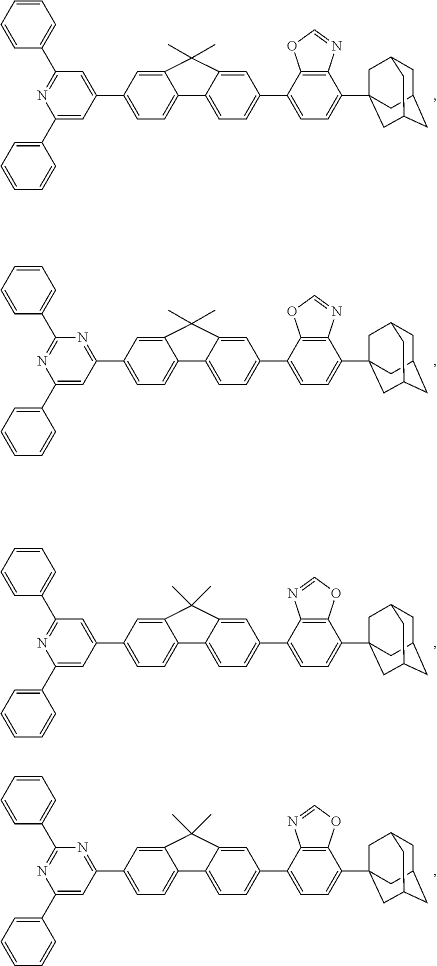 [SMILES: CC1(C)c2cc(-c3cc(-c4ccccc4)nc(-c4ccccc4)c3)ccc2-c2ccc(-c3ccc(C45CC6CC(CC(C6)C4)C5)c4ncoc34)cc21 ; CC1(C)c2cc(-c3cc(-c4ccccc4)nc(-c4ccccc4)n3)ccc2-c2ccc(-c3ccc(C45CC6CC(CC(C6)C4)C5)c4ncoc34)cc21 ; CC1(C)c2cc(-c3cc(-c4ccccc4)nc(-c4ccccc4)c3)ccc2-c2ccc(-c3ccc(C45CC6CC(CC(C6)C4)C5)c4ocnc34)cc21 ; CC1(C)c2cc(-c3cc(-c4ccccc4)nc(-c4ccccc4)n3)ccc2-c2ccc(-c3ccc(C45CC6CC(CC(C6)C4)C5)c4ocnc34)cc21]

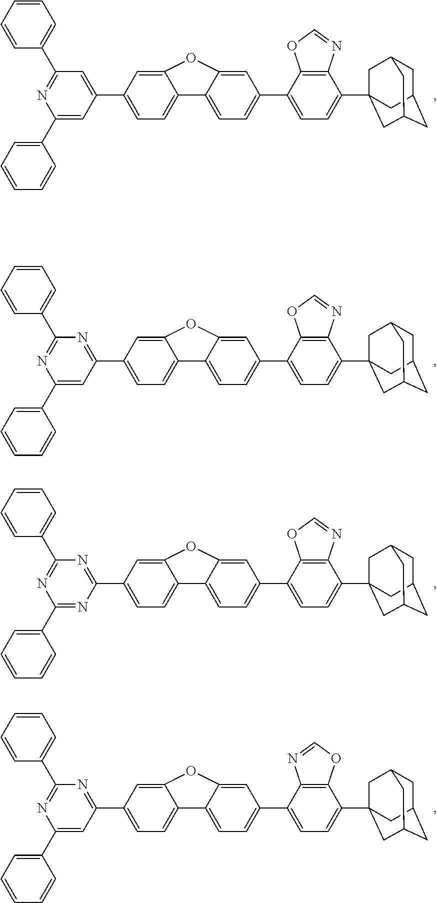

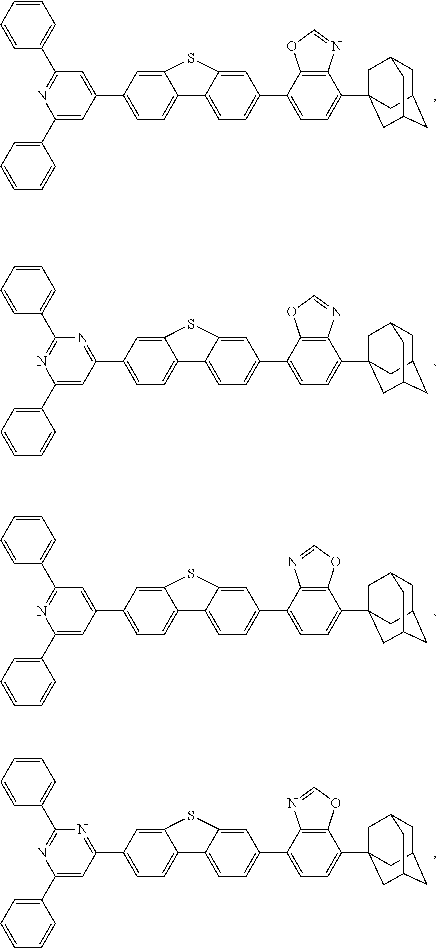 [SMILES: c1ccc(-c2cc(-c3ccc4c(c3)sc3cc(-c5ccc(C67CC8CC(CC(C8)C6)C7)c6ncoc56)ccc34)cc(-c3ccccc3)n2)cc1 ; c1ccc(-c2cc(-c3ccc4c(c3)sc3cc(-c5ccc(C67CC8CC(CC(C8)C6)C7)c6ncoc56)ccc34)nc(-c3ccccc3)n2)cc1 ; c1ccc(-c2cc(-c3ccc4c(c3)sc3cc(-c5ccc(C67CC8CC(CC(C8)C6)C7)c6ocnc56)ccc34)cc(-c3ccccc3)n2)cc1 ; c1ccc(-c2cc(-c3ccc4c(c3)sc3cc(-c5ccc(C67CC8CC(CC(C8)C6)C7)c6ocnc56)ccc34)nc(-c3ccccc3)n2)cc1]

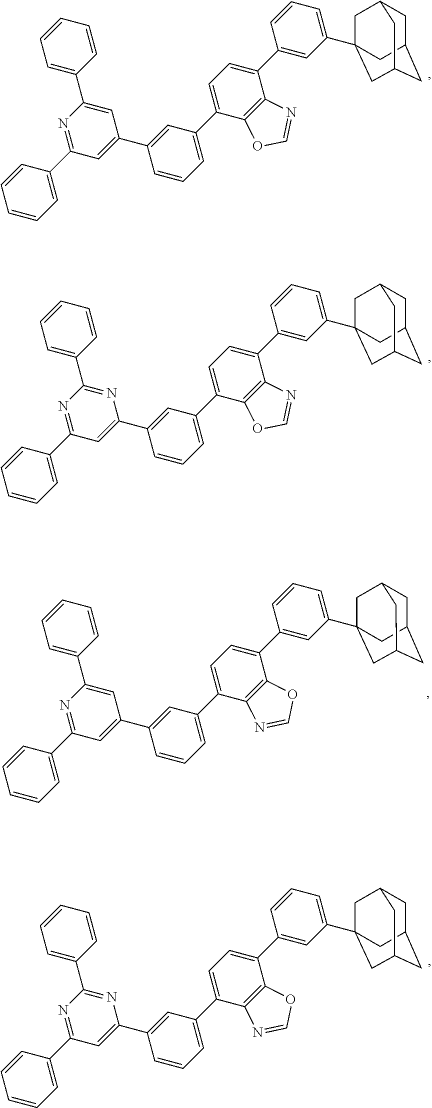

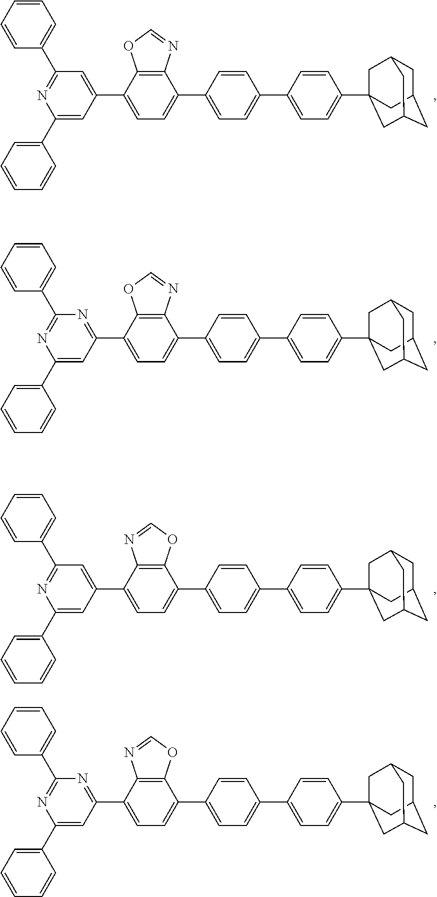

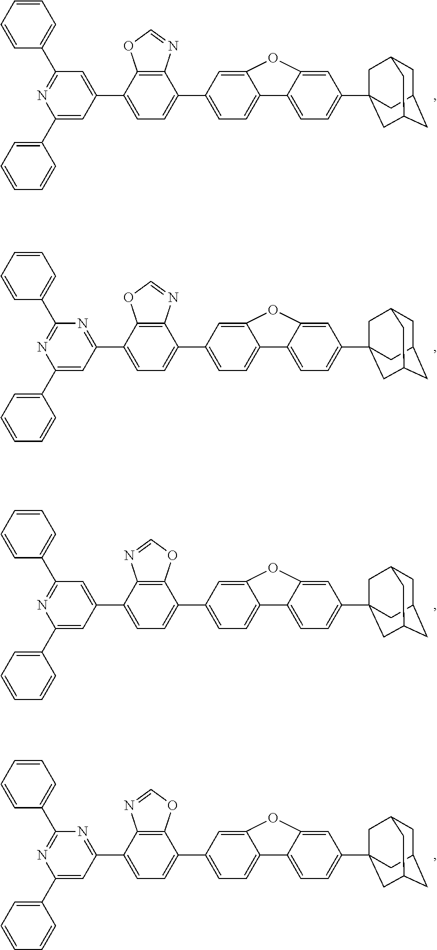 [SMILES: c1ccc(-c2cc(-c3ccc(-c4ccc5c(c4)oc4cc(C67CC8CC(CC(C8)C6)C7)ccc45)c4ncoc34)cc(-c3ccccc3)n2)cc1 ; c1ccc(-c2cc(-c3ccc(-c4ccc5c(c4)oc4cc(C67CC8CC(CC(C8)C6)C7)ccc45)c4ncoc34)nc(-c3ccccc3)n2)cc1 ; c1ccc(-c2cc(-c3ccc(-c4ccc5c(c4)oc4cc(C67CC8CC(CC(C8)C6)C7)ccc45)c4ocnc34)cc(-c3ccccc3)n2)cc1 ; c1ccc(-c2cc(-c3ccc(-c4ccc5c(c4)oc4cc(C67CC8CC(CC(C8)C6)C7)ccc45)c4ocnc34)nc(-c3ccccc3)n2)cc1]

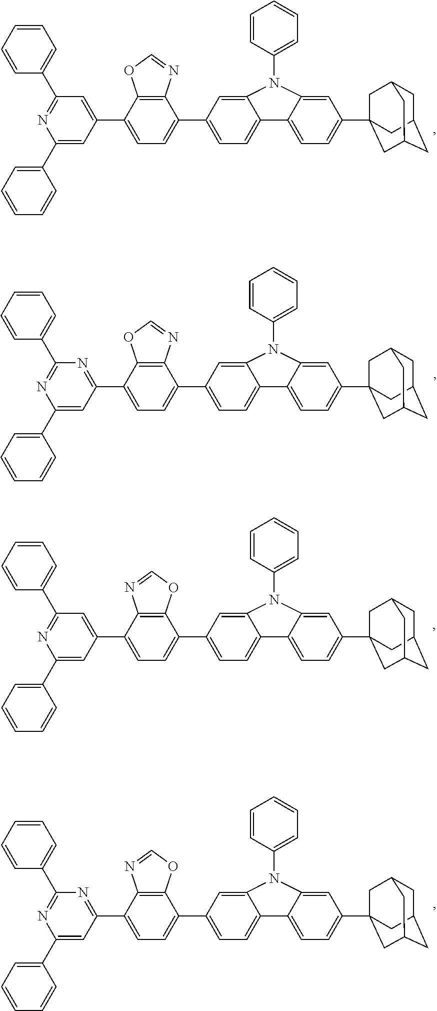 [SMILES: c1ccc(-c2cc(-c3ccc(-c4ccc5c6ccc(C78CC9CC(CC(C9)C7)C8)cc6n(-c6ccccc6)c5c4)c4ncoc34)cc(-c3ccccc3)n2)cc1 ; c1ccc(-c2cc(-c3ccc(-c4ccc5c6ccc(C78CC9CC(CC(C9)C7)C8)cc6n(-c6ccccc6)c5c4)c4ncoc34)nc(-c3ccccc3)n2)cc1 ; c1ccc(-c2cc(-c3ccc(-c4ccc5c6ccc(C78CC9CC(CC(C9)C7)C8)cc6n(-c6ccccc6)c5c4)c4ocnc34)cc(-c3ccccc3)n2)cc1 ; c1ccc(-c2cc(-c3ccc(-c4ccc5c6ccc(C78CC9CC(CC(C9)C7)C8)cc6n(-c6ccccc6)c5c4)c4ocnc34)nc(-c3ccccc3)n2)cc1]

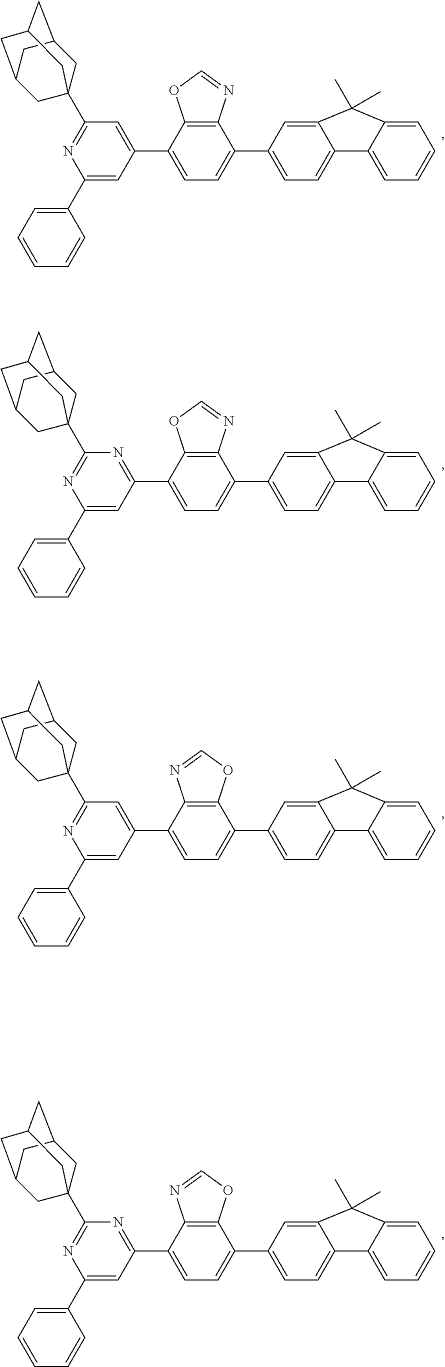 [SMILES: CC1(C)c2ccccc2-c2ccc(-c3ccc(-c4cc(-c5ccccc5)nc(C56CC7CC(CC(C7)C5)C6)c4)c4ocnc34)cc21 ; CC1(C)c2ccccc2-c2ccc(-c3ccc(-c4cc(-c5ccccc5)nc(C56CC7CC(CC(C7)C5)C6)n4)c4ocnc34)cc21 ; CC1(C)c2ccccc2-c2ccc(-c3ccc(-c4cc(-c5ccccc5)nc(C56CC7CC(CC(C7)C5)C6)c4)c4ncoc34)cc21 ; CC1(C)c2ccccc2-c2ccc(-c3ccc(-c4cc(-c5ccccc5)nc(C56CC7CC(CC(C7)C5)C6)n4)c4ncoc34)cc21]

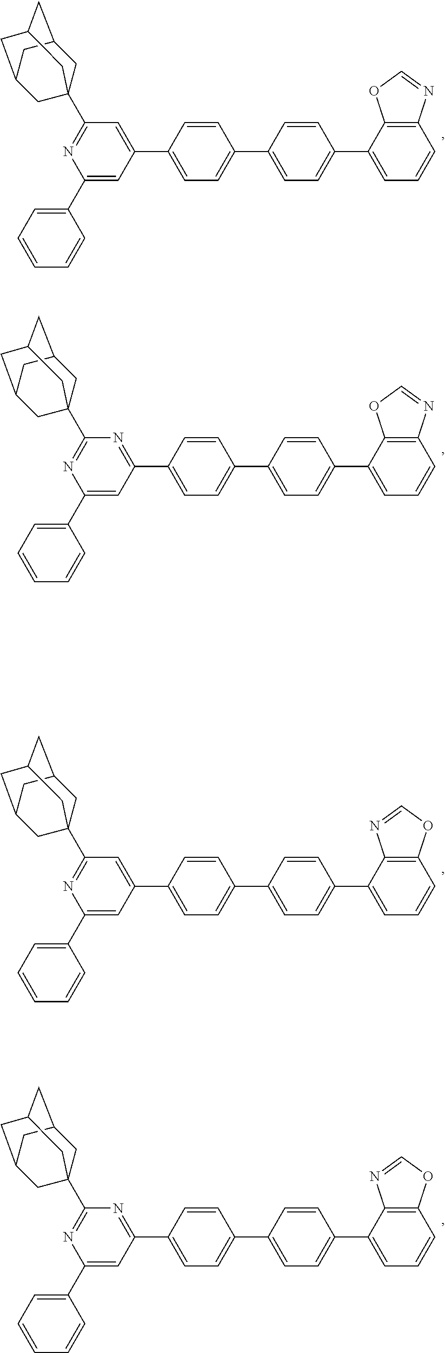 [SMILES: c1ccc(-c2cc(-c3ccc(-c4ccc(-c5cccc6ncoc56)cc4)cc3)cc(C34CC5CC(CC(C5)C3)C4)n2)cc1 ; c1ccc(-c2cc(-c3ccc(-c4ccc(-c5cccc6ncoc56)cc4)cc3)nc(C34CC5CC(CC(C5)C3)C4)n2)cc1 ; c1ccc(-c2cc(-c3ccc(-c4ccc(-c5cccc6ocnc56)cc4)cc3)cc(C34CC5CC(CC(C5)C3)C4)n2)cc1 ; c1ccc(-c2cc(-c3ccc(-c4ccc(-c5cccc6ocnc56)cc4)cc3)nc(C34CC5CC(CC(C5)C3)C4)n2)cc1]

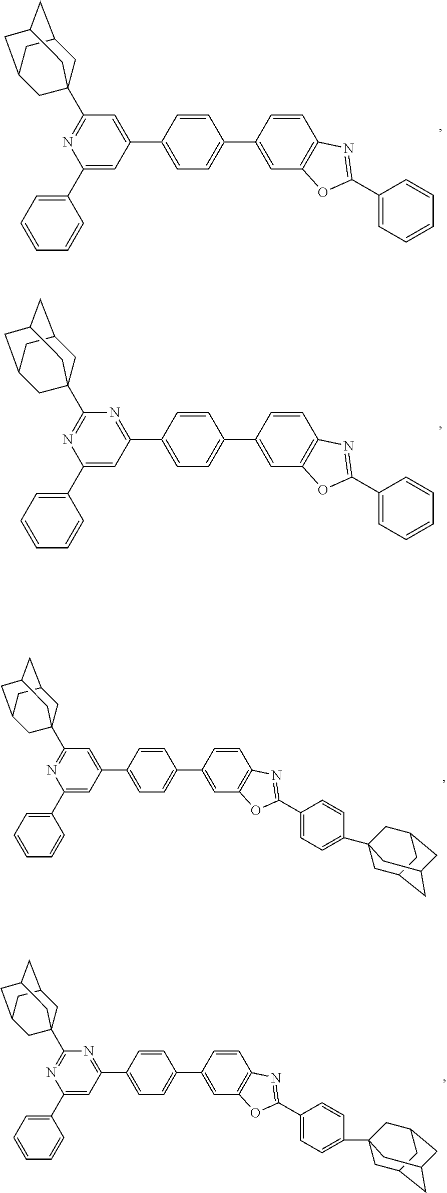 [SMILES: c1ccc(-c2cc(-c3ccc(-c4ccc5nc(-c6ccccc6)oc5c4)cc3)cc(C34CC5CC(CC(C5)C3)C4)n2)cc1 ; c1ccc(-c2cc(-c3ccc(-c4ccc5nc(-c6ccccc6)oc5c4)cc3)nc(C34CC5CC(CC(C5)C3)C4)n2)cc1 ; c1ccc(-c2cc(-c3ccc(-c4ccc5nc(-c6ccc(C78CC9CC(CC(C9)C7)C8)cc6)oc5c4)cc3)cc(C34CC5CC(CC(C5)C3)C4)n2)cc1 ; c1ccc(-c2cc(-c3ccc(-c4ccc5nc(-c6ccc(C78CC9CC(CC(C9)C7)C8)cc6)oc5c4)cc3)nc(C34CC5CC(CC(C5)C3)C4)n2)cc1]

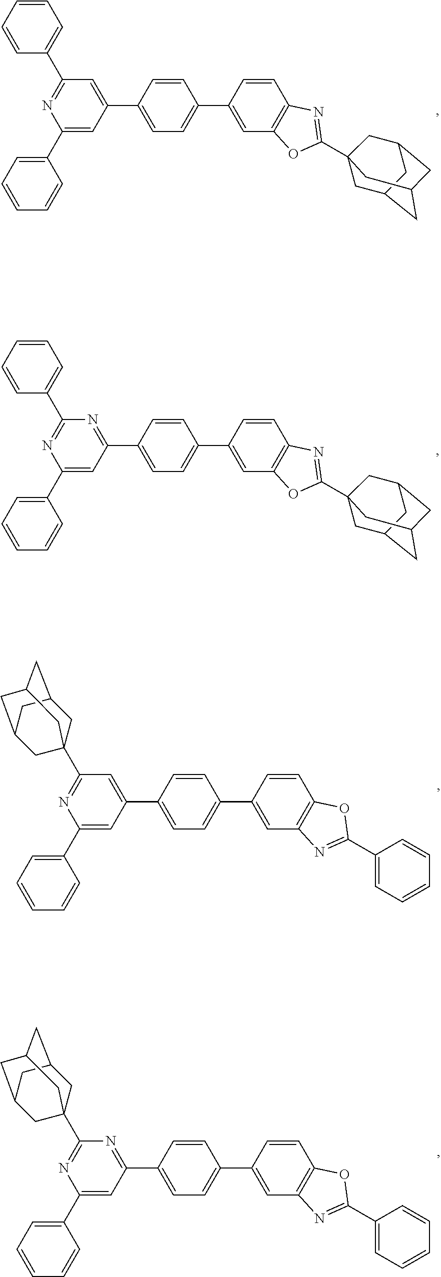 [SMILES: c1ccc(-c2cc(-c3ccc(-c4ccc5nc(C67CC8CC(CC(C8)C6)C7)oc5c4)cc3)cc(-c3ccccc3)n2)cc1 ; c1ccc(-c2cc(-c3ccc(-c4ccc5nc(C67CC8CC(CC(C8)C6)C7)oc5c4)cc3)nc(-c3ccccc3)n2)cc1 ; c1ccc(-c2cc(-c3ccc(-c4ccc5oc(-c6ccccc6)nc5c4)cc3)cc(C34CC5CC(CC(C5)C3)C4)n2)cc1 ; c1ccc(-c2cc(-c3ccc(-c4ccc5oc(-c6ccccc6)nc5c4)cc3)nc(C34CC5CC(CC(C5)C3)C4)n2)cc1]

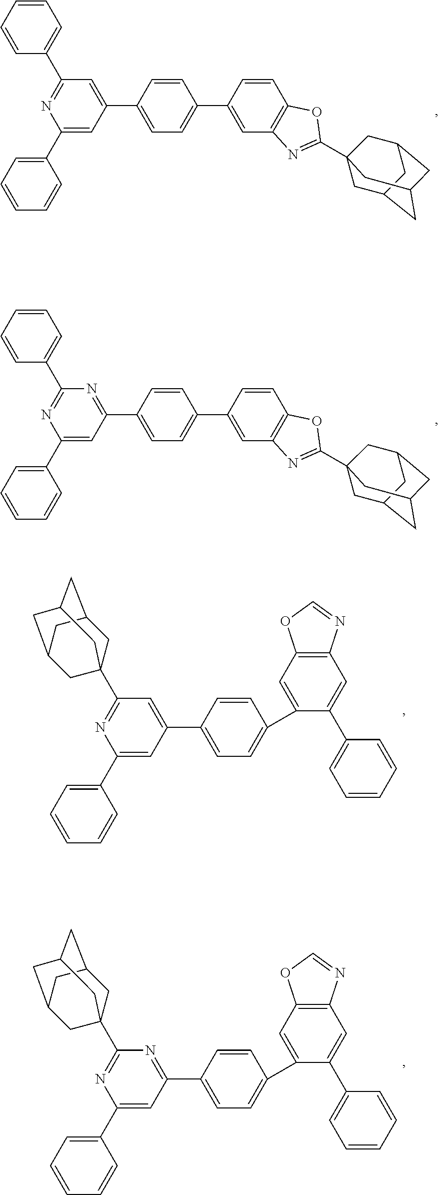 [SMILES: c1ccc(-c2cc(-c3ccc(-c4ccc5oc(C67CC8CC(CC(C8)C6)C7)nc5c4)cc3)cc(-c3ccccc3)n2)cc1 ; c1ccc(-c2cc(-c3ccc(-c4ccc5oc(C67CC8CC(CC(C8)C6)C7)nc5c4)cc3)nc(-c3ccccc3)n2)cc1 ; c1ccc(-c2cc(-c3ccc(-c4cc5ocnc5cc4-c4ccccc4)cc3)cc(C34CC5CC(CC(C5)C3)C4)n2)cc1 ; c1ccc(-c2cc(-c3ccc(-c4cc5ocnc5cc4-c4ccccc4)cc3)nc(C34CC5CC(CC(C5)C3)C4)n2)cc1]

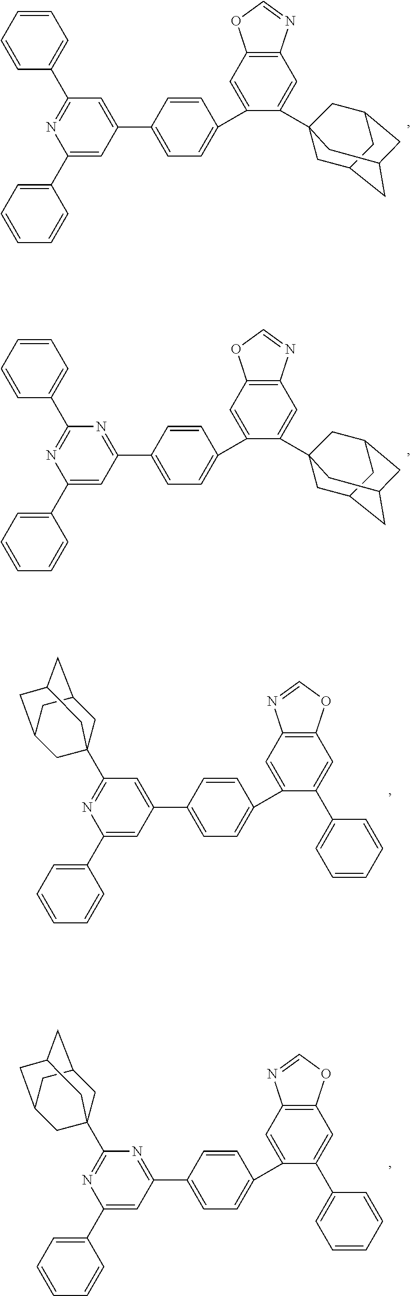 [SMILES: c1ccc(-c2cc(-c3ccc(-c4cc5ocnc5cc4C45CC6CC(CC(C6)C4)C5)cc3)cc(-c3ccccc3)n2)cc1 ; c1ccc(-c2cc(-c3ccc(-c4cc5ocnc5cc4C45CC6CC(CC(C6)C4)C5)cc3)nc(-c3ccccc3)n2)cc1 ; c1ccc(-c2cc(-c3ccc(-c4cc5ncoc5cc4-c4ccccc4)cc3)cc(C34CC5CC(CC(C5)C3)C4)n2)cc1 ; c1ccc(-c2cc(-c3ccc(-c4cc5ncoc5cc4-c4ccccc4)cc3)nc(C34CC5CC(CC(C5)C3)C4)n2)cc1]

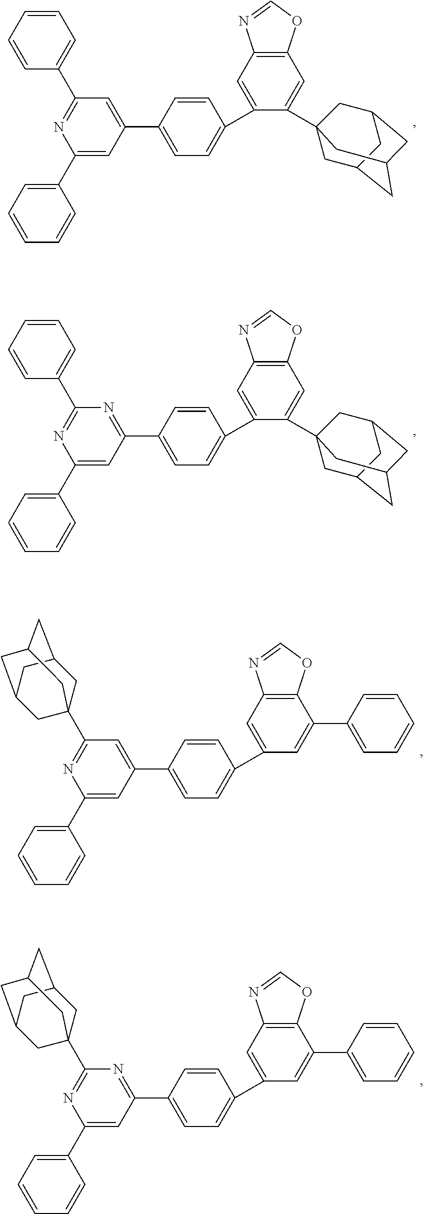 [SMILES: c1ccc(-c2cc(-c3ccc(-c4cc5ncoc5cc4C45CC6CC(CC(C6)C4)C5)cc3)cc(-c3ccccc3)n2)cc1 ; c1ccc(-c2cc(-c3ccc(-c4cc5ncoc5cc4C45CC6CC(CC(C6)C4)C5)cc3)nc(-c3ccccc3)n2)cc1 ; c1ccc(-c2cc(-c3ccc(-c4cc(-c5ccccc5)c5ocnc5c4)cc3)cc(C34CC5CC(CC(C5)C3)C4)n2)cc1 ; c1ccc(-c2cc(-c3ccc(-c4cc(-c5ccccc5)c5ocnc5c4)cc3)nc(C34CC5CC(CC(C5)C3)C4)n2)cc1]

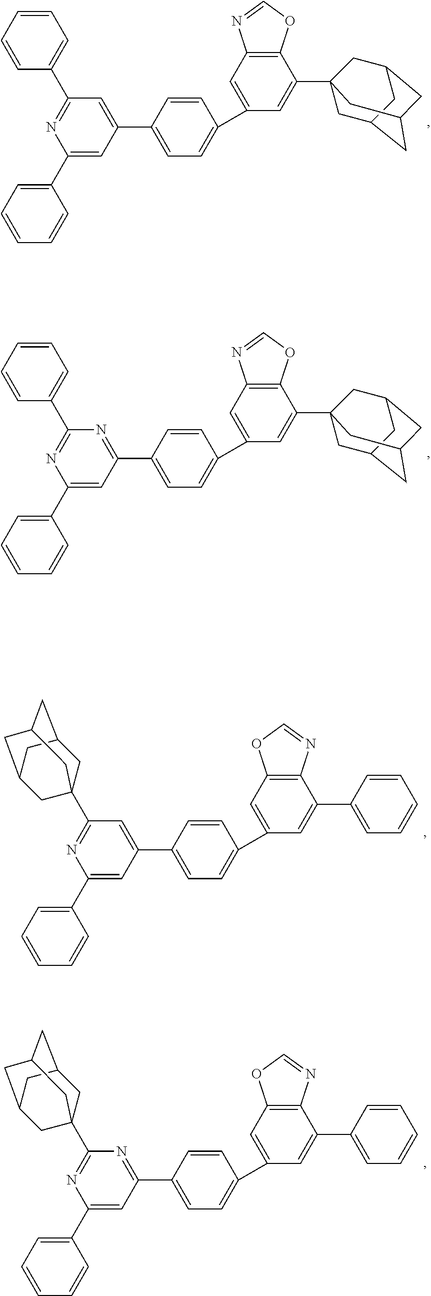 [SMILES: c1ccc(-c2cc(-c3ccc(-c4cc(C56CC7CC(CC(C7)C5)C6)c5ocnc5c4)cc3)cc(-c3ccccc3)n2)cc1 ; c1ccc(-c2cc(-c3ccc(-c4cc(C56CC7CC(CC(C7)C5)C6)c5ocnc5c4)cc3)nc(-c3ccccc3)n2)cc1 ; c1ccc(-c2cc(-c3ccc(-c4cc(-c5ccccc5)c5ncoc5c4)cc3)cc(C34CC5CC(CC(C5)C3)C4)n2)cc1 ; c1ccc(-c2cc(-c3ccc(-c4cc(-c5ccccc5)c5ncoc5c4)cc3)nc(C34CC5CC(CC(C5)C3)C4)n2)cc1]

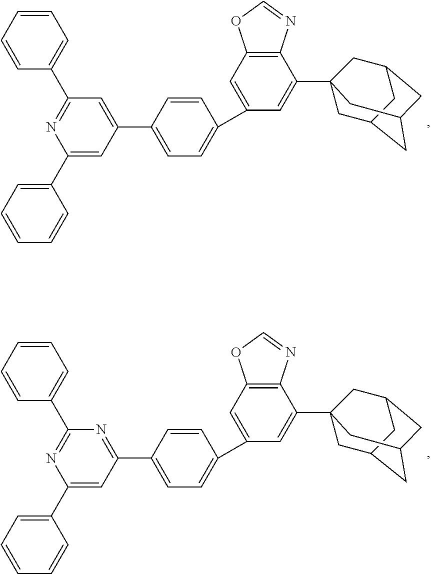 [SMILES: c1ccc(-c2cc(-c3ccc(-c4cc(C56CC7CC(CC(C7)C5)C6)c5ncoc5c4)cc3)cc(-c3ccccc3)n2)cc1 ; c1ccc(-c2cc(-c3ccc(-c4cc(C56CC7CC(CC(C7)C5)C6)c5ncoc5c4)cc3)nc(-c3ccccc3)n2)cc1]

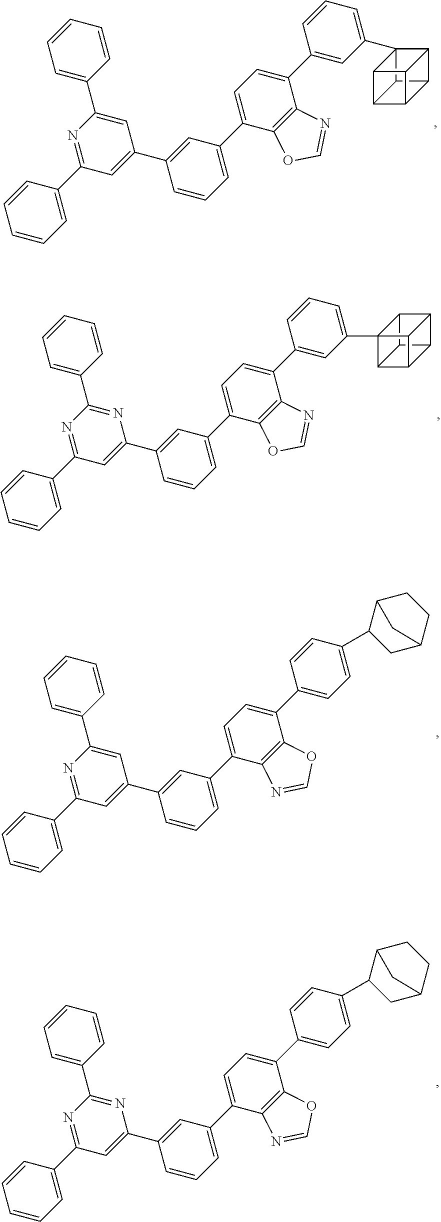 [SMILES: c1ccc(-c2cc(-c3cccc(-c4ccc(-c5cccc(C67C8C9C%10C8C6C%10C97)c5)c5ncoc45)c3)cc(-c3ccccc3)n2)cc1 ; c1ccc(-c2cc(-c3cccc(-c4ccc(-c5cccc(C67C8C9C%10C8C6C%10C97)c5)c5ncoc45)c3)nc(-c3ccccc3)n2)cc1 ; c1ccc(-c2cc(-c3cccc(-c4ccc(-c5ccc(C6CC7CCC6C7)cc5)c5ocnc45)c3)cc(-c3ccccc3)n2)cc1 ; c1ccc(-c2cc(-c3cccc(-c4ccc(-c5ccc(C6CC7CCC6C7)cc5)c5ocnc45)c3)nc(-c3ccccc3)n2)cc1]

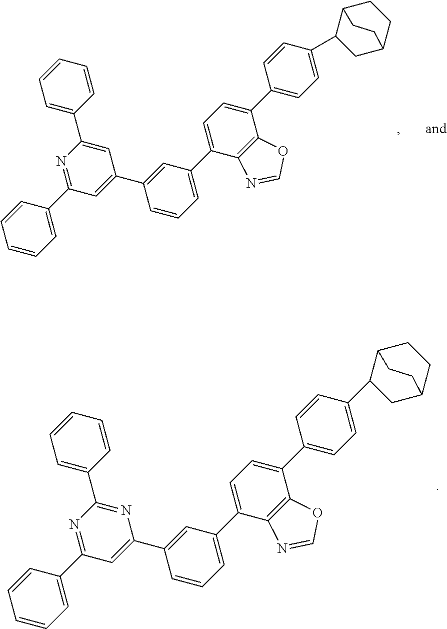 [SMILES: c1ccc(-c2cc(-c3cccc(-c4ccc(-c5ccc(C6CC7CCC6CC7)cc5)c5ocnc45)c3)cc(-c3ccccc3)n2)cc1 ; c1ccc(-c2cc(-c3cccc(-c4ccc(-c5ccc(C6CC7CCC6CC7)cc5)c5ocnc45)c3)nc(-c3ccccc3)n2)cc1]

It should be noted that the nitrogen (N) and oxygen (O) in the

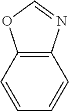

of all of the above chemical structural formulas may be replaced by nitrogen (N) and sulphur(S), or nitrogen (N) and nitrogen (N).

An embodiment of the present application further provides a compound, wherein the general structural formula of the compound is:

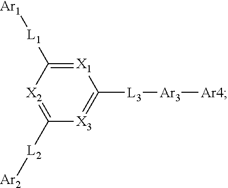

wherein X1 is C(Ra) or nitrogen, X2 is C(Rb) or nitrogen, X3 is C(Rc) or nitrogen, and at least one of X1, X2 and X3 is nitrogen, wherein each of Ra, Rb and Rc is independently any one of a group consisting of hydrogen, diplogen, fluorine, chlorine, an alkyl group whose carbon-atom quantity is 1-12, a halogenated alkyl group whose carbon-atom quantity is 1-12, an alkoxy group whose carbon-atom quantity is 1-12, a cycloalkyl group whose carbon-atom quantity is 3-10, C6-20 aryl, and a heteroaryl group whose carbon-atom quantity is 3-20; each of L1, L2 and L3 is independently any one of a group consisting of a single bond, a substituted or unsubstituted arylene group whose carbon-atom quantity is 6-60, and a substituted or unsubstituted heteroarylene whose carbon-atom quantity is 1-60; each of Ar1, Ar2 and Ar4 is independently any one of a group consisting of a substituted or unsubstituted alkyl group whose carbon-atom quantity is 1-12, a substituted or unsubstituted cycloalkyl group whose carbon-atom quantity is 3-10, a substituted or unsubstituted aralkyl group whose carbon-atom quantity is 7-30, a substituted or unsubstituted heterarylalkyl whose carbon-atom quantity is 2-60, a substituted or unsubstituted aryl whose carbon-atom quantity is 6-60, and a substituted or unsubstituted heteroaryl group whose carbon-atom quantity is 3-60, and at least one of Ar1, Ar2 and Ar4 is the three-dimensional group having a rigidity; and the general structural formula of Ar3 is:

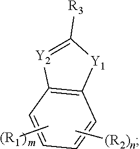

wherein Y1 is any one of carbon, oxygen, sulphur, selenium and N—R4; Y2 is any one of carbon and nitrogen; each of R1, R2 and R3 is independently any one of hydrogen, diplogen, halogen, amino, a nitrile group, nitro, C1-30 alkyl, C2-30 alkenyl, C2-30 alkynyl, C1-30 alkoxy, C6-30 aryloxy, and C6-50 aryl substituted by C6-30 aryl, or includes any one of at least one of heteroatoms selected from nitrogen, oxygen and sulphur and C2-50 heteroaryl unsubstituted or substituted by C6-30 aryloxy, and two of R1, R2 and R3 are directly connected to neighboring groups L3 and Ar4; R4 is any one of a group consisting of hydrogen, diplogen, fluorine, chlorine, an alkyl group whose carbon-atom quantity is 1-12, a halogenated alkyl group whose carbon-atom quantity is 1-12, an alkoxy group whose carbon-atom quantity is 1-12, a cycloalkyl group whose carbon-atom quantity is 3-10, C6-20 aryl, and a heteroaryl group whose carbon-atom quantity is 3-20; and each of m and n is independently 1 or 2.

In an aspect, the compound includes

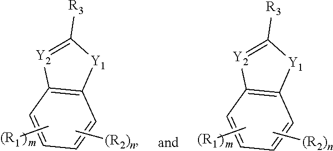

is a strong electron-withdrawing group, and has deep HOMO energy level and LUMO energy level. The low LUMO energy level can effectively reduce the injection barrier between the compound and the material of the neighboring functional layer, which facilitates the electron injection, and accordingly can reduce the lightening voltage of the light emitting device. The low HOMO energy level can effectively block holes, to increase the efficiency of the recombination of the holes and the electrons. Moreover, because

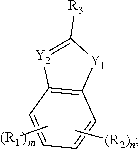

has a high T1, the compound also has a high T1, whereby the compound can prevent triplet excitons from diffusing toward the electron transport layer, thereby increasing the efficiency of the light emitting device. Additionally,

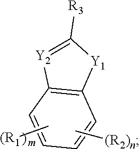

can interact with the neighboring groups, so that the compound has a stable geometrical configuration, whereby it does not easily deform by the effect of an external electric field, and has a lower reorganization energy and a higher electron mobility. In another aspect, the compound includes at least one three-dimensional group, which can effectively inhibit material crystallization. Moreover, the three-dimensional group has a good rigidity, so that the compound has a high glass-transition temperature Tg. The high Tg enables the material not to easily be cracked in the vapor deposition, which facilitates to improve the stability, the film-forming property, the durability, the heat resistance, and so on, of the compound, thereby increasing the efficiency and the life of the light emitting device. Accordingly, a stable and high-efficiency compound can be obtained, and further a light emitting device that has a good stability, a low voltage, a high luminous efficiency and a long life is obtained.

Optionally, the general structural formula of the compound is any one of

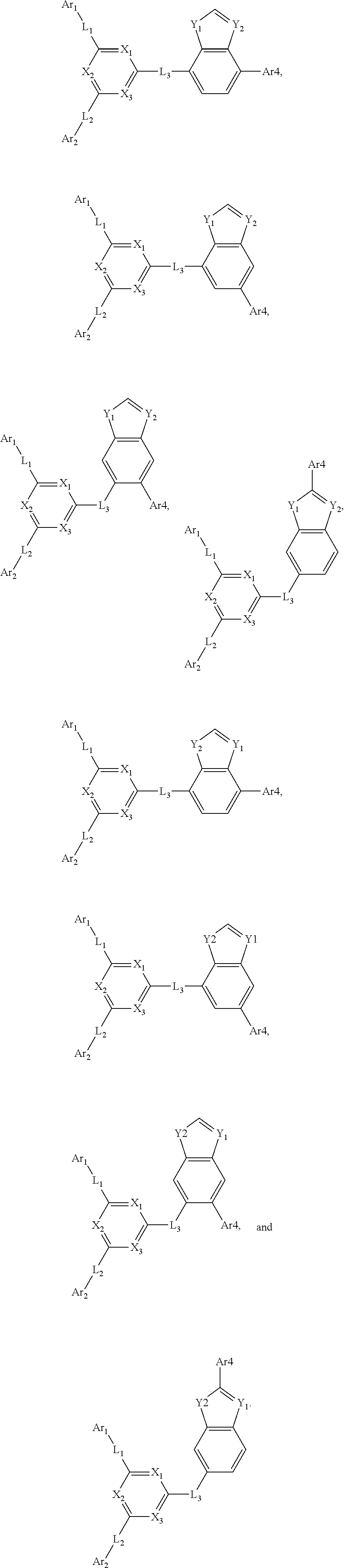

Optionally, the general structural formula of the three-dimensional group having a rigidity is any one of

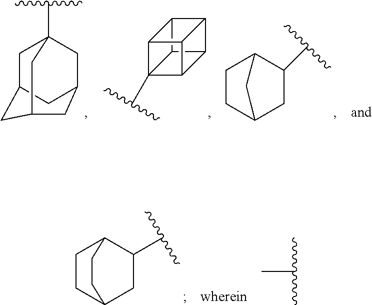

is a bond connected to a carbon atom.

Optionally, the chemical structural formula of the compound having a triazine group includes any one of

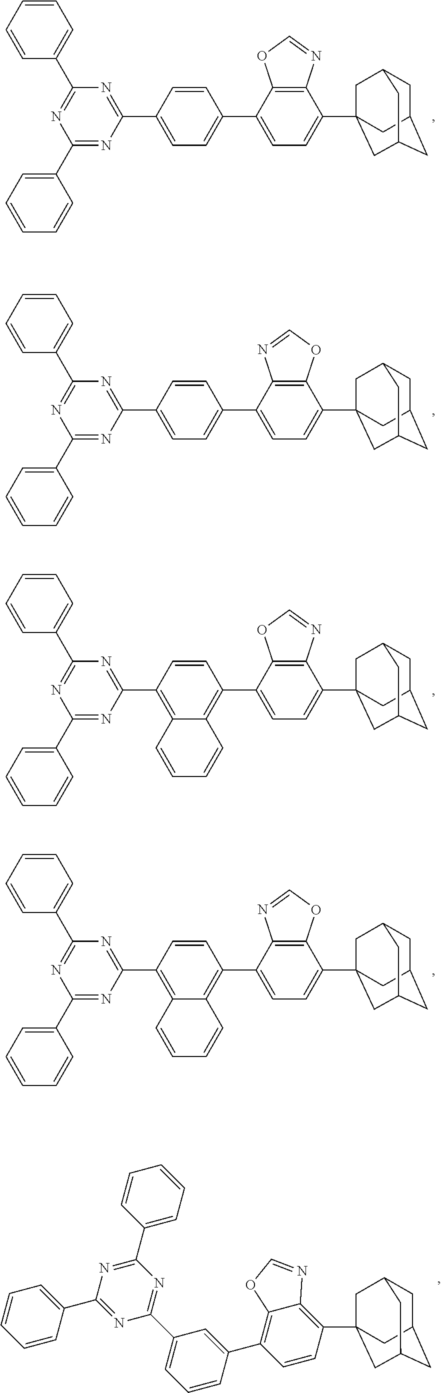 [SMILES: c1ccc(-c2nc(-c3ccccc3)nc(-c3ccc(-c4ccc(C56CC7CC(CC(C7)C5)C6)c5ncoc45)cc3)n2)cc1 ; c1ccc(-c2nc(-c3ccccc3)nc(-c3ccc(-c4ccc(C56CC7CC(CC(C7)C5)C6)c5ocnc45)cc3)n2)cc1 ; c1ccc(-c2nc(-c3ccccc3)nc(-c3ccc(-c4ccc(C56CC7CC(CC(C7)C5)C6)c5ncoc45)c4ccccc34)n2)cc1 ; c1ccc(-c2nc(-c3ccccc3)nc(-c3ccc(-c4ccc(C56CC7CC(CC(C7)C5)C6)c5ocnc45)c4ccccc34)n2)cc1 ; c1ccc(-c2nc(-c3ccccc3)nc(-c3cccc(-c4ccc(C56CC7CC(CC(C7)C5)C6)c5ncoc45)c3)n2)cc1]

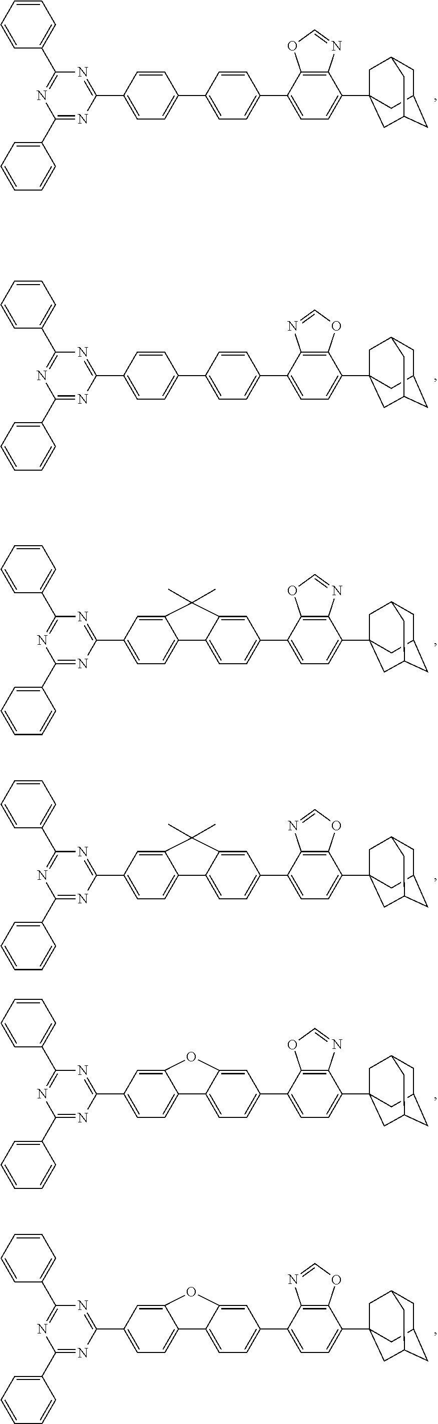

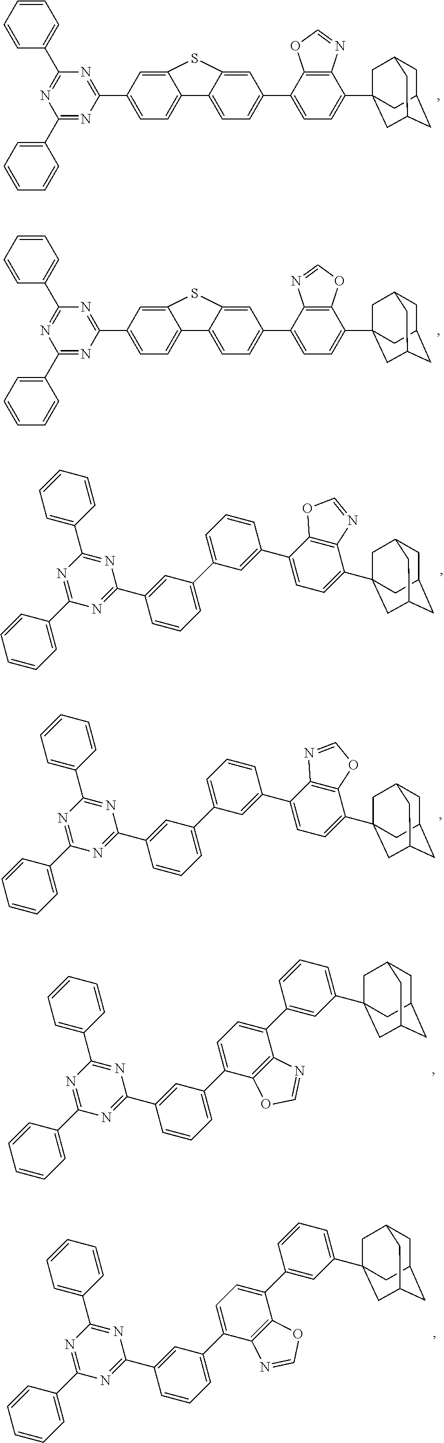

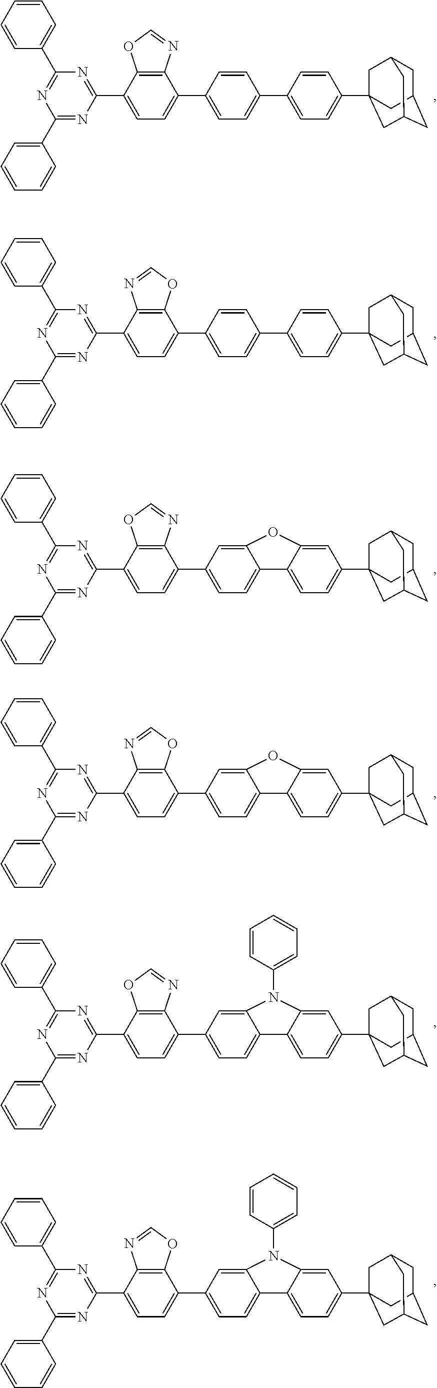

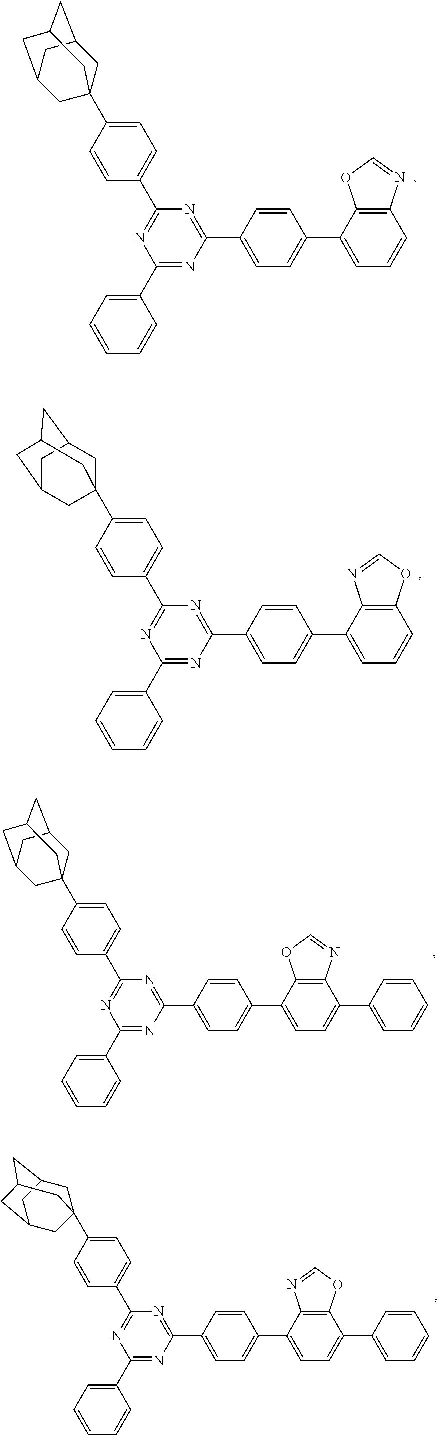 [SMILES: c1ccc(-c2nc(-c3ccc(-c4cccc5ncoc45)cc3)nc(-c3ccc(C45CC6CC(CC(C6)C4)C5)cc3)n2)cc1 ; c1ccc(-c2nc(-c3ccc(-c4cccc5ocnc45)cc3)nc(-c3ccc(C45CC6CC(CC(C6)C4)C5)cc3)n2)cc1 ; c1ccc(-c2nc(-c3ccc(-c4ccc(-c5ccccc5)c5ncoc45)cc3)nc(-c3ccc(C45CC6CC(CC(C6)C4)C5)cc3)n2)cc1 ; c1ccc(-c2nc(-c3ccc(-c4ccc(-c5ccccc5)c5ocnc45)cc3)nc(-c3ccc(C45CC6CC(CC(C6)C4)C5)cc3)n2)cc1]

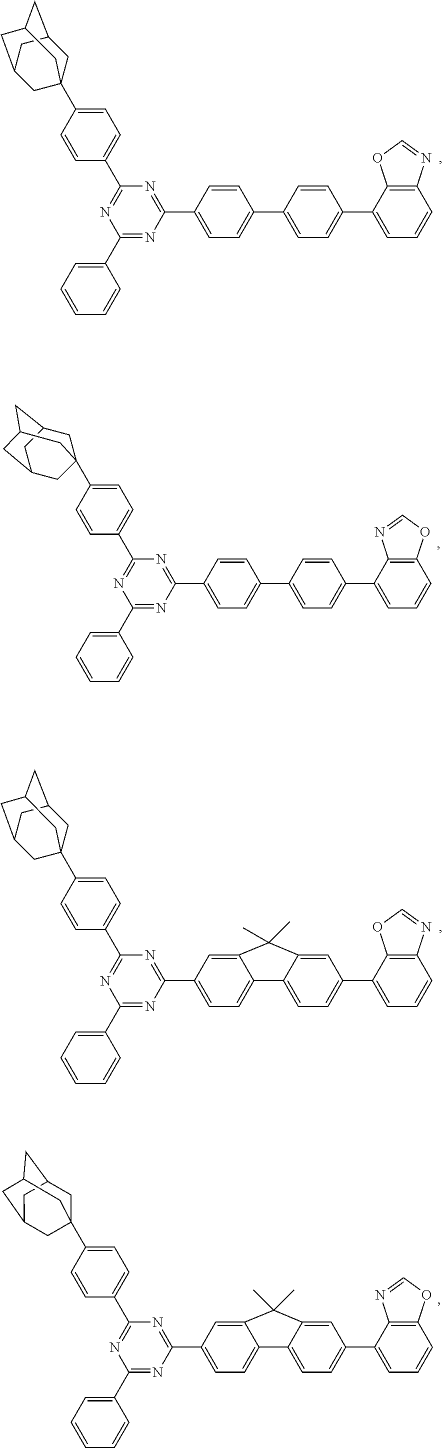 [SMILES: c1ccc(-c2nc(-c3ccc(-c4ccc(-c5cccc6ncoc56)cc4)cc3)nc(-c3ccc(C45CC6CC(CC(C6)C4)C5)cc3)n2)cc1 ; c1ccc(-c2nc(-c3ccc(-c4ccc(-c5cccc6ocnc56)cc4)cc3)nc(-c3ccc(C45CC6CC(CC(C6)C4)C5)cc3)n2)cc1 ; CC1(C)c2cc(-c3nc(-c4ccccc4)nc(-c4ccc(C56CC7CC(CC(C7)C5)C6)cc4)n3)ccc2-c2ccc(-c3cccc4ncoc34)cc21 ; CC1(C)c2cc(-c3nc(-c4ccccc4)nc(-c4ccc(C56CC7CC(CC(C7)C5)C6)cc4)n3)ccc2-c2ccc(-c3cccc4ocnc34)cc21]

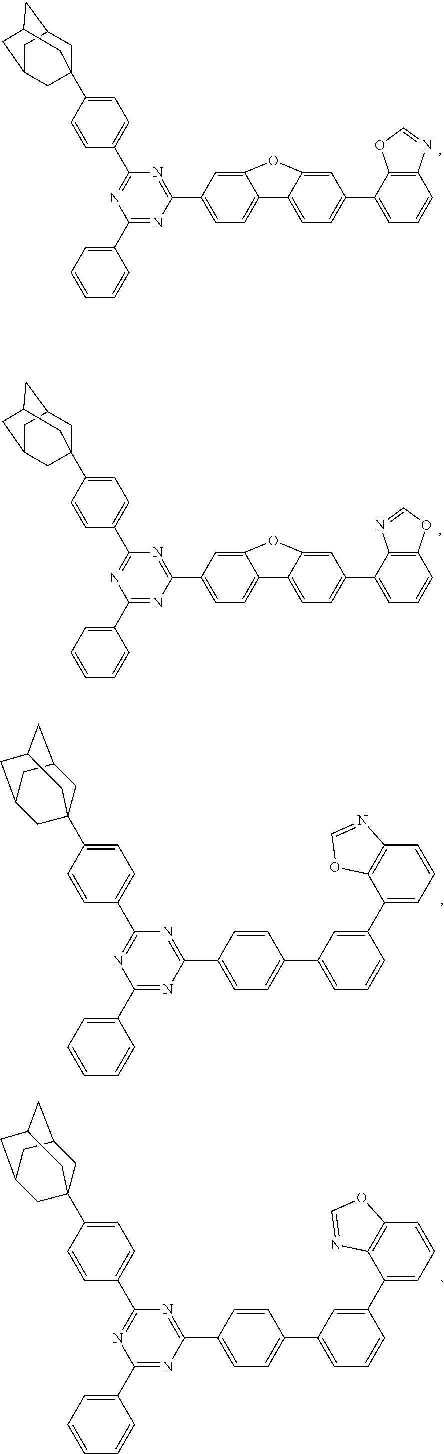 [SMILES: c1ccc(-c2nc(-c3ccc(C45CC6CC(CC(C6)C4)C5)cc3)nc(-c3ccc4c(c3)oc3cc(-c5cccc6ncoc56)ccc34)n2)cc1 ; c1ccc(-c2nc(-c3ccc(C45CC6CC(CC(C6)C4)C5)cc3)nc(-c3ccc4c(c3)oc3cc(-c5cccc6ocnc56)ccc34)n2)cc1 ; c1ccc(-c2nc(-c3ccc(-c4cccc(-c5cccc6ncoc56)c4)cc3)nc(-c3ccc(C45CC6CC(CC(C6)C4)C5)cc3)n2)cc1 ; c1ccc(-c2nc(-c3ccc(-c4cccc(-c5cccc6ocnc56)c4)cc3)nc(-c3ccc(C45CC6CC(CC(C6)C4)C5)cc3)n2)cc1]

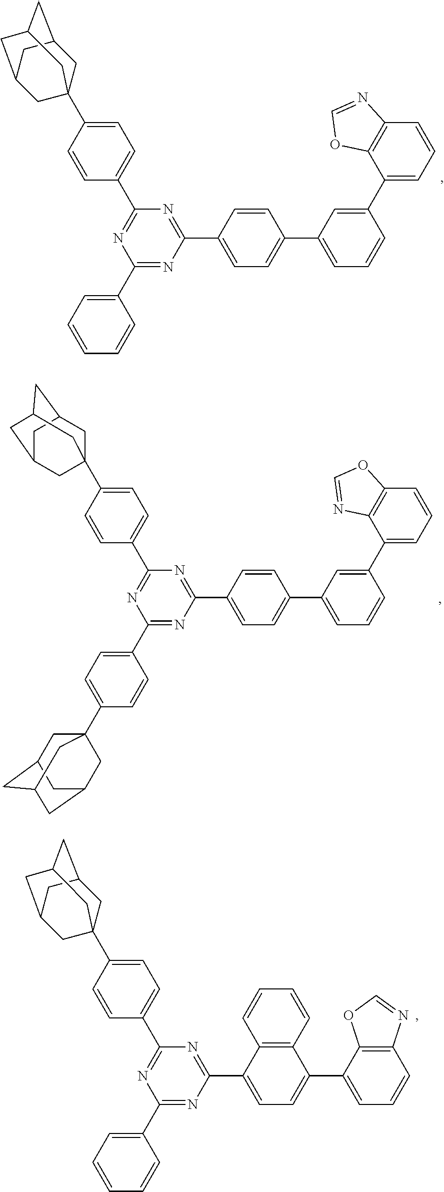

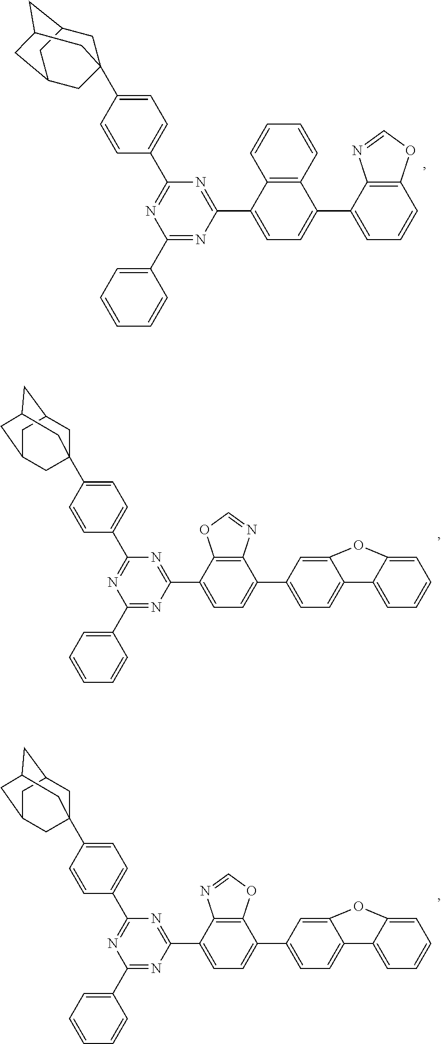 [SMILES: c1ccc(-c2nc(-c3ccc(C45CC6CC(CC(C6)C4)C5)cc3)nc(-c3ccc(-c4cccc5ocnc45)c4ccccc34)n2)cc1 ; c1ccc(-c2nc(-c3ccc(C45CC6CC(CC(C6)C4)C5)cc3)nc(-c3ccc(-c4ccc5c(c4)oc4ccccc45)c4ncoc34)n2)cc1 ; c1ccc(-c2nc(-c3ccc(C45CC6CC(CC(C6)C4)C5)cc3)nc(-c3ccc(-c4ccc5c(c4)oc4ccccc45)c4ocnc34)n2)cc1]

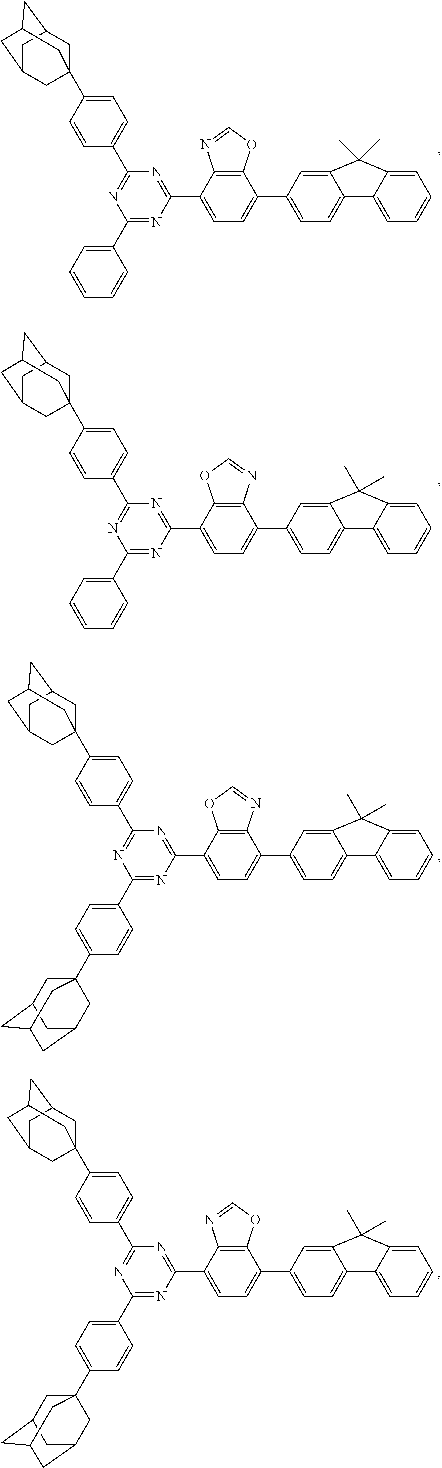

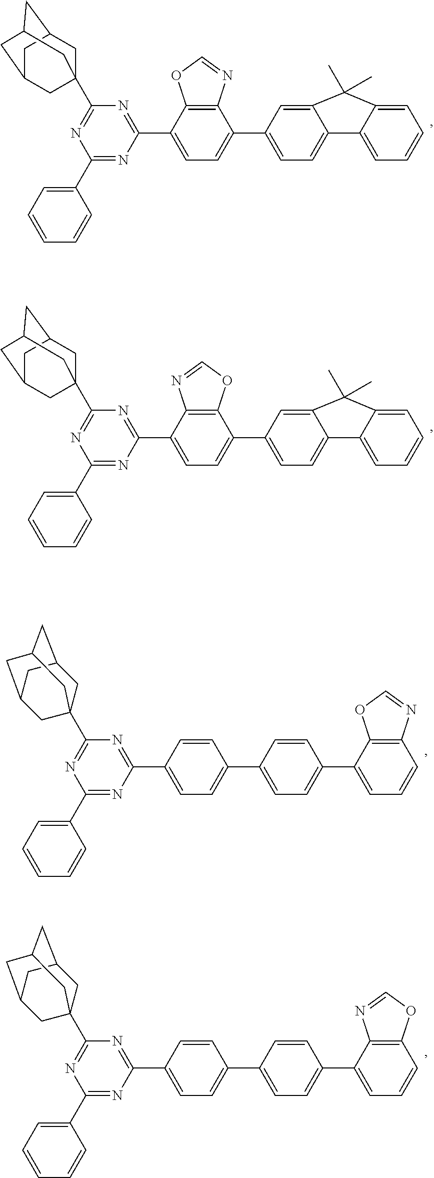 [SMILES: CC1(C)c2ccccc2-c2ccc(-c3ccc(-c4nc(-c5ccccc5)nc(C56CC7CC(CC(C7)C5)C6)n4)c4ocnc34)cc21 ; CC1(C)c2ccccc2-c2ccc(-c3ccc(-c4nc(-c5ccccc5)nc(C56CC7CC(CC(C7)C5)C6)n4)c4ncoc34)cc21 ; c1ccc(-c2nc(-c3ccc(-c4ccc(-c5cccc6ncoc56)cc4)cc3)nc(C34CC5CC(CC(C5)C3)C4)n2)cc1 ; c1ccc(-c2nc(-c3ccc(-c4ccc(-c5cccc6ocnc56)cc4)cc3)nc(C34CC5CC(CC(C5)C3)C4)n2)cc1]

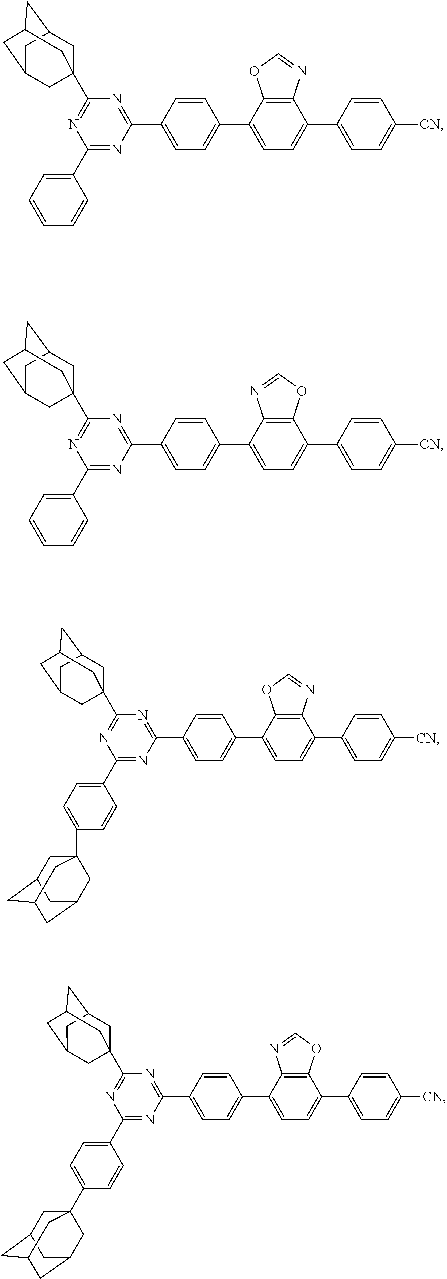 [SMILES: N#Cc1ccc(-c2ccc(-c3ccc(-c4nc(-c5ccccc5)nc(C56CC7CC(CC(C7)C5)C6)n4)cc3)c3ocnc23)cc1 ; N#Cc1ccc(-c2ccc(-c3ccc(-c4nc(-c5ccccc5)nc(C56CC7CC(CC(C7)C5)C6)n4)cc3)c3ncoc23)cc1 ; N#Cc1ccc(-c2ccc(-c3ccc(-c4nc(-c5ccc(C67CC8CC(CC(C8)C6)C7)cc5)nc(C56CC7CC(CC(C7)C5)C6)n4)cc3)c3ocnc23)cc1 ; N#Cc1ccc(-c2ccc(-c3ccc(-c4nc(-c5ccc(C67CC8CC(CC(C8)C6)C7)cc5)nc(C56CC7CC(CC(C7)C5)C6)n4)cc3)c3ncoc23)cc1]

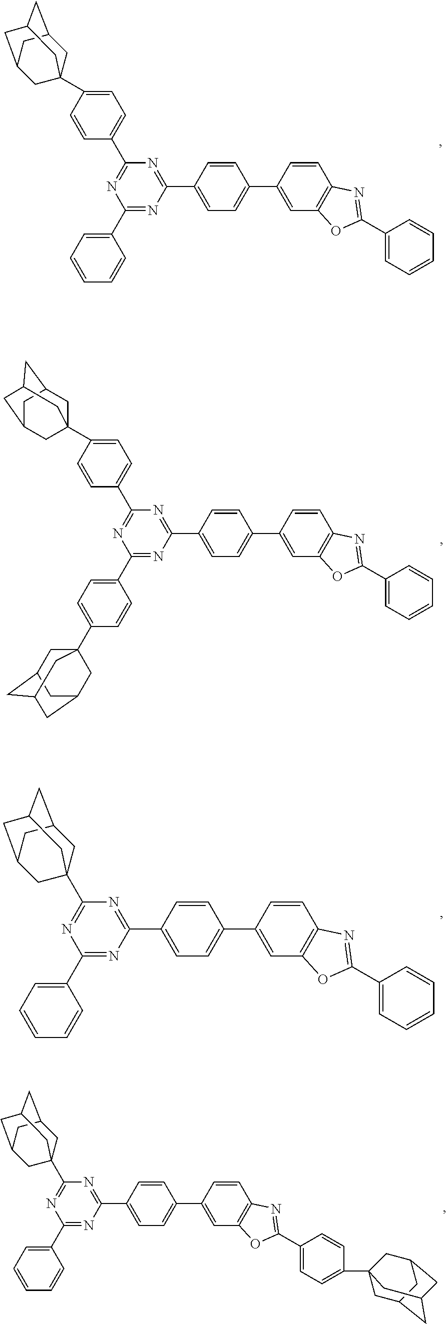 [SMILES: c1ccc(-c2nc(-c3ccc(-c4ccc5nc(-c6ccccc6)oc5c4)cc3)nc(-c3ccc(C45CC6CC(CC(C6)C4)C5)cc3)n2)cc1 ; c1ccc(-c2nc3ccc(-c4ccc(-c5nc(-c6ccc(C78CC9CC(CC(C9)C7)C8)cc6)nc(-c6ccc(C78CC9CC(CC(C9)C7)C8)cc6)n5)cc4)cc3o2)cc1 ; c1ccc(-c2nc(-c3ccc(-c4ccc5nc(-c6ccccc6)oc5c4)cc3)nc(C34CC5CC(CC(C5)C3)C4)n2)cc1 ; c1ccc(-c2nc(-c3ccc(-c4ccc5nc(-c6ccc(C78CC9CC(CC(C9)C7)C8)cc6)oc5c4)cc3)nc(C34CC5CC(CC(C5)C3)C4)n2)cc1]

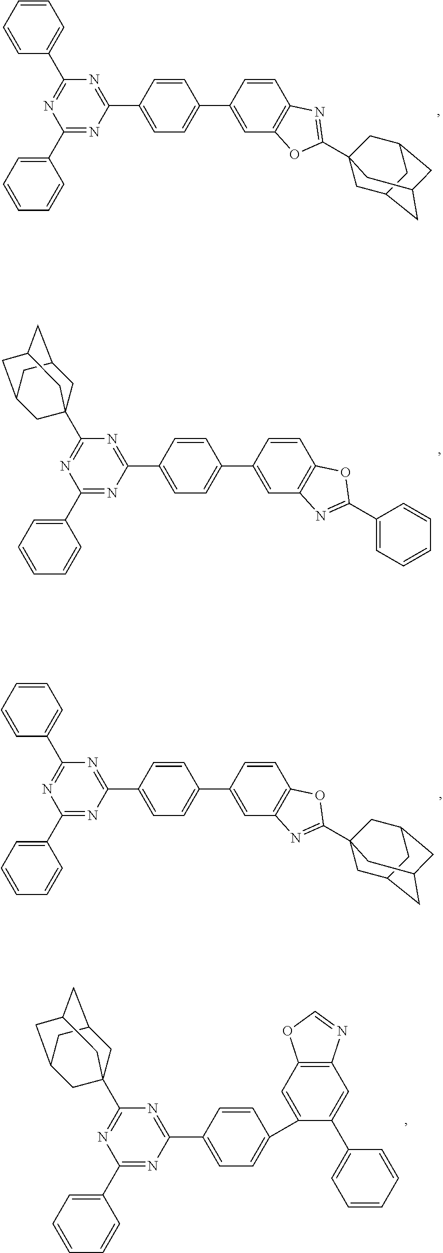 [SMILES: c1ccc(-c2nc(-c3ccccc3)nc(-c3ccc(-c4ccc5nc(C67CC8CC(CC(C8)C6)C7)oc5c4)cc3)n2)cc1 ; c1ccc(-c2nc(-c3ccc(-c4ccc5oc(-c6ccccc6)nc5c4)cc3)nc(C34CC5CC(CC(C5)C3)C4)n2)cc1 ; c1ccc(-c2nc(-c3ccccc3)nc(-c3ccc(-c4ccc5oc(C67CC8CC(CC(C8)C6)C7)nc5c4)cc3)n2)cc1 ; c1ccc(-c2nc(-c3ccc(-c4cc5ocnc5cc4-c4ccccc4)cc3)nc(C34CC5CC(CC(C5)C3)C4)n2)cc1]

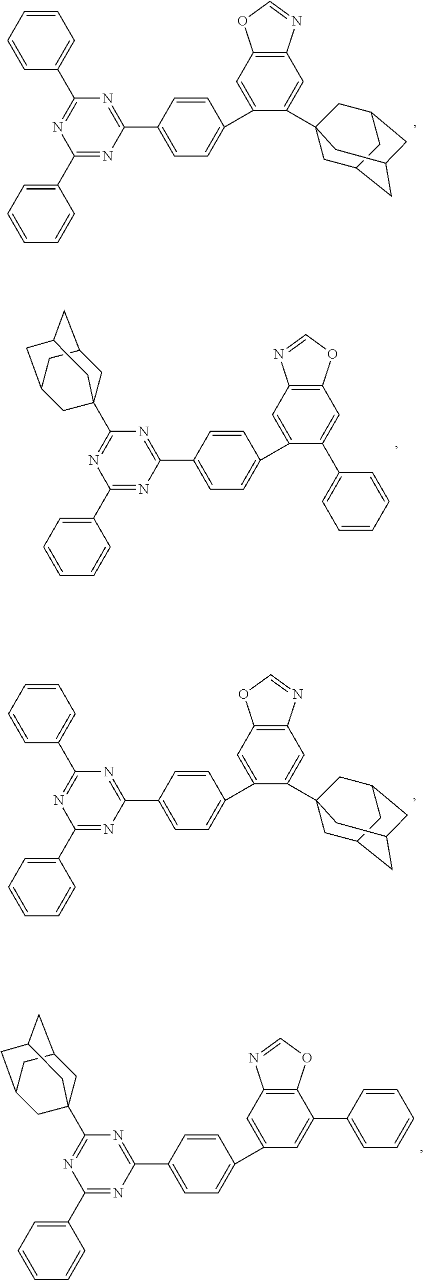 [SMILES: c1ccc(-c2nc(-c3ccccc3)nc(-c3ccc(-c4cc5ocnc5cc4C45CC6CC(CC(C6)C4)C5)cc3)n2)cc1 ; c1ccc(-c2nc(-c3ccc(-c4cc5ncoc5cc4-c4ccccc4)cc3)nc(C34CC5CC(CC(C5)C3)C4)n2)cc1 ; c1ccc(-c2nc(-c3ccccc3)nc(-c3ccc(-c4cc5ocnc5cc4C45CC6CC(CC(C6)C4)C5)cc3)n2)cc1 ; c1ccc(-c2nc(-c3ccc(-c4cc(-c5ccccc5)c5ocnc5c4)cc3)nc(C34CC5CC(CC(C5)C3)C4)n2)cc1]

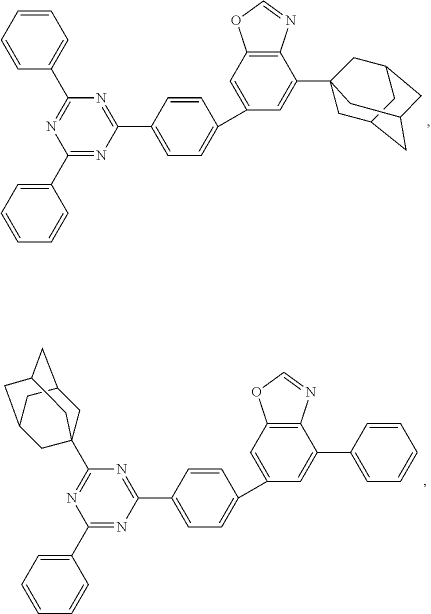 [SMILES: c1ccc(-c2nc(-c3ccccc3)nc(-c3ccc(-c4cc(C56CC7CC(CC(C7)C5)C6)c5ncoc5c4)cc3)n2)cc1 ; c1ccc(-c2nc(-c3ccc(-c4cc(-c5ccccc5)c5ncoc5c4)cc3)nc(C34CC5CC(CC(C5)C3)C4)n2)cc1]

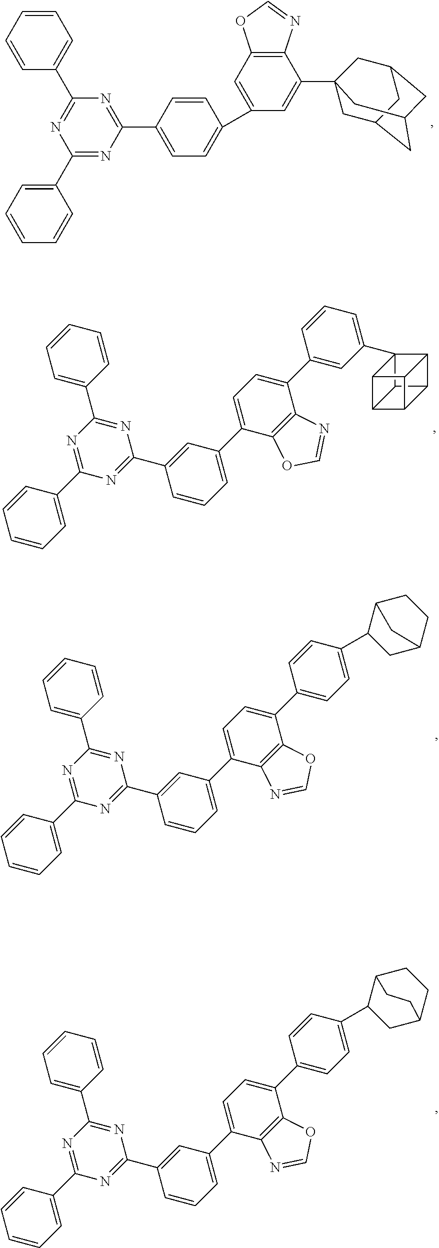 [SMILES: c1ccc(-c2nc(-c3ccccc3)nc(-c3ccc(-c4cc(C56CC7CC(CC(C7)C5)C6)c5ncoc5c4)cc3)n2)cc1 ; c1ccc(-c2nc(-c3ccccc3)nc(-c3cccc(-c4ccc(-c5cccc(C67C8C9C%10C8C6C%10C97)c5)c5ncoc45)c3)n2)cc1 ; c1ccc(-c2nc(-c3ccccc3)nc(-c3cccc(-c4ccc(-c5ccc(C6CC7CCC6C7)cc5)c5ocnc45)c3)n2)cc1 ; c1ccc(-c2nc(-c3ccccc3)nc(-c3cccc(-c4ccc(-c5ccc(C6CC7CCC6CC7)cc5)c5ocnc45)c3)n2)cc1]

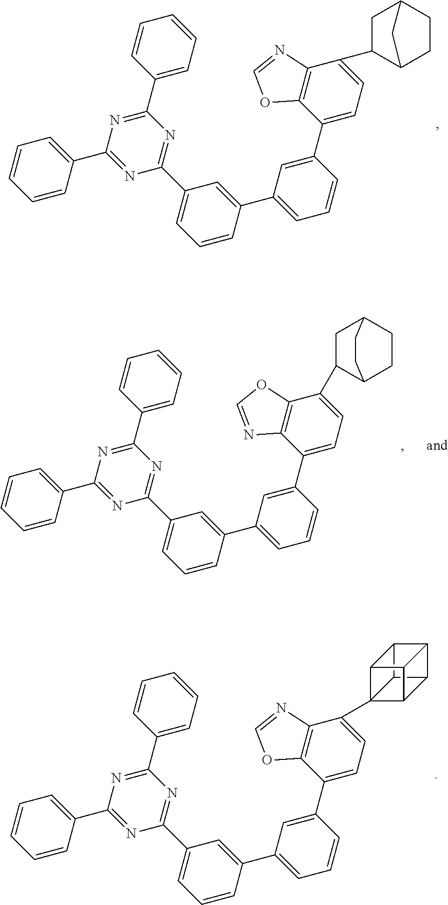 [SMILES: c1ccc(-c2nc(-c3ccccc3)nc(-c3cccc(-c4cccc(-c5ccc(C6CC7CCC6C7)c6ncoc56)c4)c3)n2)cc1 ; c1ccc(-c2nc(-c3ccccc3)nc(-c3cccc(-c4cccc(-c5ccc(C6CC7CCC6CC7)c6ocnc56)c4)c3)n2)cc1 ; c1ccc(-c2nc(-c3ccccc3)nc(-c3cccc(-c4cccc(-c5ccc(C67C8C9C%10C8C6C%10C97)c6ncoc56)c4)c3)n2)cc1]

Optionally, the chemical structural formula of the compound includes any one of

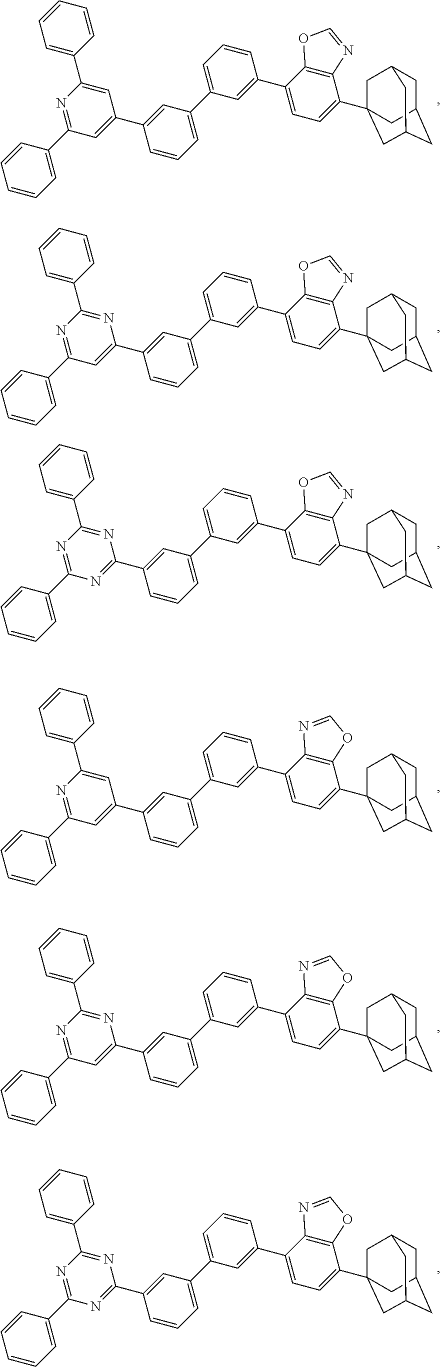

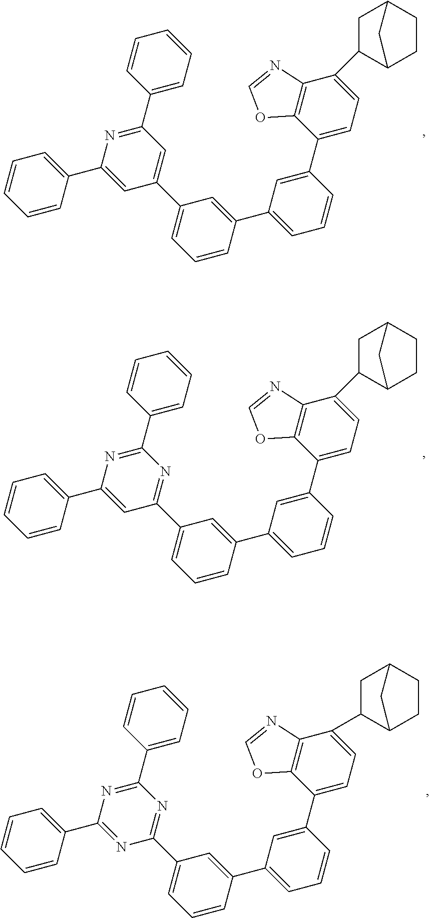 [SMILES: c1ccc(-c2cc(-c3cccc(-c4cccc(-c5ccc(C6CC7CCC6C7)c6ncoc56)c4)c3)cc(-c3ccccc3)n2)cc1 ; c1ccc(-c2cc(-c3cccc(-c4cccc(-c5ccc(C6CC7CCC6C7)c6ncoc56)c4)c3)nc(-c3ccccc3)n2)cc1 ; c1ccc(-c2nc(-c3ccccc3)nc(-c3cccc(-c4cccc(-c5ccc(C6CC7CCC6C7)c6ncoc56)c4)c3)n2)cc1]

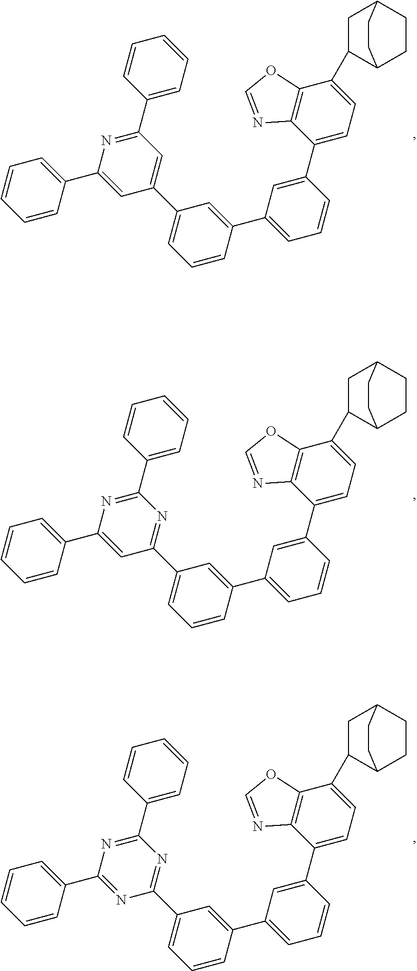 [SMILES: c1ccc(-c2cc(-c3cccc(-c4cccc(-c5ccc(C6CC7CCC6CC7)c6ocnc56)c4)c3)cc(-c3ccccc3)n2)cc1 ; c1ccc(-c2cc(-c3cccc(-c4cccc(-c5ccc(C6CC7CCC6CC7)c6ocnc56)c4)c3)nc(-c3ccccc3)n2)cc1 ; c1ccc(-c2nc(-c3ccccc3)nc(-c3cccc(-c4cccc(-c5ccc(C6CC7CCC6CC7)c6ocnc56)c4)c3)n2)cc1]

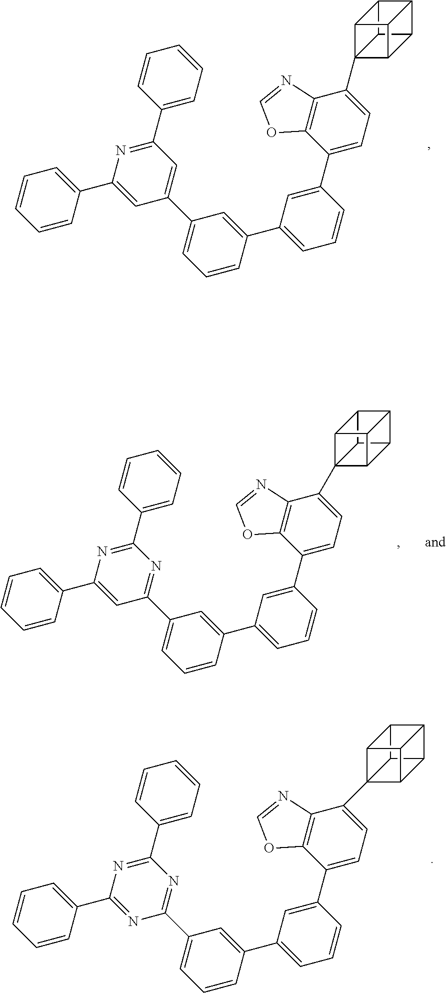 [SMILES: c1ccc(-c2cc(-c3cccc(-c4cccc(-c5ccc(C67C8C9C%10C8C6C%10C97)c6ncoc56)c4)c3)cc(-c3ccccc3)n2)cc1 ; c1ccc(-c2cc(-c3cccc(-c4cccc(-c5ccc(C67C8C9C%10C8C6C%10C97)c6ncoc56)c4)c3)nc(-c3ccccc3)n2)cc1 ; c1ccc(-c2nc(-c3ccccc3)nc(-c3cccc(-c4cccc(-c5ccc(C67C8C9C%10C8C6C%10C97)c6ncoc56)c4)c3)n2)cc1]

Optionally, the chemical structural formula of the compound having cyano includes any one of

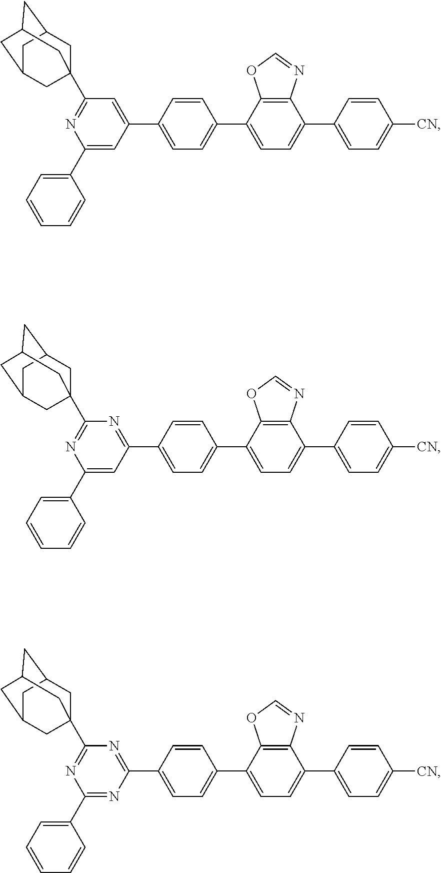 [SMILES: N#Cc1ccc(-c2ccc(-c3ccc(-c4cc(-c5ccccc5)nc(C56CC7CC(CC(C7)C5)C6)c4)cc3)c3ocnc23)cc1 ; N#Cc1ccc(-c2ccc(-c3ccc(-c4cc(-c5ccccc5)nc(C56CC7CC(CC(C7)C5)C6)n4)cc3)c3ocnc23)cc1 ; N#Cc1ccc(-c2ccc(-c3ccc(-c4nc(-c5ccccc5)nc(C56CC7CC(CC(C7)C5)C6)n4)cc3)c3ocnc23)cc1]

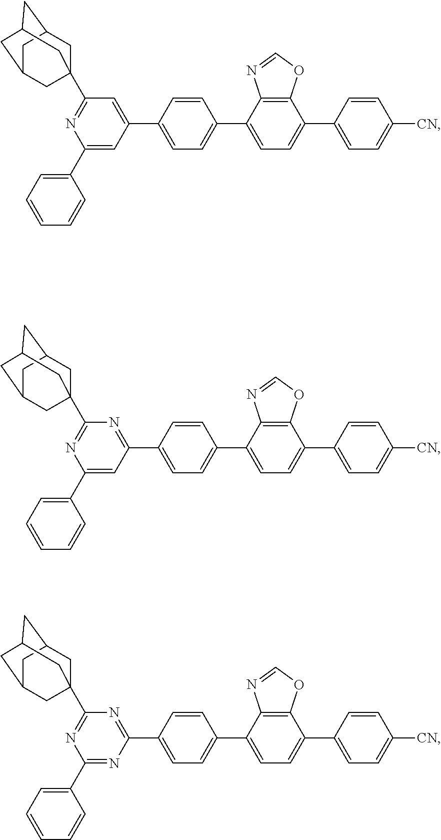 [SMILES: N#Cc1ccc(-c2ccc(-c3ccc(-c4cc(-c5ccccc5)nc(C56CC7CC(CC(C7)C5)C6)c4)cc3)c3ncoc23)cc1 ; N#Cc1ccc(-c2ccc(-c3ccc(-c4cc(-c5ccccc5)nc(C56CC7CC(CC(C7)C5)C6)n4)cc3)c3ncoc23)cc1 ; N#Cc1ccc(-c2ccc(-c3ccc(-c4nc(-c5ccccc5)nc(C56CC7CC(CC(C7)C5)C6)n4)cc3)c3ncoc23)cc1]

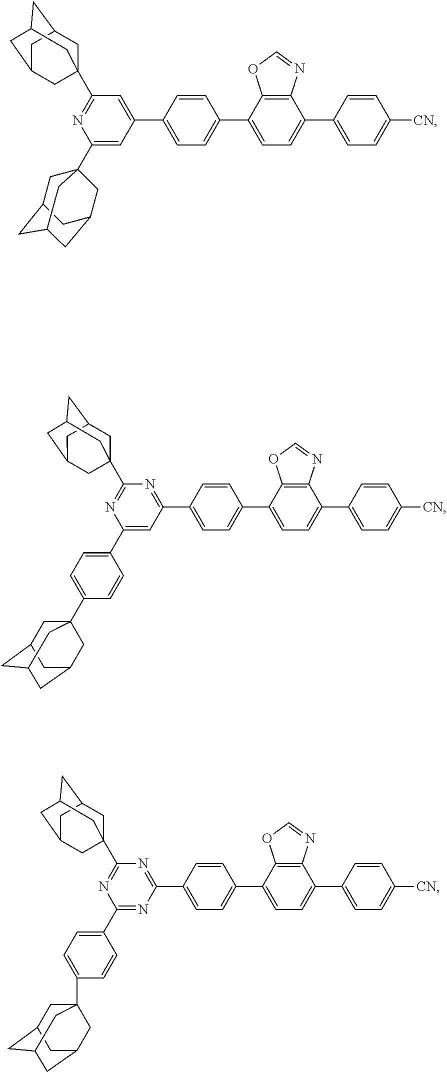 [SMILES: N#Cc1ccc(-c2ccc(-c3ccc(-c4cc(C56CC7CC(CC(C7)C5)C6)nc(C56CC7CC(CC(C7)C5)C6)c4)cc3)c3ocnc23)cc1 ; N#Cc1ccc(-c2ccc(-c3ccc(-c4cc(-c5ccc(C67CC8CC(CC(C8)C6)C7)cc5)nc(C56CC7CC(CC(C7)C5)C6)n4)cc3)c3ocnc23)cc1 ; N#Cc1ccc(-c2ccc(-c3ccc(-c4nc(-c5ccc(C67CC8CC(CC(C8)C6)C7)cc5)nc(C56CC7CC(CC(C7)C5)C6)n4)cc3)c3ocnc23)cc1]

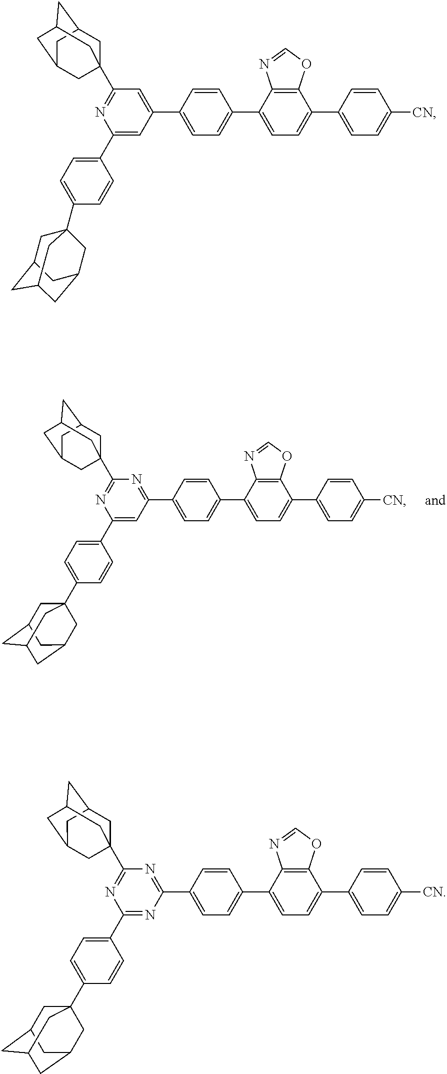 [SMILES: N#Cc1ccc(-c2ccc(-c3ccc(-c4cc(-c5ccc(C67CC8CC(CC(C8)C6)C7)cc5)nc(C56CC7CC(CC(C7)C5)C6)c4)cc3)c3ncoc23)cc1 ; N#Cc1ccc(-c2ccc(-c3ccc(-c4cc(-c5ccc(C67CC8CC(CC(C8)C6)C7)cc5)nc(C56CC7CC(CC(C7)C5)C6)n4)cc3)c3ncoc23)cc1 ; N#Cc1ccc(-c2ccc(-c3ccc(-c4nc(-c5ccc(C67CC8CC(CC(C8)C6)C7)cc5)nc(C56CC7CC(CC(C7)C5)C6)n4)cc3)c3ncoc23)cc1]

Optionally, the chemical structural formula of the compound includes any one of

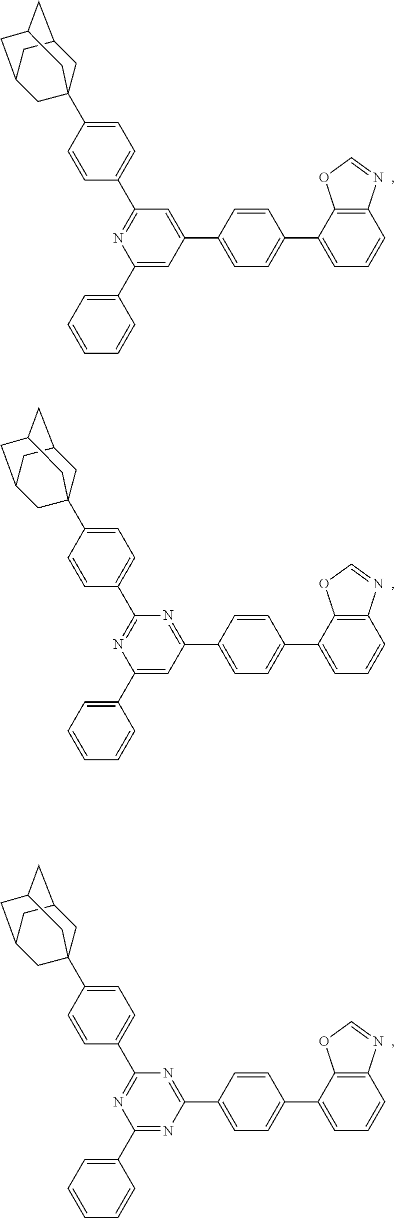 [SMILES: c1ccc(-c2cc(-c3ccc(-c4cccc5ncoc45)cc3)cc(-c3ccc(C45CC6CC(CC(C6)C4)C5)cc3)n2)cc1 ; c1ccc(-c2cc(-c3ccc(-c4cccc5ncoc45)cc3)nc(-c3ccc(C45CC6CC(CC(C6)C4)C5)cc3)n2)cc1 ; c1ccc(-c2nc(-c3ccc(-c4cccc5ncoc45)cc3)nc(-c3ccc(C45CC6CC(CC(C6)C4)C5)cc3)n2)cc1]

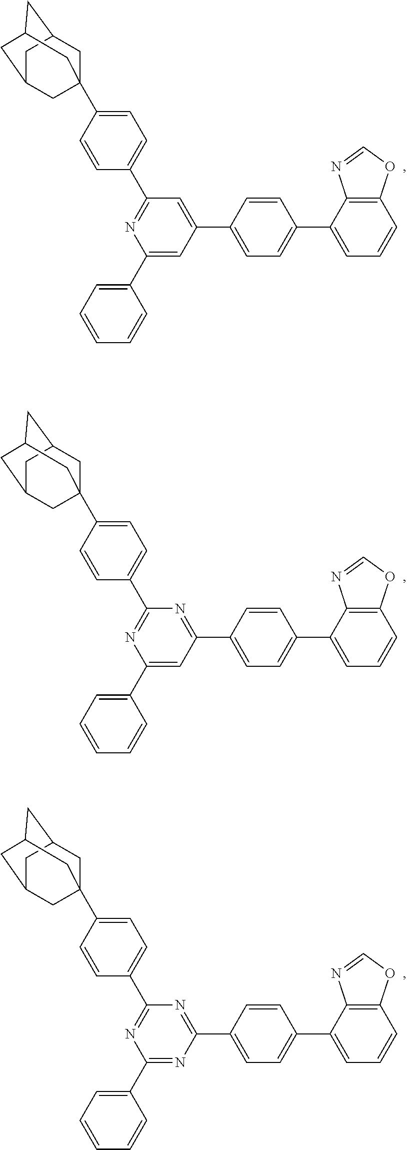 [SMILES: c1ccc(-c2cc(-c3ccc(-c4cccc5ocnc45)cc3)cc(-c3ccc(C45CC6CC(CC(C6)C4)C5)cc3)n2)cc1 ; c1ccc(-c2cc(-c3ccc(-c4cccc5ocnc45)cc3)nc(-c3ccc(C45CC6CC(CC(C6)C4)C5)cc3)n2)cc1 ; c1ccc(-c2nc(-c3ccc(-c4cccc5ocnc45)cc3)nc(-c3ccc(C45CC6CC(CC(C6)C4)C5)cc3)n2)cc1]

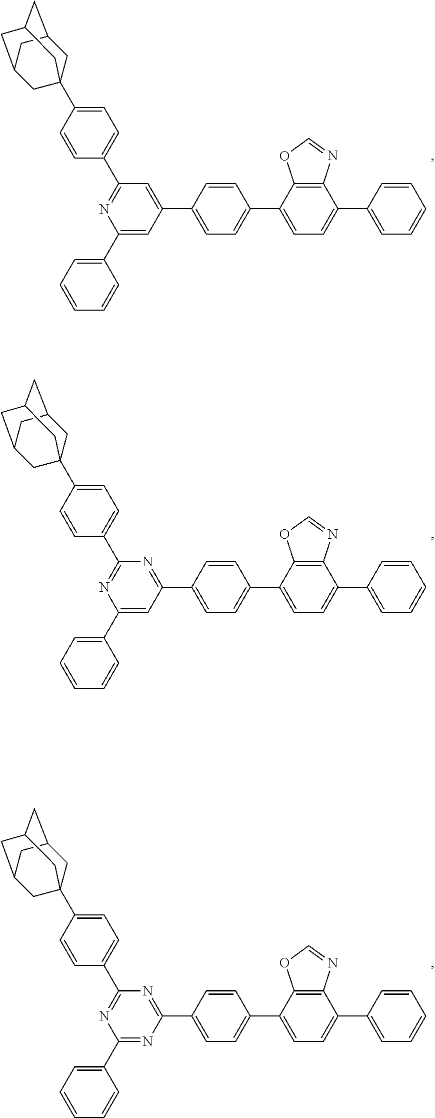 [SMILES: c1ccc(-c2cc(-c3ccc(-c4ccc(-c5ccccc5)c5ncoc45)cc3)cc(-c3ccc(C45CC6CC(CC(C6)C4)C5)cc3)n2)cc1 ; c1ccc(-c2cc(-c3ccc(-c4ccc(-c5ccccc5)c5ncoc45)cc3)nc(-c3ccc(C45CC6CC(CC(C6)C4)C5)cc3)n2)cc1 ; c1ccc(-c2nc(-c3ccc(-c4ccc(-c5ccccc5)c5ncoc45)cc3)nc(-c3ccc(C45CC6CC(CC(C6)C4)C5)cc3)n2)cc1]

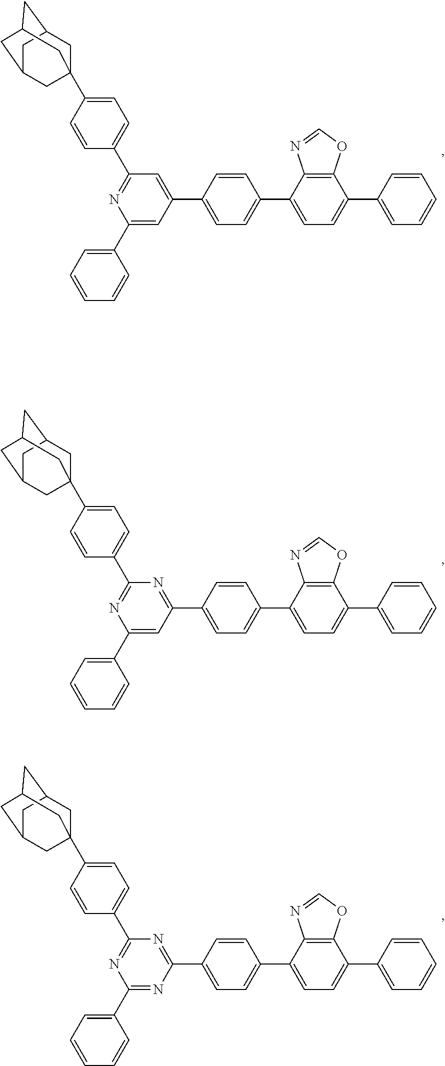 [SMILES: c1ccc(-c2cc(-c3ccc(-c4ccc(-c5ccccc5)c5ocnc45)cc3)cc(-c3ccc(C45CC6CC(CC(C6)C4)C5)cc3)n2)cc1 ; c1ccc(-c2cc(-c3ccc(-c4ccc(-c5ccccc5)c5ocnc45)cc3)nc(-c3ccc(C45CC6CC(CC(C6)C4)C5)cc3)n2)cc1 ; c1ccc(-c2nc(-c3ccc(-c4ccc(-c5ccccc5)c5ocnc45)cc3)nc(-c3ccc(C45CC6CC(CC(C6)C4)C5)cc3)n2)cc1]

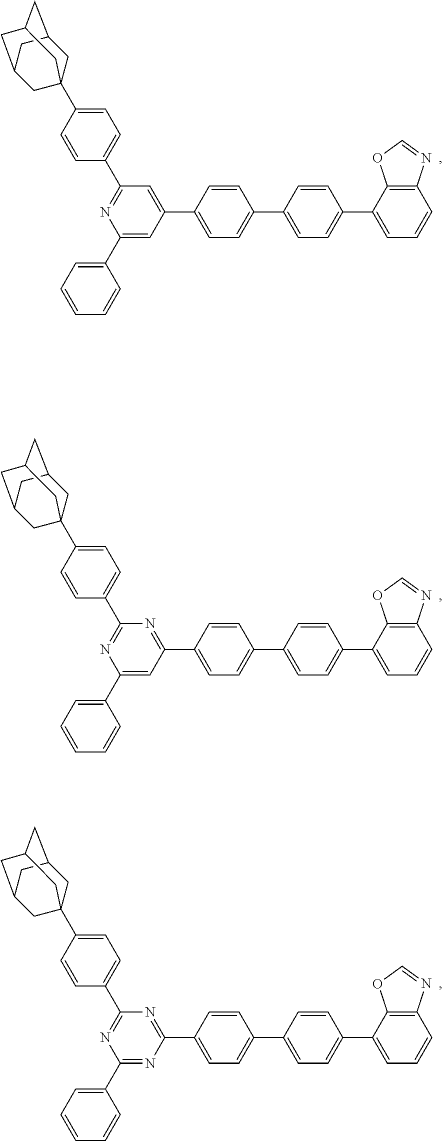 [SMILES: c1ccc(-c2cc(-c3ccc(-c4ccc(-c5cccc6ncoc56)cc4)cc3)cc(-c3ccc(C45CC6CC(CC(C6)C4)C5)cc3)n2)cc1 ; c1ccc(-c2cc(-c3ccc(-c4ccc(-c5cccc6ncoc56)cc4)cc3)nc(-c3ccc(C45CC6CC(CC(C6)C4)C5)cc3)n2)cc1 ; c1ccc(-c2nc(-c3ccc(-c4ccc(-c5cccc6ncoc56)cc4)cc3)nc(-c3ccc(C45CC6CC(CC(C6)C4)C5)cc3)n2)cc1]

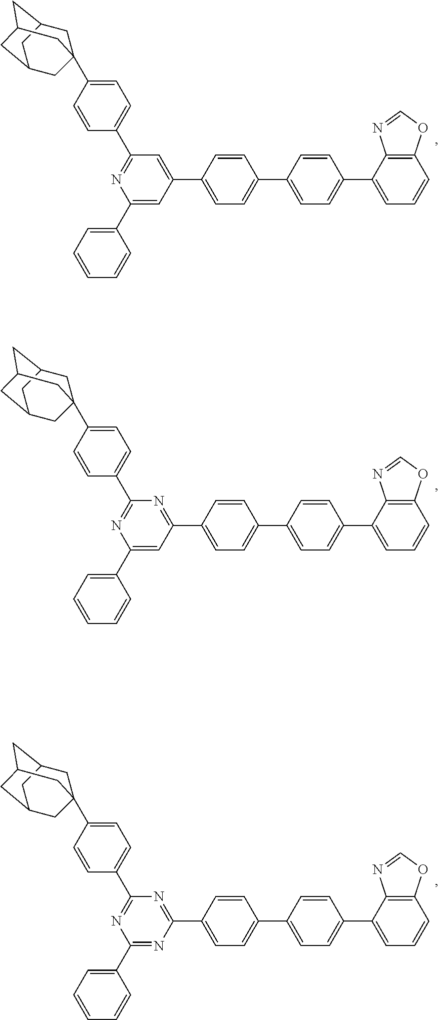 [SMILES: c1ccc(-c2cc(-c3ccc(-c4ccc(-c5cccc6ocnc56)cc4)cc3)cc(-c3ccc(C45CC6CC(CC(C6)C4)C5)cc3)n2)cc1 ; c1ccc(-c2cc(-c3ccc(-c4ccc(-c5cccc6ocnc56)cc4)cc3)nc(-c3ccc(C45CC6CC(CC(C6)C4)C5)cc3)n2)cc1 ; c1ccc(-c2nc(-c3ccc(-c4ccc(-c5cccc6ocnc56)cc4)cc3)nc(-c3ccc(C45CC6CC(CC(C6)C4)C5)cc3)n2)cc1]

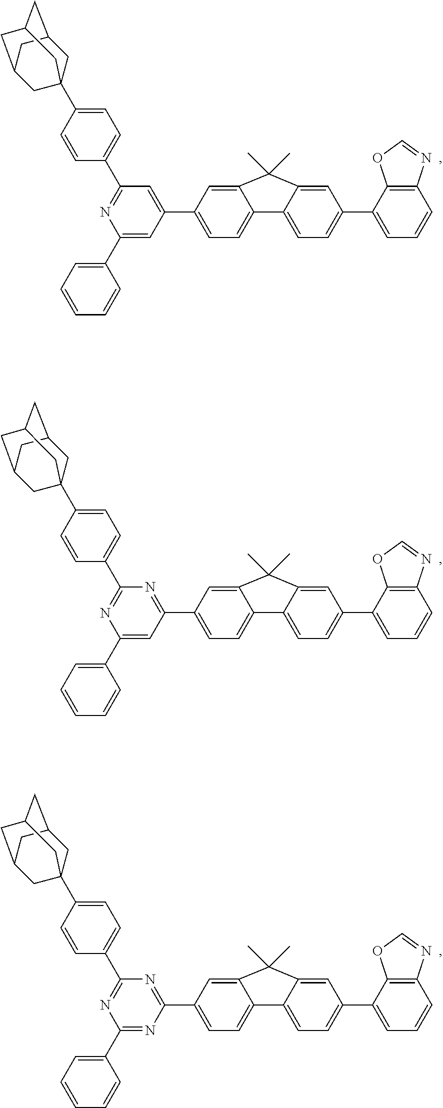 [SMILES: CC1(C)c2cc(-c3cc(-c4ccccc4)nc(-c4ccc(C56CC7CC(CC(C7)C5)C6)cc4)c3)ccc2-c2ccc(-c3cccc4ncoc34)cc21 ; CC1(C)c2cc(-c3cc(-c4ccccc4)nc(-c4ccc(C56CC7CC(CC(C7)C5)C6)cc4)n3)ccc2-c2ccc(-c3cccc4ncoc34)cc21 ; CC1(C)c2cc(-c3nc(-c4ccccc4)nc(-c4ccc(C56CC7CC(CC(C7)C5)C6)cc4)n3)ccc2-c2ccc(-c3cccc4ncoc34)cc21]

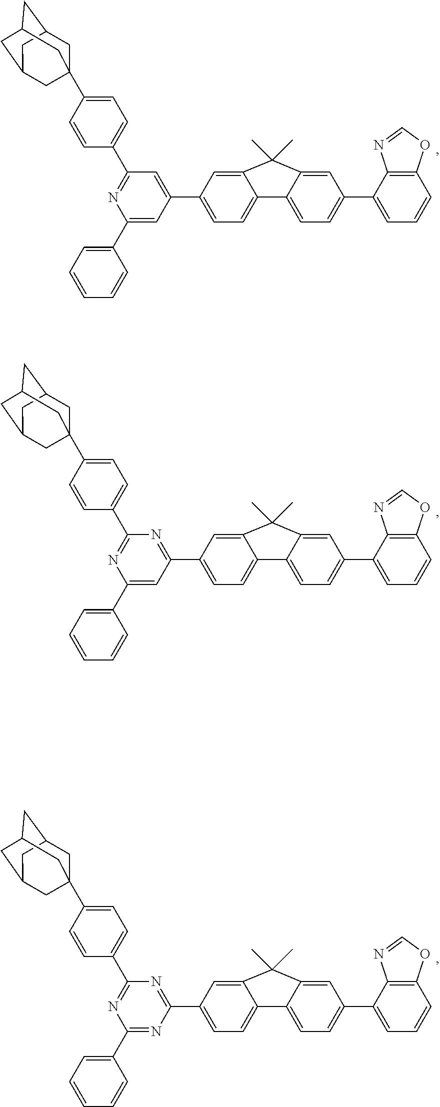 [SMILES: CC1(C)c2cc(-c3cc(-c4ccccc4)nc(-c4ccc(C56CC7CC(CC(C7)C5)C6)cc4)c3)ccc2-c2ccc(-c3cccc4ocnc34)cc21 ; CC1(C)c2cc(-c3cc(-c4ccccc4)nc(-c4ccc(C56CC7CC(CC(C7)C5)C6)cc4)n3)ccc2-c2ccc(-c3cccc4ocnc34)cc21 ; CC1(C)c2cc(-c3nc(-c4ccccc4)nc(-c4ccc(C56CC7CC(CC(C7)C5)C6)cc4)n3)ccc2-c2ccc(-c3cccc4ocnc34)cc21]

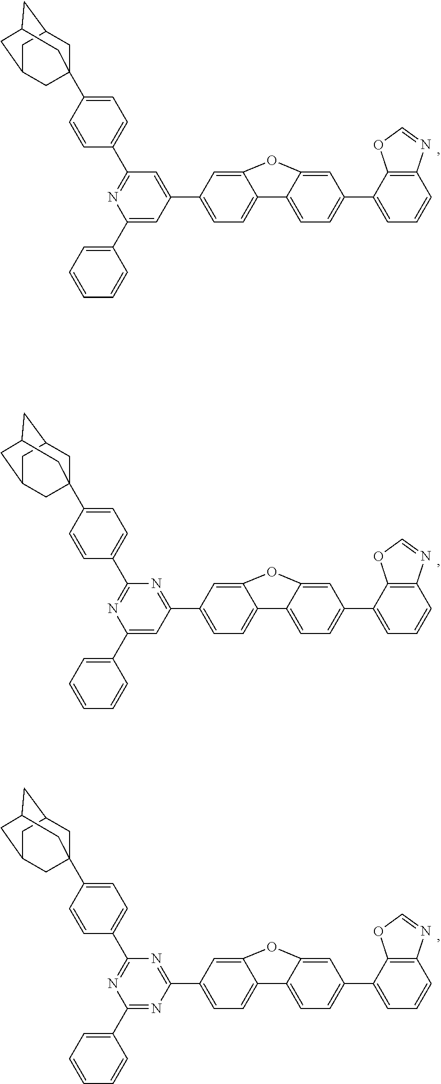 [SMILES: c1ccc(-c2cc(-c3ccc4c(c3)oc3cc(-c5cccc6ncoc56)ccc34)cc(-c3ccc(C45CC6CC(CC(C6)C4)C5)cc3)n2)cc1 ; c1ccc(-c2cc(-c3ccc4c(c3)oc3cc(-c5cccc6ncoc56)ccc34)nc(-c3ccc(C45CC6CC(CC(C6)C4)C5)cc3)n2)cc1 ; c1ccc(-c2nc(-c3ccc(C45CC6CC(CC(C6)C4)C5)cc3)nc(-c3ccc4c(c3)oc3cc(-c5cccc6ncoc56)ccc34)n2)cc1]

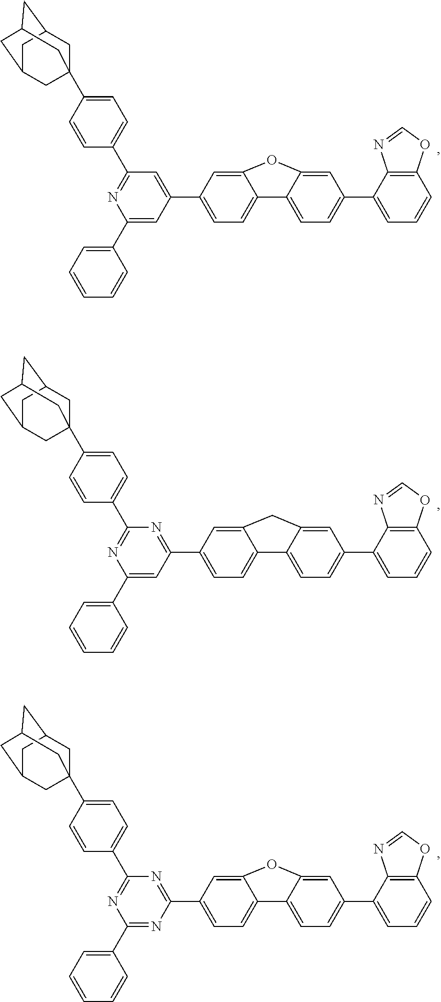 [SMILES: c1ccc(-c2cc(-c3ccc4c(c3)oc3cc(-c5cccc6ocnc56)ccc34)cc(-c3ccc(C45CC6CC(CC(C6)C4)C5)cc3)n2)cc1 ; c1ccc(-c2cc(-c3ccc4c(c3)Cc3cc(-c5cccc6ocnc56)ccc3-4)nc(-c3ccc(C45CC6CC(CC(C6)C4)C5)cc3)n2)cc1 ; c1ccc(-c2nc(-c3ccc(C45CC6CC(CC(C6)C4)C5)cc3)nc(-c3ccc4c(c3)oc3cc(-c5cccc6ocnc56)ccc34)n2)cc1]

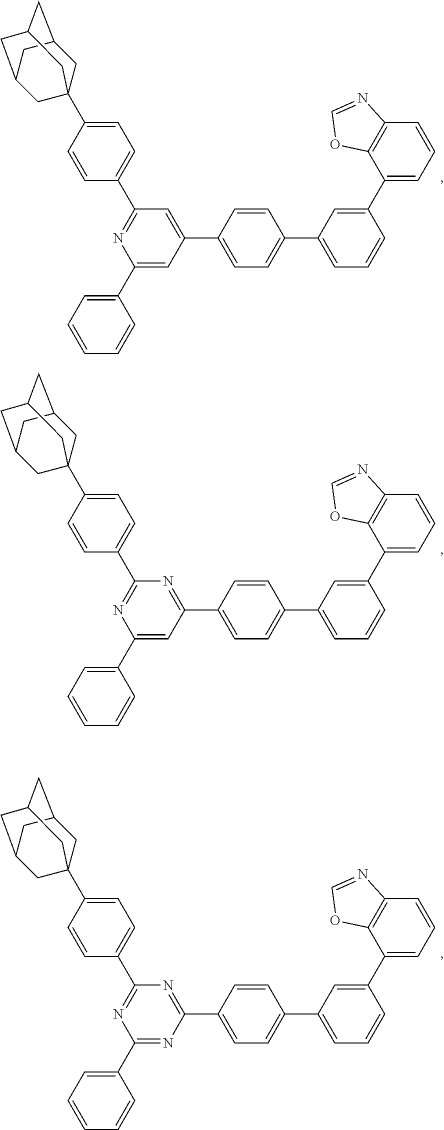 [SMILES: c1ccc(-c2cc(-c3ccc(-c4cccc(-c5cccc6ncoc56)c4)cc3)cc(-c3ccc(C45CC6CC(CC(C6)C4)C5)cc3)n2)cc1 ; c1ccc(-c2cc(-c3ccc(-c4cccc(-c5cccc6ncoc56)c4)cc3)nc(-c3ccc(C45CC6CC(CC(C6)C4)C5)cc3)n2)cc1 ; c1ccc(-c2nc(-c3ccc(-c4cccc(-c5cccc6ncoc56)c4)cc3)nc(-c3ccc(C45CC6CC(CC(C6)C4)C5)cc3)n2)cc1]

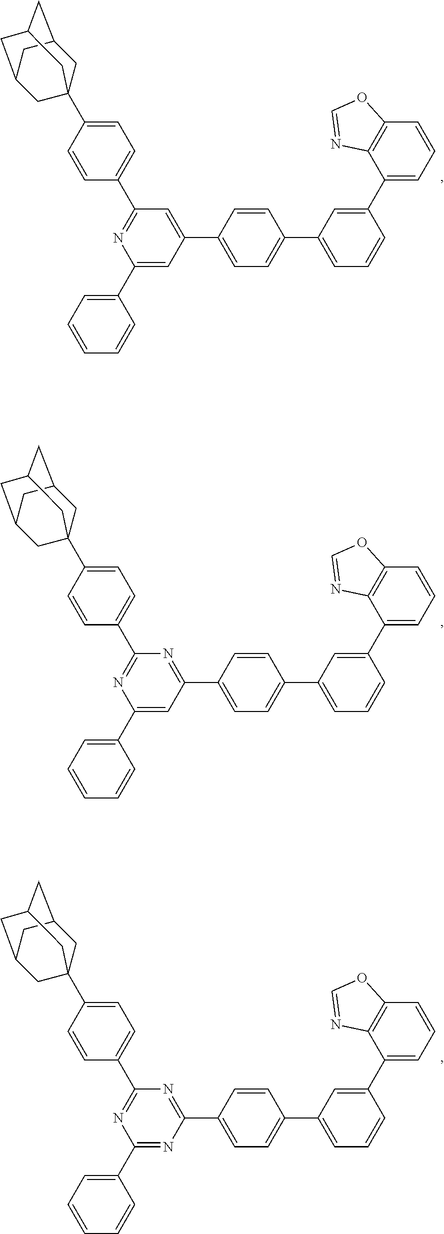 [SMILES: c1ccc(-c2cc(-c3ccc(-c4cccc(-c5cccc6ocnc56)c4)cc3)cc(-c3ccc(C45CC6CC(CC(C6)C4)C5)cc3)n2)cc1 ; c1ccc(-c2cc(-c3ccc(-c4cccc(-c5cccc6ocnc56)c4)cc3)nc(-c3ccc(C45CC6CC(CC(C6)C4)C5)cc3)n2)cc1 ; c1ccc(-c2nc(-c3ccc(-c4cccc(-c5cccc6ocnc56)c4)cc3)nc(-c3ccc(C45CC6CC(CC(C6)C4)C5)cc3)n2)cc1]

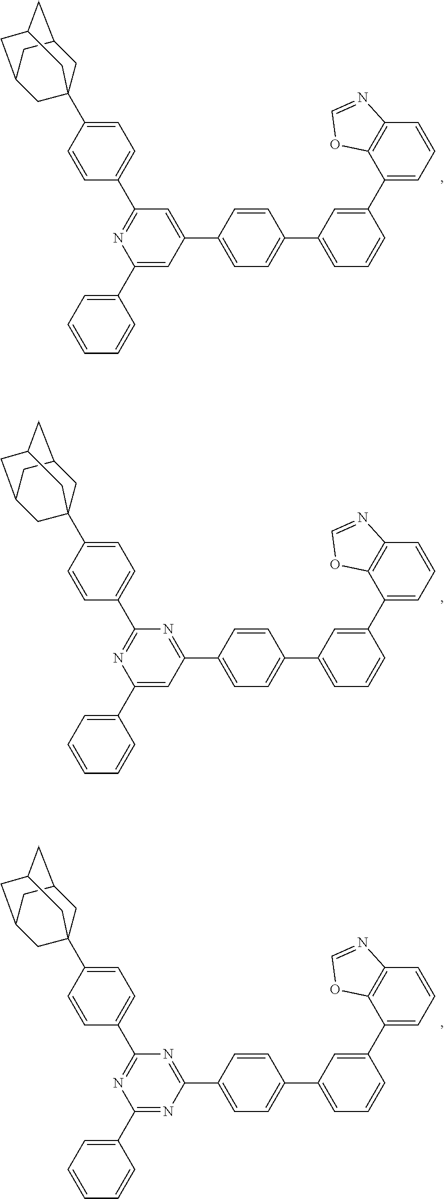 [SMILES: c1ccc(-c2cc(-c3ccc(-c4cccc(-c5cccc6ncoc56)c4)cc3)cc(-c3ccc(C45CC6CC(CC(C6)C4)C5)cc3)n2)cc1 ; c1ccc(-c2cc(-c3ccc(-c4cccc(-c5cccc6ncoc56)c4)cc3)nc(-c3ccc(C45CC6CC(CC(C6)C4)C5)cc3)n2)cc1 ; c1ccc(-c2nc(-c3ccc(-c4cccc(-c5cccc6ncoc56)c4)cc3)nc(-c3ccc(C45CC6CC(CC(C6)C4)C5)cc3)n2)cc1]

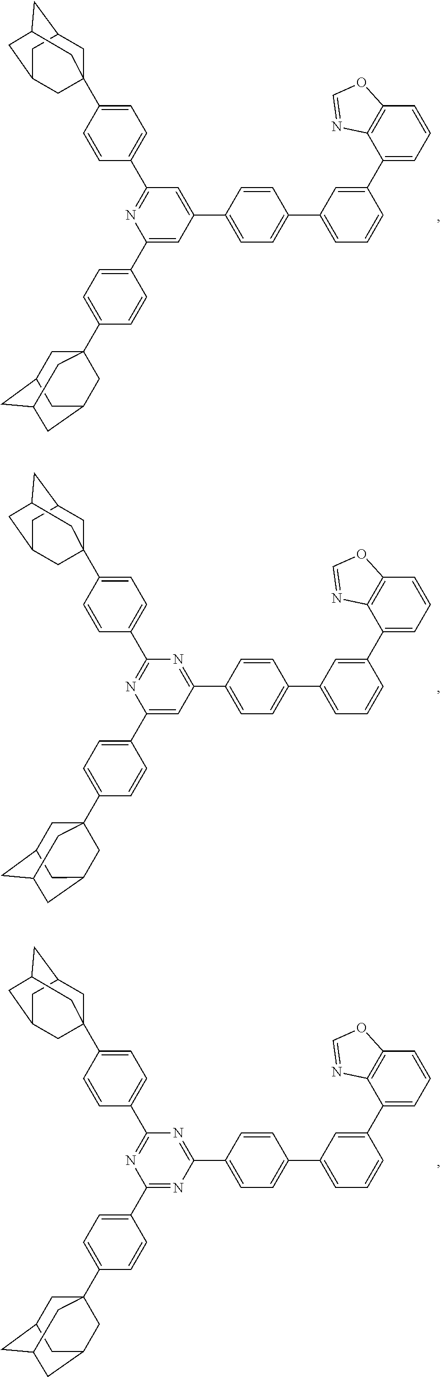 [SMILES: c1cc(-c2ccc(-c3cc(-c4ccc(C56CC7CC(CC(C7)C5)C6)cc4)nc(-c4ccc(C56CC7CC(CC(C7)C5)C6)cc4)c3)cc2)cc(-c2cccc3ocnc23)c1 ; c1cc(-c2ccc(-c3cc(-c4ccc(C56CC7CC(CC(C7)C5)C6)cc4)nc(-c4ccc(C56CC7CC(CC(C7)C5)C6)cc4)n3)cc2)cc(-c2cccc3ocnc23)c1 ; c1cc(-c2ccc(-c3nc(-c4ccc(C56CC7CC(CC(C7)C5)C6)cc4)nc(-c4ccc(C56CC7CC(CC(C7)C5)C6)cc4)n3)cc2)cc(-c2cccc3ocnc23)c1]

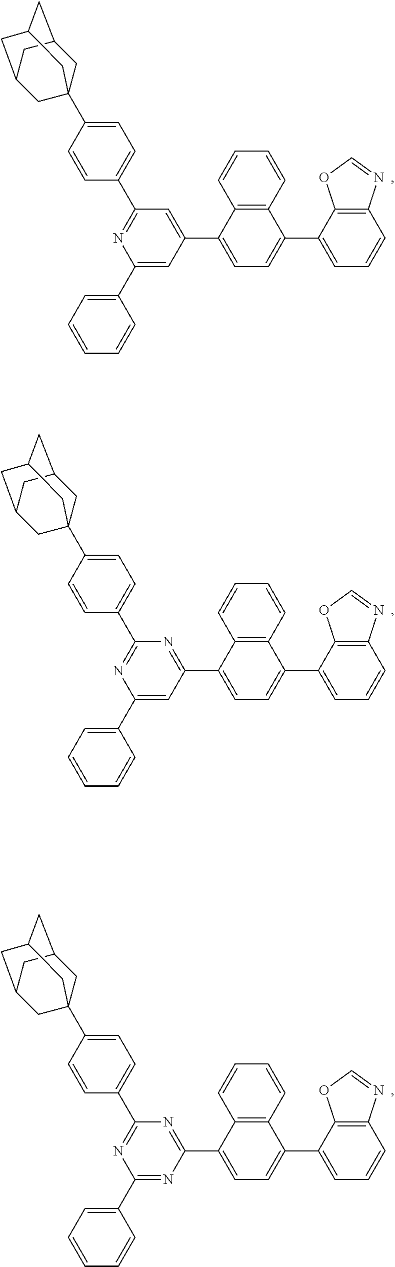 [SMILES: c1ccc(-c2cc(-c3ccc(-c4cccc5ncoc45)c4ccccc34)cc(-c3ccc(C45CC6CC(CC(C6)C4)C5)cc3)n2)cc1 ; c1ccc(-c2cc(-c3ccc(-c4cccc5ncoc45)c4ccccc34)nc(-c3ccc(C45CC6CC(CC(C6)C4)C5)cc3)n2)cc1 ; c1ccc(-c2nc(-c3ccc(C45CC6CC(CC(C6)C4)C5)cc3)nc(-c3ccc(-c4cccc5ncoc45)c4ccccc34)n2)cc1]

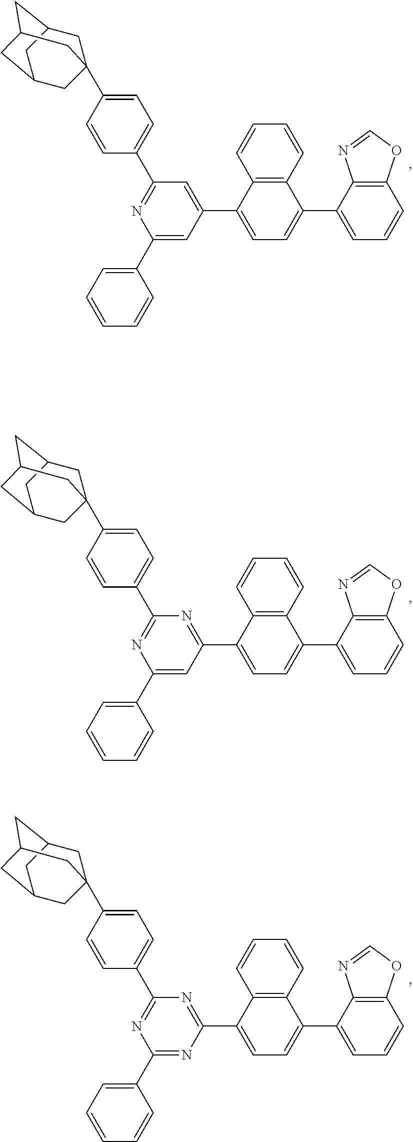 [SMILES: c1ccc(-c2cc(-c3ccc(-c4cccc5ocnc45)c4ccccc34)cc(-c3ccc(C45CC6CC(CC(C6)C4)C5)cc3)n2)cc1 ; c1ccc(-c2cc(-c3ccc(-c4cccc5ocnc45)c4ccccc34)nc(-c3ccc(C45CC6CC(CC(C6)C4)C5)cc3)n2)cc1 ; c1ccc(-c2nc(-c3ccc(C45CC6CC(CC(C6)C4)C5)cc3)nc(-c3ccc(-c4cccc5ocnc45)c4ccccc34)n2)cc1]

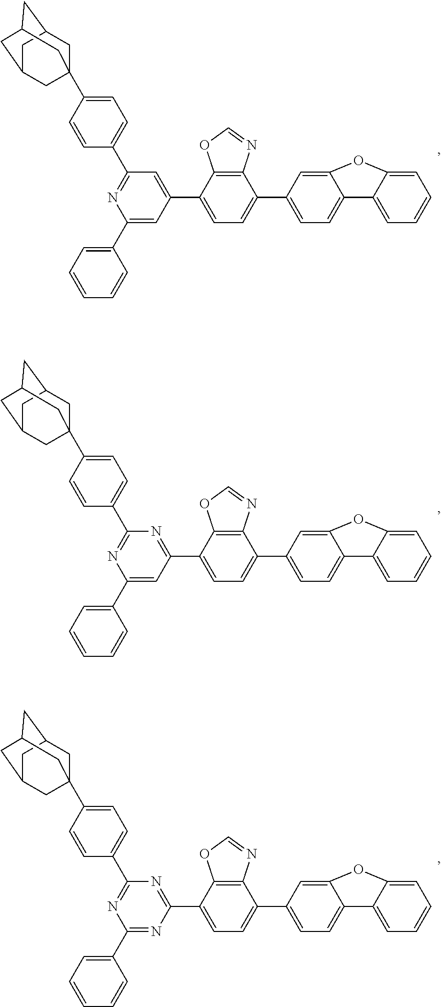 [SMILES: c1ccc(-c2cc(-c3ccc(-c4ccc5c(c4)oc4ccccc45)c4ncoc34)cc(-c3ccc(C45CC6CC(CC(C6)C4)C5)cc3)n2)cc1 ; c1ccc(-c2cc(-c3ccc(-c4ccc5c(c4)oc4ccccc45)c4ncoc34)nc(-c3ccc(C45CC6CC(CC(C6)C4)C5)cc3)n2)cc1 ; c1ccc(-c2nc(-c3ccc(C45CC6CC(CC(C6)C4)C5)cc3)nc(-c3ccc(-c4ccc5c(c4)oc4ccccc45)c4ncoc34)n2)cc1]

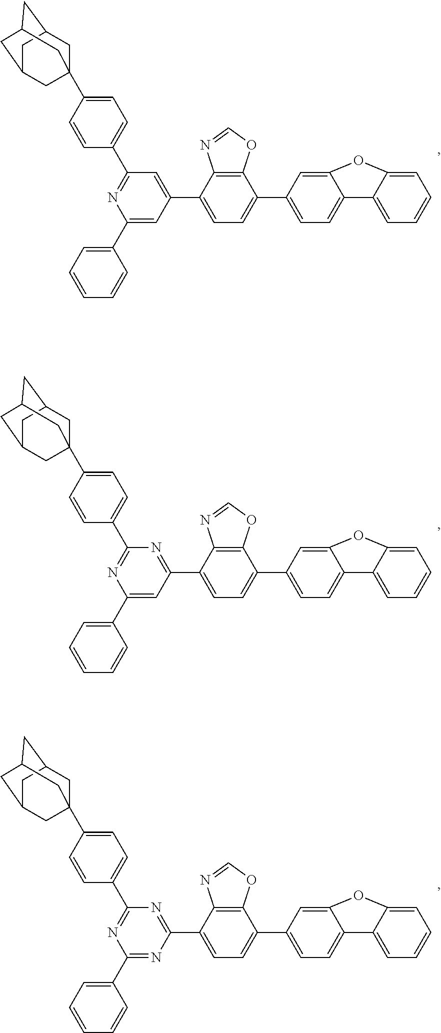 [SMILES: c1ccc(-c2cc(-c3ccc(-c4ccc5c(c4)oc4ccccc45)c4ocnc34)cc(-c3ccc(C45CC6CC(CC(C6)C4)C5)cc3)n2)cc1 ; c1ccc(-c2cc(-c3ccc(-c4ccc5c(c4)oc4ccccc45)c4ocnc34)nc(-c3ccc(C45CC6CC(CC(C6)C4)C5)cc3)n2)cc1 ; c1ccc(-c2nc(-c3ccc(C45CC6CC(CC(C6)C4)C5)cc3)nc(-c3ccc(-c4ccc5c(c4)oc4ccccc45)c4ocnc34)n2)cc1]

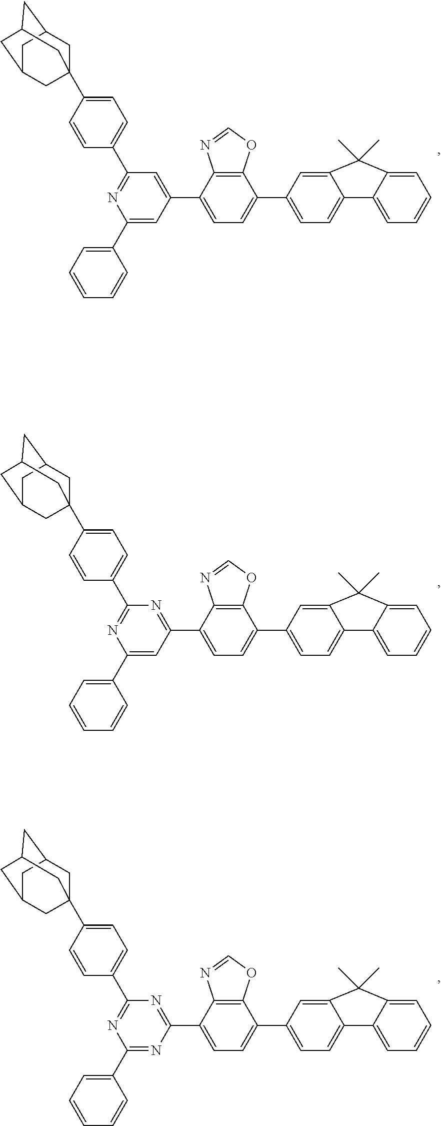 [SMILES: CC1(C)c2ccccc2-c2ccc(-c3ccc(-c4cc(-c5ccccc5)nc(-c5ccc(C67CC8CC(CC(C8)C6)C7)cc5)c4)c4ncoc34)cc21 ; CC1(C)c2ccccc2-c2ccc(-c3ccc(-c4cc(-c5ccccc5)nc(-c5ccc(C67CC8CC(CC(C8)C6)C7)cc5)n4)c4ncoc34)cc21 ; CC1(C)c2ccccc2-c2ccc(-c3ccc(-c4nc(-c5ccccc5)nc(-c5ccc(C67CC8CC(CC(C8)C6)C7)cc5)n4)c4ncoc34)cc21]

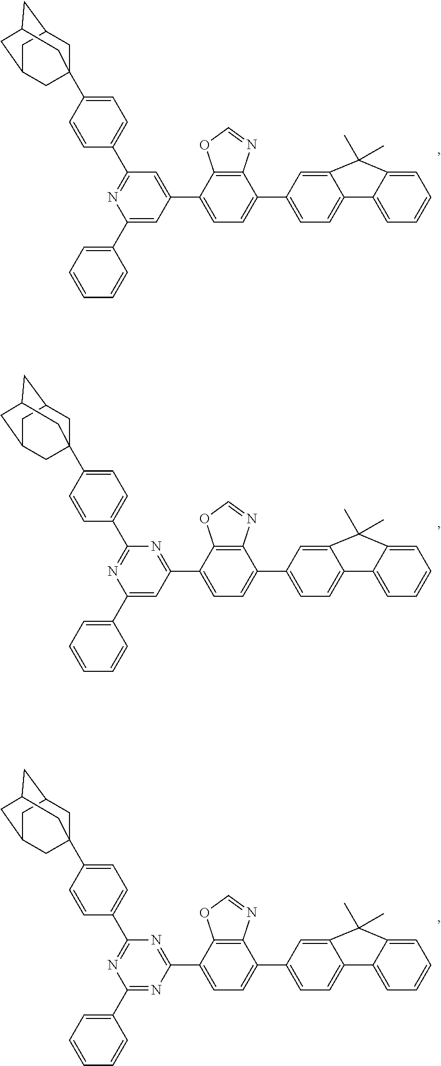 [SMILES: CC1(C)c2ccccc2-c2ccc(-c3ccc(-c4cc(-c5ccccc5)nc(-c5ccc(C67CC8CC(CC(C8)C6)C7)cc5)c4)c4ocnc34)cc21 ; CC1(C)c2ccccc2-c2ccc(-c3ccc(-c4cc(-c5ccccc5)nc(-c5ccc(C67CC8CC(CC(C8)C6)C7)cc5)n4)c4ocnc34)cc21 ; CC1(C)c2ccccc2-c2ccc(-c3ccc(-c4nc(-c5ccccc5)nc(-c5ccc(C67CC8CC(CC(C8)C6)C7)cc5)n4)c4ocnc34)cc21]

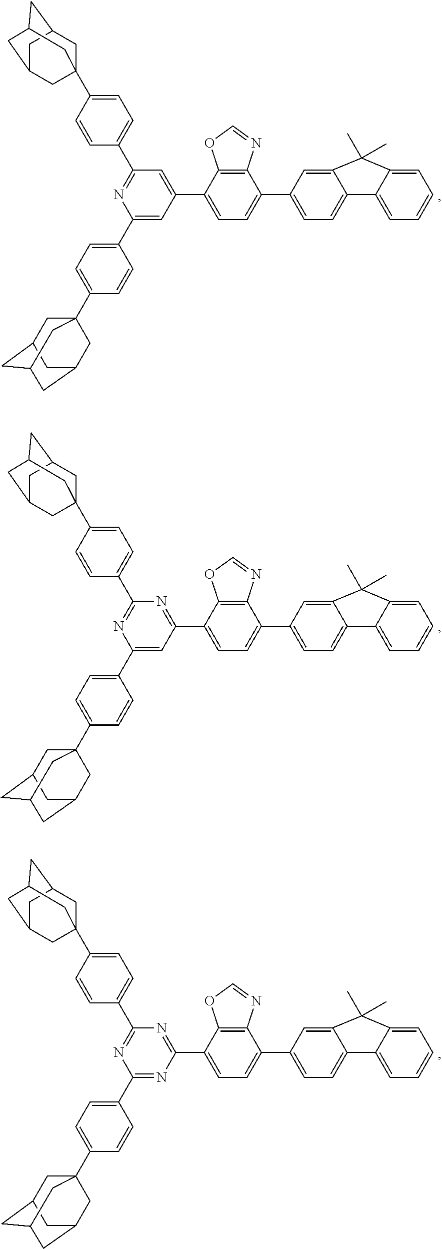

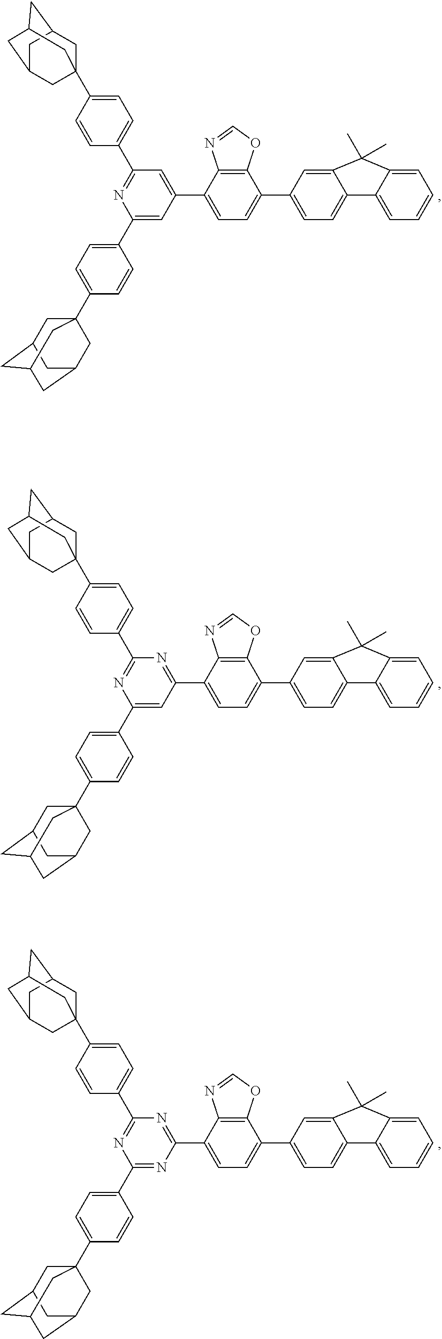 [SMILES: CC1(C)c2ccccc2-c2ccc(-c3ccc(-c4cc(-c5ccc(C67CC8CC(CC(C8)C6)C7)cc5)nc(-c5ccc(C67CC8CC(CC(C8)C6)C7)cc5)c4)c4ncoc34)cc21 ; CC1(C)c2ccccc2-c2ccc(-c3ccc(-c4cc(-c5ccc(C67CC8CC(CC(C8)C6)C7)cc5)nc(-c5ccc(C67CC8CC(CC(C8)C6)C7)cc5)n4)c4ncoc34)cc21 ; CC1(C)c2ccccc2-c2ccc(-c3ccc(-c4nc(-c5ccc(C67CC8CC(CC(C8)C6)C7)cc5)nc(-c5ccc(C67CC8CC(CC(C8)C6)C7)cc5)n4)c4ncoc34)cc21]

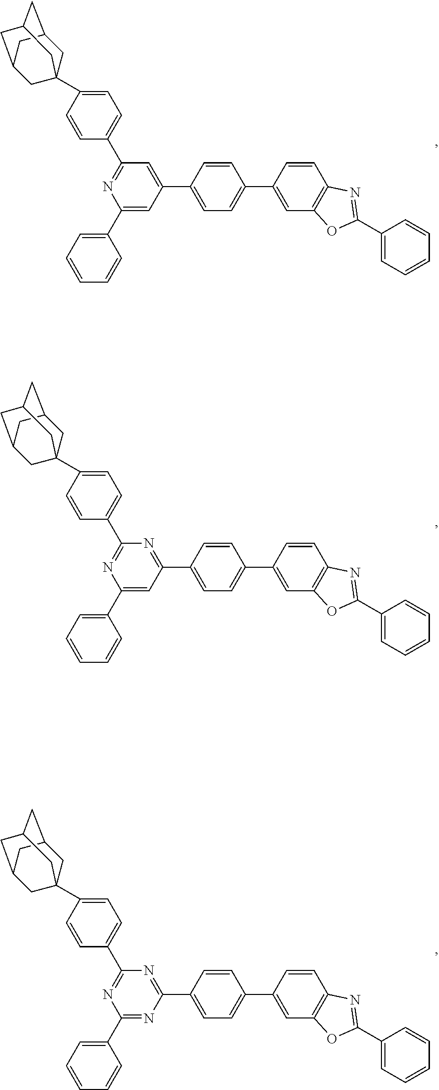 [SMILES: c1ccc(-c2cc(-c3ccc(-c4ccc5nc(-c6ccccc6)oc5c4)cc3)cc(-c3ccc(C45CC6CC(CC(C6)C4)C5)cc3)n2)cc1 ; c1ccc(-c2cc(-c3ccc(-c4ccc5nc(-c6ccccc6)oc5c4)cc3)nc(-c3ccc(C45CC6CC(CC(C6)C4)C5)cc3)n2)cc1 ; c1ccc(-c2nc(-c3ccc(-c4ccc5nc(-c6ccccc6)oc5c4)cc3)nc(-c3ccc(C45CC6CC(CC(C6)C4)C5)cc3)n2)cc1]

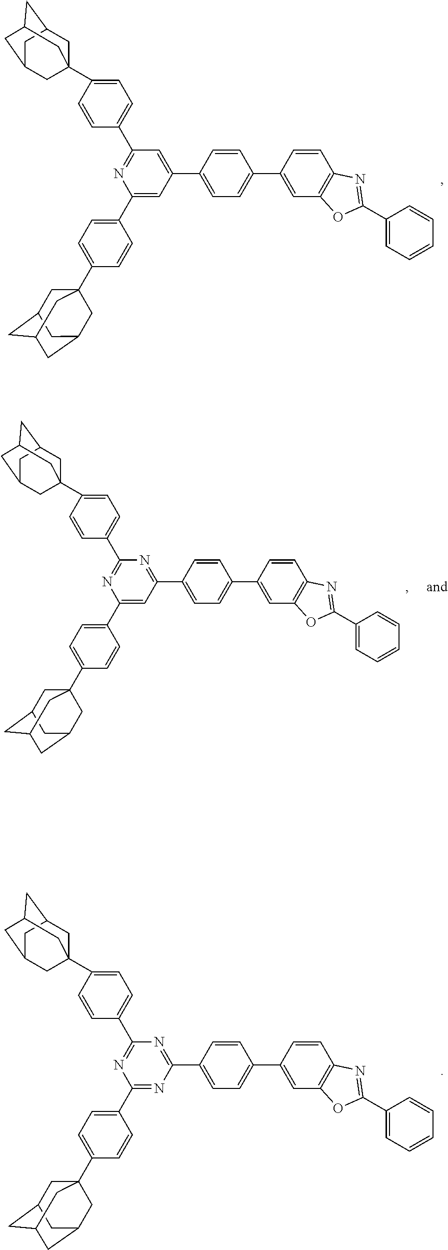 [SMILES: c1ccc(-c2nc3ccc(-c4ccc(-c5cc(-c6ccc(C78CC9CC(CC(C9)C7)C8)cc6)nc(-c6ccc(C78CC9CC(CC(C9)C7)C8)cc6)c5)cc4)cc3o2)cc1 ; c1ccc(-c2nc3ccc(-c4ccc(-c5cc(-c6ccc(C78CC9CC(CC(C9)C7)C8)cc6)nc(-c6ccc(C78CC9CC(CC(C9)C7)C8)cc6)n5)cc4)cc3o2)cc1 ; c1ccc(-c2nc3ccc(-c4ccc(-c5nc(-c6ccc(C78CC9CC(CC(C9)C7)C8)cc6)nc(-c6ccc(C78CC9CC(CC(C9)C7)C8)cc6)n5)cc4)cc3o2)cc1]

Optionally, the chemical structural formula of the compound includes any one of

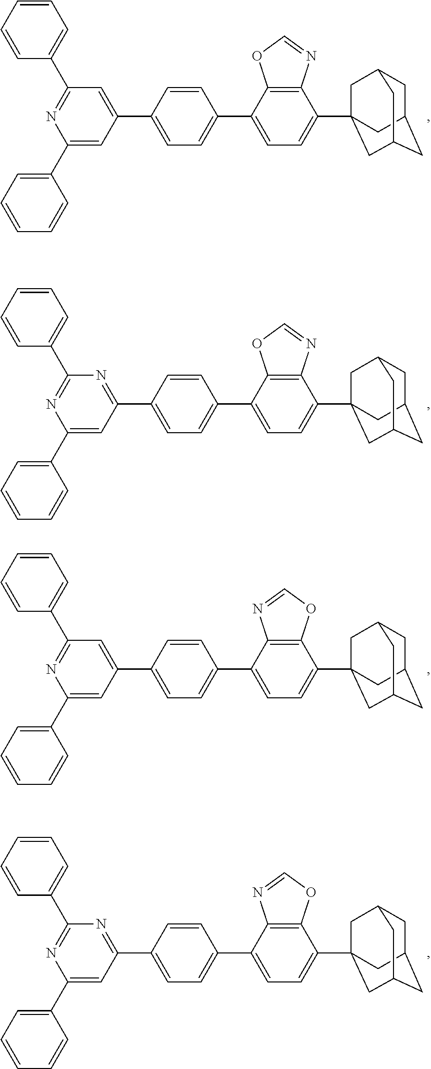

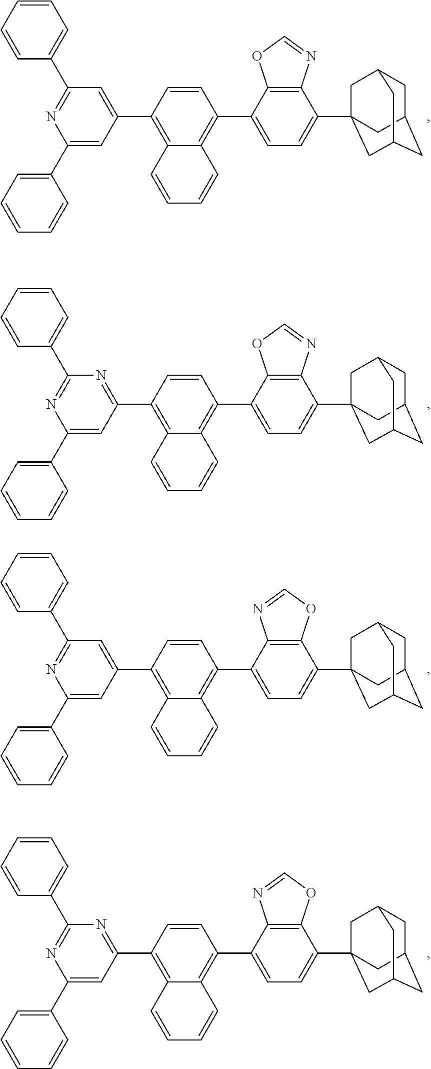

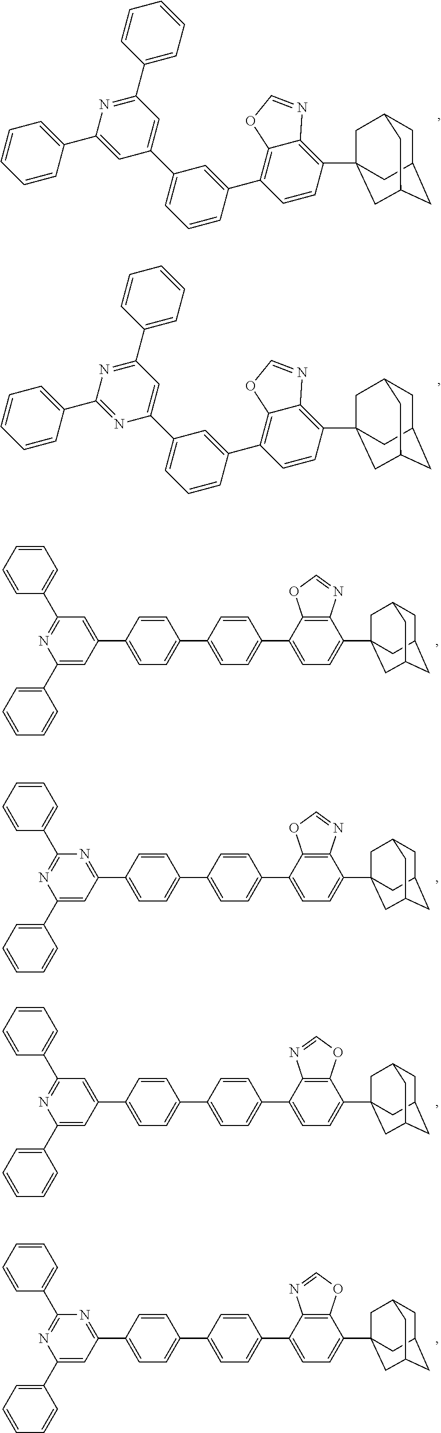

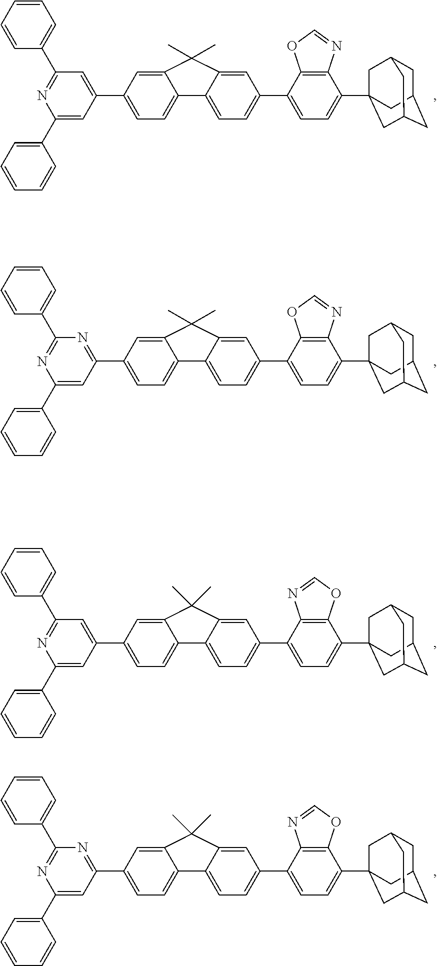 [SMILES: CC1(C)c2cc(-c3cc(-c4ccccc4)nc(-c4ccccc4)c3)ccc2-c2ccc(-c3ccc(C45CC6CC(CC(C6)C4)C5)c4ncoc34)cc21 ; CC1(C)c2cc(-c3cc(-c4ccccc4)nc(-c4ccccc4)n3)ccc2-c2ccc(-c3ccc(C45CC6CC(CC(C6)C4)C5)c4ncoc34)cc21 ; CC1(C)c2cc(-c3cc(-c4ccccc4)nc(-c4ccccc4)c3)ccc2-c2ccc(-c3ccc(C45CC6CC(CC(C6)C4)C5)c4ocnc34)cc21 ; CC1(C)c2cc(-c3cc(-c4ccccc4)nc(-c4ccccc4)n3)ccc2-c2ccc(-c3ccc(C45CC6CC(CC(C6)C4)C5)c4ocnc34)cc21]

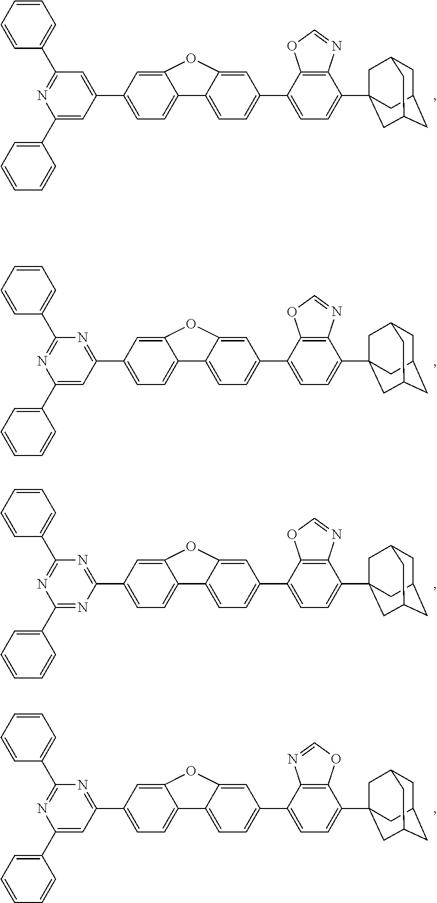

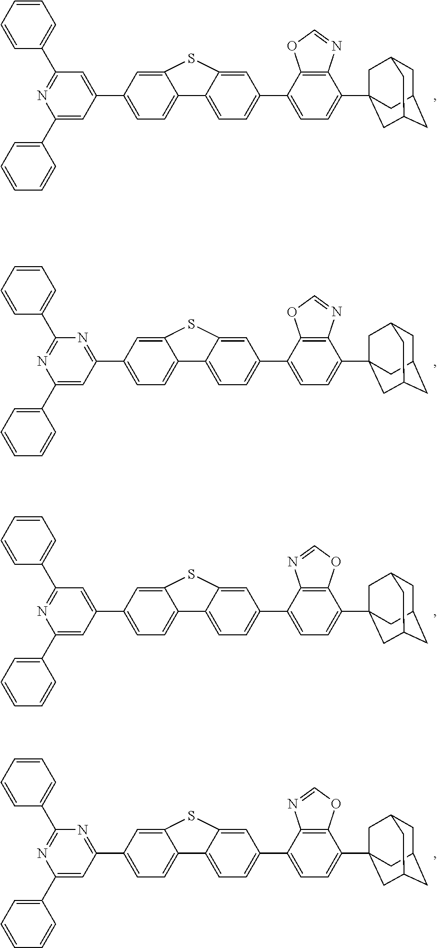 [SMILES: c1ccc(-c2cc(-c3ccc4c(c3)sc3cc(-c5ccc(C67CC8CC(CC(C8)C6)C7)c6ncoc56)ccc34)cc(-c3ccccc3)n2)cc1 ; c1ccc(-c2cc(-c3ccc4c(c3)sc3cc(-c5ccc(C67CC8CC(CC(C8)C6)C7)c6ncoc56)ccc34)nc(-c3ccccc3)n2)cc1 ; c1ccc(-c2cc(-c3ccc4c(c3)sc3cc(-c5ccc(C67CC8CC(CC(C8)C6)C7)c6ocnc56)ccc34)cc(-c3ccccc3)n2)cc1 ; c1ccc(-c2cc(-c3ccc4c(c3)sc3cc(-c5ccc(C67CC8CC(CC(C8)C6)C7)c6ocnc56)ccc34)nc(-c3ccccc3)n2)cc1]

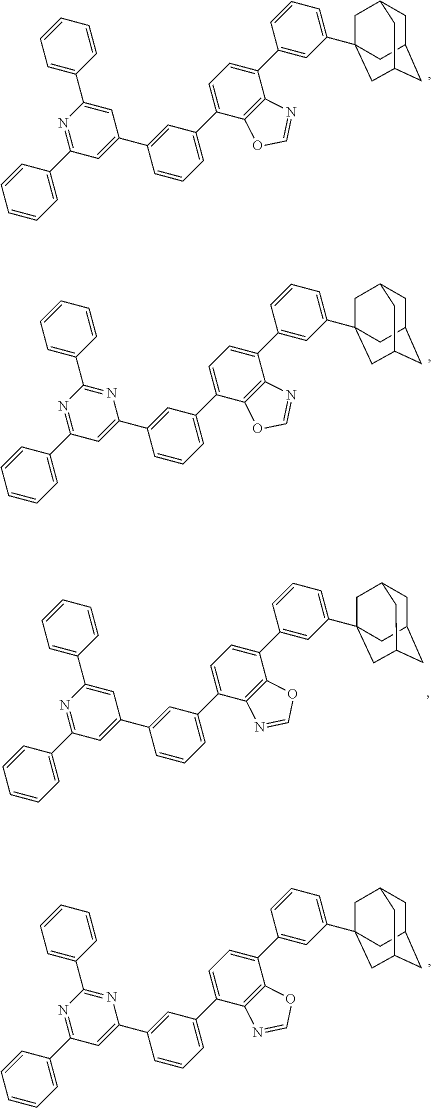

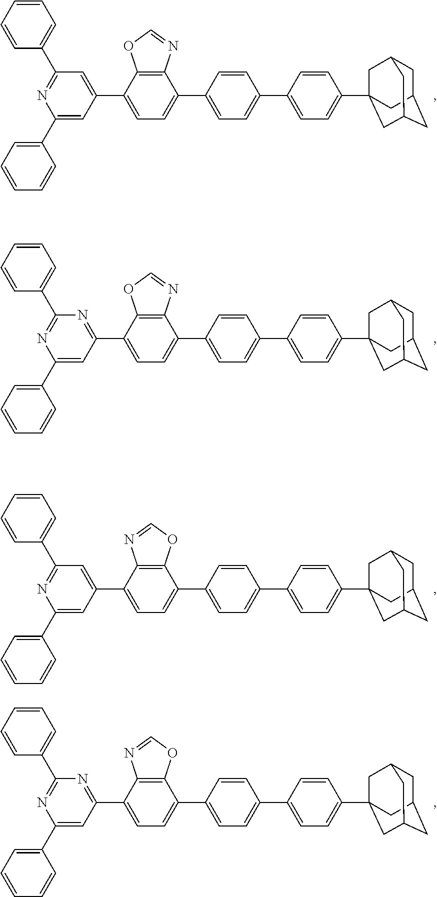

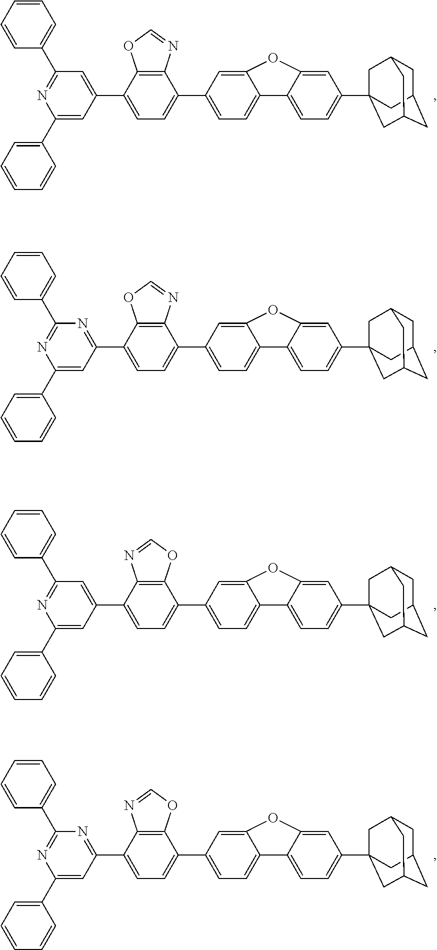 [SMILES: c1ccc(-c2cc(-c3ccc(-c4ccc5c(c4)oc4cc(C67CC8CC(CC(C8)C6)C7)ccc45)c4ncoc34)cc(-c3ccccc3)n2)cc1 ; c1ccc(-c2cc(-c3ccc(-c4ccc5c(c4)oc4cc(C67CC8CC(CC(C8)C6)C7)ccc45)c4ncoc34)nc(-c3ccccc3)n2)cc1 ; c1ccc(-c2cc(-c3ccc(-c4ccc5c(c4)oc4cc(C67CC8CC(CC(C8)C6)C7)ccc45)c4ocnc34)cc(-c3ccccc3)n2)cc1 ; c1ccc(-c2cc(-c3ccc(-c4ccc5c(c4)oc4cc(C67CC8CC(CC(C8)C6)C7)ccc45)c4ocnc34)nc(-c3ccccc3)n2)cc1]

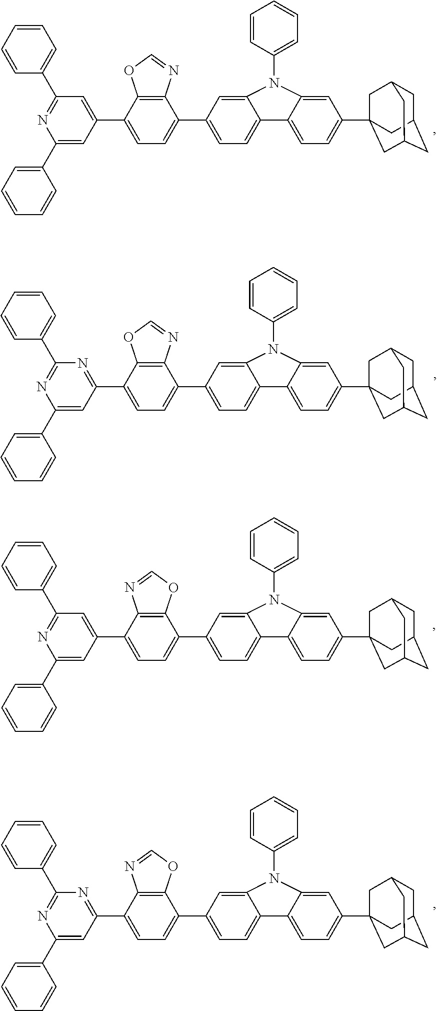 [SMILES: c1ccc(-c2cc(-c3ccc(-c4ccc5c6ccc(C78CC9CC(CC(C9)C7)C8)cc6n(-c6ccccc6)c5c4)c4ncoc34)cc(-c3ccccc3)n2)cc1 ; c1ccc(-c2cc(-c3ccc(-c4ccc5c6ccc(C78CC9CC(CC(C9)C7)C8)cc6n(-c6ccccc6)c5c4)c4ncoc34)nc(-c3ccccc3)n2)cc1 ; c1ccc(-c2cc(-c3ccc(-c4ccc5c6ccc(C78CC9CC(CC(C9)C7)C8)cc6n(-c6ccccc6)c5c4)c4ocnc34)cc(-c3ccccc3)n2)cc1 ; c1ccc(-c2cc(-c3ccc(-c4ccc5c6ccc(C78CC9CC(CC(C9)C7)C8)cc6n(-c6ccccc6)c5c4)c4ocnc34)nc(-c3ccccc3)n2)cc1]

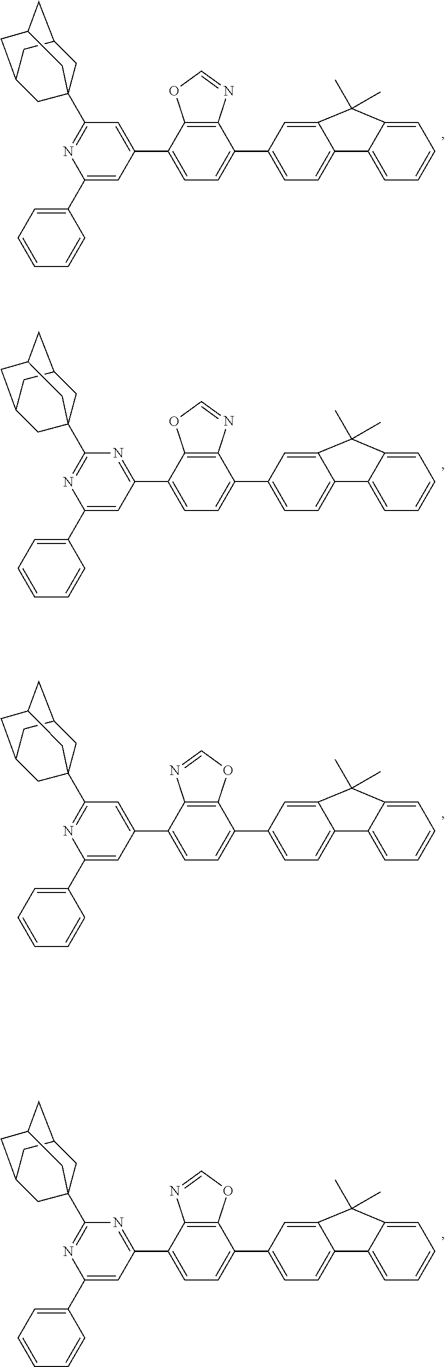 [SMILES: CC1(C)c2ccccc2-c2ccc(-c3ccc(-c4cc(-c5ccccc5)nc(C56CC7CC(CC(C7)C5)C6)c4)c4ocnc34)cc21 ; CC1(C)c2ccccc2-c2ccc(-c3ccc(-c4cc(-c5ccccc5)nc(C56CC7CC(CC(C7)C5)C6)n4)c4ocnc34)cc21 ; CC1(C)c2ccccc2-c2ccc(-c3ccc(-c4cc(-c5ccccc5)nc(C56CC7CC(CC(C7)C5)C6)c4)c4ncoc34)cc21 ; CC1(C)c2ccccc2-c2ccc(-c3ccc(-c4cc(-c5ccccc5)nc(C56CC7CC(CC(C7)C5)C6)n4)c4ncoc34)cc21]

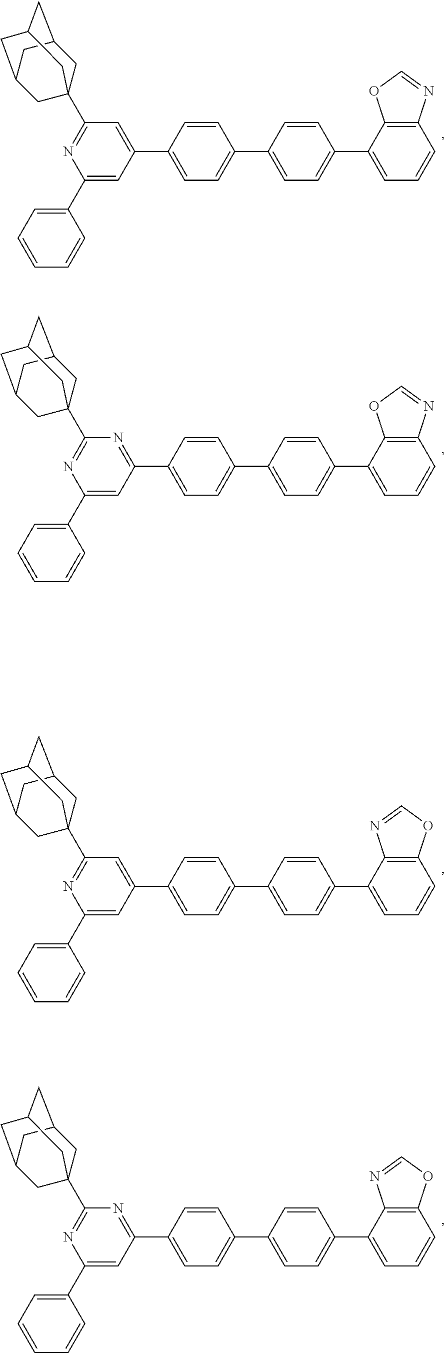 [SMILES: c1ccc(-c2cc(-c3ccc(-c4ccc(-c5cccc6ncoc56)cc4)cc3)cc(C34CC5CC(CC(C5)C3)C4)n2)cc1 ; c1ccc(-c2cc(-c3ccc(-c4ccc(-c5cccc6ncoc56)cc4)cc3)nc(C34CC5CC(CC(C5)C3)C4)n2)cc1 ; c1ccc(-c2cc(-c3ccc(-c4ccc(-c5cccc6ocnc56)cc4)cc3)cc(C34CC5CC(CC(C5)C3)C4)n2)cc1 ; c1ccc(-c2cc(-c3ccc(-c4ccc(-c5cccc6ocnc56)cc4)cc3)nc(C34CC5CC(CC(C5)C3)C4)n2)cc1]

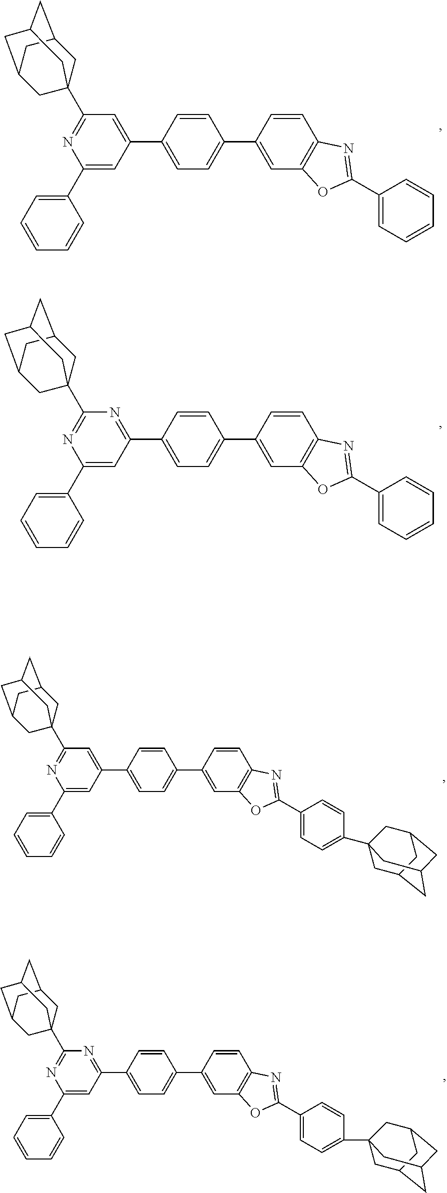 [SMILES: c1ccc(-c2cc(-c3ccc(-c4ccc5nc(-c6ccccc6)oc5c4)cc3)cc(C34CC5CC(CC(C5)C3)C4)n2)cc1 ; c1ccc(-c2cc(-c3ccc(-c4ccc5nc(-c6ccccc6)oc5c4)cc3)nc(C34CC5CC(CC(C5)C3)C4)n2)cc1 ; c1ccc(-c2cc(-c3ccc(-c4ccc5nc(-c6ccc(C78CC9CC(CC(C9)C7)C8)cc6)oc5c4)cc3)cc(C34CC5CC(CC(C5)C3)C4)n2)cc1 ; c1ccc(-c2cc(-c3ccc(-c4ccc5nc(-c6ccc(C78CC9CC(CC(C9)C7)C8)cc6)oc5c4)cc3)nc(C34CC5CC(CC(C5)C3)C4)n2)cc1]

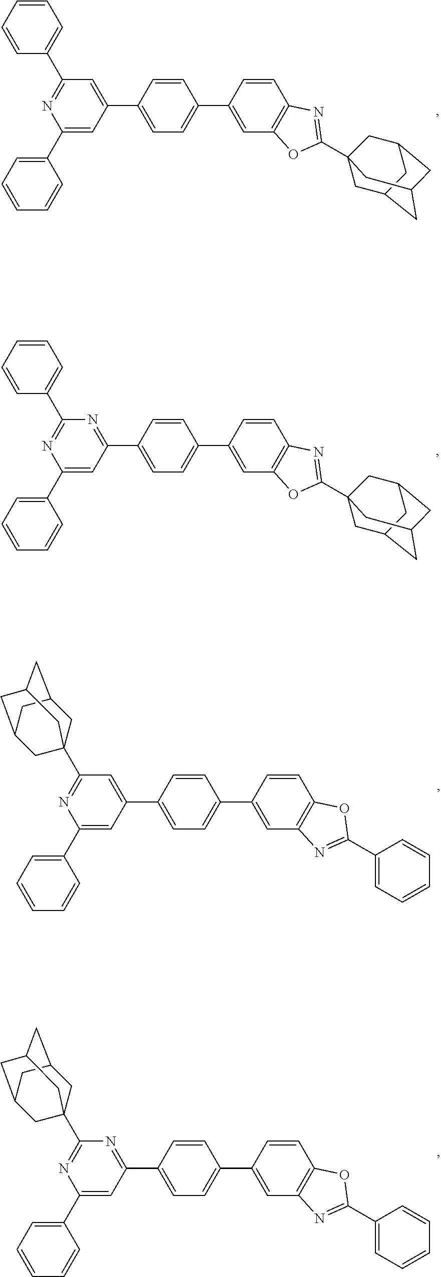 [SMILES: c1ccc(-c2cc(-c3ccc(-c4ccc5nc(C67CC8CC(CC(C8)C6)C7)oc5c4)cc3)cc(-c3ccccc3)n2)cc1 ; c1ccc(-c2cc(-c3ccc(-c4ccc5nc(C67CC8CC(CC(C8)C6)C7)oc5c4)cc3)nc(-c3ccccc3)n2)cc1 ; c1ccc(-c2cc(-c3ccc(-c4ccc5oc(-c6ccccc6)nc5c4)cc3)cc(C34CC5CC(CC(C5)C3)C4)n2)cc1 ; c1ccc(-c2cc(-c3ccc(-c4ccc5oc(-c6ccccc6)nc5c4)cc3)nc(C34CC5CC(CC(C5)C3)C4)n2)cc1]

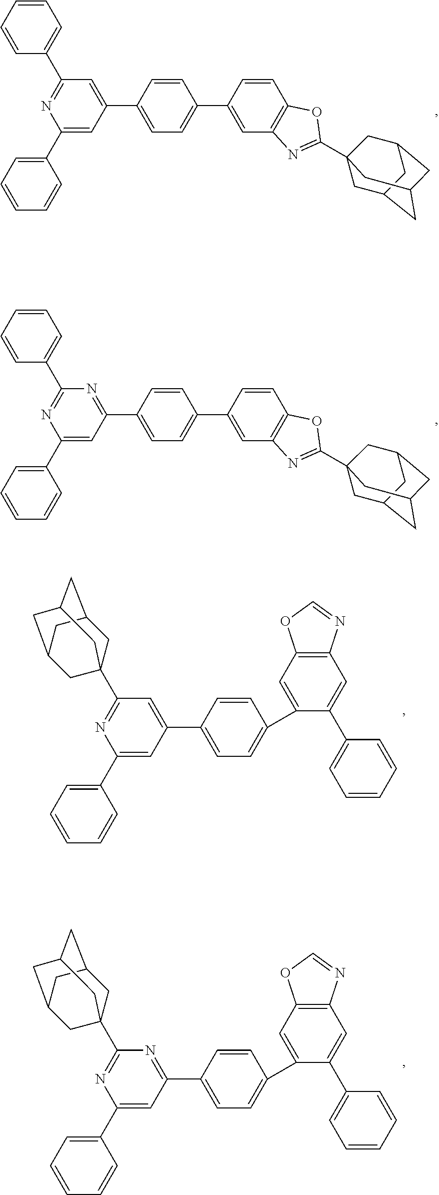 [SMILES: c1ccc(-c2cc(-c3ccc(-c4ccc5oc(C67CC8CC(CC(C8)C6)C7)nc5c4)cc3)cc(-c3ccccc3)n2)cc1 ; c1ccc(-c2cc(-c3ccc(-c4ccc5oc(C67CC8CC(CC(C8)C6)C7)nc5c4)cc3)nc(-c3ccccc3)n2)cc1 ; c1ccc(-c2cc(-c3ccc(-c4cc5ocnc5cc4-c4ccccc4)cc3)cc(C34CC5CC(CC(C5)C3)C4)n2)cc1 ; c1ccc(-c2cc(-c3ccc(-c4cc5ocnc5cc4-c4ccccc4)cc3)nc(C34CC5CC(CC(C5)C3)C4)n2)cc1]

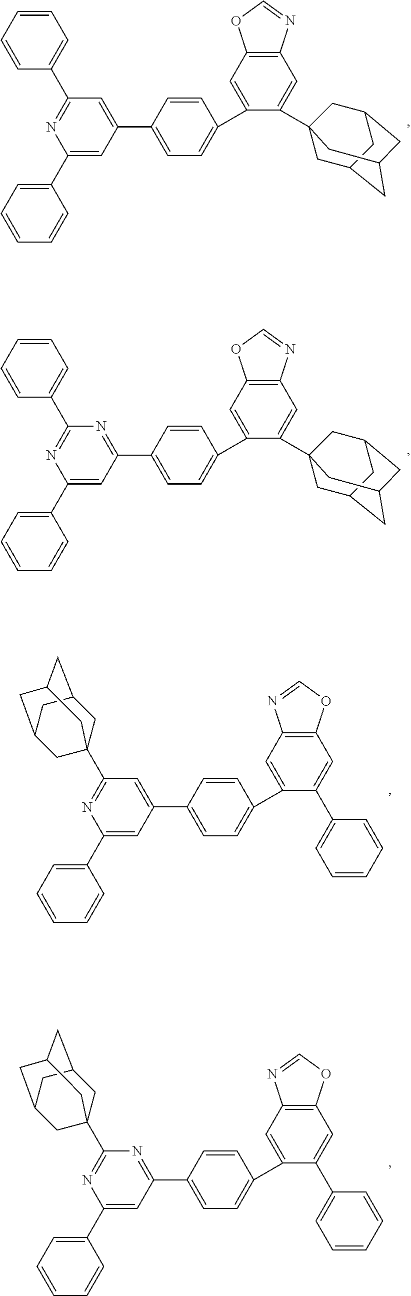 [SMILES: c1ccc(-c2cc(-c3ccc(-c4cc5ocnc5cc4C45CC6CC(CC(C6)C4)C5)cc3)cc(-c3ccccc3)n2)cc1 ; c1ccc(-c2cc(-c3ccc(-c4cc5ocnc5cc4C45CC6CC(CC(C6)C4)C5)cc3)nc(-c3ccccc3)n2)cc1 ; c1ccc(-c2cc(-c3ccc(-c4cc5ncoc5cc4-c4ccccc4)cc3)cc(C34CC5CC(CC(C5)C3)C4)n2)cc1 ; c1ccc(-c2cc(-c3ccc(-c4cc5ncoc5cc4-c4ccccc4)cc3)nc(C34CC5CC(CC(C5)C3)C4)n2)cc1]

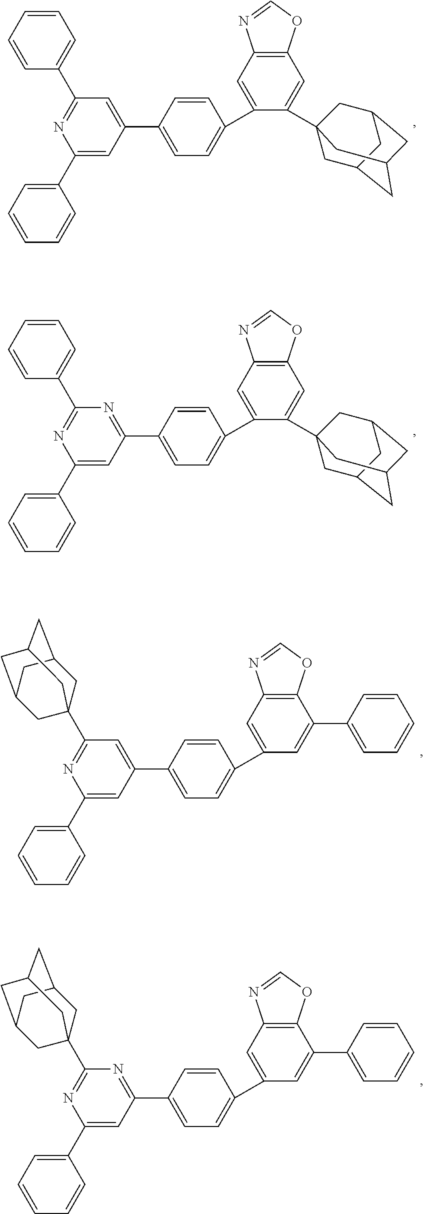 [SMILES: c1ccc(-c2cc(-c3ccc(-c4cc5ncoc5cc4C45CC6CC(CC(C6)C4)C5)cc3)cc(-c3ccccc3)n2)cc1 ; c1ccc(-c2cc(-c3ccc(-c4cc5ncoc5cc4C45CC6CC(CC(C6)C4)C5)cc3)nc(-c3ccccc3)n2)cc1 ; c1ccc(-c2cc(-c3ccc(-c4cc(-c5ccccc5)c5ocnc5c4)cc3)cc(C34CC5CC(CC(C5)C3)C4)n2)cc1 ; c1ccc(-c2cc(-c3ccc(-c4cc(-c5ccccc5)c5ocnc5c4)cc3)nc(C34CC5CC(CC(C5)C3)C4)n2)cc1]

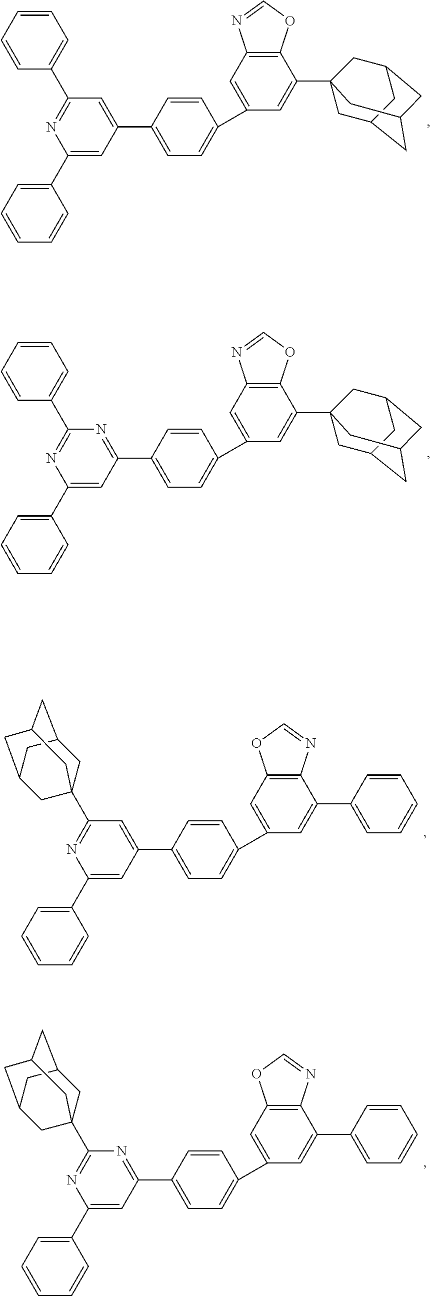 [SMILES: c1ccc(-c2cc(-c3ccc(-c4cc(C56CC7CC(CC(C7)C5)C6)c5ocnc5c4)cc3)cc(-c3ccccc3)n2)cc1 ; c1ccc(-c2cc(-c3ccc(-c4cc(C56CC7CC(CC(C7)C5)C6)c5ocnc5c4)cc3)nc(-c3ccccc3)n2)cc1 ; c1ccc(-c2cc(-c3ccc(-c4cc(-c5ccccc5)c5ncoc5c4)cc3)cc(C34CC5CC(CC(C5)C3)C4)n2)cc1 ; c1ccc(-c2cc(-c3ccc(-c4cc(-c5ccccc5)c5ncoc5c4)cc3)nc(C34CC5CC(CC(C5)C3)C4)n2)cc1]

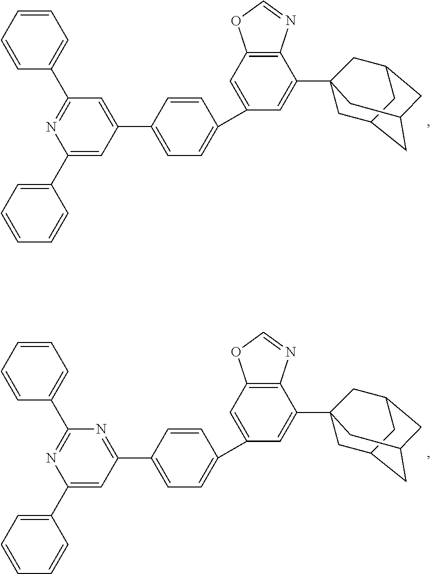 [SMILES: c1ccc(-c2cc(-c3ccc(-c4cc(C56CC7CC(CC(C7)C5)C6)c5ncoc5c4)cc3)cc(-c3ccccc3)n2)cc1 ; c1ccc(-c2cc(-c3ccc(-c4cc(C56CC7CC(CC(C7)C5)C6)c5ncoc5c4)cc3)nc(-c3ccccc3)n2)cc1]

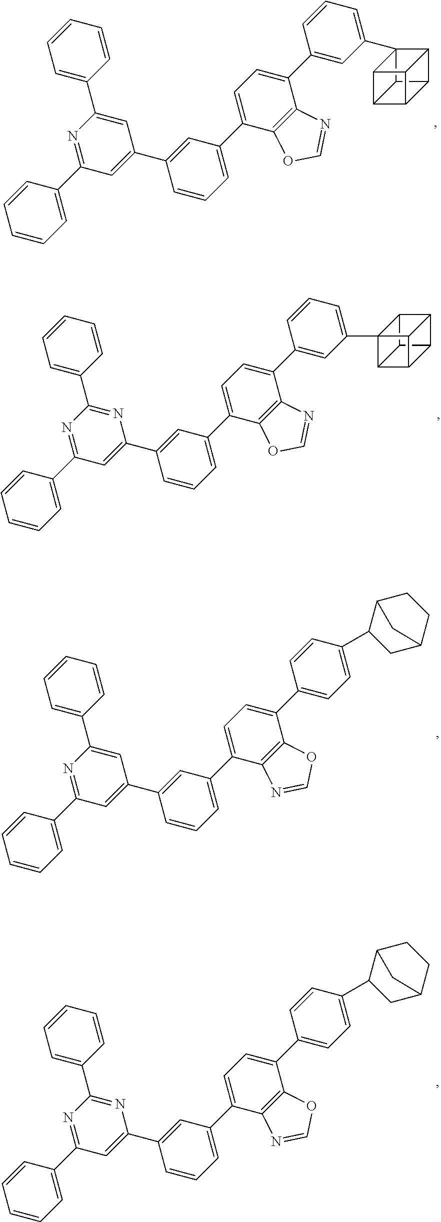 [SMILES: c1ccc(-c2cc(-c3cccc(-c4ccc(-c5cccc(C67C8C9C%10C8C6C%10C97)c5)c5ncoc45)c3)cc(-c3ccccc3)n2)cc1 ; c1ccc(-c2cc(-c3cccc(-c4ccc(-c5cccc(C67C8C9C%10C8C6C%10C97)c5)c5ncoc45)c3)nc(-c3ccccc3)n2)cc1 ; c1ccc(-c2cc(-c3cccc(-c4ccc(-c5ccc(C6CC7CCC6C7)cc5)c5ocnc45)c3)cc(-c3ccccc3)n2)cc1 ; c1ccc(-c2cc(-c3cccc(-c4ccc(-c5ccc(C6CC7CCC6C7)cc5)c5ocnc45)c3)nc(-c3ccccc3)n2)cc1]

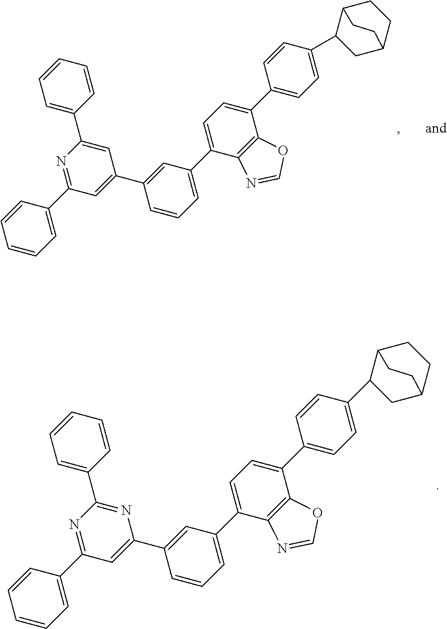 [SMILES: c1ccc(-c2cc(-c3cccc(-c4ccc(-c5ccc(C6CC7CCC6CC7)cc5)c5ocnc45)c3)cc(-c3ccccc3)n2)cc1 ; c1ccc(-c2cc(-c3cccc(-c4ccc(-c5ccc(C6CC7CCC6CC7)cc5)c5ocnc45)c3)nc(-c3ccccc3)n2)cc1]

The processes of preparing a compound E1 (whose chemical formula is

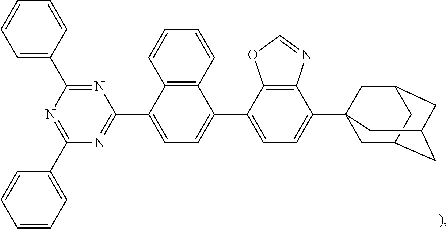 [SMILES: c1ccc(-c2nc(-c3ccccc3)nc(-c3ccc(-c4ccc(C56CC7CC(CC(C7)C5)C6)c5ncoc45)c4ccccc34)n2)cc1]

a compound E2 (whose chemical formula is

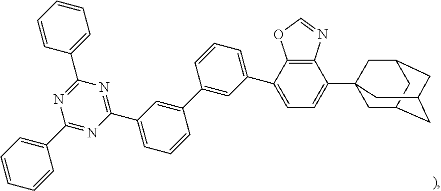 [SMILES: c1ccc(-c2nc(-c3ccccc3)nc(-c3cccc(-c4cccc(-c5ccc(C67CC8CC(CC(C8)C6)C7)c6ncoc56)c4)c3)n2)cc1]

a compound E3 (whose chemical formula is

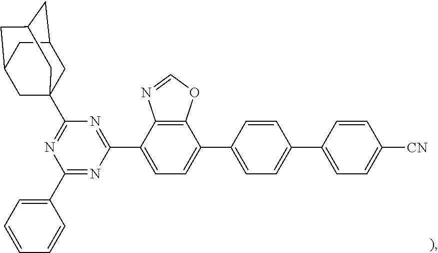 [SMILES: N#Cc1ccc(-c2ccc(-c3ccc(-c4nc(-c5ccccc5)nc(C56CC7CC(CC(C7)C5)C6)n4)c4ncoc34)cc2)cc1]

a compound E4 (whose chemical formula is

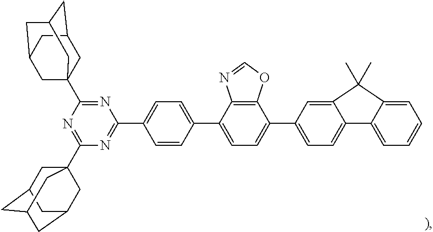 [SMILES: CC1(C)c2ccccc2-c2ccc(-c3ccc(-c4ccc(-c5nc(C67CC8CC(CC(C8)C6)C7)nc(C67CC8CC(CC(C8)C6)C7)n5)cc4)c4ncoc34)cc21]

a compound E5 (whose chemical formula is

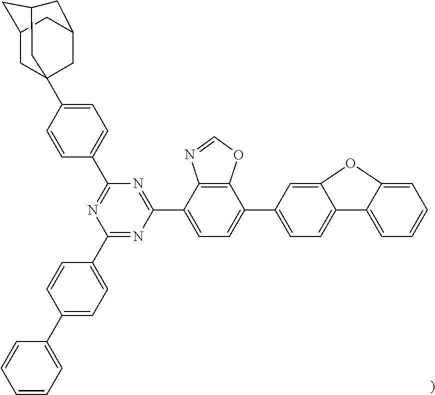 [SMILES: c1ccc(-c2ccc(-c3nc(-c4ccc(C56CC7CC(CC(C7)C5)C6)cc4)nc(-c4ccc(-c5ccc6c(c5)oc5ccccc56)c5ocnc45)n3)cc2)cc1]

and a compound E6 (whose chemical formula is

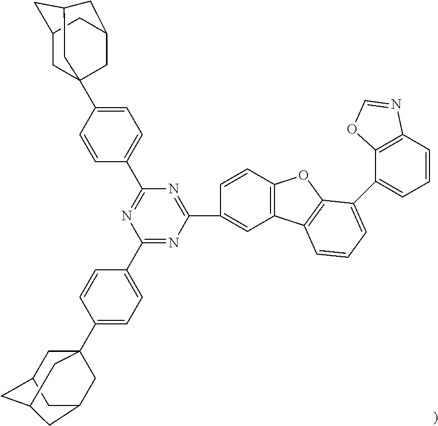 [SMILES: c1cc(-c2cccc3c2oc2ccc(-c4nc(-c5ccc(C67CC8CC(CC(C8)C6)C7)cc5)nc(-c5ccc(C67CC8CC(CC(C8)C6)C7)cc5)n4)cc23)c2ocnc2c1]

will be individually described in detail below.

The particular process of preparing the compound E1 is as follows:

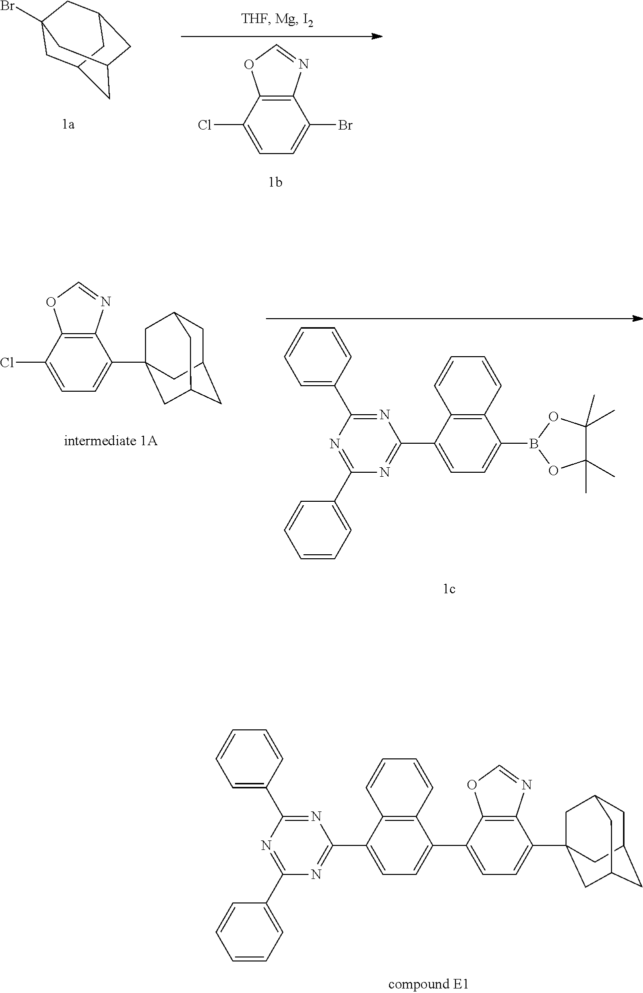 [SMILES: BrC12CC3CC(CC(C3)C1)C2 ; Clc1ccc(Br)c2ncoc12 ; Clc1ccc(C23CC4CC(CC(C4)C2)C3)c2ncoc12 ; CC1(C)OB(c2ccc(-c3nc(-c4ccccc4)nc(-c4ccccc4)n3)c3ccccc23)OC1(C)C ; c1ccc(-c2nc(-c3ccccc3)nc(-c3ccc(-c4ccc(C56CC7CC(CC(C7)C5)C6)c5ncoc45)c4ccccc34)n2)cc1]

Under protection of nitrogen N2, a magnesium sheet Mg (138.9 mmol) and a tetrahydrofuran THF (15 ml) solution are added into a reaction bottle, the temperature of the system is increased to 60° C., and iodine I₂ (1.75 mmol) are added into the system. The compound 1a (50.0 g, 232.4 mmol) is completely dissolved into a 480 ml solution, and dripped into the system of the reaction bottle slowly within 30 min, wherein during the dripping the temperature is controlled at 60° C. After the dripping has completed, the reaction proceeds under stirring at 60° C. for 2 h, after the system has cooled to normal temperature, 1b (126.4 mmol) dissolved in 40 ml of THF is dripped into the mixed solution, and, after stirring for 3 h, the reaction ends. After the reaction has ended, toluene (200 ml) and water (100 ml) are added to extract the reaction solution, the organic phases are combined, the organic layer is dried by using anhydrous magnesium sulfate, and the system is filtered and reduced-pressure-distilled for concentration. The crude product is purified by using silica-gel column chromatography, recrystallized, and filtered to obtain a solid intermediate 1A (30.4 g, 76%).

In an N2 atmosphere, the intermediate 1A (12.93 mmol) and the compound 1c (88 mmol) are placed into 150 ml of THE, and the obtained mixture is stirred and refluxed. Subsequently, a 2M aqueous solution of potassium carbonate (70 mmol) is added, the obtained mixture is stirred sufficiently, and subsequently tetra(triphenylphosphine) palladium (0.39 mmol) is introduced thereinto. After reacting for 3 hours, the temperature of the product is reduced to normal temperature, the water layer is removed, and, after drying by using anhydrous magnesium sulfate, recrystallization is performed by using 500 ml of ethyl acetate, to obtain the compound E1, wherein the yield is approximately 82.46%.

The particular process of preparing the compound E2 is as follows:

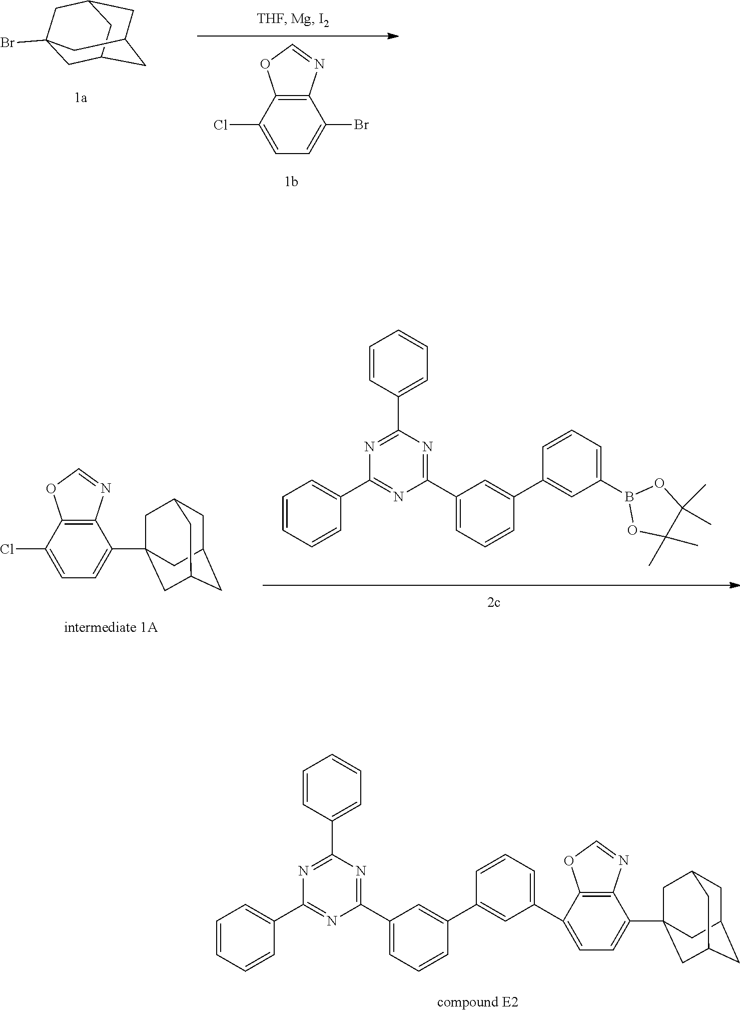 [SMILES: BrC12CC3CC(CC(C3)C1)C2 ; Clc1ccc(Br)c2ncoc12 ; Clc1ccc(C23CC4CC(CC(C4)C2)C3)c2ncoc12 ; CC1(C)OB(c2cccc(-c3cccc(-c4nc(-c5ccccc5)nc(-c5ccccc5)n4)c3)c2)OC1(C)C ; c1ccc(-c2nc(-c3ccccc3)nc(-c3cccc(-c4cccc(-c5ccc(C67CC8CC(CC(C8)C6)C7)c6ncoc56)c4)c3)n2)cc1]

1a 1b intermediate 1A 2c compound E2

The steps of synthesizing the compound E2 are substantially the same as the steps of synthesizing the compound E1, and differ in that the 1c in the step of synthesizing the compound E1 is replaced by 2c. The other reagents are not changed, to obtain the compound E2, wherein the yield is approximately 80.67%.

The particular process of preparing the compound E3 is as follows:

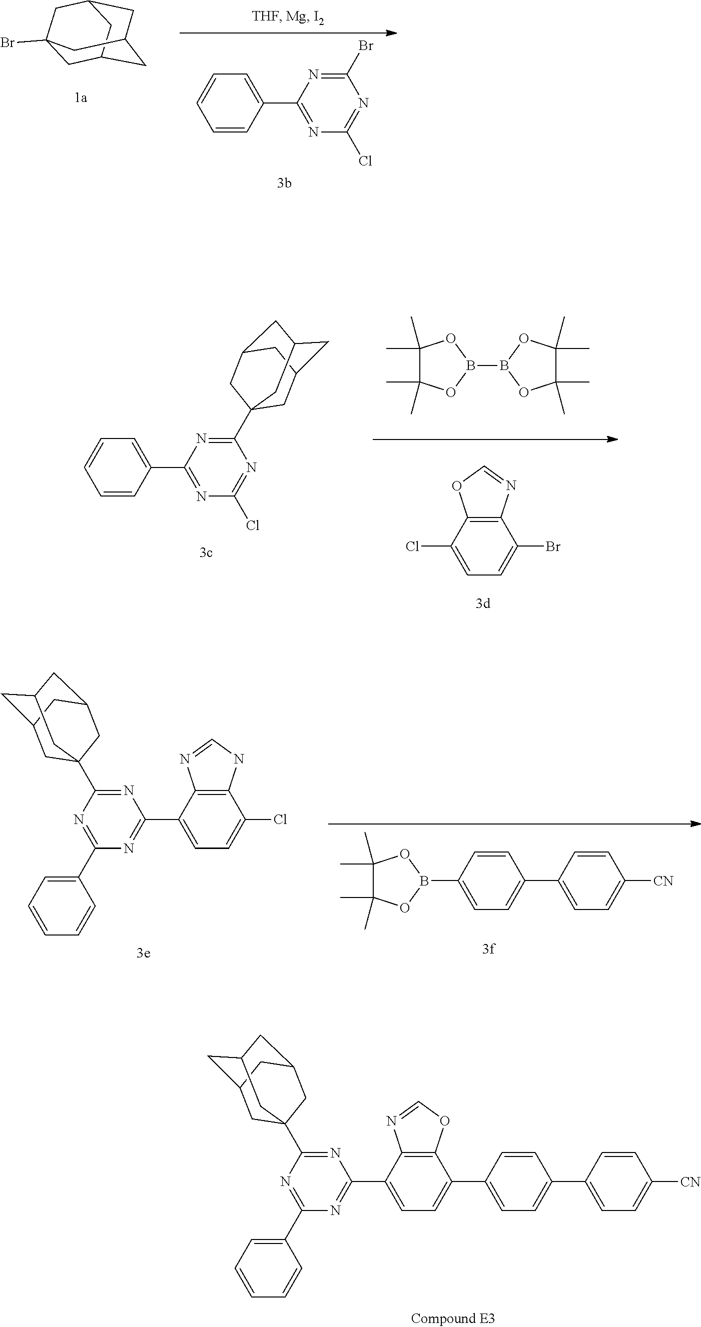

The steps of synthesizing an intermediate 3c are substantially the same as the steps of synthesizing the intermediate 1A, and differ in that the 1b in the step of synthesizing the intermediate 1A is replaced by 3b. The other reagents are not changed, to obtain the intermediate 3c.

Subsequently, 3c (25 mmol), 3d (25 mmol), potassium acetate (50 mmol), dioxane (200 ml) and ditriphenylphosphine palladium dichloride (0.5 g) are heated under reflux in a three-neck flask under nitrogen protection for 5 hours, cooled, and concentrated. Subsequently, in the nitrogen atmosphere, the above reaction product and 3d at a ratio of 1:1 (55.57 mmol) are placed into 300 ml of tetrahydrofuran, and the obtained mixture is stirred and refluxed. Subsequently, potassium carbonate (167.70 mmol) is dissolved in 800 ml of water, the obtained solution is introduced into the mixture, the obtained mixture is stirred sufficiently, and subsequently tetra(triphenylphosphine) palladium (1.67 mmol) is introduced thereinto. After reacting for 12 hours, the temperature of the product is reduced to normal temperature, and the generated solid is filtered. After the filtering, the solid is washed by using 100 ml of tetrahydrofuran, 500 ml of ethyl acetate, 500 ml of water and 300 ml of ethanol, and the obtained product is dried to prepare an intermediate 3e (78.7%).

The steps of synthesizing the compound E3 are substantially the same as the steps of synthesizing the compound E1, and differ in that the intermediate 1A in the step of synthesizing the compound E1 is replaced by 3e, and the 1c is replaced by 3f. The other reagents are not changed, to obtain the compound E3, wherein the yield is approximately 79.6%.

The particular process of preparing the compound E4 is as follows:

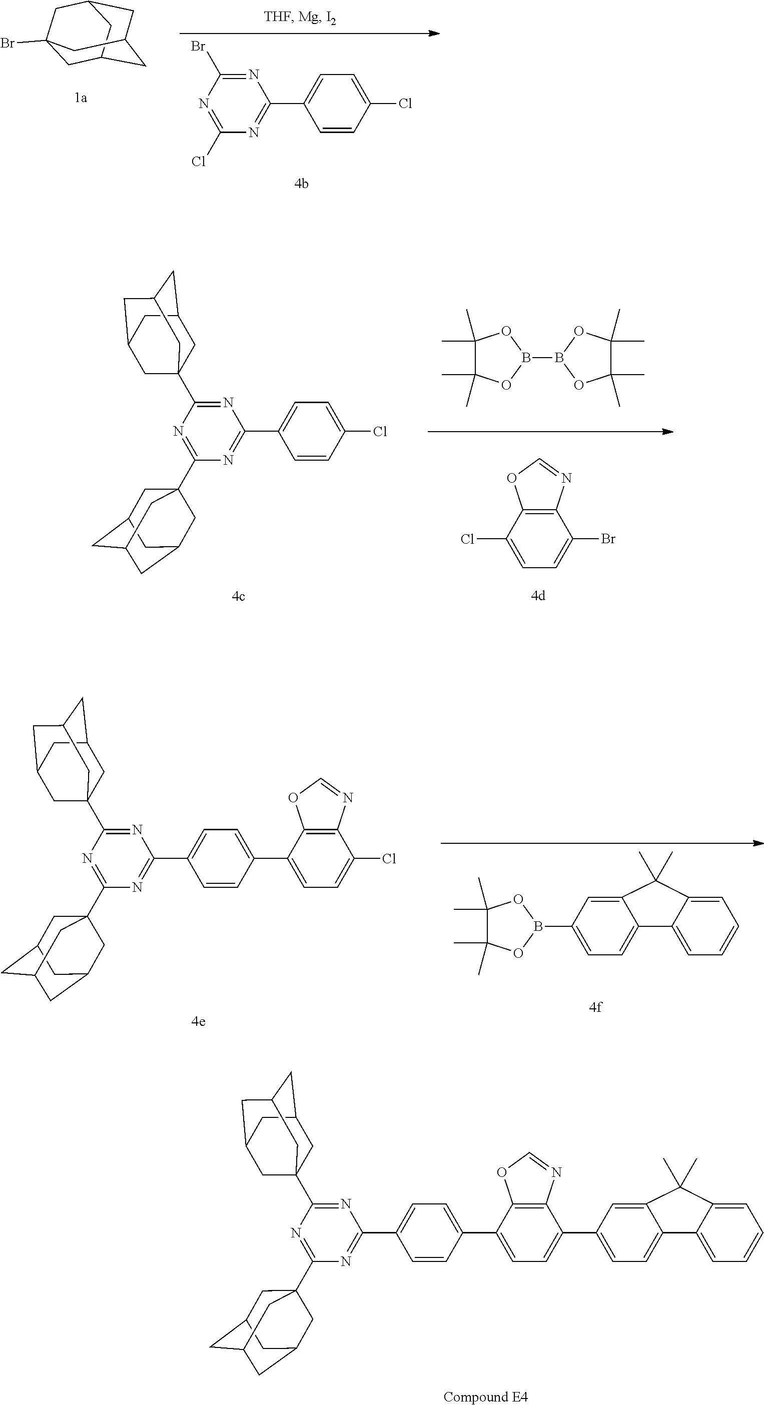

The steps of synthesizing the compound E4 may refer to the steps of synthesizing the compound E3, and differ in that the 3b in the step of synthesizing the compound E3 is replaced by 4b, to obtain an intermediate 4c; subsequently, an intermediate 4e is obtained; and subsequently the 3f in the step of synthesizing the compound E3 is replaced by 4f, to obtain the compound E4. The yield of the compound E4 is approximately 81.7%.

The particular process of preparing the compound E5 is as follows:

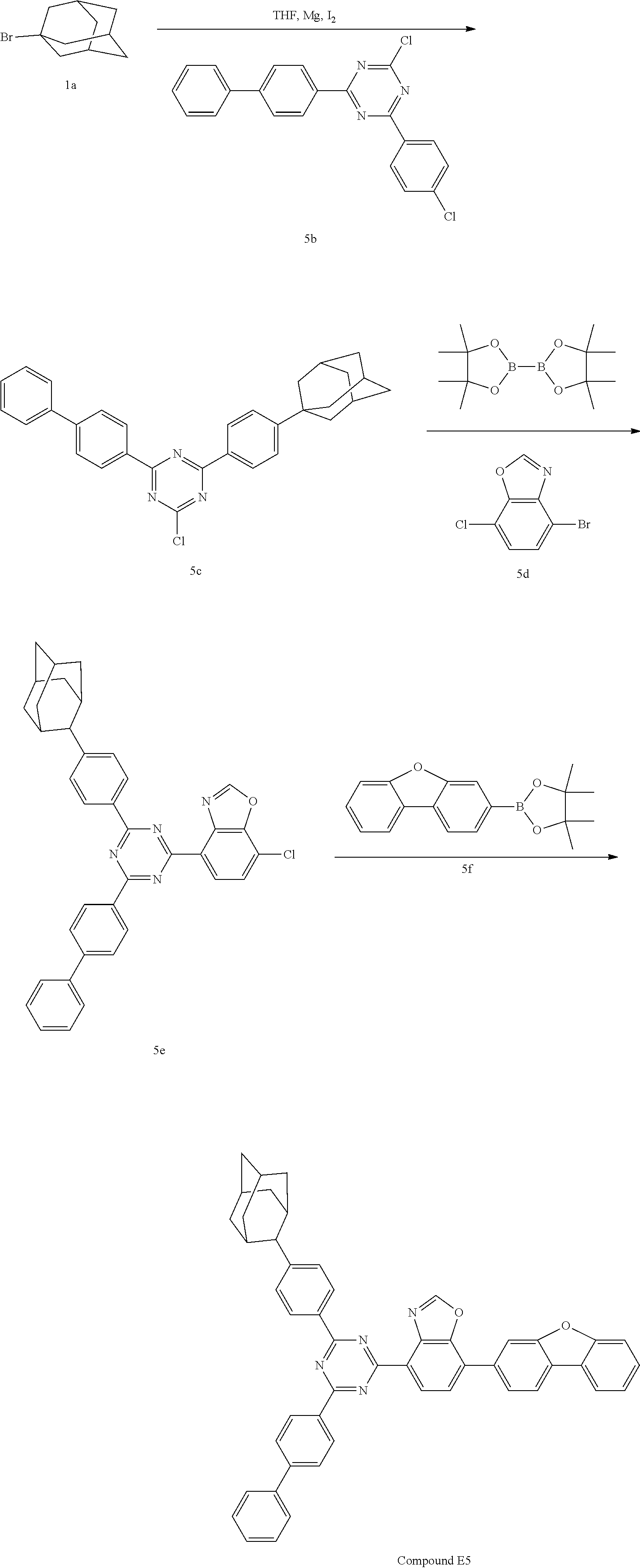

The steps of synthesizing the compound E5 may refer to the steps of synthesizing the compound E3, and differ in that the 3b in the step of synthesizing the compound E3 is replaced by 5b, to obtain an intermediate 5c; subsequently, an intermediate 5e is obtained; and subsequently the 3f in the step of synthesizing the compound E3 is replaced by 5f, to obtain the compound E5. The yield of the compound E5 is approximately 85.4%.

The particular process of preparing the compound E6 is as follows:

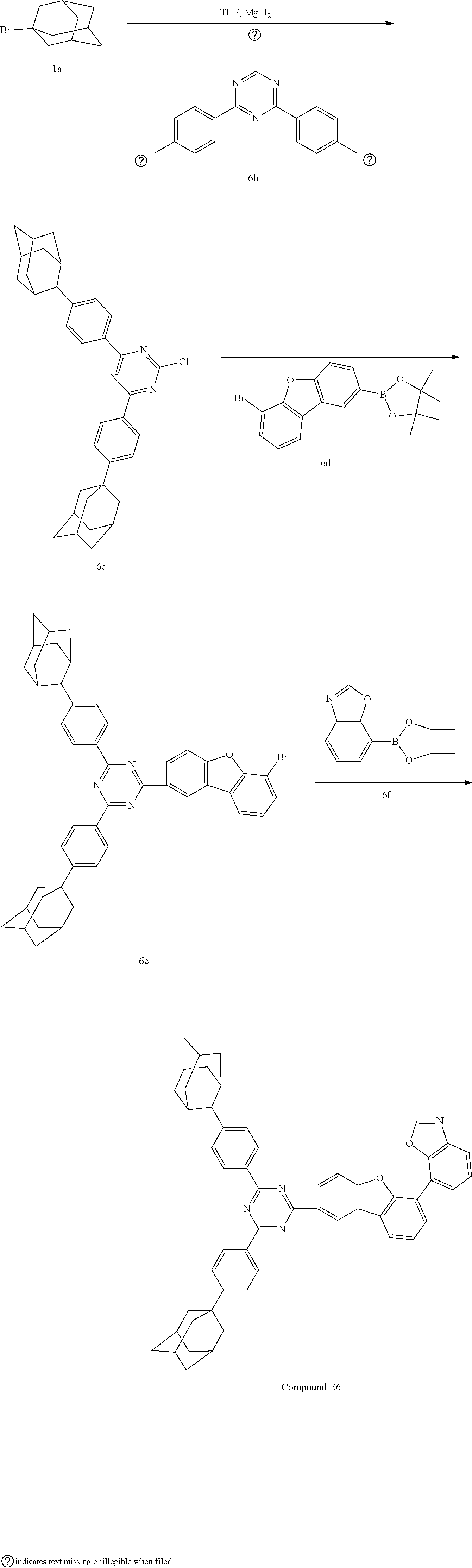

The steps of synthesizing the compound E6 may refer to the steps of synthesizing the compound E3, and differ in that the 3b in the step of synthesizing the compound E3 is replaced by 6b, to obtain an intermediate 6c; subsequently, the 3d in the step of synthesizing the compound E3 is replaced by 6d, to obtain an intermediate 6e; and subsequently the 3f in the step of synthesizing the compound E3 is replaced by 6f, to obtain the compound E6. The yield of the compound E6 is approximately 79.4%.

The compounds E1-E6 and Comparative Example (whose chemical formula is

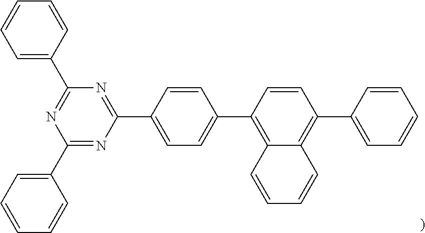 [SMILES: c1ccc(-c2nc(-c3ccccc3)nc(-c3ccc(-c4ccc(-c5ccccc5)c5ccccc45)cc3)n2)cc1]

are test, to obtain the electron mobilities, the reorganization energies, the HOMO energy levels and the LUMO energy levels in the following Table 1.

TABLE 1

| | Electron | | HOMO | LUMO |
|---|---|---|---|---|
| | mobility/ | Reorganization | energy | energy |
| Compound | m/V · S | energy/eV | level/eV | level/eV |
| Compound E1 | 6.62 × 10⁻⁵ | 0.22 | 6.71 | 3.24 |
| Compound E2 | 1.23 × 10⁻⁵ | 0.25 | 6.46 | 3.18 |
| Compound E3 | 1.12 × 10⁻⁵ | 0.19 | 6.72 | 3.39 |
| Compound E4 | 1.23 × 10⁻⁵ | 0.20 | 6.56 | 3.29 |
| Compound E5 | 1.23 × 10⁻⁵ | 0.19 | 6.67 | 3.22 |
| Compound E6 | 8.75 × 10⁻⁵ | 0.24 | 6.55 | 3.25 |
| Comparative | 1.23 × 10⁻⁵ | 0.33 | 6.22 | 3.02 |
| Example | | | | |

From Table 1, it can be known that, as compared with Comparative Example, the compounds E1-E6 according to the present application include the first electron-withdrawing group, and have deep HOMO energy level and LUMO energy level, whereby they can transmit electrons better, thereby reducing the operation voltage of the light emitting device. Moreover, the first electron-withdrawing group can interact with the neighboring groups, so that the molecules have stable geometrical configurations, whereby they do not easily deform by the effect of an external electric field, and have lower reorganization energies and higher electron mobilities.

As compared with the comparative compound, the three-dimensional group having a rigidity of the compounds E1-E6 according to the present application can regulate the intermolecular force of the material, reduce the intermolecular stacking effect, and reduce the crystallization caused by Joule heat, thereby improving the life of the light emitting device.

The compounds E1-E2, a compound E7 (whose chemical formula is

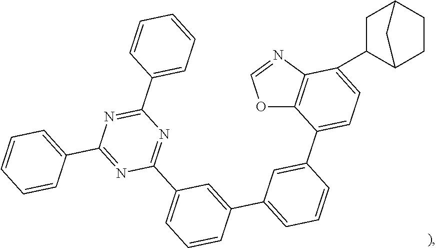 [SMILES: c1ccc(-c2nc(-c3ccccc3)nc(-c3cccc(-c4cccc(-c5ccc(C6CC7CCC6C7)c6ncoc56)c4)c3)n2)cc1]

a compound E8 (whose chemical formula is

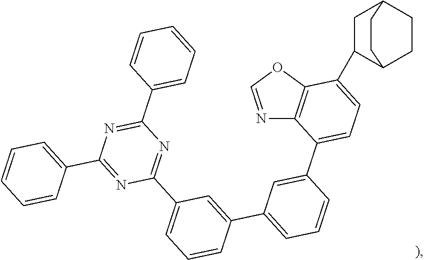 [SMILES: c1ccc(-c2nc(-c3ccccc3)nc(-c3cccc(-c4cccc(-c5ccc(C6CC7CCC6CC7)c6ocnc56)c4)c3)n2)cc1]

a compound E9 (whose chemical formula is

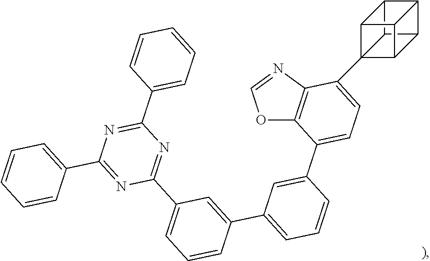 [SMILES: c1ccc(-c2nc(-c3ccccc3)nc(-c3cccc(-c4cccc(-c5ccc(C67C8C9C%10C8C6C%10C97)c6ncoc56)c4)c3)n2)cc1]

a compound E10 (whose chemical formula is

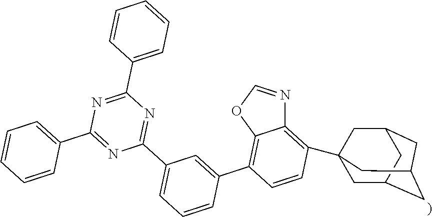 [SMILES: c1ccc(-c2nc(-c3ccccc3)nc(-c3cccc(-c4ccc(C56CC7CC(CC(C7)C5)C6)c5ncoc45)c3)n2)cc1]

and Comparative Example (whose chemical formula is

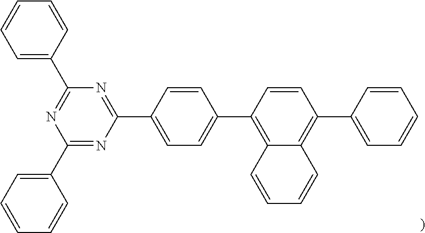 [SMILES: c1ccc(-c2nc(-c3ccccc3)nc(-c3ccc(-c4ccc(-c5ccccc5)c5ccccc45)cc3)n2)cc1]

are test, to obtain the glass-transition temperatures Tg and the triplet-state energy levels T1 in the following Table 2.

TABLE 2

| Compound | Tg | T1/eV |
|---|---|---|
| Compound E1 | 139 | 2.36 |
| Compound E2 | 146 | 2.52 |
| Compound E7 | 143 | 2.55 |
| Compound E8 | 143 | 2.51 |
| Compound E9 | 141 | 2.51 |
| Compound E10 | 140 | 2.48 |
| Comparative Example | 122 | 2.2 |

From Table 2, it can be known that, as compared with the comparative compound, all of the compounds E1-E6 according to the present application include a three-dimensional group. The three-dimensional group is of a rigid three-dimensional structure, and the materials have a high Tg. The high Tg facilitates to improve the thermodynamic stability of the materials, to enable the materials not to easily be cracked in the vapor deposition, and have good film-forming property, durability, heat resistance and so on, whereby the life of the light emitting device is significantly improved.

As compared with Comparative Example, all of the compounds E1-E6 according to the present application include a benzoxazole-type group, wherein the benzoxazole-type group has a high T1, and, when introduced, can increase the T1 of the materials to a large extent. Moreover, the groups are connected in a special connection mode, for example, meta-position connection, which can effectively destroy or reduce the conjugation among the molecules, so as to further increase the T1 of the material, thereby further increasing the utilization ratio of the excitons.

Optionally, referring to FIG. 2, the light emitting device further comprises an anode 1 and a cathode 3, and the electron transport layer 8 is located between the anode 1 and the cathode 3. The light emitting device further comprises a luminescent layer 2 and an electron blocking layer 6, and the luminescent layer 2 is located between the anode 1 and the electron transport layer 8. The electron blocking layer 6 is located between the anode 1 and the luminescent layer 2.

The material of the anode is not particularly limited herein. As an example, the material of the anode may include ITO (Indium Tin Oxides).

The process of fabricating the anode is not particularly limited herein. As an example, the process may comprise sonicating a glass plate having ITO in deionized water, and subsequently drying at 100° C., to obtain the anode.

The material of the cathode is not particularly limited herein. As an example, the material of the cathode may include a metal, for example, any one of magnesium, calcium, sodium, potassium, titanium, indium, yttrium, lithium, gadolinium, aluminum, silver, tin and lead, or an alloy thereof.

The electron blocking layer can block the electrons in the luminescent layer from leaving the luminescent layer, to ensure that more electrons recombine with the holes in the luminescent layer, thereby increasing the quantity of the excitons, to increase the luminous efficiency.

The material of the luminescent layer is not particularly limited herein. As an example, the material of the luminescent layer may include a host material and a guest material, wherein the guest material is doped in the host material. Alternatively, the material of the luminescent layer may include a single material.

The luminescent layer may be any one of a red luminescent layer, a green luminescent layer and a blue luminescent layer, in which case the luminescent layer may be used for luminescence of a single color. The light emitting device may comprise all of the three types of the red luminescent layer, the green luminescent layer and the blue luminescent layer. Certainly, it may also comprise merely one type of the luminescent layer; for example, it comprises merely a plurality of red luminescent layers, or comprises merely a plurality of green luminescent layers, or comprises merely a plurality of blue luminescent layers, which may be decided particularly according to practical demands. The red luminescent layer is taken as an example for the description herein, and the luminescent layers of the other colors may refer to the red luminescent layer, which is not particularly discussed further herein. The red luminescent layer comprises a hole-type host material, an electron-type host material and a guest material emitting red light.

Optionally, referring to FIG. 2, the light emitting device further comprises a hole injection layer 4, an electron injection layer 7, a hole blocking layer 9 and a hole transport layer 5, the hole injection layer 4 is located between the anode 1 and the hole transport layer 5, the hole transport layer 5 is located between the hole injection layer 4 and the electron blocking layer 6, the electron injection layer 7 is located between the cathode 3 and the hole blocking layer 9, and the hole blocking layer 9 is located between the luminescent layer 2 and the electron transport layer 8.

The T1 of the host material (Host) in the luminescent layer and the T1 of the material of the electron blocking layer (EBL) satisfy: T1 (EBL)>T1 (Host).

The T1 of the host material (Host) in the luminescent layer and the T1 of the material of the hole blocking layer (HBL) satisfy: T1 (HBL)>T1 (Host), which facilitates to restrict the excitons generated by the holes and the electrons inside the luminescent layer.

The T1 of the material of the hole blocking layer (HBL) and the T1 of the material of the electron transport layer (ETL) satisfy: T1 (ETL)>T1 (HBL), thereby preventing that, because of a too thin HBL, the holes pass through the HBL to cause quenching of the excitons.

The T1 of the material of the electron blocking layer (EBL) and the T1 of the material of the hole transport layer (HTL) satisfy: T1 (HTL)>T1 (EBL), thereby preventing that, because of a too thin EBL, the electrons pass through the EBL to cause quenching of the excitons.

The LUMO energy level of the hole blocking layer (HBL) and the LUMO energy level of the electron transport layer (ETL) satisfy: 0.4 eV≤LUMO (HBL)−LUMO (ETL)≤1 eV, which can increase the energy-level barrier potential between the HBL and the ETL, and reduce the transmission speed of the electrons.

The HOMO energy level of the hole transport layer (HTL) and the HOMO energy level of the electron blocking layer (EBL) satisfy: 0.3 eV≤|HOMO (HTL)−HOMO (EBL)|≤1 eV, which can eliminate the problem of a slow hole transmission caused by the energy-level barrier potential.

The LUMO energy level of the hole blocking layer (HBL) and the LUMO energy level of the host material (Host) in the luminescent layer satisfy: |LUMO (HBL)−LUMO (Host)|≤0.3 eV, which facilitates the electron transmission.

The electron injection layer can transmit the electrons, and can inject the electrons injected by the cathode into the luminescent layer. The material of the electron injection layer herein may be an alkali metal or a metal, for example, LiF, Yb, Mg and Ca, or a compound thereof. The name in English of LiF herein is lithium fluoride. The name in English of Yb is ytterbium.

The hole blocking layer can block the holes in the luminescent layer from leaving the luminescent layer, to ensure that more holes recombine with the electrons in the luminescent layer, thereby increasing the quantity of the excitons, to increase the luminous efficiency. The material of the hole blocking layer herein may be an aromatic heterocyclic compound, for example, any one or more of benzimidazole, triazine, pyrimidine, pyridine, pyrazine, quinoxaline, quinoline, diazole, diazephosphoracyclopentadiene, phosphine oxide, an aromatic ketone, lactam and borane and derivatives thereof. As an example, the material of the hole blocking layer may be TPBI, wherein the name in English of TPBI is 1,3,5-tris(1-phenyl-1H-benzimidazol-2-yl)benzene, and the chemical structural formula is

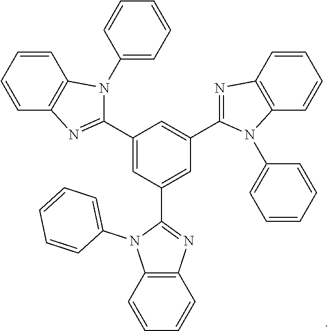 [SMILES: c1ccc(-n2c(-c3cc(-c4nc5ccccc5n4-c4ccccc4)cc(-c4nc5ccccc5n4-c4ccccc4)c3)nc3ccccc32)cc1]

Particular examples and comparative example will be provided below to describe the effect of improvement.

An ITO baseplate (used as the material of the Anode layer) that was prepared in advance is washed and dried. On the ITO baseplate are sequentially vapor-deposited

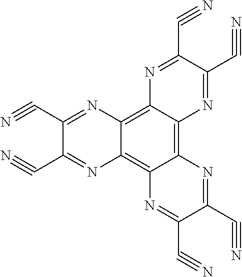 [SMILES: N#Cc1nc2c3nc(C#N)c(C#N)nc3c3nc(C#N)c(C#N)nc3c2nc1C#N]

(used as the material of the HIL layer),

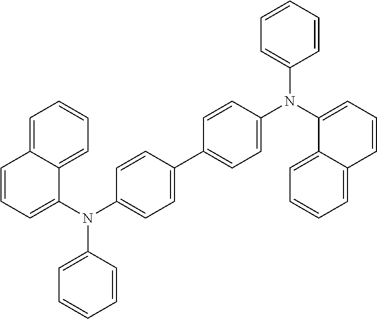 [SMILES: c1ccc(N(c2ccc(-c3ccc(N(c4ccccc4)c4cccc5ccccc45)cc3)cc2)c2cccc3ccccc23)cc1]

(used as the material of the HTL layer),

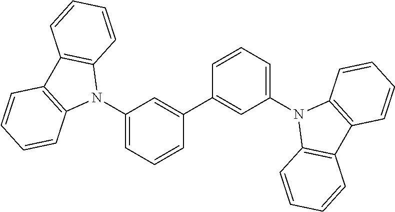 [SMILES: c1cc(-c2cccc(-n3c4ccccc4c4ccccc43)c2)cc(-n2c3ccccc3c3ccccc32)c1]

(used as the material of the EBL layer),

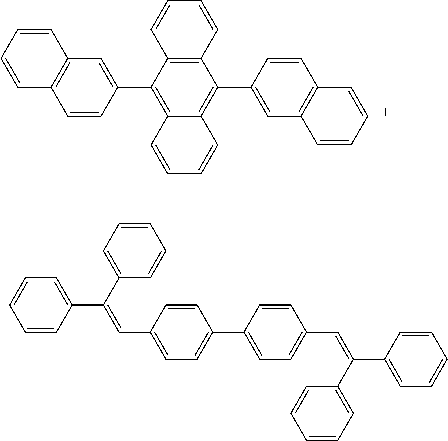

(used as the blue-light host material BH+the blue-light guest material BD of the EML layer),

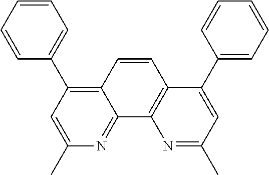 [SMILES: Cc1cc(-c2ccccc2)c2ccc3c(-c4ccccc4)cc(C)nc3c2n1]

(used as the material of the HBL layer), LiF (used as the material of the EIL layer), the compound E1 (used as the material of the ETL layer), and Al (used as the material of the Cathode layer), to obtain a first light emitting device.

The structure of the first light emitting device is:
Anode (10 nm)/HIL (10 nm)/HTL (100 nm)/EBL (35 nm)/BH:BD (20 nm, 3 wt %)/HBL (5 nm)/ETL:Liq (30 nm, 50%)/EIL (1 nm)/Cathode (100 nm)

By replacing the material in the ETL of the first light emitting device with the compound E2, a second light emitting device is obtained.

By replacing the material in the ETL of the first light emitting device with the compound E3, a third light emitting device is obtained.

By replacing the material in the ETL of the first light emitting device with the compound E4, a fourth light emitting device is obtained.

By replacing the material in the ETL of the first light emitting device with the compound E5, a fifth light emitting device is obtained.

By replacing the material in the ETL of the first light emitting device with the compound E6, a sixth light emitting device is obtained.

By replacing the material in the ETL of the first light emitting device with the compound E7, a seventh light emitting device is obtained.

By replacing the material in the ETL of the first light emitting device with the compound E8, an eighth light emitting device is obtained.

By replacing the material in the ETL of the first light emitting device with the compound E9, a ninth light emitting device is obtained.

By replacing the material in the ETL of the first light emitting device with a comparative example compound (whose chemical formula is

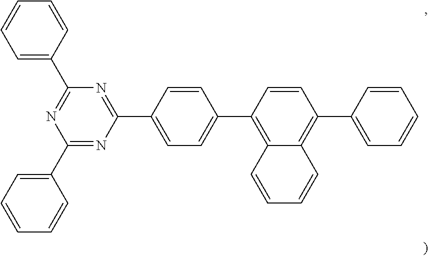 [SMILES: c1ccc(-c2nc(-c3ccccc3)nc(-c3ccc(-c4ccc(-c5ccccc5)c5ccccc45)cc3)n2)cc1]

a tenth light emitting device is obtained.

The first light emitting device, the second light emitting device, the third light emitting device, the fourth light emitting device, the fifth light emitting device, the sixth light emitting device, the seventh light emitting device, the eighth light emitting device, the ninth light emitting device and the tenth light emitting device are test individually, to obtain the voltages, the luminescence peaks, the luminous efficiencies and the lives of the light emitting devices, as shown in the following Table 3.

TABLE 3

| | | Luminescence | Luminous | |
|---|---|---|---|---|
| Light emitting device | Voltage/V | peak/nm | efficiency/cd/cm² | Life/h |
| First light emitting device | 96% | 458 | 117% | 124% |
| Second light emitting device | 98% | 462 | 109% | 115% |
| Third light emitting device | 97% | 459 | 110% | 107% |
| Fourth light emitting device | 97% | 457 | 108% | 104% |
| Fifth light emitting device | 96% | 458 | 112% | 128% |
| Sixth light emitting device | 98% | 460 | 117% | 123% |
| Seventh light emitting device | 98% | 459 | 110% | 114% |
| Eighth light emitting device | 98% | 459 | 108% | 115% |
| Ninth light emitting device | 97% | 458 | 109% | 117% |
| Tenth light emitting device | 100% | 461 | 100% | 100% |

From Table 3, it can be seen that, as compared with the tenth light emitting device, the first light emitting device to the ninth light emitting device have lower voltages, higher luminous efficiencies, and longer lives.

The light emitting device may be applied to a displaying device. The particular structure of the displaying device is not limited herein.

As an example, the displaying device may comprise a displaying baseplate and the light emitting device. The displaying baseplate comprises a plurality of pixel units that are arranged in an array. The light emitting device comprises a red light emitting device, a green light emitting device and a blue light emitting device that are arranged in an array. Each of the pixel units comprises a red sub-pixel, a green sub-pixel and a blue sub-pixel. The red sub-pixel is electrically connected to the red light emitting device, the green sub-pixel is electrically connected to the green light emitting device, and the blue sub-pixel is electrically connected to the blue light emitting device.

Referring to FIG. 3, the red sub-pixel is electrically connected to the red light emitting device 100, the green sub-pixel is electrically connected to the green light emitting device 200, and the blue sub-pixel is electrically connected to the blue light emitting device 300. Referring to FIG. 3, the particular structure will be described by taking the leftmost red sub-pixel as an example. The red sub-pixel comprises: a buffer layer 11, an active layer 210, a gate insulating layer 12, a gate metal layer (comprising a gate 110 and a first electrode 212), an insulating layer 13, an electrode layer (comprising a second electrode 213), an interlayer dielectric layer 14, a source-drain metal layer (comprising a source 111 and a drain 112), a planarization layer 15 and a pixel definition layer 302 that are located on a substrate 10 and are sequentially stacked. The first electrode 212 and the second electrode 213 are used to form a storage capacitor. The pixel definition layer 302 comprises an opening, the red light emitting device 100 is provided in the opening, and the anode 1 of the red light emitting device 100 is electrically connected to the drain 112 of the thin-film transistor. The displaying baseplate further comprises a separator 34 located on the pixel definition layer 302. It should be noted that, in the displaying baseplate, the separator may be provided on part of the pixel definition layer, and may also be provided on the whole pixel definition layer, which is not limited herein.

The red light emitting device 100 comprises the anode 1, and the hole injection layer 4, the hole transport layer 5, the electron blocking layer 6, a red luminescent layer 113, the hole blocking layer 9, the electron transport layer 8, the electron injection layer 7 and the cathode 3 that are located on the anode 1 and are sequentially stacked.

It should be noted that the materials of the luminescent layers of the green light emitting device 200 and the blue light emitting device 300 shown in FIG. 3 are different from the material of the luminescent layer of the red light emitting device 100. The luminescent layer of the green light emitting device is used to emit a green light, the luminescent layer of the blue light emitting device is used to emit a blue light, and the luminescent layer of the red light emitting device is used to emit a red light. Moreover, the materials of the electron blocking layers of the green light emitting device and the blue light emitting device are different from the material of the electron blocking layer of the red light emitting device. Except for the luminescent layers and the electron blocking layers, all of the other film layers of the green light emitting device and the blue light emitting device are the same as those of the red light emitting device, and are not discussed further herein.

An embodiment of the present application further provides a displaying device, wherein the displaying device comprises the light emitting device stated above.

The displaying device may be a flexible displaying device (also referred to as a flexible screen), and may also be a rigid displaying device (i.e., a display screen that cannot be bent), which is not limited herein. The displaying device may be an OLED displaying device, and may also be an LCD (Liquid-Crystal Display) displaying device. The displaying device may be any product or component that has the function of displaying, such as a television set, a digital camera, a mobile phone and a tablet personal computer. The displaying device may also be applied in fields such as identity identification and medical equipment, and the products that have already been promoted or have a good prospect of promotion include security identity authentication, smart door locks, medical video collection and so on. The displaying device has the advantages such as a high stability, a high luminous efficiency, a long life, a low voltage, a good effect of displaying, a high contrast, a good imaging quality and a high product quality.

The “embodiment” as used herein means that particular features, structures or characteristics described with reference to an embodiment are included in at least one embodiment of the present application.

The description provided herein describes many concrete details. However, it can be understood that the embodiments of the present application may be implemented without those concrete details. In some of the embodiments, well-known processes, structures and techniques are not described in detail, so as not to affect the understanding of the description.

Finally, it should be noted that the above embodiments are merely intended to explain the technical solutions of the present application, and not to limit them. Although the present application is explained in detail with reference to the above embodiments, a person skilled in the art should understand that he can still modify the technical solutions set forth by the above embodiments, or make equivalent substitutions to part of the technical features of them. However, those modifications or substitutions do not make the essence of the corresponding technical solutions depart from the spirit and scope of the technical solutions of the embodiments of the present application.

1. A light emitting device, wherein the light emitting device comprises an electron transport layer, and a material of the electron transport layer comprises a heteroaromatic ring group comprising at least one nitrogen atom, a first electron-withdrawing group, and at least one three-dimensional group having a rigidity;
a triplet-state energy level T1 of a material of the electron transport layer satisfies T1≤2.3 eV;
an absolute value of an energy value of a highest occupied molecular orbital HOMO of the material of the electron transport layer satisfies 6.0 eV≤|HOMO|≤7.0 eV; and
an absolute value of an energy value of a lowest unoccupied molecular orbital LUMO of the material of the electron transport layer satisfies 2.6 eV≤|LUMO|≤3.6 eV.

2. The light emitting device according to claim 1, wherein a general structural formula of the material of the electron transport layer is: 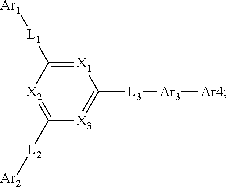
wherein X1 is C(Ra) or nitrogen, X2 is C(Rb) or nitrogen, X3 is C(Rc) or nitrogen, and at least one of X1, X2 and X3 is nitrogen, wherein each of Ra, Rb and Rc is independently any one of a group consisting of hydrogen, diplogen, fluorine, chlorine, an alkyl group whose carbon-atom quantity is 1-12, a halogenated alkyl group whose carbon-atom quantity is 1-12, an alkoxy group whose carbon-atom quantity is 1-12, a cycloalkyl group whose carbon-atom quantity is 3-10, C6-20 aryl, and a heteroaryl group whose carbon-atom quantity is 3-20;
each of L1, L2 and L3 is independently any one of a group consisting of a single bond, a substituted or unsubstituted arylene group whose carbon-atom quantity is 6-60, and a substituted or unsubstituted heteroarylene whose carbon-atom quantity is 1-60;
each of Ar1, Ar2 and Ar4 is independently any one of a group consisting of a substituted or unsubstituted alkyl group whose carbon-atom quantity is 1-12, a substituted or unsubstituted cycloalkyl group whose carbon-atom quantity is 3-10, a substituted or unsubstituted aralkyl group whose carbon-atom quantity is 7-30, a substituted or unsubstituted heterarylalkyl whose carbon-atom quantity is 2-60, a substituted or unsubstituted aryl whose carbon-atom quantity is 6-60, and a substituted or unsubstituted heteroaryl group whose carbon-atom quantity is 3-60, and at least one of Ar1, Ar2 and Ar4 is the three-dimensional group having a rigidity; and
Ar3 is the first electron-withdrawing group.

3. The light emitting device according to claim 2, wherein in the material of the electron transport layer, a general structural formula of Ar3 is: 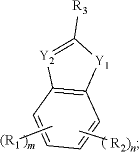
wherein Y1 is any one of carbon, oxygen, sulphur, selenium and N—R4;
Y2 is any one of carbon and nitrogen;
each of R1, R2 and R3 is independently any one of hydrogen, diplogen, halogen, amino, a nitrile group, nitro, C1-30 alkyl, C2-30 alkenyl, C2-30 alkynyl, C1-30 alkoxy, C6-30 aryloxy, and C6-50 aryl substituted by C6-30 aryl, or comprises any one of at least one of heteroatoms selected from nitrogen, oxygen and sulphur and C2-50 heteroaryl unsubstituted or substituted by C6-30 aryloxy, and two of R1, R2 and R3 are directly connected to neighboring groups L3 and Ar4;
R4 is any one of a group consisting of hydrogen, diplogen, fluorine, chlorine, an alkyl group whose carbon-atom quantity is 1-12, a halogenated alkyl group whose carbon-atom quantity is 1-12, an alkoxy group whose carbon-atom quantity is 1-12, a cycloalkyl group whose carbon-atom quantity is 3-10, C6-20 aryl, and a heteroaryl group whose carbon-atom quantity is 3-20; and
each of m and n is independently 1 or 2.

4. The light emitting device according to claim 3, wherein a general structural formula of the material of the electron transport layer is any one of

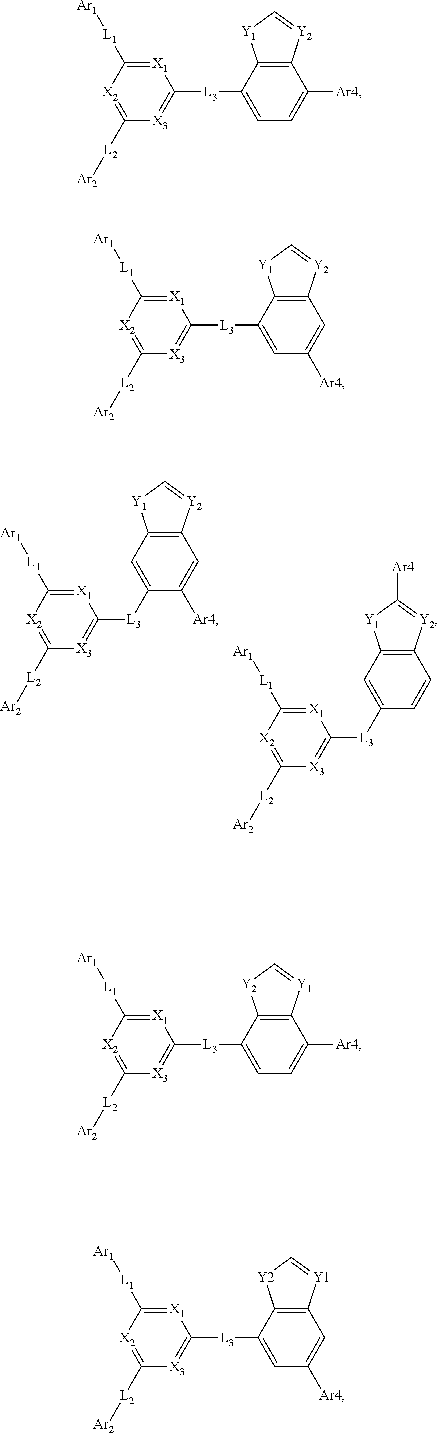

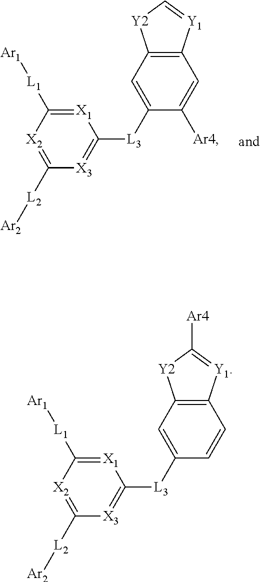

5. The light emitting device according to claim 4, wherein Ar3 comprises benzoxazole.

6. The light emitting device according to claim 3, wherein a general structural formula of the three-dimensional group having a rigidity is any one of 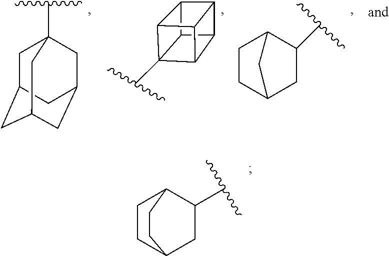
wherein 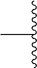
is a bond connected to a carbon atom.

7. The light emitting device according to claim 2, wherein in the material of the electron transport layer, the heteroaromatic ring group comprising at least one nitrogen atom comprises a triazine group.

8. The light emitting device according to claim 7, wherein a chemical structural formula of a material of the electron transport layer having the triazine group includes any one of

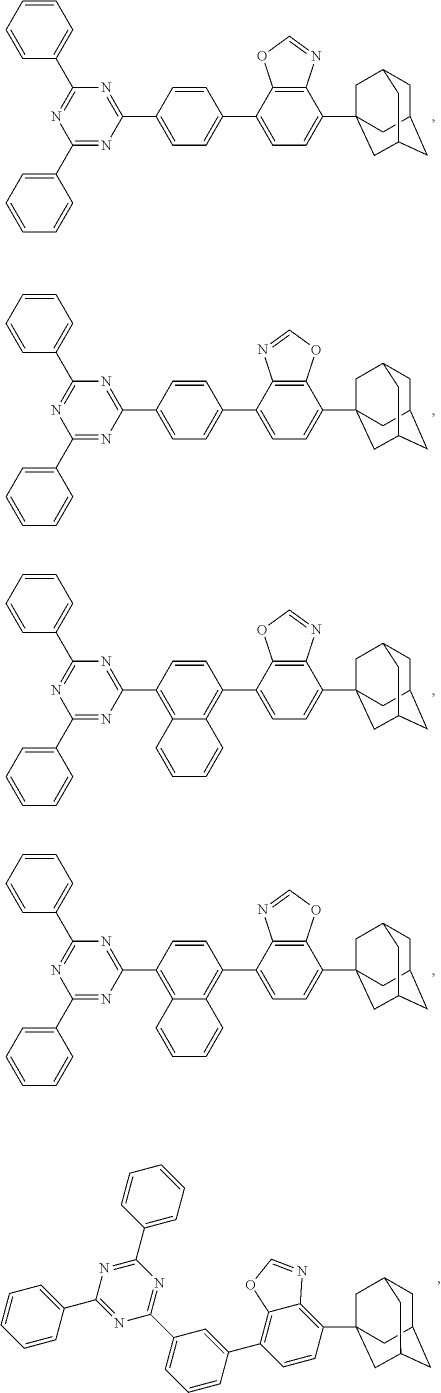 [SMILES: c1ccc(-c2nc(-c3ccccc3)nc(-c3ccc(-c4ccc(C56CC7CC(CC(C7)C5)C6)c5ncoc45)cc3)n2)cc1 ; c1ccc(-c2nc(-c3ccccc3)nc(-c3ccc(-c4ccc(C56CC7CC(CC(C7)C5)C6)c5ocnc45)cc3)n2)cc1 ; c1ccc(-c2nc(-c3ccccc3)nc(-c3ccc(-c4ccc(C56CC7CC(CC(C7)C5)C6)c5ncoc45)c4ccccc34)n2)cc1 ; c1ccc(-c2nc(-c3ccccc3)nc(-c3ccc(-c4ccc(C56CC7CC(CC(C7)C5)C6)c5ocnc45)c4ccccc34)n2)cc1 ; c1ccc(-c2nc(-c3ccccc3)nc(-c3cccc(-c4ccc(C56CC7CC(CC(C7)C5)C6)c5ncoc45)c3)n2)cc1]

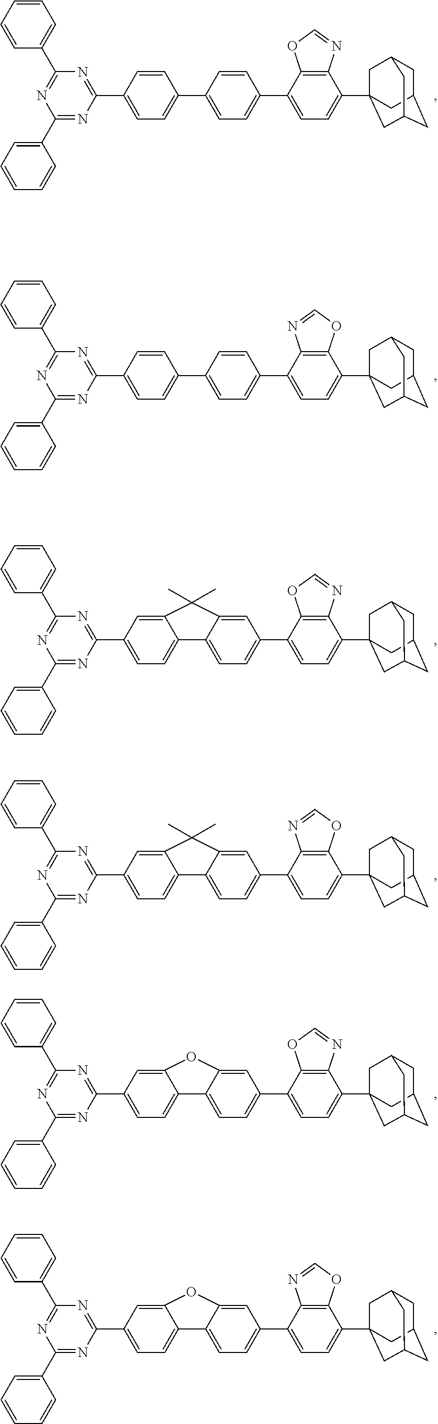

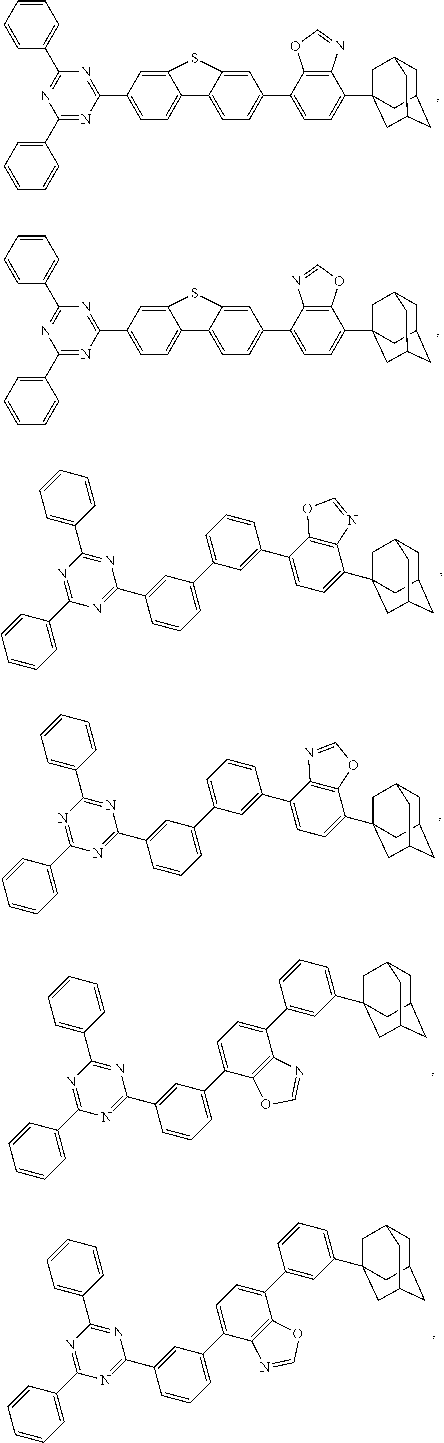

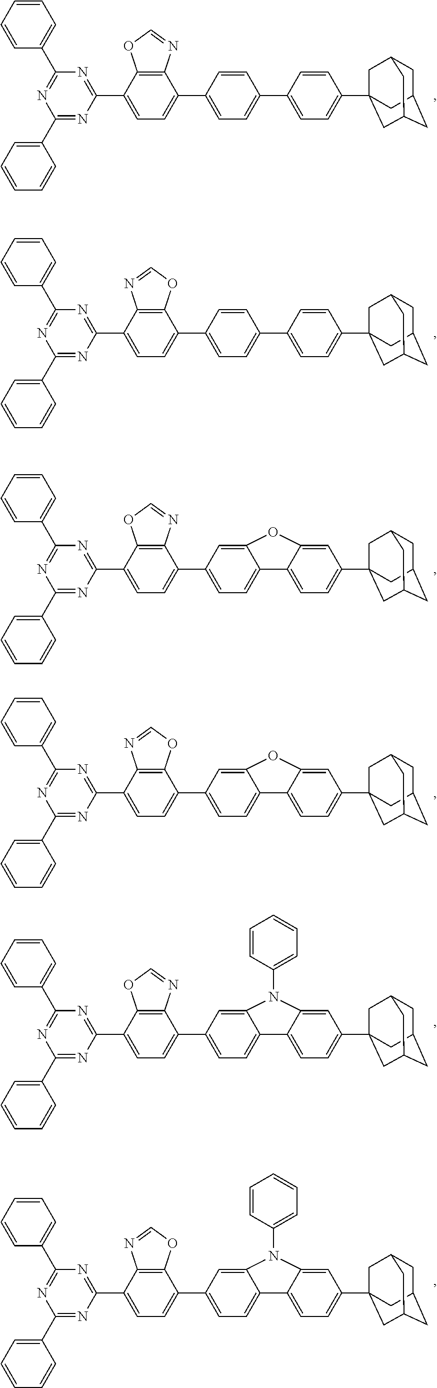

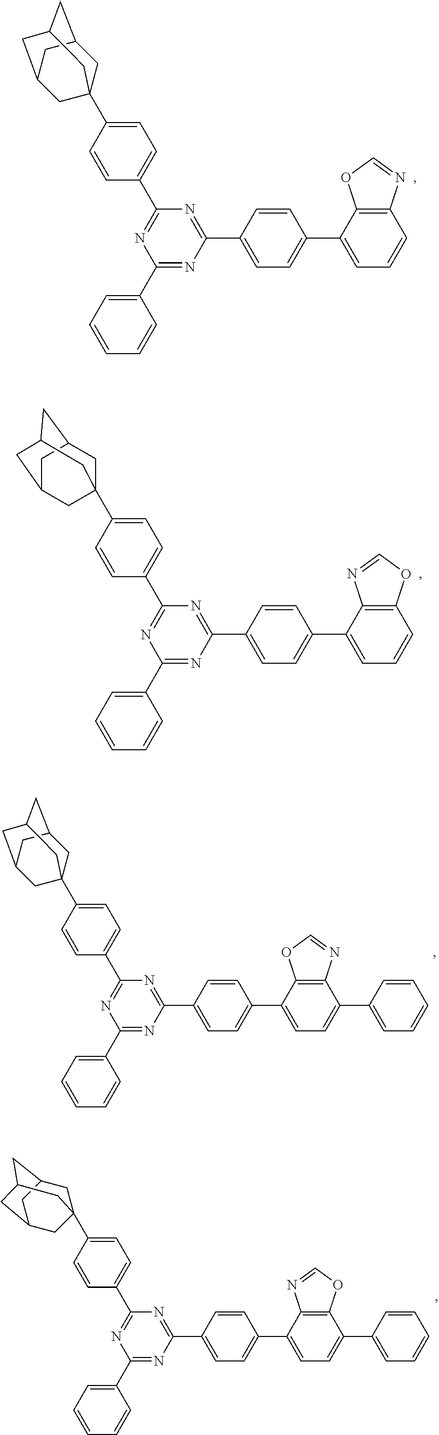 [SMILES: c1ccc(-c2nc(-c3ccc(-c4cccc5ncoc45)cc3)nc(-c3ccc(C45CC6CC(CC(C6)C4)C5)cc3)n2)cc1 ; c1ccc(-c2nc(-c3ccc(-c4cccc5ocnc45)cc3)nc(-c3ccc(C45CC6CC(CC(C6)C4)C5)cc3)n2)cc1 ; c1ccc(-c2nc(-c3ccc(-c4ccc(-c5ccccc5)c5ncoc45)cc3)nc(-c3ccc(C45CC6CC(CC(C6)C4)C5)cc3)n2)cc1 ; c1ccc(-c2nc(-c3ccc(-c4ccc(-c5ccccc5)c5ocnc45)cc3)nc(-c3ccc(C45CC6CC(CC(C6)C4)C5)cc3)n2)cc1]

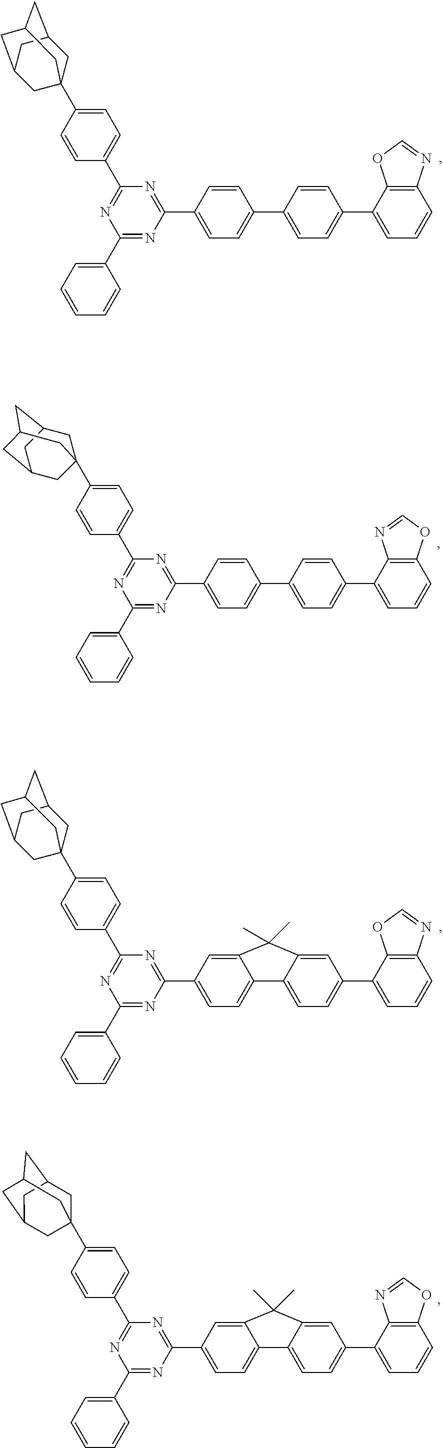 [SMILES: c1ccc(-c2nc(-c3ccc(-c4ccc(-c5cccc6ncoc56)cc4)cc3)nc(-c3ccc(C45CC6CC(CC(C6)C4)C5)cc3)n2)cc1 ; c1ccc(-c2nc(-c3ccc(-c4ccc(-c5cccc6ocnc56)cc4)cc3)nc(-c3ccc(C45CC6CC(CC(C6)C4)C5)cc3)n2)cc1 ; CC1(C)c2cc(-c3nc(-c4ccccc4)nc(-c4ccc(C56CC7CC(CC(C7)C5)C6)cc4)n3)ccc2-c2ccc(-c3cccc4ncoc34)cc21 ; CC1(C)c2cc(-c3nc(-c4ccccc4)nc(-c4ccc(C56CC7CC(CC(C7)C5)C6)cc4)n3)ccc2-c2ccc(-c3cccc4ocnc34)cc21]

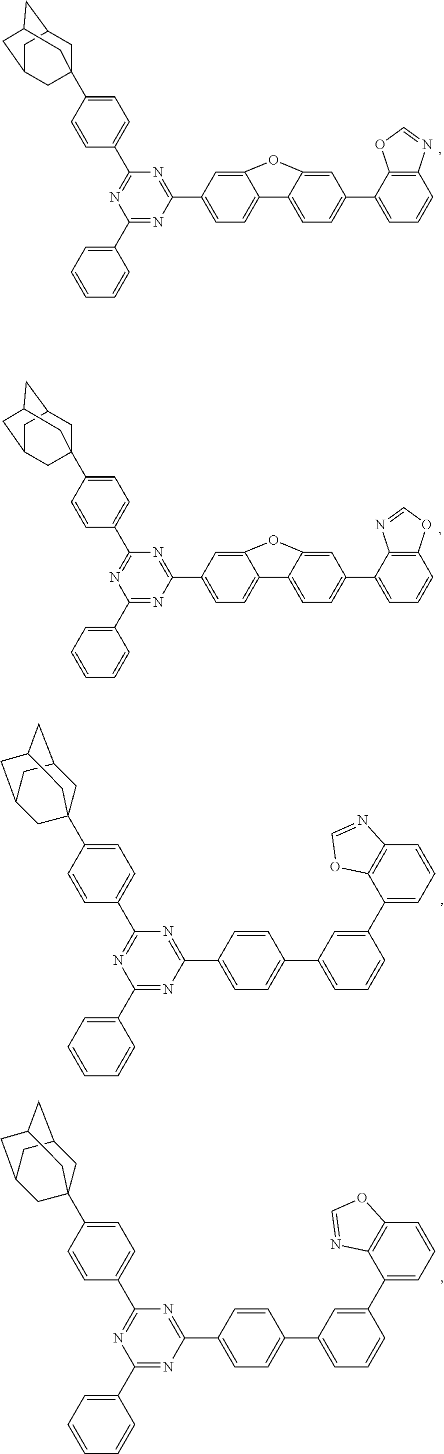 [SMILES: c1ccc(-c2nc(-c3ccc(C45CC6CC(CC(C6)C4)C5)cc3)nc(-c3ccc4c(c3)oc3cc(-c5cccc6ncoc56)ccc34)n2)cc1 ; c1ccc(-c2nc(-c3ccc(C45CC6CC(CC(C6)C4)C5)cc3)nc(-c3ccc4c(c3)oc3cc(-c5cccc6ocnc56)ccc34)n2)cc1 ; c1ccc(-c2nc(-c3ccc(-c4cccc(-c5cccc6ncoc56)c4)cc3)nc(-c3ccc(C45CC6CC(CC(C6)C4)C5)cc3)n2)cc1 ; c1ccc(-c2nc(-c3ccc(-c4cccc(-c5cccc6ocnc56)c4)cc3)nc(-c3ccc(C45CC6CC(CC(C6)C4)C5)cc3)n2)cc1]

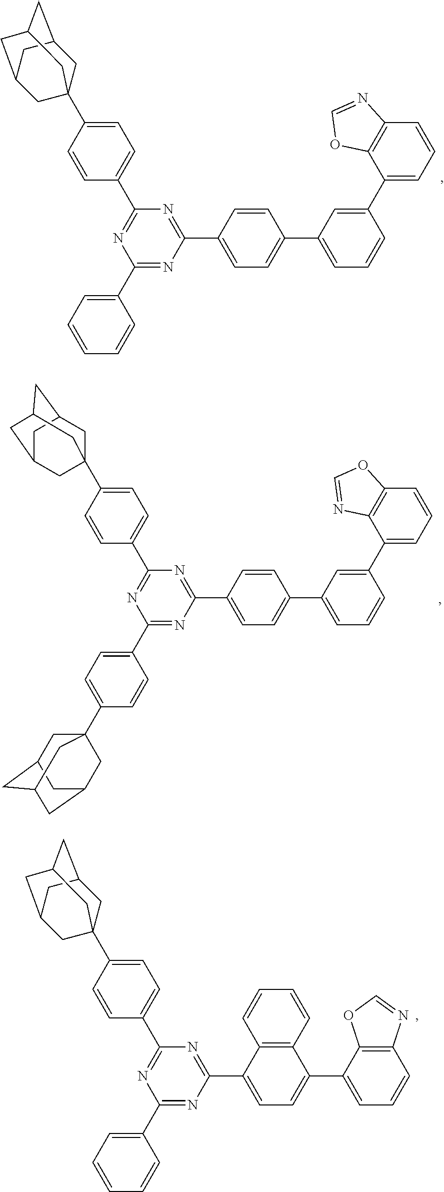

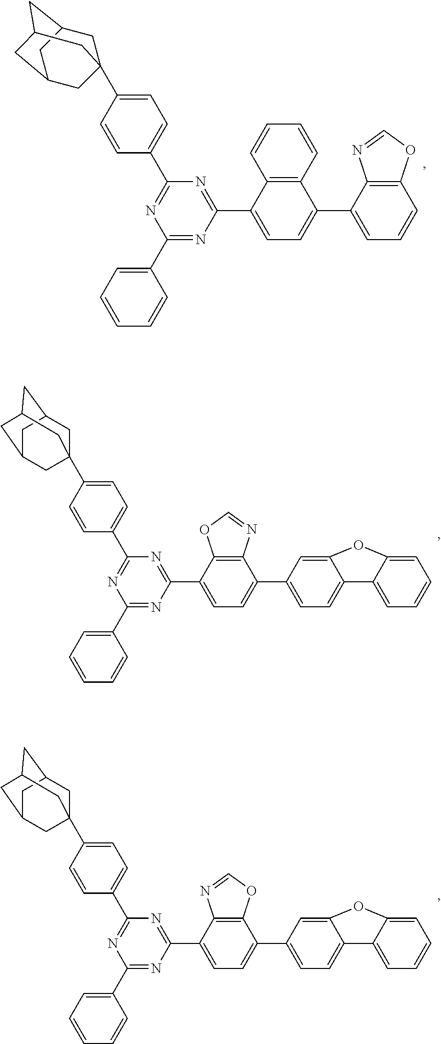 [SMILES: c1ccc(-c2nc(-c3ccc(C45CC6CC(CC(C6)C4)C5)cc3)nc(-c3ccc(-c4cccc5ocnc45)c4ccccc34)n2)cc1 ; c1ccc(-c2nc(-c3ccc(C45CC6CC(CC(C6)C4)C5)cc3)nc(-c3ccc(-c4ccc5c(c4)oc4ccccc45)c4ncoc34)n2)cc1 ; c1ccc(-c2nc(-c3ccc(C45CC6CC(CC(C6)C4)C5)cc3)nc(-c3ccc(-c4ccc5c(c4)oc4ccccc45)c4ocnc34)n2)cc1]

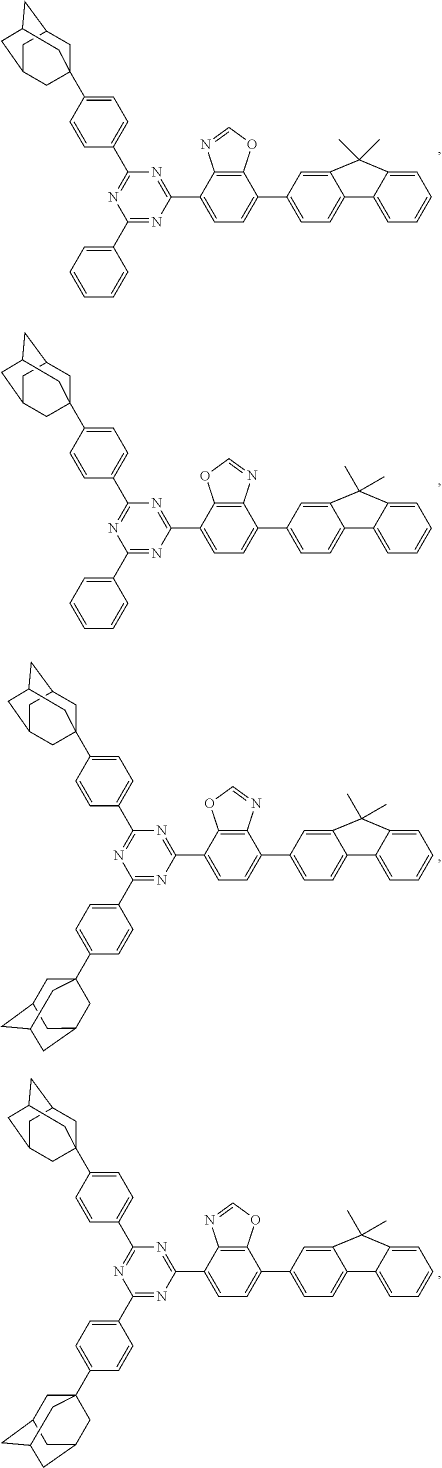

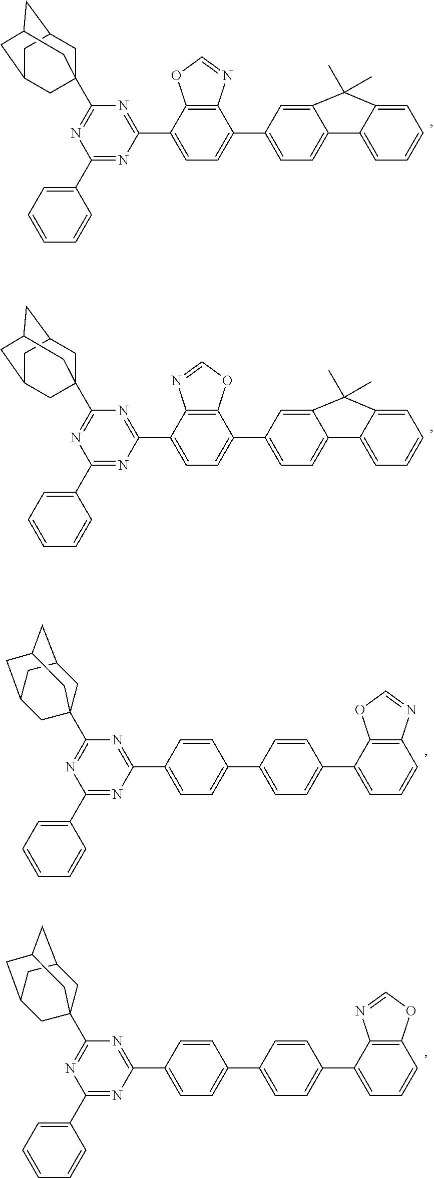 [SMILES: CC1(C)c2ccccc2-c2ccc(-c3ccc(-c4nc(-c5ccccc5)nc(C56CC7CC(CC(C7)C5)C6)n4)c4ocnc34)cc21 ; CC1(C)c2ccccc2-c2ccc(-c3ccc(-c4nc(-c5ccccc5)nc(C56CC7CC(CC(C7)C5)C6)n4)c4ncoc34)cc21 ; c1ccc(-c2nc(-c3ccc(-c4ccc(-c5cccc6ncoc56)cc4)cc3)nc(C34CC5CC(CC(C5)C3)C4)n2)cc1 ; c1ccc(-c2nc(-c3ccc(-c4ccc(-c5cccc6ocnc56)cc4)cc3)nc(C34CC5CC(CC(C5)C3)C4)n2)cc1]

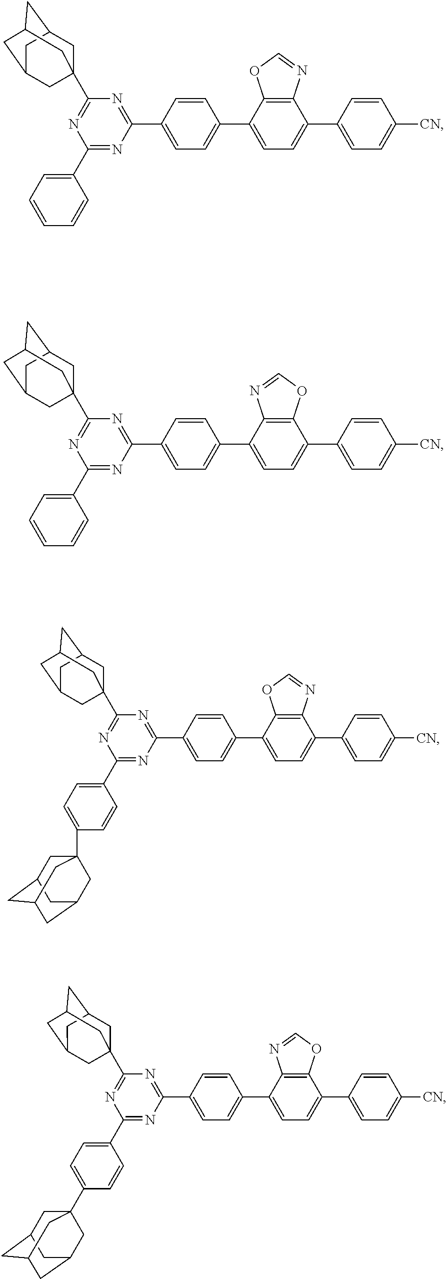 [SMILES: N#Cc1ccc(-c2ccc(-c3ccc(-c4nc(-c5ccccc5)nc(C56CC7CC(CC(C7)C5)C6)n4)cc3)c3ocnc23)cc1 ; N#Cc1ccc(-c2ccc(-c3ccc(-c4nc(-c5ccccc5)nc(C56CC7CC(CC(C7)C5)C6)n4)cc3)c3ncoc23)cc1 ; N#Cc1ccc(-c2ccc(-c3ccc(-c4nc(-c5ccc(C67CC8CC(CC(C8)C6)C7)cc5)nc(C56CC7CC(CC(C7)C5)C6)n4)cc3)c3ocnc23)cc1 ; N#Cc1ccc(-c2ccc(-c3ccc(-c4nc(-c5ccc(C67CC8CC(CC(C8)C6)C7)cc5)nc(C56CC7CC(CC(C7)C5)C6)n4)cc3)c3ncoc23)cc1]

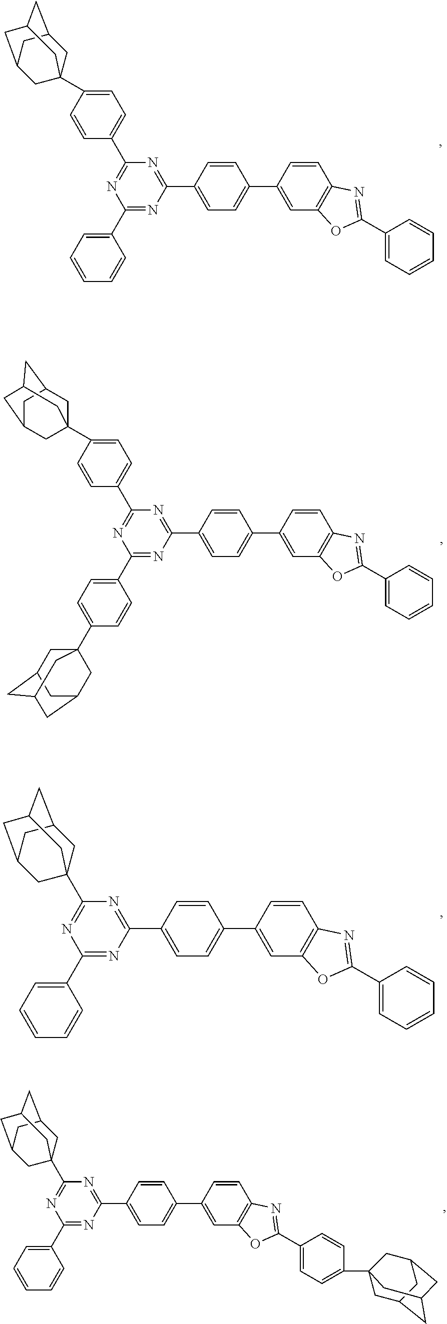 [SMILES: c1ccc(-c2nc(-c3ccc(-c4ccc5nc(-c6ccccc6)oc5c4)cc3)nc(-c3ccc(C45CC6CC(CC(C6)C4)C5)cc3)n2)cc1 ; c1ccc(-c2nc3ccc(-c4ccc(-c5nc(-c6ccc(C78CC9CC(CC(C9)C7)C8)cc6)nc(-c6ccc(C78CC9CC(CC(C9)C7)C8)cc6)n5)cc4)cc3o2)cc1 ; c1ccc(-c2nc(-c3ccc(-c4ccc5nc(-c6ccccc6)oc5c4)cc3)nc(C34CC5CC(CC(C5)C3)C4)n2)cc1 ; c1ccc(-c2nc(-c3ccc(-c4ccc5nc(-c6ccc(C78CC9CC(CC(C9)C7)C8)cc6)oc5c4)cc3)nc(C34CC5CC(CC(C5)C3)C4)n2)cc1]

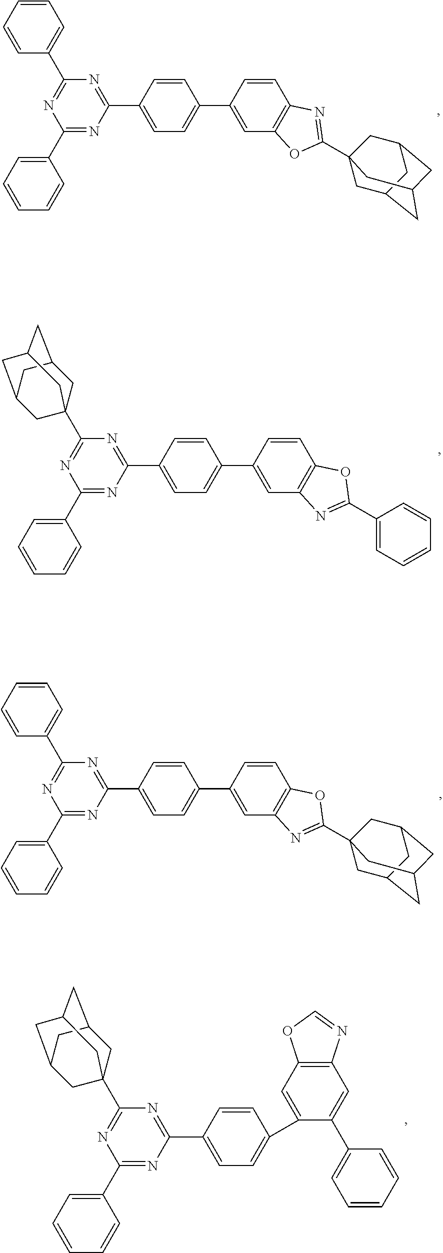 [SMILES: c1ccc(-c2nc(-c3ccccc3)nc(-c3ccc(-c4ccc5nc(C67CC8CC(CC(C8)C6)C7)oc5c4)cc3)n2)cc1 ; c1ccc(-c2nc(-c3ccc(-c4ccc5oc(-c6ccccc6)nc5c4)cc3)nc(C34CC5CC(CC(C5)C3)C4)n2)cc1 ; c1ccc(-c2nc(-c3ccccc3)nc(-c3ccc(-c4ccc5oc(C67CC8CC(CC(C8)C6)C7)nc5c4)cc3)n2)cc1 ; c1ccc(-c2nc(-c3ccc(-c4cc5ocnc5cc4-c4ccccc4)cc3)nc(C34CC5CC(CC(C5)C3)C4)n2)cc1]

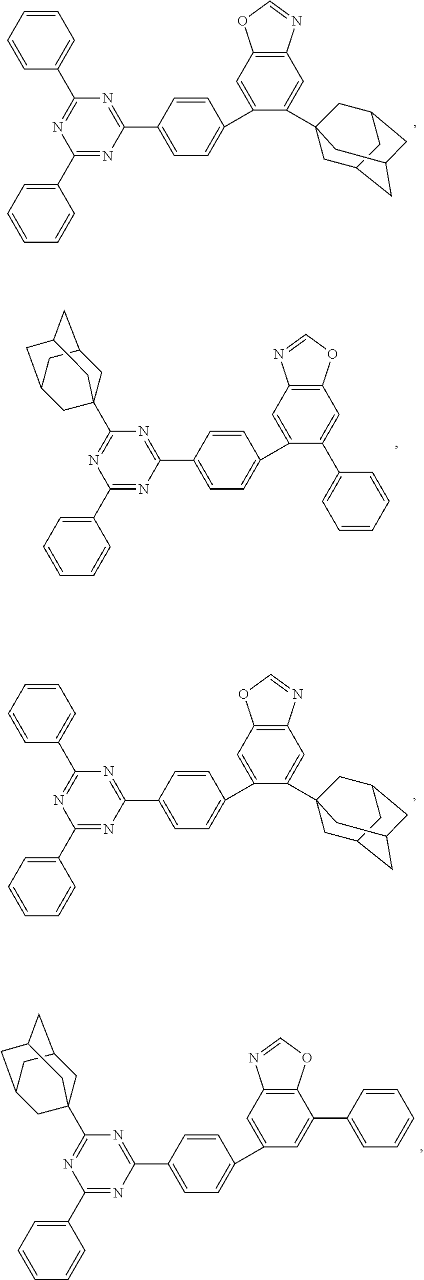 [SMILES: c1ccc(-c2nc(-c3ccccc3)nc(-c3ccc(-c4cc5ocnc5cc4C45CC6CC(CC(C6)C4)C5)cc3)n2)cc1 ; c1ccc(-c2nc(-c3ccc(-c4cc5ncoc5cc4-c4ccccc4)cc3)nc(C34CC5CC(CC(C5)C3)C4)n2)cc1 ; c1ccc(-c2nc(-c3ccccc3)nc(-c3ccc(-c4cc5ocnc5cc4C45CC6CC(CC(C6)C4)C5)cc3)n2)cc1 ; c1ccc(-c2nc(-c3ccc(-c4cc(-c5ccccc5)c5ocnc5c4)cc3)nc(C34CC5CC(CC(C5)C3)C4)n2)cc1]

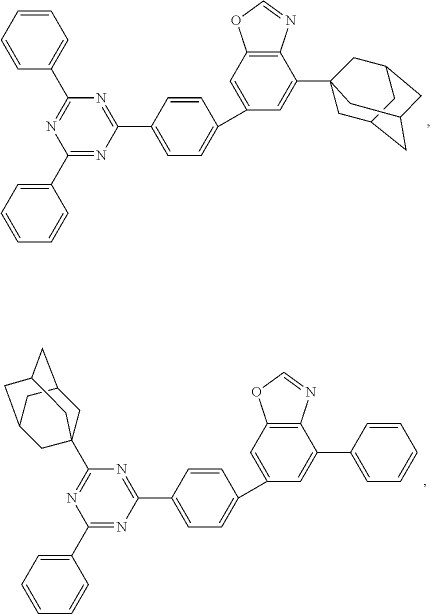 [SMILES: c1ccc(-c2nc(-c3ccccc3)nc(-c3ccc(-c4cc(C56CC7CC(CC(C7)C5)C6)c5ncoc5c4)cc3)n2)cc1 ; c1ccc(-c2nc(-c3ccc(-c4cc(-c5ccccc5)c5ncoc5c4)cc3)nc(C34CC5CC(CC(C5)C3)C4)n2)cc1]

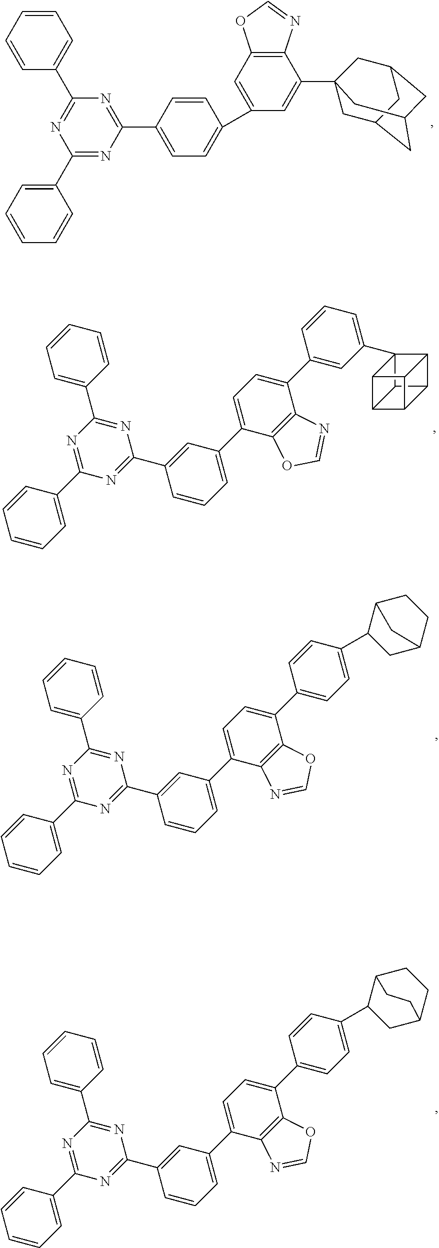 [SMILES: c1ccc(-c2nc(-c3ccccc3)nc(-c3ccc(-c4cc(C56CC7CC(CC(C7)C5)C6)c5ncoc5c4)cc3)n2)cc1 ; c1ccc(-c2nc(-c3ccccc3)nc(-c3cccc(-c4ccc(-c5cccc(C67C8C9C%10C8C6C%10C97)c5)c5ncoc45)c3)n2)cc1 ; c1ccc(-c2nc(-c3ccccc3)nc(-c3cccc(-c4ccc(-c5ccc(C6CC7CCC6C7)cc5)c5ocnc45)c3)n2)cc1 ; c1ccc(-c2nc(-c3ccccc3)nc(-c3cccc(-c4ccc(-c5ccc(C6CC7CCC6CC7)cc5)c5ocnc45)c3)n2)cc1]

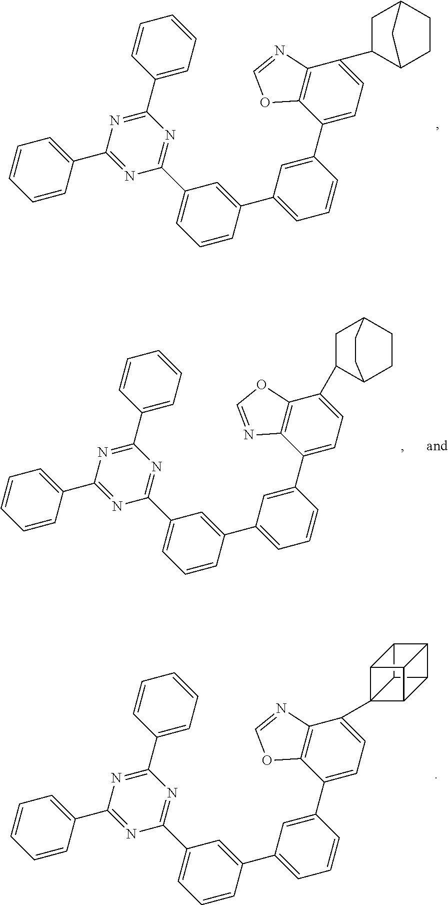 [SMILES: c1ccc(-c2nc(-c3ccccc3)nc(-c3cccc(-c4cccc(-c5ccc(C6CC7CCC6C7)c6ncoc56)c4)c3)n2)cc1 ; c1ccc(-c2nc(-c3ccccc3)nc(-c3cccc(-c4cccc(-c5ccc(C6CC7CCC6CC7)c6ocnc56)c4)c3)n2)cc1 ; c1ccc(-c2nc(-c3ccccc3)nc(-c3cccc(-c4cccc(-c5ccc(C67C8C9C%10C8C6C%10C97)c6ncoc56)c4)c3)n2)cc1]

9. The light emitting device according to claim 2, wherein in the material of the electron transport layer, L3 comprises a first benzene ring and a second benzene ring, and the first benzene ring is bonded to the second benzene ring; and
the first benzene ring is bonded to the heteroaromatic ring group comprising at least one nitrogen atom, and the heteroaromatic ring group comprising at least one nitrogen atom is located at a meta-position of the second benzene ring.

10. The light emitting device according to claim 9, wherein a chemical structural formula of the material of the electron transport layer includes any one of

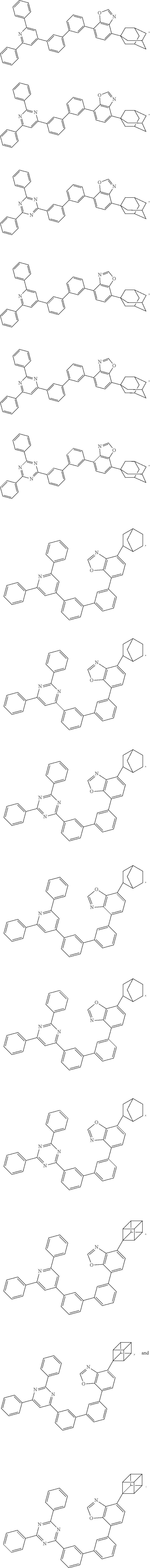

11. The light emitting device according to claim 2, wherein the material of the electron transport layer has cyano.

12. The light emitting device according to claim 11, wherein a chemical structural formula of a material of the electron transport layer having the cyano includes any one of

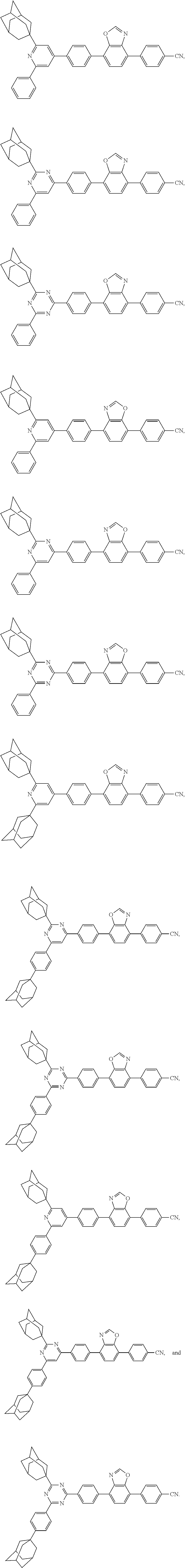

13. The light emitting device according to claim 2, wherein in the material of the electron transport layer, the adamantane is connected to the heteroaromatic ring group comprising at least one nitrogen atom by a phenyl group, and is located at an ortho-position of the nitrogen atom in the heteroaromatic ring group.

14. The light emitting device according to claim 13, wherein a chemical structural formula of the material of the electron transport layer includes any one of

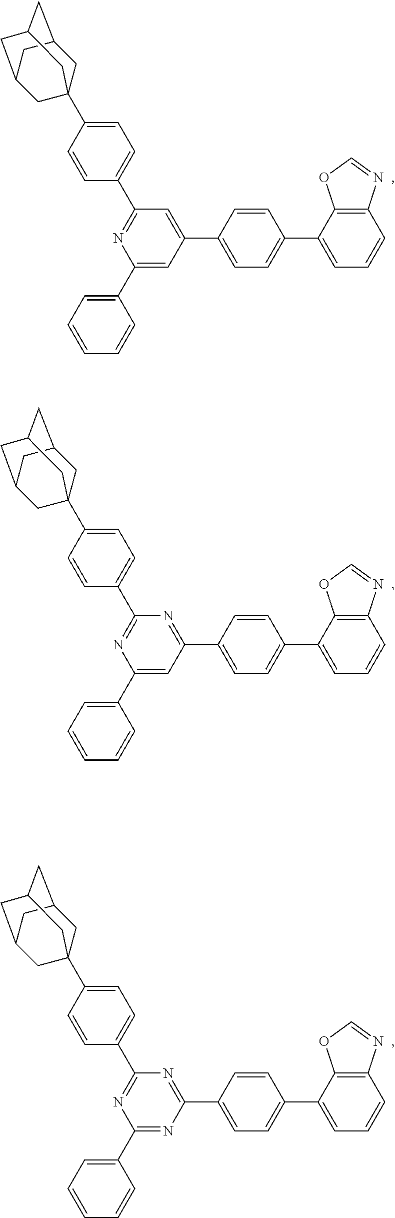 [SMILES: c1ccc(-c2cc(-c3ccc(-c4cccc5ncoc45)cc3)cc(-c3ccc(C45CC6CC(CC(C6)C4)C5)cc3)n2)cc1 ; c1ccc(-c2cc(-c3ccc(-c4cccc5ncoc45)cc3)nc(-c3ccc(C45CC6CC(CC(C6)C4)C5)cc3)n2)cc1 ; c1ccc(-c2nc(-c3ccc(-c4cccc5ncoc45)cc3)nc(-c3ccc(C45CC6CC(CC(C6)C4)C5)cc3)n2)cc1]

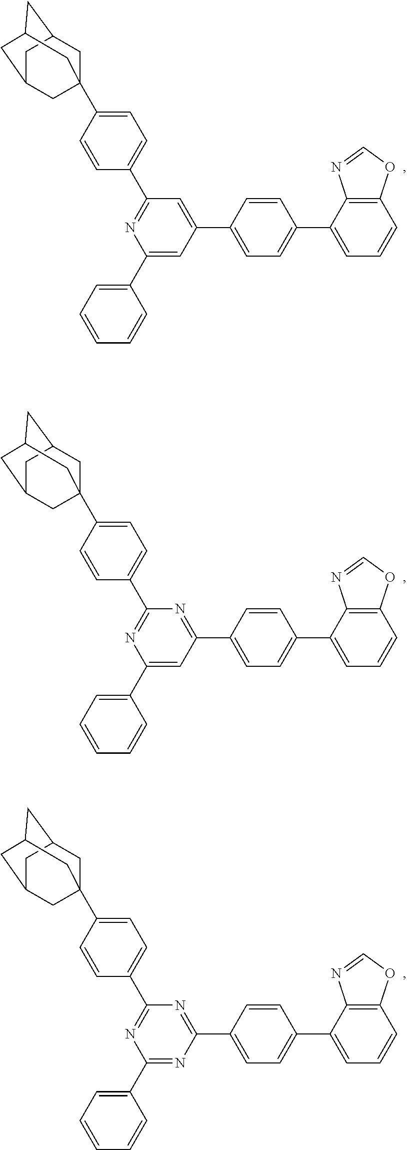 [SMILES: c1ccc(-c2cc(-c3ccc(-c4cccc5ocnc45)cc3)cc(-c3ccc(C45CC6CC(CC(C6)C4)C5)cc3)n2)cc1 ; c1ccc(-c2cc(-c3ccc(-c4cccc5ocnc45)cc3)nc(-c3ccc(C45CC6CC(CC(C6)C4)C5)cc3)n2)cc1 ; c1ccc(-c2nc(-c3ccc(-c4cccc5ocnc45)cc3)nc(-c3ccc(C45CC6CC(CC(C6)C4)C5)cc3)n2)cc1]

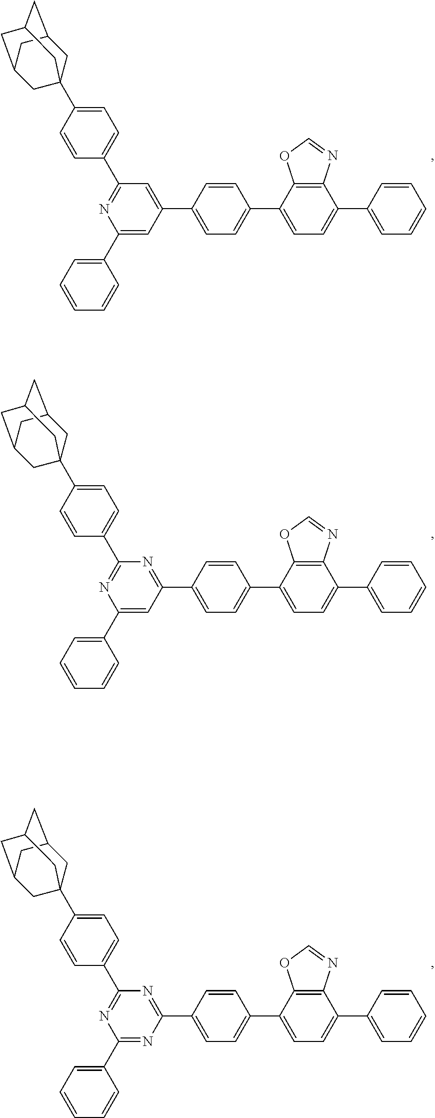 [SMILES: c1ccc(-c2cc(-c3ccc(-c4ccc(-c5ccccc5)c5ncoc45)cc3)cc(-c3ccc(C45CC6CC(CC(C6)C4)C5)cc3)n2)cc1 ; c1ccc(-c2cc(-c3ccc(-c4ccc(-c5ccccc5)c5ncoc45)cc3)nc(-c3ccc(C45CC6CC(CC(C6)C4)C5)cc3)n2)cc1 ; c1ccc(-c2nc(-c3ccc(-c4ccc(-c5ccccc5)c5ncoc45)cc3)nc(-c3ccc(C45CC6CC(CC(C6)C4)C5)cc3)n2)cc1]

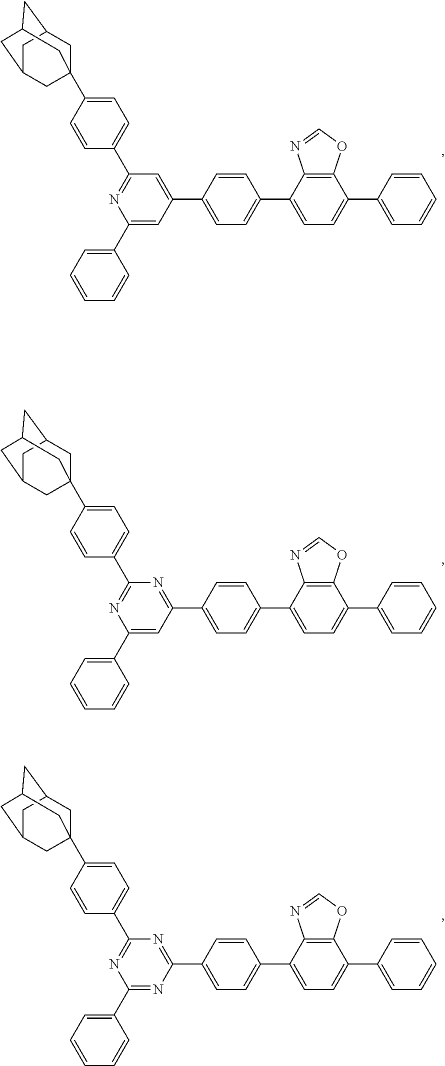 [SMILES: c1ccc(-c2cc(-c3ccc(-c4ccc(-c5ccccc5)c5ocnc45)cc3)cc(-c3ccc(C45CC6CC(CC(C6)C4)C5)cc3)n2)cc1 ; c1ccc(-c2cc(-c3ccc(-c4ccc(-c5ccccc5)c5ocnc45)cc3)nc(-c3ccc(C45CC6CC(CC(C6)C4)C5)cc3)n2)cc1 ; c1ccc(-c2nc(-c3ccc(-c4ccc(-c5ccccc5)c5ocnc45)cc3)nc(-c3ccc(C45CC6CC(CC(C6)C4)C5)cc3)n2)cc1]

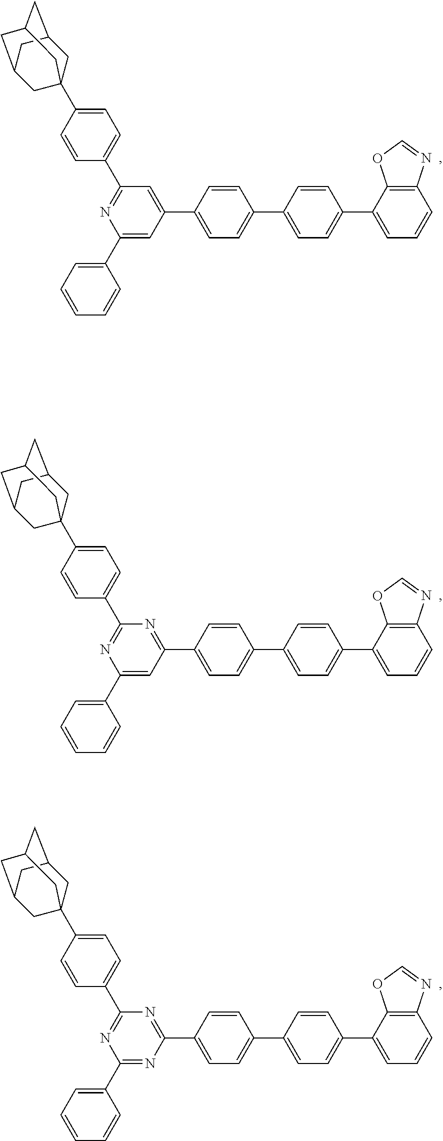 [SMILES: c1ccc(-c2cc(-c3ccc(-c4ccc(-c5cccc6ncoc56)cc4)cc3)cc(-c3ccc(C45CC6CC(CC(C6)C4)C5)cc3)n2)cc1 ; c1ccc(-c2cc(-c3ccc(-c4ccc(-c5cccc6ncoc56)cc4)cc3)nc(-c3ccc(C45CC6CC(CC(C6)C4)C5)cc3)n2)cc1 ; c1ccc(-c2nc(-c3ccc(-c4ccc(-c5cccc6ncoc56)cc4)cc3)nc(-c3ccc(C45CC6CC(CC(C6)C4)C5)cc3)n2)cc1]

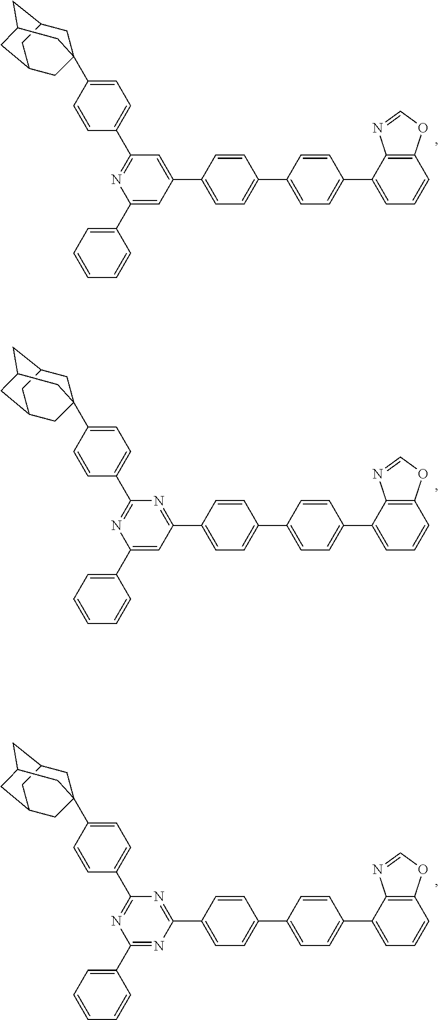 [SMILES: c1ccc(-c2cc(-c3ccc(-c4ccc(-c5cccc6ocnc56)cc4)cc3)cc(-c3ccc(C45CC6CC(CC(C6)C4)C5)cc3)n2)cc1 ; c1ccc(-c2cc(-c3ccc(-c4ccc(-c5cccc6ocnc56)cc4)cc3)nc(-c3ccc(C45CC6CC(CC(C6)C4)C5)cc3)n2)cc1 ; c1ccc(-c2nc(-c3ccc(-c4ccc(-c5cccc6ocnc56)cc4)cc3)nc(-c3ccc(C45CC6CC(CC(C6)C4)C5)cc3)n2)cc1]

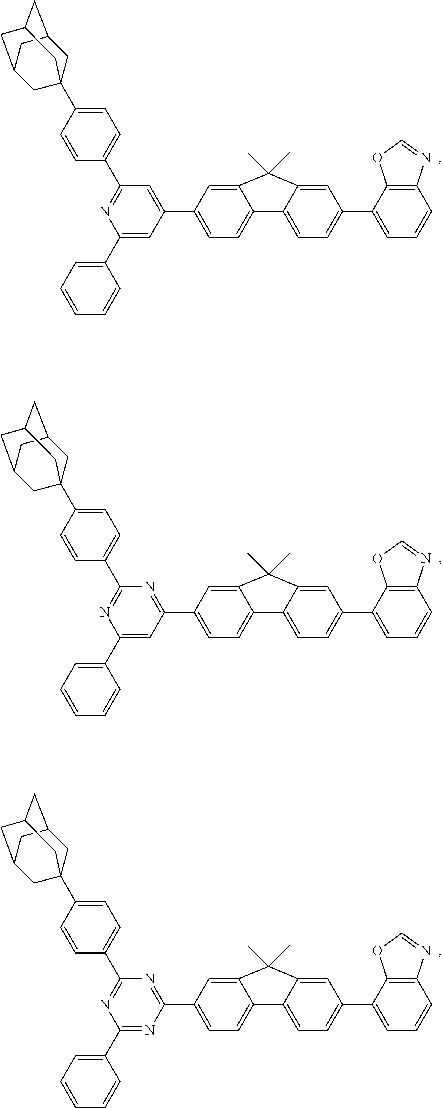 [SMILES: CC1(C)c2cc(-c3cc(-c4ccccc4)nc(-c4ccc(C56CC7CC(CC(C7)C5)C6)cc4)c3)ccc2-c2ccc(-c3cccc4ncoc34)cc21 ; CC1(C)c2cc(-c3cc(-c4ccccc4)nc(-c4ccc(C56CC7CC(CC(C7)C5)C6)cc4)n3)ccc2-c2ccc(-c3cccc4ncoc34)cc21 ; CC1(C)c2cc(-c3nc(-c4ccccc4)nc(-c4ccc(C56CC7CC(CC(C7)C5)C6)cc4)n3)ccc2-c2ccc(-c3cccc4ncoc34)cc21]

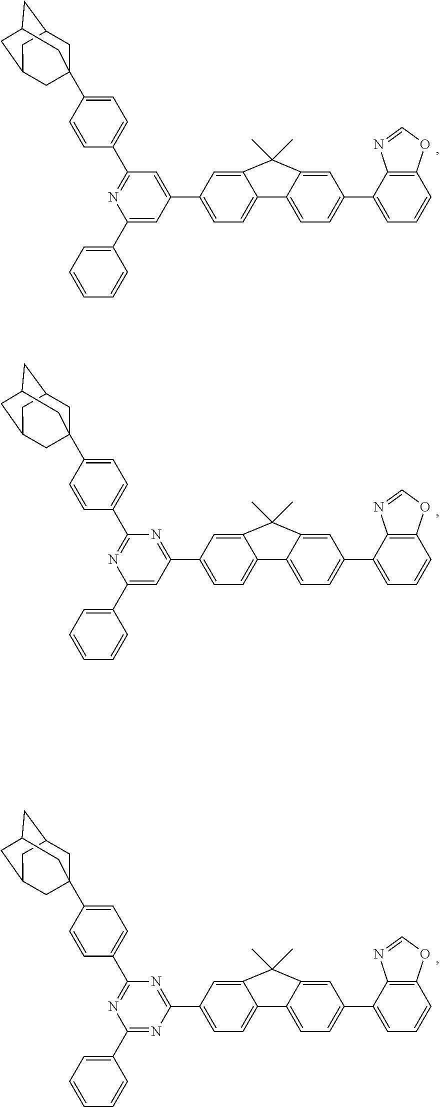 [SMILES: CC1(C)c2cc(-c3cc(-c4ccccc4)nc(-c4ccc(C56CC7CC(CC(C7)C5)C6)cc4)c3)ccc2-c2ccc(-c3cccc4ocnc34)cc21 ; CC1(C)c2cc(-c3cc(-c4ccccc4)nc(-c4ccc(C56CC7CC(CC(C7)C5)C6)cc4)n3)ccc2-c2ccc(-c3cccc4ocnc34)cc21 ; CC1(C)c2cc(-c3nc(-c4ccccc4)nc(-c4ccc(C56CC7CC(CC(C7)C5)C6)cc4)n3)ccc2-c2ccc(-c3cccc4ocnc34)cc21]

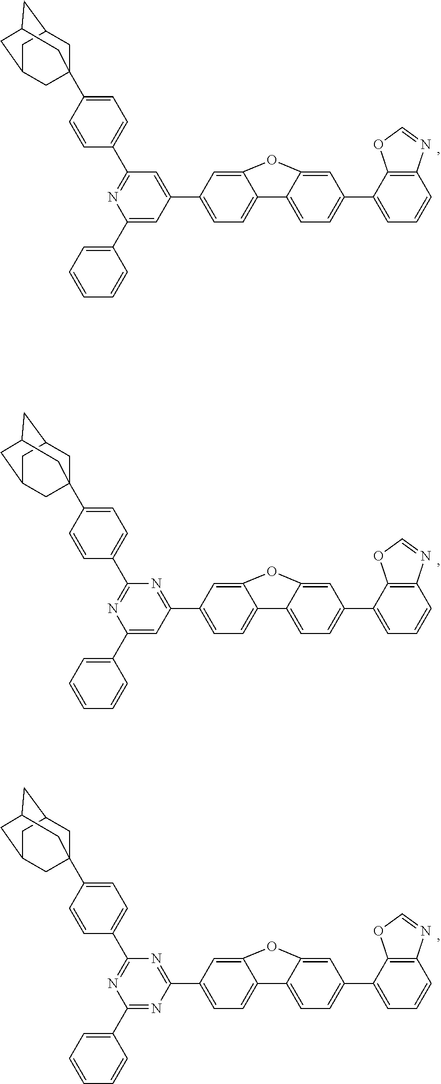 [SMILES: c1ccc(-c2cc(-c3ccc4c(c3)oc3cc(-c5cccc6ncoc56)ccc34)cc(-c3ccc(C45CC6CC(CC(C6)C4)C5)cc3)n2)cc1 ; c1ccc(-c2cc(-c3ccc4c(c3)oc3cc(-c5cccc6ncoc56)ccc34)nc(-c3ccc(C45CC6CC(CC(C6)C4)C5)cc3)n2)cc1 ; c1ccc(-c2nc(-c3ccc(C45CC6CC(CC(C6)C4)C5)cc3)nc(-c3ccc4c(c3)oc3cc(-c5cccc6ncoc56)ccc34)n2)cc1]

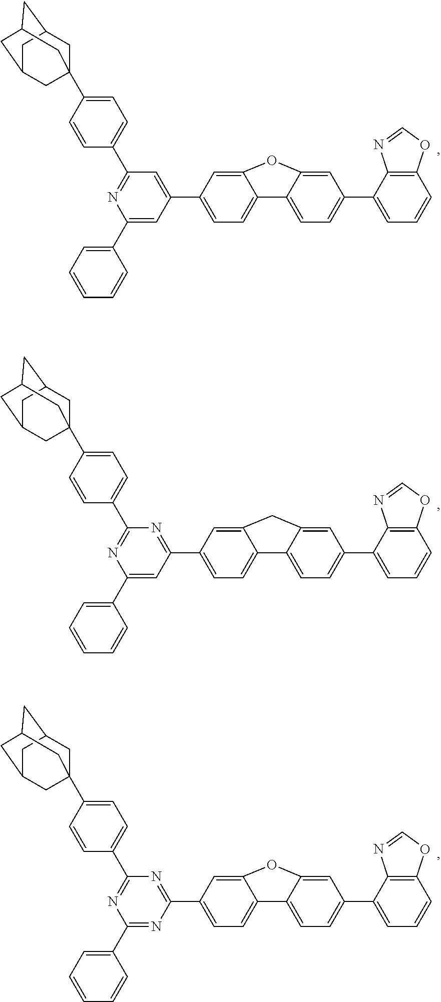 [SMILES: c1ccc(-c2cc(-c3ccc4c(c3)oc3cc(-c5cccc6ocnc56)ccc34)cc(-c3ccc(C45CC6CC(CC(C6)C4)C5)cc3)n2)cc1 ; c1ccc(-c2cc(-c3ccc4c(c3)Cc3cc(-c5cccc6ocnc56)ccc3-4)nc(-c3ccc(C45CC6CC(CC(C6)C4)C5)cc3)n2)cc1 ; c1ccc(-c2nc(-c3ccc(C45CC6CC(CC(C6)C4)C5)cc3)nc(-c3ccc4c(c3)oc3cc(-c5cccc6ocnc56)ccc34)n2)cc1]

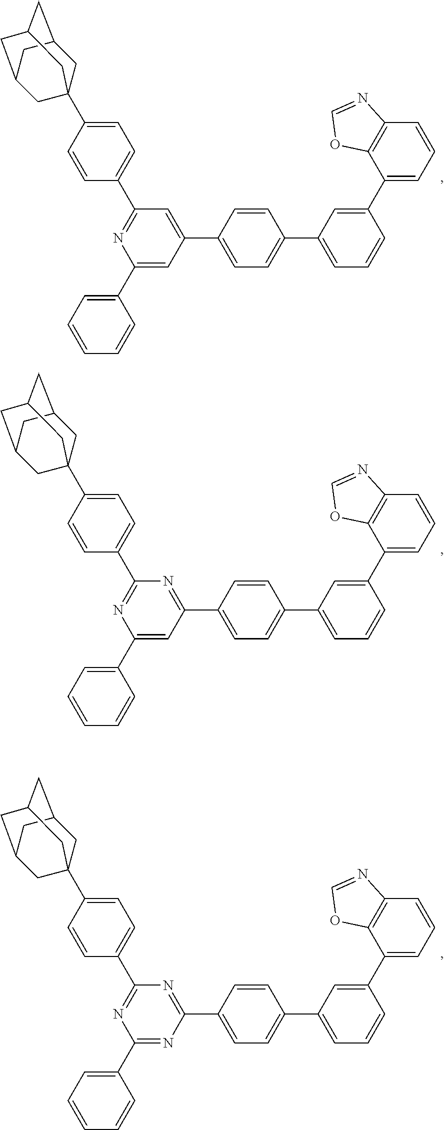 [SMILES: c1ccc(-c2cc(-c3ccc(-c4cccc(-c5cccc6ncoc56)c4)cc3)cc(-c3ccc(C45CC6CC(CC(C6)C4)C5)cc3)n2)cc1 ; c1ccc(-c2cc(-c3ccc(-c4cccc(-c5cccc6ncoc56)c4)cc3)nc(-c3ccc(C45CC6CC(CC(C6)C4)C5)cc3)n2)cc1 ; c1ccc(-c2nc(-c3ccc(-c4cccc(-c5cccc6ncoc56)c4)cc3)nc(-c3ccc(C45CC6CC(CC(C6)C4)C5)cc3)n2)cc1]

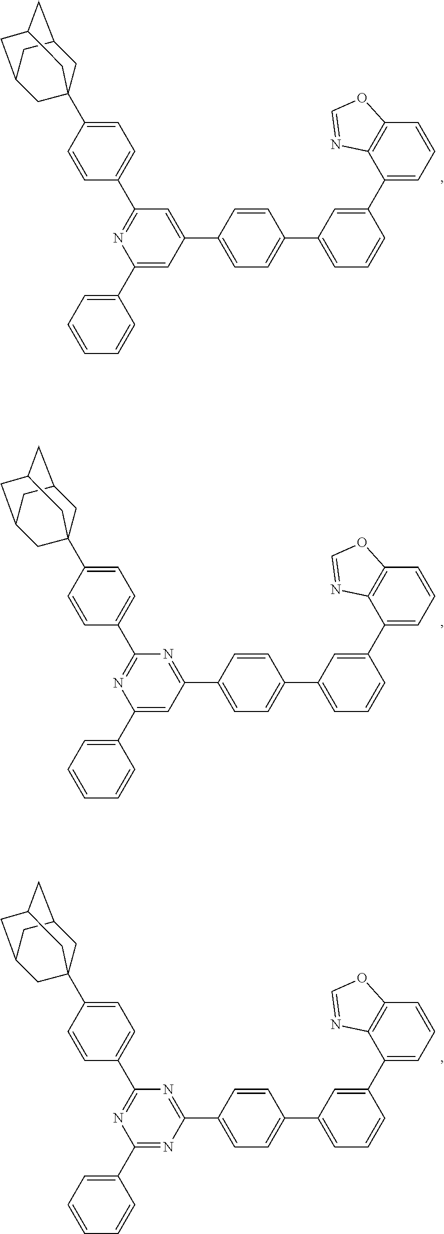 [SMILES: c1ccc(-c2cc(-c3ccc(-c4cccc(-c5cccc6ocnc56)c4)cc3)cc(-c3ccc(C45CC6CC(CC(C6)C4)C5)cc3)n2)cc1 ; c1ccc(-c2cc(-c3ccc(-c4cccc(-c5cccc6ocnc56)c4)cc3)nc(-c3ccc(C45CC6CC(CC(C6)C4)C5)cc3)n2)cc1 ; c1ccc(-c2nc(-c3ccc(-c4cccc(-c5cccc6ocnc56)c4)cc3)nc(-c3ccc(C45CC6CC(CC(C6)C4)C5)cc3)n2)cc1]

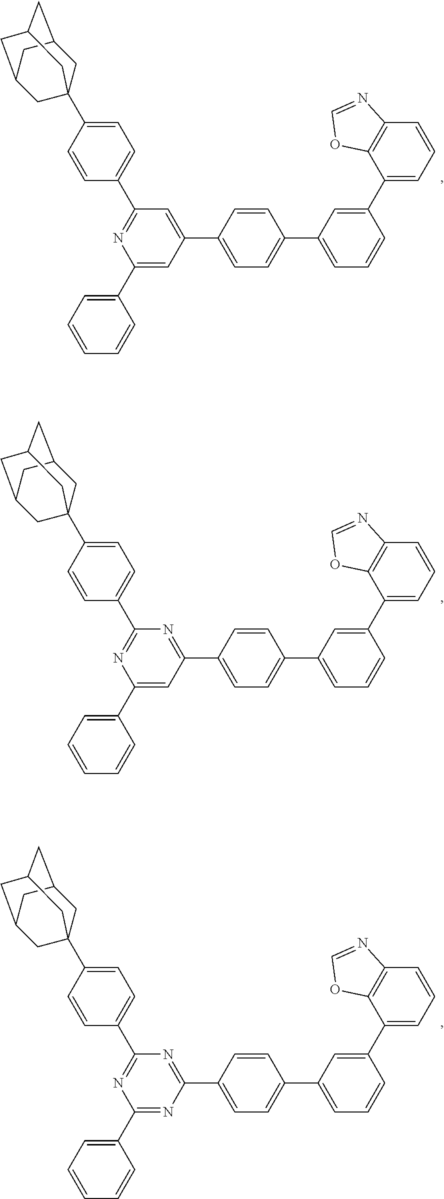 [SMILES: c1ccc(-c2cc(-c3ccc(-c4cccc(-c5cccc6ncoc56)c4)cc3)cc(-c3ccc(C45CC6CC(CC(C6)C4)C5)cc3)n2)cc1 ; c1ccc(-c2cc(-c3ccc(-c4cccc(-c5cccc6ncoc56)c4)cc3)nc(-c3ccc(C45CC6CC(CC(C6)C4)C5)cc3)n2)cc1 ; c1ccc(-c2nc(-c3ccc(-c4cccc(-c5cccc6ncoc56)c4)cc3)nc(-c3ccc(C45CC6CC(CC(C6)C4)C5)cc3)n2)cc1]

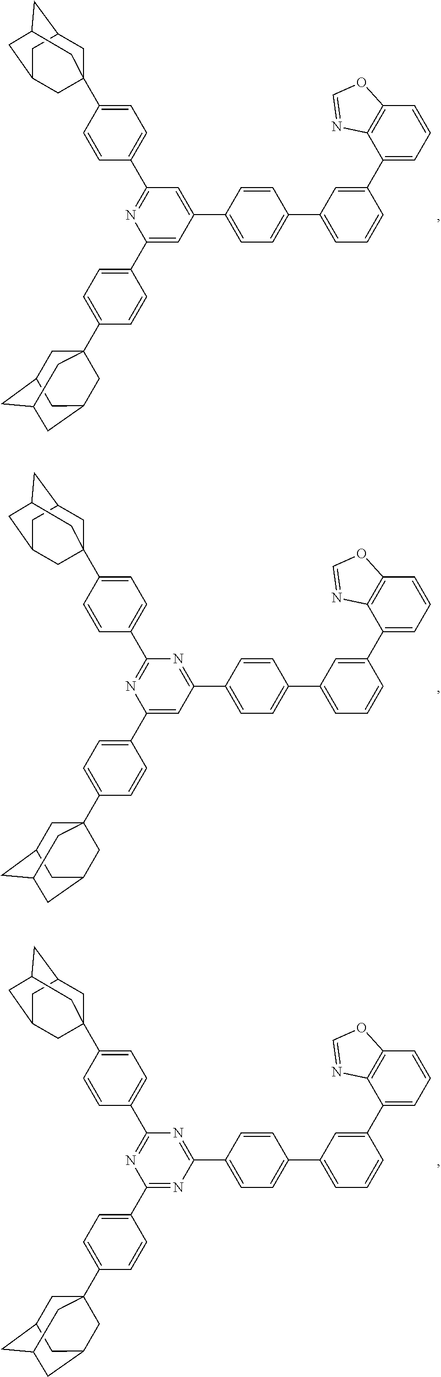 [SMILES: c1cc(-c2ccc(-c3cc(-c4ccc(C56CC7CC(CC(C7)C5)C6)cc4)nc(-c4ccc(C56CC7CC(CC(C7)C5)C6)cc4)c3)cc2)cc(-c2cccc3ocnc23)c1 ; c1cc(-c2ccc(-c3cc(-c4ccc(C56CC7CC(CC(C7)C5)C6)cc4)nc(-c4ccc(C56CC7CC(CC(C7)C5)C6)cc4)n3)cc2)cc(-c2cccc3ocnc23)c1 ; c1cc(-c2ccc(-c3nc(-c4ccc(C56CC7CC(CC(C7)C5)C6)cc4)nc(-c4ccc(C56CC7CC(CC(C7)C5)C6)cc4)n3)cc2)cc(-c2cccc3ocnc23)c1]

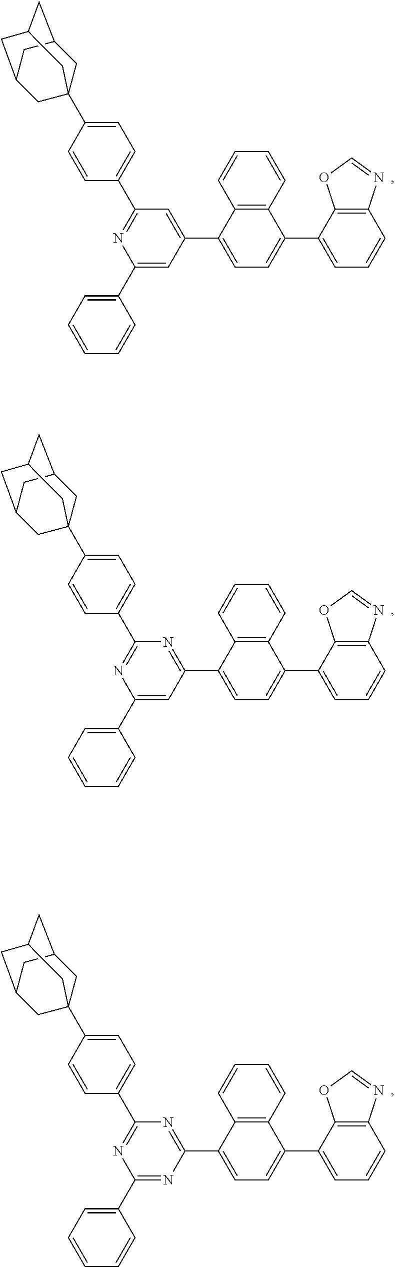 [SMILES: c1ccc(-c2cc(-c3ccc(-c4cccc5ncoc45)c4ccccc34)cc(-c3ccc(C45CC6CC(CC(C6)C4)C5)cc3)n2)cc1 ; c1ccc(-c2cc(-c3ccc(-c4cccc5ncoc45)c4ccccc34)nc(-c3ccc(C45CC6CC(CC(C6)C4)C5)cc3)n2)cc1 ; c1ccc(-c2nc(-c3ccc(C45CC6CC(CC(C6)C4)C5)cc3)nc(-c3ccc(-c4cccc5ncoc45)c4ccccc34)n2)cc1]

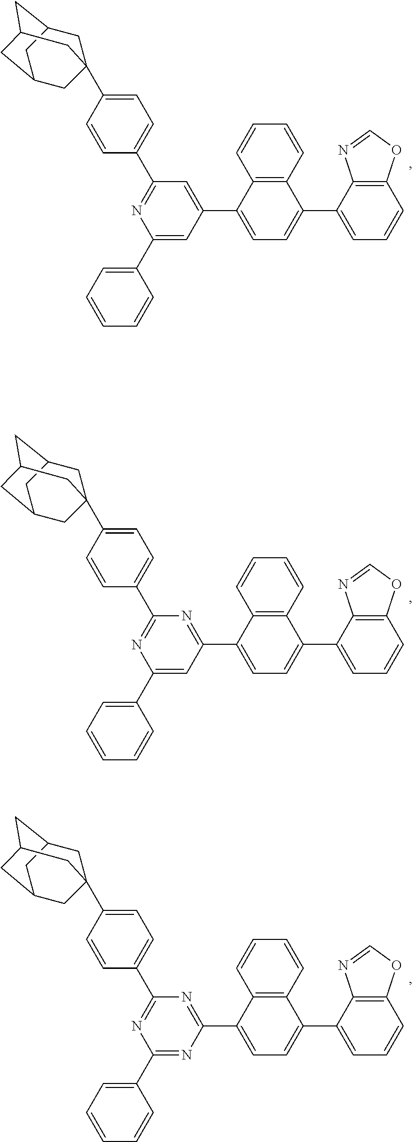 [SMILES: c1ccc(-c2cc(-c3ccc(-c4cccc5ocnc45)c4ccccc34)cc(-c3ccc(C45CC6CC(CC(C6)C4)C5)cc3)n2)cc1 ; c1ccc(-c2cc(-c3ccc(-c4cccc5ocnc45)c4ccccc34)nc(-c3ccc(C45CC6CC(CC(C6)C4)C5)cc3)n2)cc1 ; c1ccc(-c2nc(-c3ccc(C45CC6CC(CC(C6)C4)C5)cc3)nc(-c3ccc(-c4cccc5ocnc45)c4ccccc34)n2)cc1]

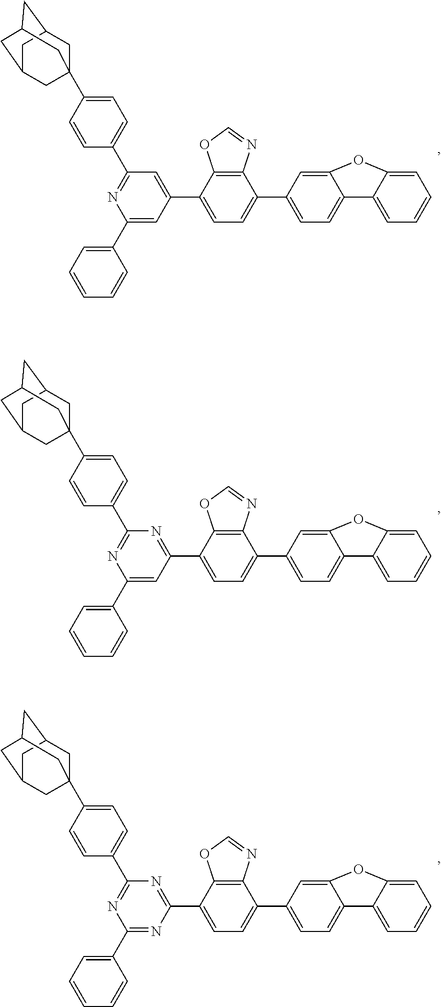 [SMILES: c1ccc(-c2cc(-c3ccc(-c4ccc5c(c4)oc4ccccc45)c4ncoc34)cc(-c3ccc(C45CC6CC(CC(C6)C4)C5)cc3)n2)cc1 ; c1ccc(-c2cc(-c3ccc(-c4ccc5c(c4)oc4ccccc45)c4ncoc34)nc(-c3ccc(C45CC6CC(CC(C6)C4)C5)cc3)n2)cc1 ; c1ccc(-c2nc(-c3ccc(C45CC6CC(CC(C6)C4)C5)cc3)nc(-c3ccc(-c4ccc5c(c4)oc4ccccc45)c4ncoc34)n2)cc1]

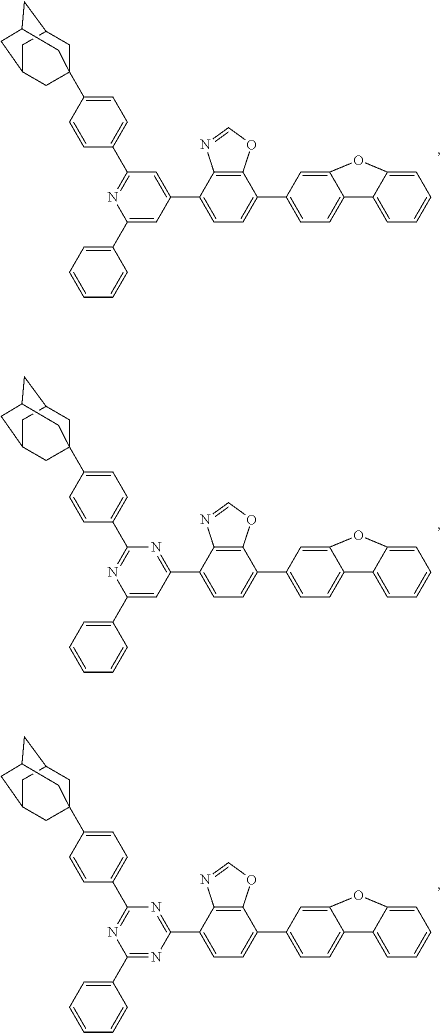 [SMILES: c1ccc(-c2cc(-c3ccc(-c4ccc5c(c4)oc4ccccc45)c4ocnc34)cc(-c3ccc(C45CC6CC(CC(C6)C4)C5)cc3)n2)cc1 ; c1ccc(-c2cc(-c3ccc(-c4ccc5c(c4)oc4ccccc45)c4ocnc34)nc(-c3ccc(C45CC6CC(CC(C6)C4)C5)cc3)n2)cc1 ; c1ccc(-c2nc(-c3ccc(C45CC6CC(CC(C6)C4)C5)cc3)nc(-c3ccc(-c4ccc5c(c4)oc4ccccc45)c4ocnc34)n2)cc1]

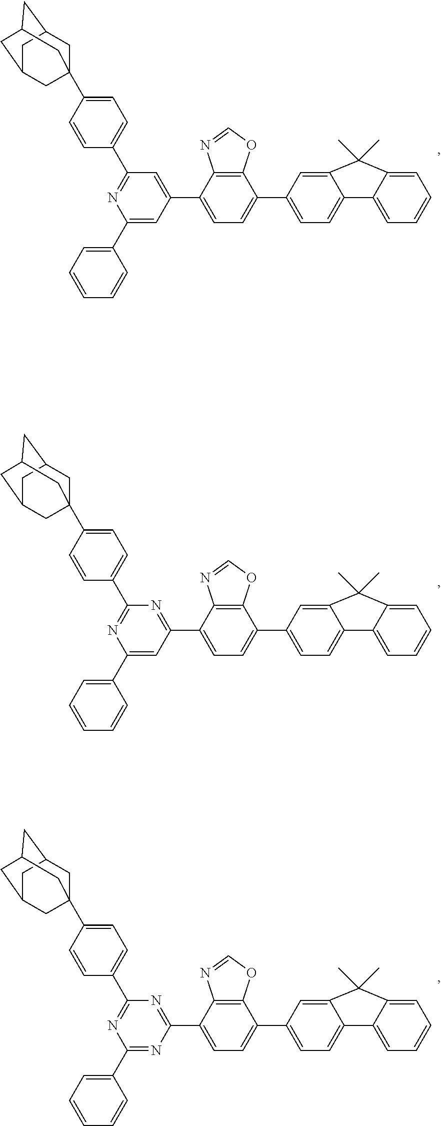 [SMILES: CC1(C)c2ccccc2-c2ccc(-c3ccc(-c4cc(-c5ccccc5)nc(-c5ccc(C67CC8CC(CC(C8)C6)C7)cc5)c4)c4ncoc34)cc21 ; CC1(C)c2ccccc2-c2ccc(-c3ccc(-c4cc(-c5ccccc5)nc(-c5ccc(C67CC8CC(CC(C8)C6)C7)cc5)n4)c4ncoc34)cc21 ; CC1(C)c2ccccc2-c2ccc(-c3ccc(-c4nc(-c5ccccc5)nc(-c5ccc(C67CC8CC(CC(C8)C6)C7)cc5)n4)c4ncoc34)cc21]

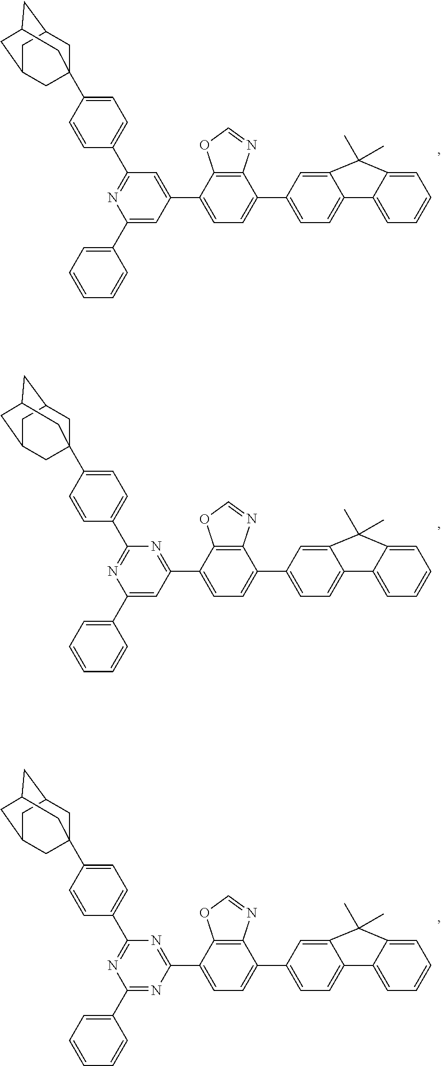 [SMILES: CC1(C)c2ccccc2-c2ccc(-c3ccc(-c4cc(-c5ccccc5)nc(-c5ccc(C67CC8CC(CC(C8)C6)C7)cc5)c4)c4ocnc34)cc21 ; CC1(C)c2ccccc2-c2ccc(-c3ccc(-c4cc(-c5ccccc5)nc(-c5ccc(C67CC8CC(CC(C8)C6)C7)cc5)n4)c4ocnc34)cc21 ; CC1(C)c2ccccc2-c2ccc(-c3ccc(-c4nc(-c5ccccc5)nc(-c5ccc(C67CC8CC(CC(C8)C6)C7)cc5)n4)c4ocnc34)cc21]

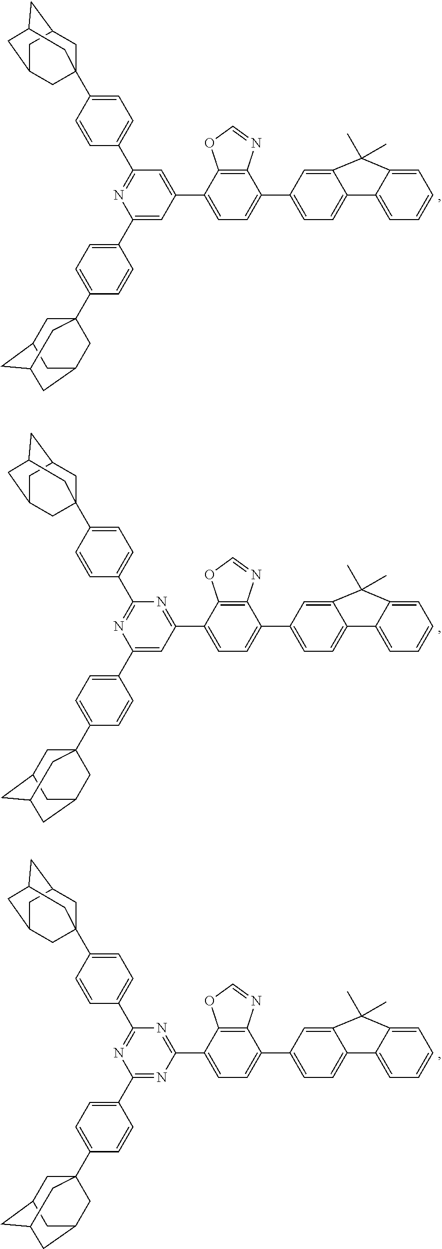

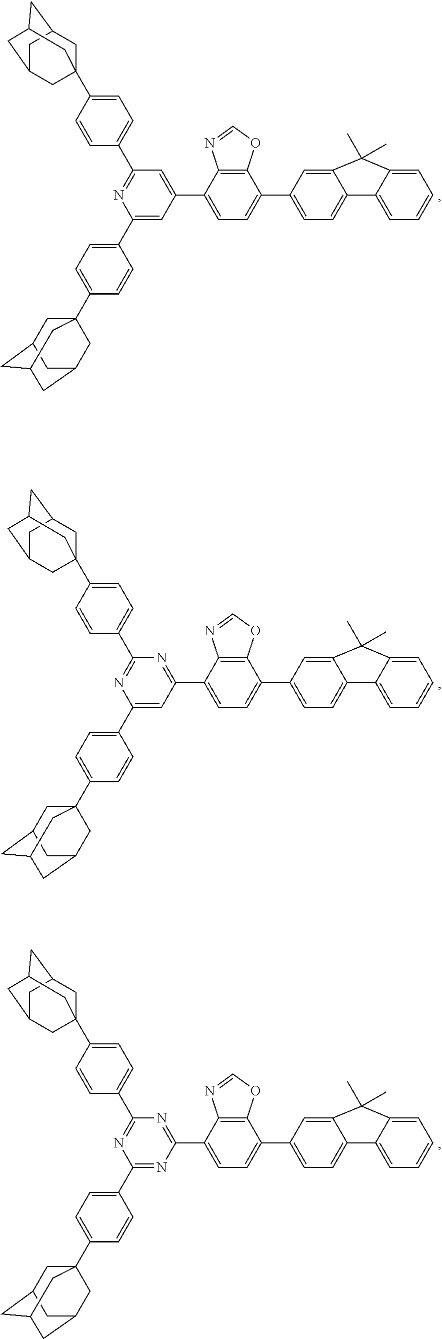 [SMILES: CC1(C)c2ccccc2-c2ccc(-c3ccc(-c4cc(-c5ccc(C67CC8CC(CC(C8)C6)C7)cc5)nc(-c5ccc(C67CC8CC(CC(C8)C6)C7)cc5)c4)c4ncoc34)cc21 ; CC1(C)c2ccccc2-c2ccc(-c3ccc(-c4cc(-c5ccc(C67CC8CC(CC(C8)C6)C7)cc5)nc(-c5ccc(C67CC8CC(CC(C8)C6)C7)cc5)n4)c4ncoc34)cc21 ; CC1(C)c2ccccc2-c2ccc(-c3ccc(-c4nc(-c5ccc(C67CC8CC(CC(C8)C6)C7)cc5)nc(-c5ccc(C67CC8CC(CC(C8)C6)C7)cc5)n4)c4ncoc34)cc21]

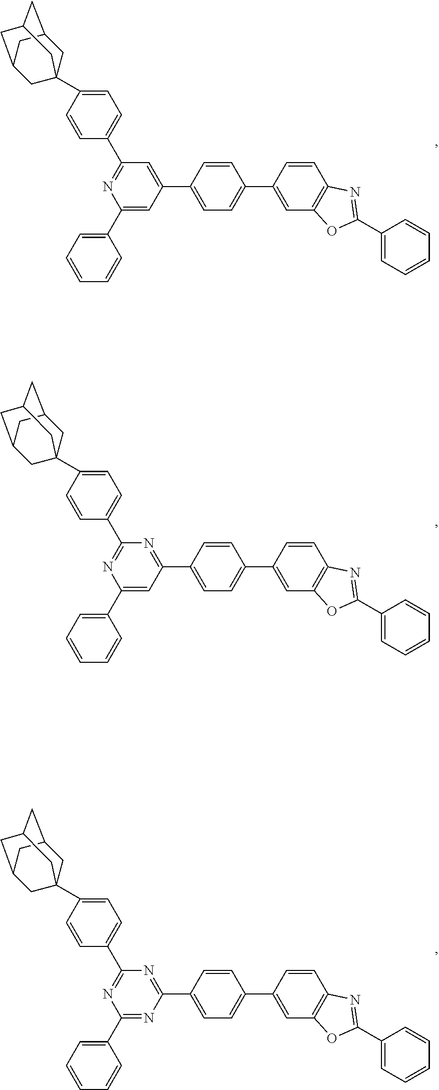 [SMILES: c1ccc(-c2cc(-c3ccc(-c4ccc5nc(-c6ccccc6)oc5c4)cc3)cc(-c3ccc(C45CC6CC(CC(C6)C4)C5)cc3)n2)cc1 ; c1ccc(-c2cc(-c3ccc(-c4ccc5nc(-c6ccccc6)oc5c4)cc3)nc(-c3ccc(C45CC6CC(CC(C6)C4)C5)cc3)n2)cc1 ; c1ccc(-c2nc(-c3ccc(-c4ccc5nc(-c6ccccc6)oc5c4)cc3)nc(-c3ccc(C45CC6CC(CC(C6)C4)C5)cc3)n2)cc1]

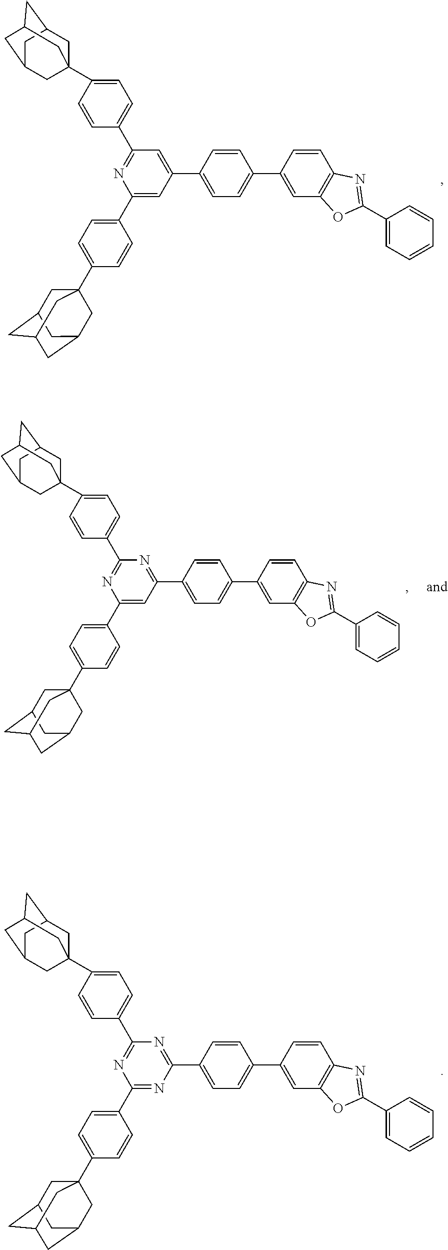 [SMILES: c1ccc(-c2nc3ccc(-c4ccc(-c5cc(-c6ccc(C78CC9CC(CC(C9)C7)C8)cc6)nc(-c6ccc(C78CC9CC(CC(C9)C7)C8)cc6)c5)cc4)cc3o2)cc1 ; c1ccc(-c2nc3ccc(-c4ccc(-c5cc(-c6ccc(C78CC9CC(CC(C9)C7)C8)cc6)nc(-c6ccc(C78CC9CC(CC(C9)C7)C8)cc6)n5)cc4)cc3o2)cc1 ; c1ccc(-c2nc3ccc(-c4ccc(-c5nc(-c6ccc(C78CC9CC(CC(C9)C7)C8)cc6)nc(-c6ccc(C78CC9CC(CC(C9)C7)C8)cc6)n5)cc4)cc3o2)cc1]

15. The light emitting device according to claim 2, wherein a chemical structural formula of the material of the electron transport layer includes any one of

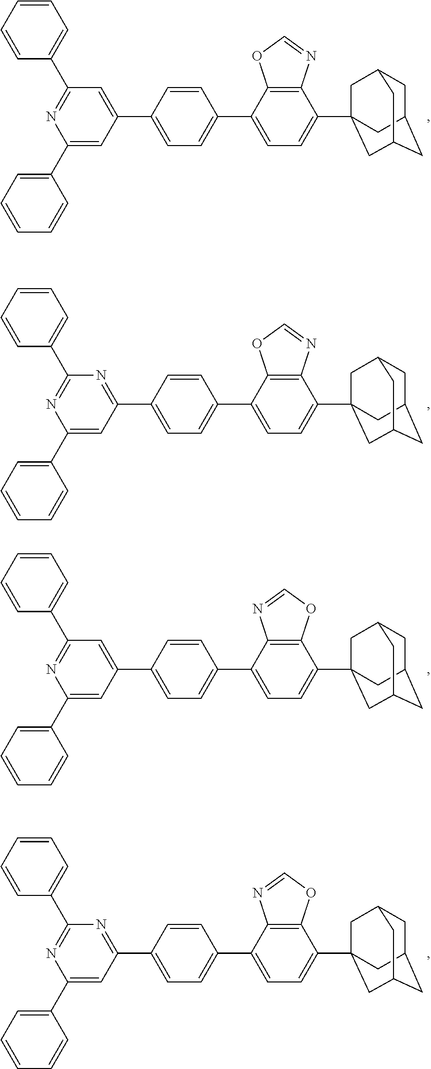

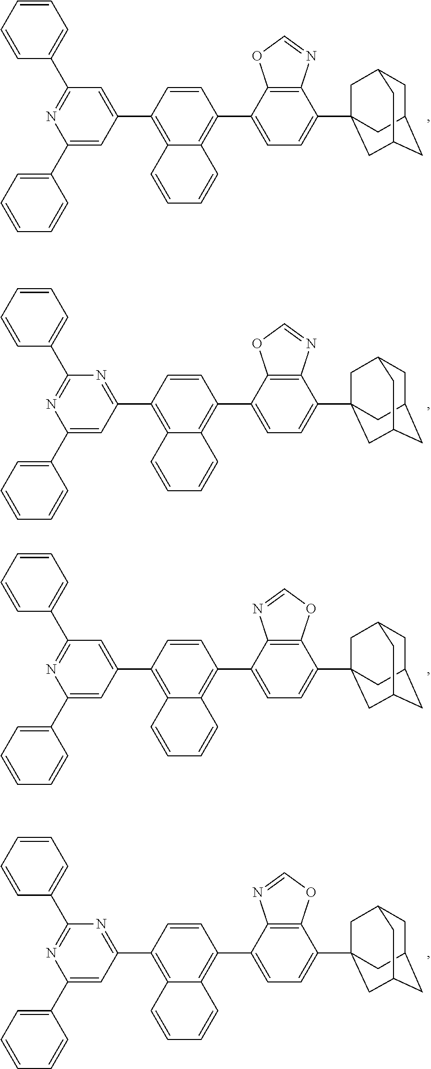

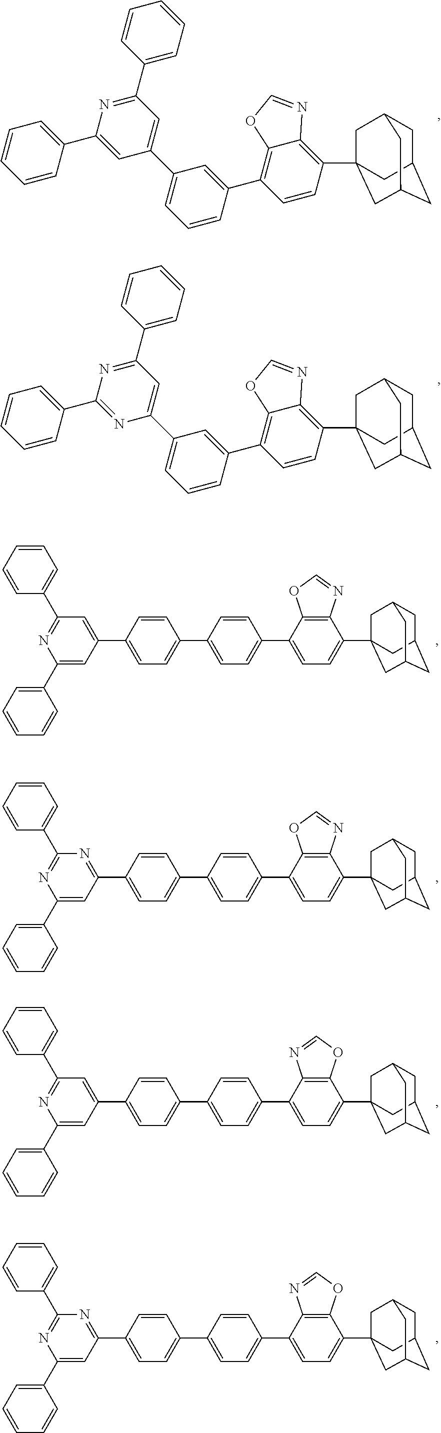

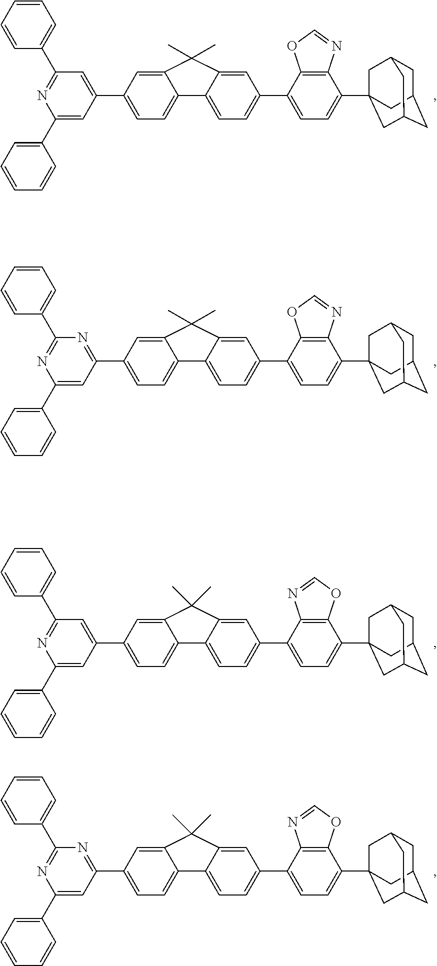 [SMILES: CC1(C)c2cc(-c3cc(-c4ccccc4)nc(-c4ccccc4)c3)ccc2-c2ccc(-c3ccc(C45CC6CC(CC(C6)C4)C5)c4ncoc34)cc21 ; CC1(C)c2cc(-c3cc(-c4ccccc4)nc(-c4ccccc4)n3)ccc2-c2ccc(-c3ccc(C45CC6CC(CC(C6)C4)C5)c4ncoc34)cc21 ; CC1(C)c2cc(-c3cc(-c4ccccc4)nc(-c4ccccc4)c3)ccc2-c2ccc(-c3ccc(C45CC6CC(CC(C6)C4)C5)c4ocnc34)cc21 ; CC1(C)c2cc(-c3cc(-c4ccccc4)nc(-c4ccccc4)n3)ccc2-c2ccc(-c3ccc(C45CC6CC(CC(C6)C4)C5)c4ocnc34)cc21]

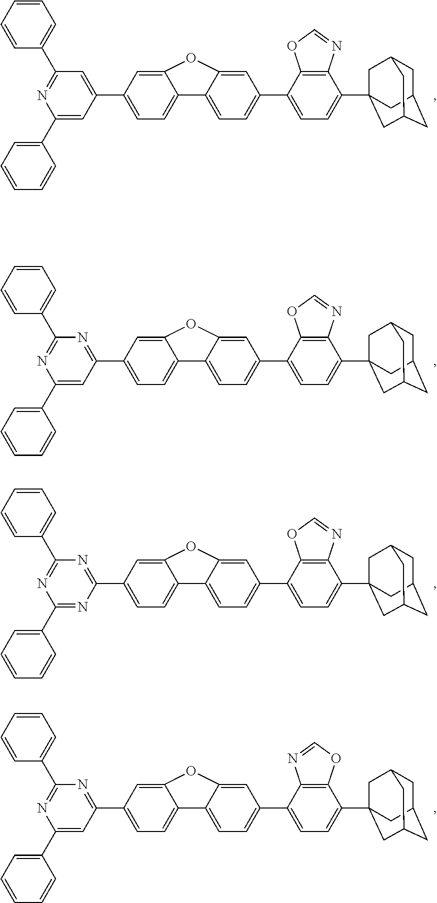

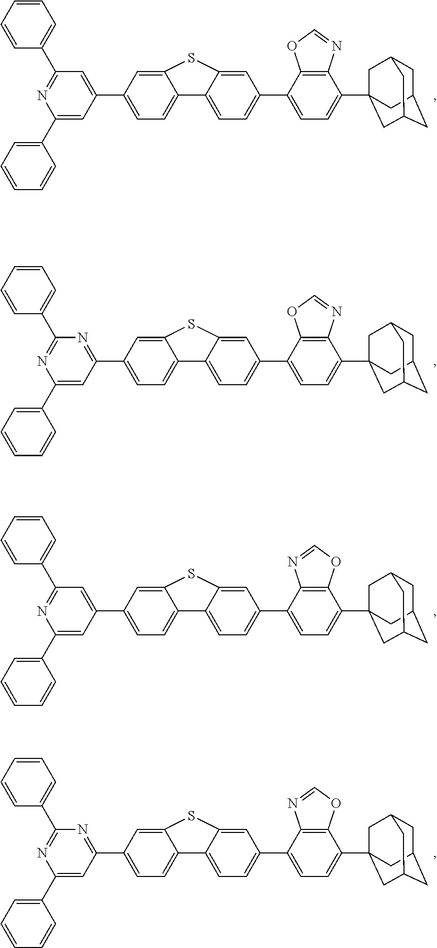 [SMILES: c1ccc(-c2cc(-c3ccc4c(c3)sc3cc(-c5ccc(C67CC8CC(CC(C8)C6)C7)c6ncoc56)ccc34)cc(-c3ccccc3)n2)cc1 ; c1ccc(-c2cc(-c3ccc4c(c3)sc3cc(-c5ccc(C67CC8CC(CC(C8)C6)C7)c6ncoc56)ccc34)nc(-c3ccccc3)n2)cc1 ; c1ccc(-c2cc(-c3ccc4c(c3)sc3cc(-c5ccc(C67CC8CC(CC(C8)C6)C7)c6ocnc56)ccc34)cc(-c3ccccc3)n2)cc1 ; c1ccc(-c2cc(-c3ccc4c(c3)sc3cc(-c5ccc(C67CC8CC(CC(C8)C6)C7)c6ocnc56)ccc34)nc(-c3ccccc3)n2)cc1]

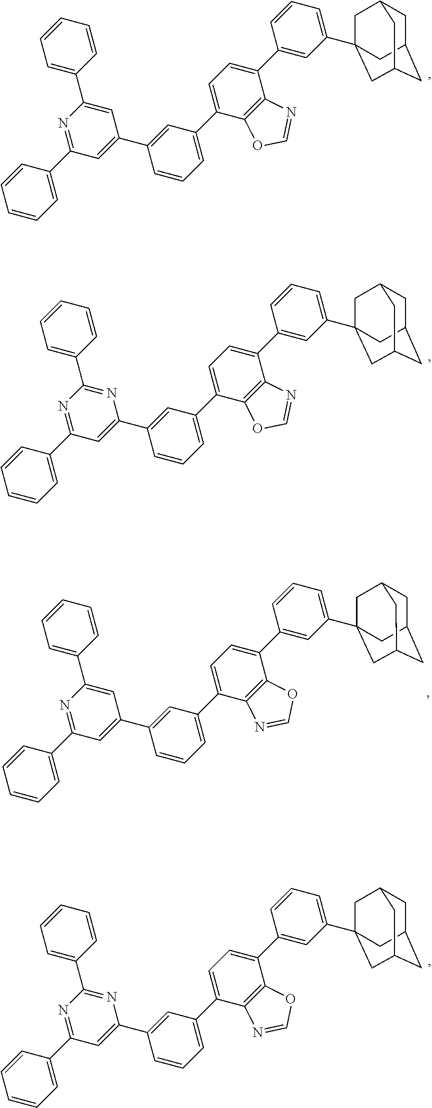

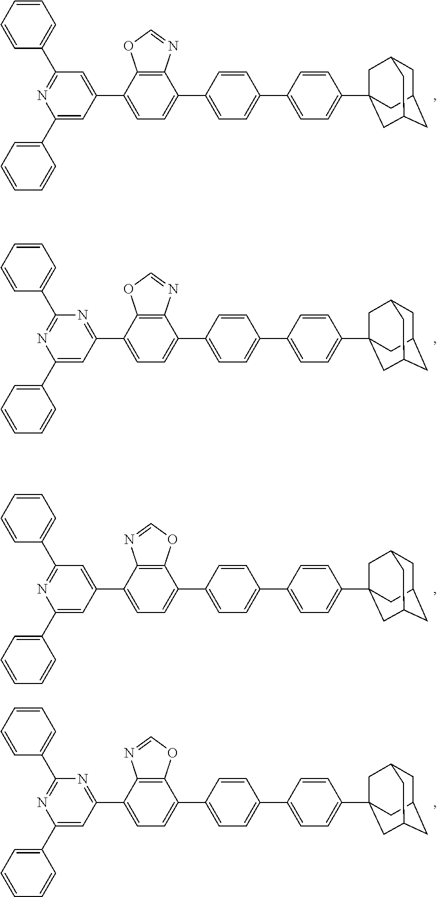

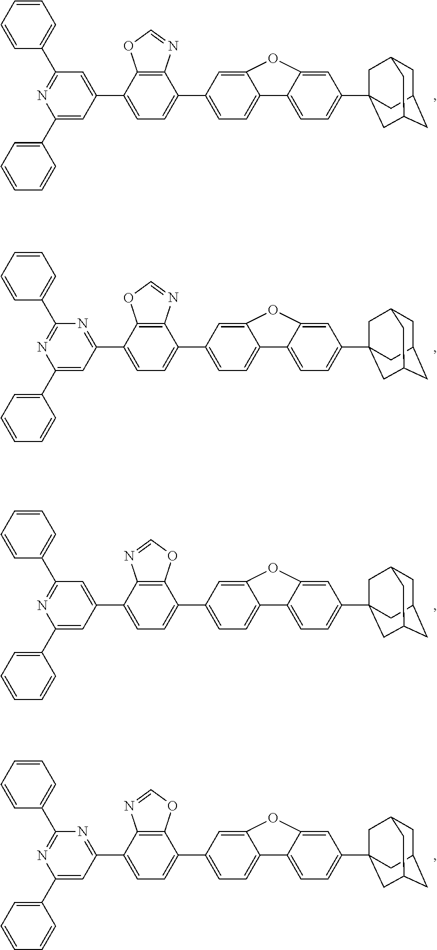 [SMILES: c1ccc(-c2cc(-c3ccc(-c4ccc5c(c4)oc4cc(C67CC8CC(CC(C8)C6)C7)ccc45)c4ncoc34)cc(-c3ccccc3)n2)cc1 ; c1ccc(-c2cc(-c3ccc(-c4ccc5c(c4)oc4cc(C67CC8CC(CC(C8)C6)C7)ccc45)c4ncoc34)nc(-c3ccccc3)n2)cc1 ; c1ccc(-c2cc(-c3ccc(-c4ccc5c(c4)oc4cc(C67CC8CC(CC(C8)C6)C7)ccc45)c4ocnc34)cc(-c3ccccc3)n2)cc1 ; c1ccc(-c2cc(-c3ccc(-c4ccc5c(c4)oc4cc(C67CC8CC(CC(C8)C6)C7)ccc45)c4ocnc34)nc(-c3ccccc3)n2)cc1]

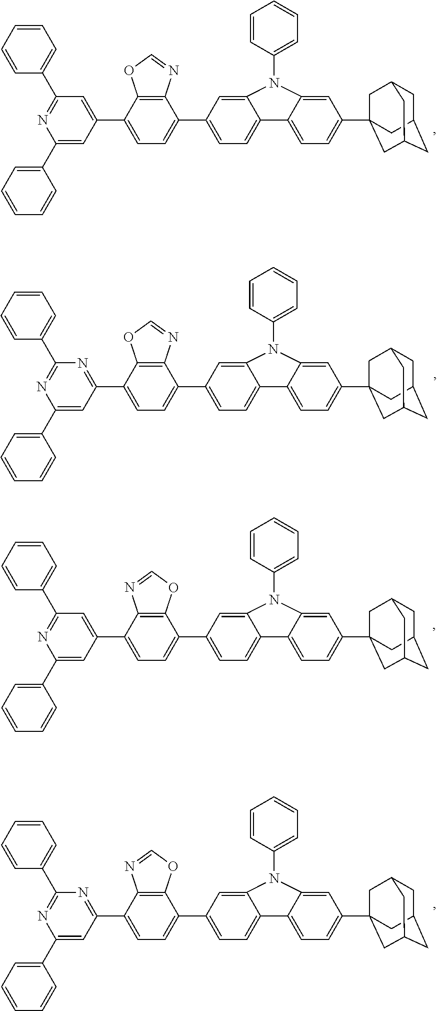 [SMILES: c1ccc(-c2cc(-c3ccc(-c4ccc5c6ccc(C78CC9CC(CC(C9)C7)C8)cc6n(-c6ccccc6)c5c4)c4ncoc34)cc(-c3ccccc3)n2)cc1 ; c1ccc(-c2cc(-c3ccc(-c4ccc5c6ccc(C78CC9CC(CC(C9)C7)C8)cc6n(-c6ccccc6)c5c4)c4ncoc34)nc(-c3ccccc3)n2)cc1 ; c1ccc(-c2cc(-c3ccc(-c4ccc5c6ccc(C78CC9CC(CC(C9)C7)C8)cc6n(-c6ccccc6)c5c4)c4ocnc34)cc(-c3ccccc3)n2)cc1 ; c1ccc(-c2cc(-c3ccc(-c4ccc5c6ccc(C78CC9CC(CC(C9)C7)C8)cc6n(-c6ccccc6)c5c4)c4ocnc34)nc(-c3ccccc3)n2)cc1]

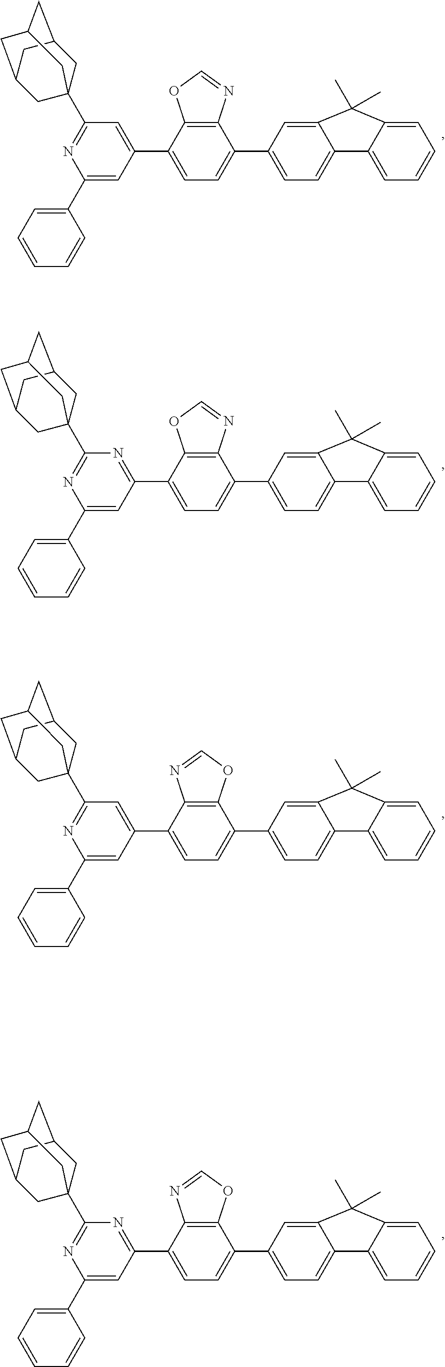 [SMILES: CC1(C)c2ccccc2-c2ccc(-c3ccc(-c4cc(-c5ccccc5)nc(C56CC7CC(CC(C7)C5)C6)c4)c4ocnc34)cc21 ; CC1(C)c2ccccc2-c2ccc(-c3ccc(-c4cc(-c5ccccc5)nc(C56CC7CC(CC(C7)C5)C6)n4)c4ocnc34)cc21 ; CC1(C)c2ccccc2-c2ccc(-c3ccc(-c4cc(-c5ccccc5)nc(C56CC7CC(CC(C7)C5)C6)c4)c4ncoc34)cc21 ; CC1(C)c2ccccc2-c2ccc(-c3ccc(-c4cc(-c5ccccc5)nc(C56CC7CC(CC(C7)C5)C6)n4)c4ncoc34)cc21]

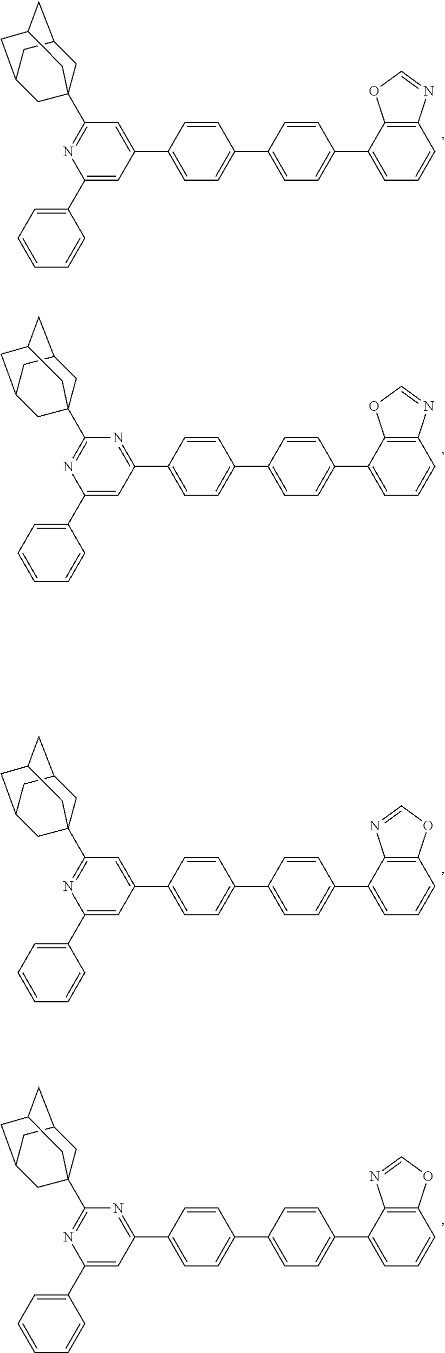 [SMILES: c1ccc(-c2cc(-c3ccc(-c4ccc(-c5cccc6ncoc56)cc4)cc3)cc(C34CC5CC(CC(C5)C3)C4)n2)cc1 ; c1ccc(-c2cc(-c3ccc(-c4ccc(-c5cccc6ncoc56)cc4)cc3)nc(C34CC5CC(CC(C5)C3)C4)n2)cc1 ; c1ccc(-c2cc(-c3ccc(-c4ccc(-c5cccc6ocnc56)cc4)cc3)cc(C34CC5CC(CC(C5)C3)C4)n2)cc1 ; c1ccc(-c2cc(-c3ccc(-c4ccc(-c5cccc6ocnc56)cc4)cc3)nc(C34CC5CC(CC(C5)C3)C4)n2)cc1]

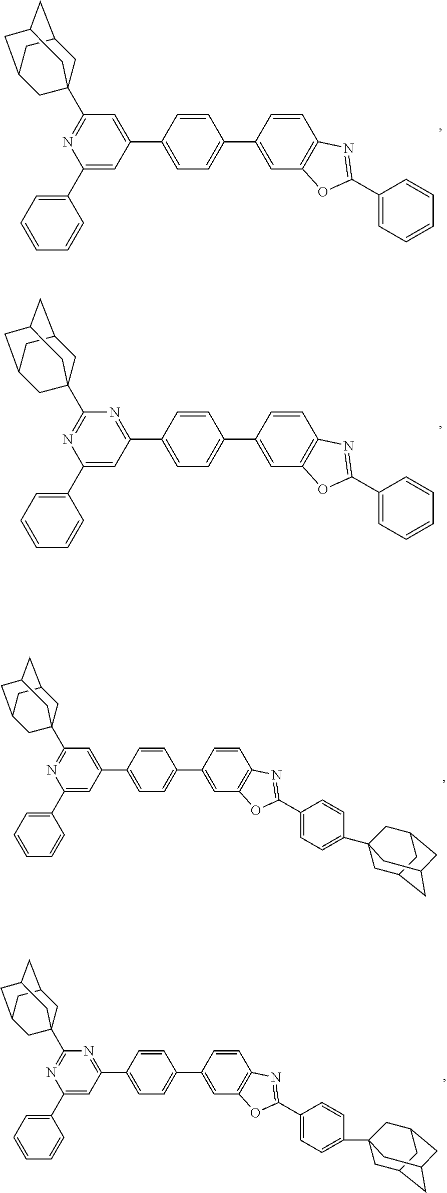 [SMILES: c1ccc(-c2cc(-c3ccc(-c4ccc5nc(-c6ccccc6)oc5c4)cc3)cc(C34CC5CC(CC(C5)C3)C4)n2)cc1 ; c1ccc(-c2cc(-c3ccc(-c4ccc5nc(-c6ccccc6)oc5c4)cc3)nc(C34CC5CC(CC(C5)C3)C4)n2)cc1 ; c1ccc(-c2cc(-c3ccc(-c4ccc5nc(-c6ccc(C78CC9CC(CC(C9)C7)C8)cc6)oc5c4)cc3)cc(C34CC5CC(CC(C5)C3)C4)n2)cc1 ; c1ccc(-c2cc(-c3ccc(-c4ccc5nc(-c6ccc(C78CC9CC(CC(C9)C7)C8)cc6)oc5c4)cc3)nc(C34CC5CC(CC(C5)C3)C4)n2)cc1]

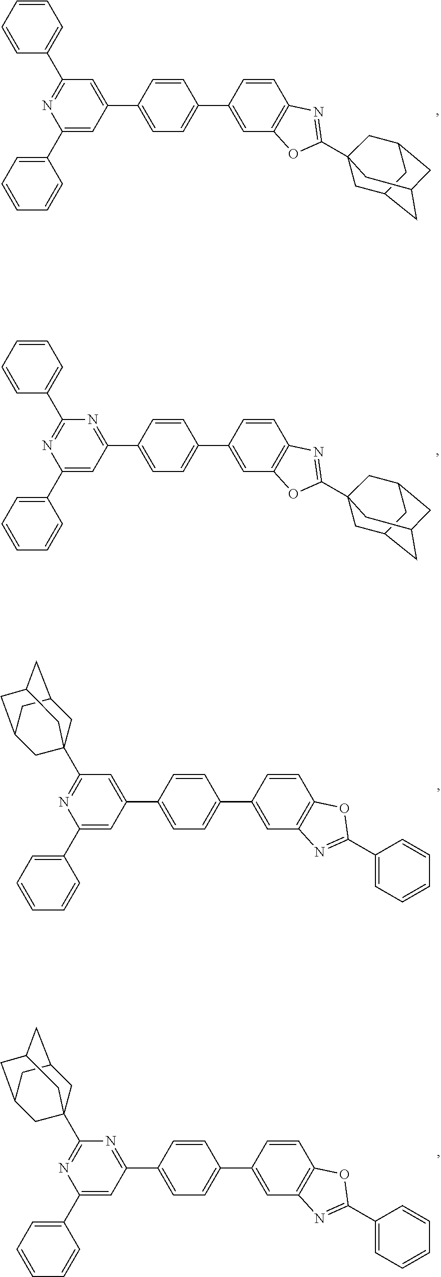 [SMILES: c1ccc(-c2cc(-c3ccc(-c4ccc5nc(C67CC8CC(CC(C8)C6)C7)oc5c4)cc3)cc(-c3ccccc3)n2)cc1 ; c1ccc(-c2cc(-c3ccc(-c4ccc5nc(C67CC8CC(CC(C8)C6)C7)oc5c4)cc3)nc(-c3ccccc3)n2)cc1 ; c1ccc(-c2cc(-c3ccc(-c4ccc5oc(-c6ccccc6)nc5c4)cc3)cc(C34CC5CC(CC(C5)C3)C4)n2)cc1 ; c1ccc(-c2cc(-c3ccc(-c4ccc5oc(-c6ccccc6)nc5c4)cc3)nc(C34CC5CC(CC(C5)C3)C4)n2)cc1]

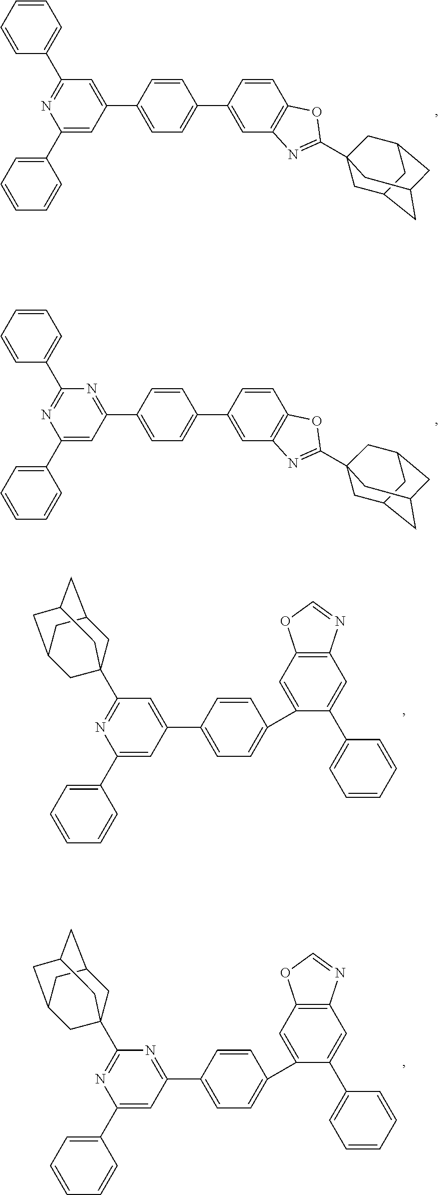 [SMILES: c1ccc(-c2cc(-c3ccc(-c4ccc5oc(C67CC8CC(CC(C8)C6)C7)nc5c4)cc3)cc(-c3ccccc3)n2)cc1 ; c1ccc(-c2cc(-c3ccc(-c4ccc5oc(C67CC8CC(CC(C8)C6)C7)nc5c4)cc3)nc(-c3ccccc3)n2)cc1 ; c1ccc(-c2cc(-c3ccc(-c4cc5ocnc5cc4-c4ccccc4)cc3)cc(C34CC5CC(CC(C5)C3)C4)n2)cc1 ; c1ccc(-c2cc(-c3ccc(-c4cc5ocnc5cc4-c4ccccc4)cc3)nc(C34CC5CC(CC(C5)C3)C4)n2)cc1]

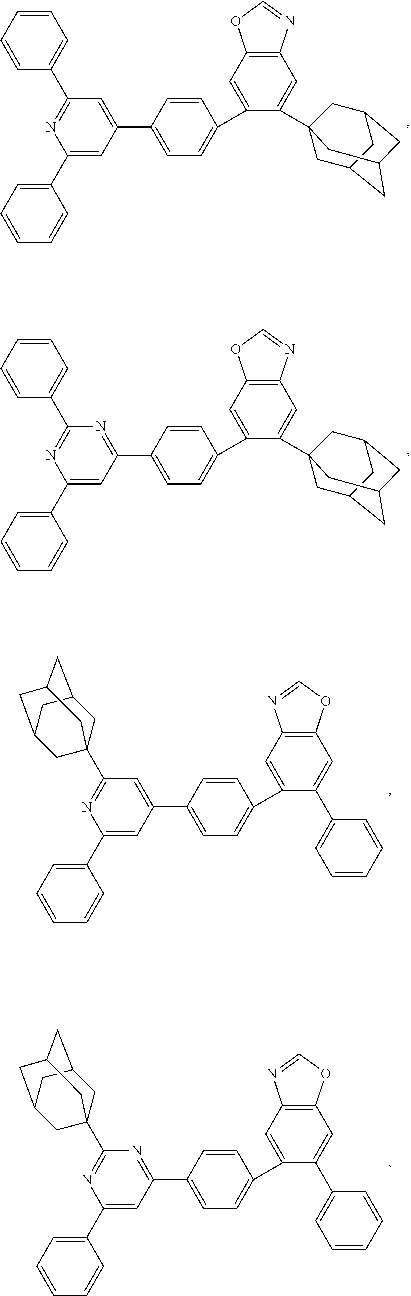 [SMILES: c1ccc(-c2cc(-c3ccc(-c4cc5ocnc5cc4C45CC6CC(CC(C6)C4)C5)cc3)cc(-c3ccccc3)n2)cc1 ; c1ccc(-c2cc(-c3ccc(-c4cc5ocnc5cc4C45CC6CC(CC(C6)C4)C5)cc3)nc(-c3ccccc3)n2)cc1 ; c1ccc(-c2cc(-c3ccc(-c4cc5ncoc5cc4-c4ccccc4)cc3)cc(C34CC5CC(CC(C5)C3)C4)n2)cc1 ; c1ccc(-c2cc(-c3ccc(-c4cc5ncoc5cc4-c4ccccc4)cc3)nc(C34CC5CC(CC(C5)C3)C4)n2)cc1]

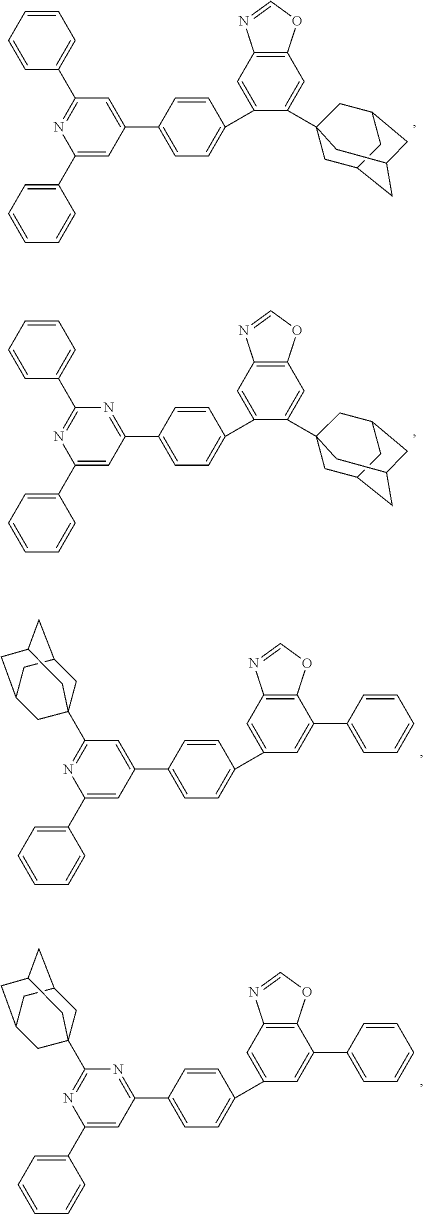 [SMILES: c1ccc(-c2cc(-c3ccc(-c4cc5ncoc5cc4C45CC6CC(CC(C6)C4)C5)cc3)cc(-c3ccccc3)n2)cc1 ; c1ccc(-c2cc(-c3ccc(-c4cc5ncoc5cc4C45CC6CC(CC(C6)C4)C5)cc3)nc(-c3ccccc3)n2)cc1 ; c1ccc(-c2cc(-c3ccc(-c4cc(-c5ccccc5)c5ocnc5c4)cc3)cc(C34CC5CC(CC(C5)C3)C4)n2)cc1 ; c1ccc(-c2cc(-c3ccc(-c4cc(-c5ccccc5)c5ocnc5c4)cc3)nc(C34CC5CC(CC(C5)C3)C4)n2)cc1]

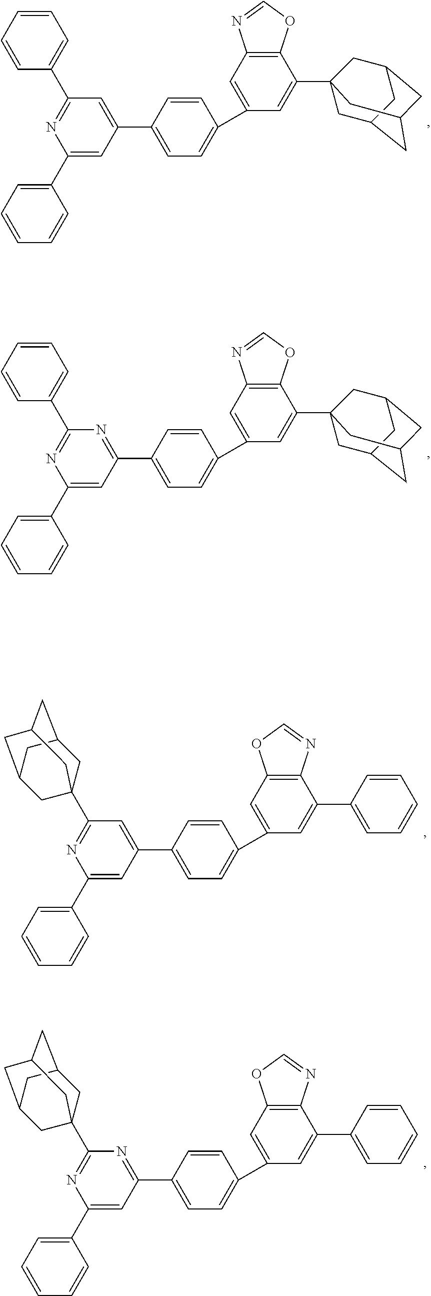 [SMILES: c1ccc(-c2cc(-c3ccc(-c4cc(C56CC7CC(CC(C7)C5)C6)c5ocnc5c4)cc3)cc(-c3ccccc3)n2)cc1 ; c1ccc(-c2cc(-c3ccc(-c4cc(C56CC7CC(CC(C7)C5)C6)c5ocnc5c4)cc3)nc(-c3ccccc3)n2)cc1 ; c1ccc(-c2cc(-c3ccc(-c4cc(-c5ccccc5)c5ncoc5c4)cc3)cc(C34CC5CC(CC(C5)C3)C4)n2)cc1 ; c1ccc(-c2cc(-c3ccc(-c4cc(-c5ccccc5)c5ncoc5c4)cc3)nc(C34CC5CC(CC(C5)C3)C4)n2)cc1]

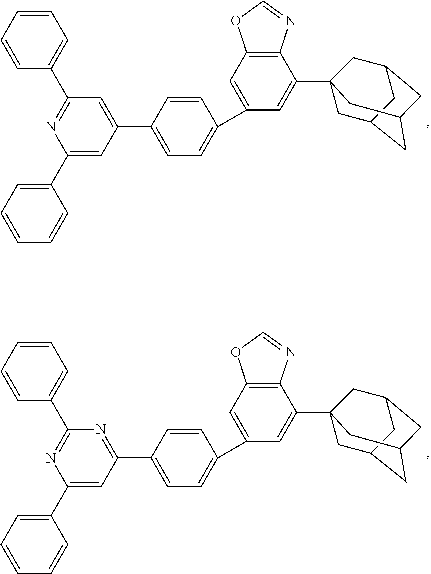 [SMILES: c1ccc(-c2cc(-c3ccc(-c4cc(C56CC7CC(CC(C7)C5)C6)c5ncoc5c4)cc3)cc(-c3ccccc3)n2)cc1 ; c1ccc(-c2cc(-c3ccc(-c4cc(C56CC7CC(CC(C7)C5)C6)c5ncoc5c4)cc3)nc(-c3ccccc3)n2)cc1]

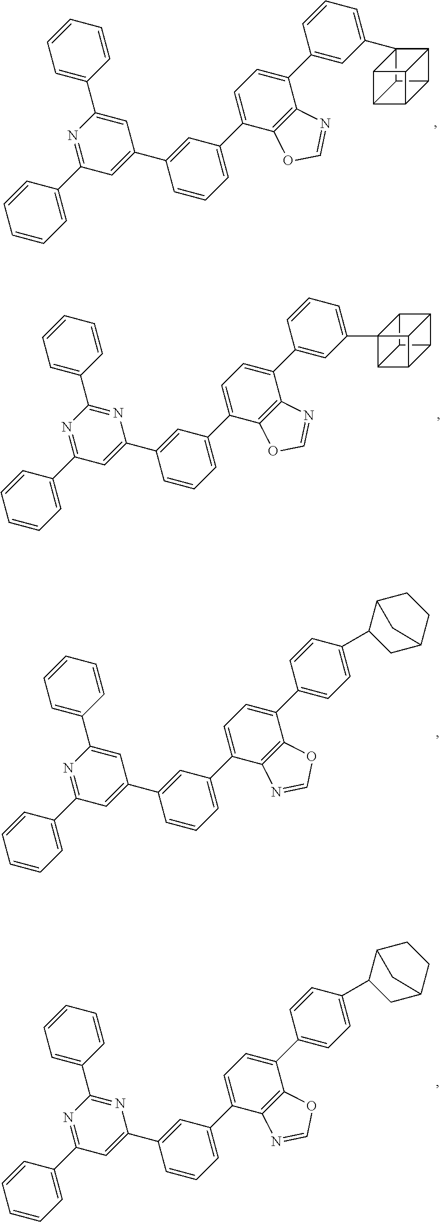 [SMILES: c1ccc(-c2cc(-c3cccc(-c4ccc(-c5cccc(C67C8C9C%10C8C6C%10C97)c5)c5ncoc45)c3)cc(-c3ccccc3)n2)cc1 ; c1ccc(-c2cc(-c3cccc(-c4ccc(-c5cccc(C67C8C9C%10C8C6C%10C97)c5)c5ncoc45)c3)nc(-c3ccccc3)n2)cc1 ; c1ccc(-c2cc(-c3cccc(-c4ccc(-c5ccc(C6CC7CCC6C7)cc5)c5ocnc45)c3)cc(-c3ccccc3)n2)cc1 ; c1ccc(-c2cc(-c3cccc(-c4ccc(-c5ccc(C6CC7CCC6C7)cc5)c5ocnc45)c3)nc(-c3ccccc3)n2)cc1]

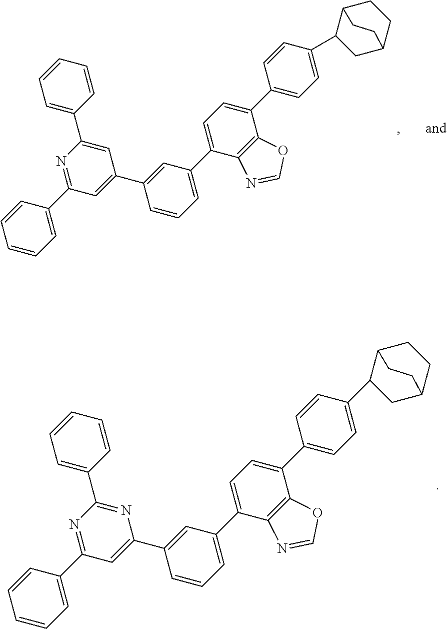 [SMILES: c1ccc(-c2cc(-c3cccc(-c4ccc(-c5ccc(C6CC7CCC6CC7)cc5)c5ocnc45)c3)cc(-c3ccccc3)n2)cc1 ; c1ccc(-c2cc(-c3cccc(-c4ccc(-c5ccc(C6CC7CCC6CC7)cc5)c5ocnc45)c3)nc(-c3ccccc3)n2)cc1]

16. The light emitting device according to claim 1, wherein the light emitting device further comprises an anode and a cathode, and the electron transport layer is located between the anode and the cathode;
the light emitting device further comprises a luminescent layer and an electron blocking layer, and the luminescent layer is located between the anode and the electron transport layer; and
the electron blocking layer is located between the anode and the luminescent layer.

17. A displaying device, wherein the displaying device comprises the light emitting device according to claim 1.

18. A compound, wherein a general structural formula of the compound is: 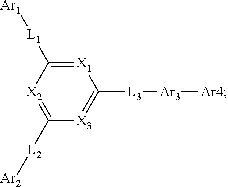
wherein X1 is C(Ra) or nitrogen, X2 is C(Rb) or nitrogen, X3 is C(Rc) or nitrogen, and at least one of X1, X2 and X3 is nitrogen, wherein each of Ra, Rb and Rc is independently any one of a group consisting of hydrogen, diplogen, fluorine, chlorine, an alkyl group whose carbon-atom quantity is 1-12, a halogenated alkyl group whose carbon-atom quantity is 1-12, an alkoxy group whose carbon-atom quantity is 1-12, a cycloalkyl group whose carbon-atom quantity is 3-10, C6-20 aryl, and a heteroaryl group whose carbon-atom quantity is 3-20;
each of L1, L2 and L3 is independently any one of a group consisting of a single bond, a substituted or unsubstituted arylene group whose carbon-atom quantity is 6-60, and a substituted or unsubstituted heteroarylene whose carbon-atom quantity is 1-60;
each of Ar1, Ar2 and Ar4 is independently any one of a group consisting of a substituted or unsubstituted alkyl group whose carbon-atom quantity is 1-12, a substituted or unsubstituted cycloalkyl group whose carbon-atom quantity is 3-10, a substituted or unsubstituted aralkyl group whose carbon-atom quantity is 7-30, a substituted or unsubstituted heterarylalkyl whose carbon-atom quantity is 2-60, a substituted or unsubstituted aryl whose carbon-atom quantity is 6-60, and a substituted or unsubstituted heteroaryl group whose carbon-atom quantity is 3-60, and at least one of Ar1, Ar2 and Ar4 is the three-dimensional group having a rigidity; and
a general structural formula of Ar3 is: 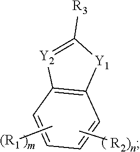
wherein Y1 is any one of carbon, oxygen, sulphur, selenium and N—R4;
Y2 is any one of carbon and nitrogen;
each of R1, R2 and R3 is independently any one of hydrogen, diplogen, halogen, amino, a nitrile group, nitro, C1-30 alkyl, C2-30 alkenyl, C2-30 alkynyl, C1-30 alkoxy, C6-30 aryloxy, and C6-50 aryl substituted by C6-30 aryl, or comprises any one of at least one of heteroatoms selected from nitrogen, oxygen and sulphur and C2-50 heteroaryl unsubstituted or substituted by C6-30 aryloxy, and two of R1, R2 and R3 are directly connected to neighboring groups L3 and Ar4;
R4 is any one of a group consisting of hydrogen, diplogen, fluorine, chlorine, an alkyl group whose carbon-atom quantity is 1-12, a halogenated alkyl group whose carbon-atom quantity is 1-12, an alkoxy group whose carbon-atom quantity is 1-12, a cycloalkyl group whose carbon-atom quantity is 3-10, C6-20 aryl, and a heteroaryl group whose carbon-atom quantity is 3-20; and
each of m and n is independently 1 or 2.

19. The compound according to claim 18, wherein the general structural formula of the compound is any one of

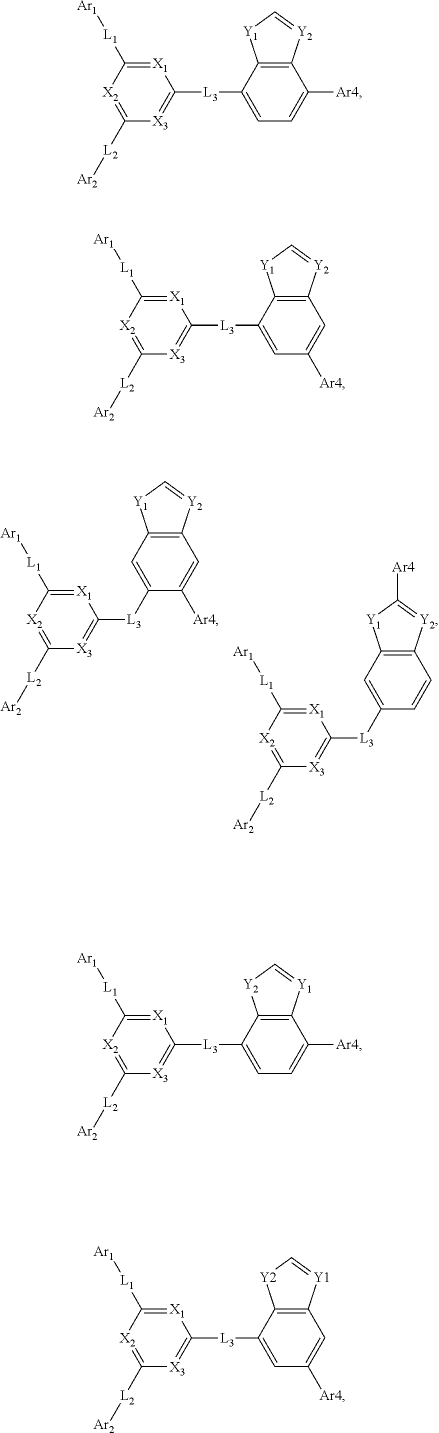

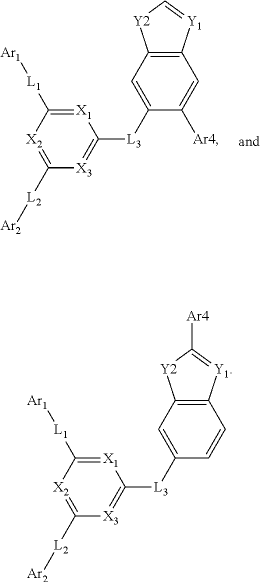

20. The compound according to claim 19, wherein a general structural formula of the three-dimensional group having a rigidity is any one of 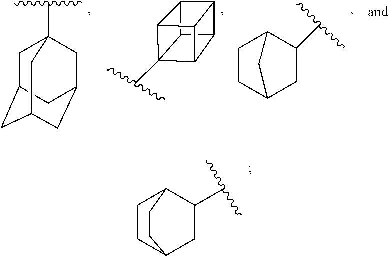
wherein 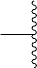
is a bond connected to a carbon atom.

21-25. (canceled)
